SEQUENCE LISTING (829 sequences)

SEQ ID NO: 1 - protein, 11 aa, Artificial Sequence
  Believed to be a synthetic construct
YGRKKRRQRRR

SEQ ID NO: 2 - protein, 8 aa, Artificial Sequence
  Believed to be a synthetic construct
RRRRRRRR

SEQ ID NO: 3 - protein, 15 aa, Artificial Sequence
  Believed to be a synthetic construct
RQIKIWFQNRRMKWK

SEQ ID NO: 4 - protein, 15 aa, Artificial Sequence
  Believed to be a synthetic construct
SAIGKIQDSLSSTAS

SEQ ID NO: 5 - protein, 12 aa, Artificial Sequence
  Believed to be a synthetic construct
PQEESEEEVEEP

SEQ ID NO: 6 - protein, 12 aa, Artificial Sequence
  Believed to be a synthetic construct
  UNSURE (8)..(9)
GGKGLGKACGGA

SEQ ID NO: 7 - protein, 13 aa, Homo sapiens
DTMVGWDKDARTK

SEQ ID NO: 8 - protein, 10 aa, Artificial Sequence
  Believed to be a synthetic construct
FNGARSFIDI

SEQ ID NO: 9 - protein, 10 aa, Artificial Sequence
  Believed to be a synthetic construct
WARLWNYLYR

SEQ ID NO: 10 - protein, 10 aa, Artificial Sequence
  Believed to be a synthetic construct
RSFIDIGSGT

SEQ ID NO: 11 - protein, 12 aa, Artificial Sequence
  Believed to be a synthetic construct
  UNSURE (6)..(6): (p)
KAVDGXYVKPQI

SEQ ID NO: 12 - protein, 10 aa, Artificial Sequence
  Believed to be a synthetic construct
WIDPVNGDTE

SEQ ID NO: 13 - protein, 14 aa, Artificial Sequence
  Believed to be a synthetic construct
ARHPSWYRPFEGCG

SEQ ID NO: 14 - protein, 16 aa, Artificial Sequence
  Believed to be a synthetic construct
  VARIANT (11)..(11): (homoR)
MESFPGWNLVXIGLLR

SEQ ID NO: 15 - protein, 17 aa, Artificial Sequence
  Believed to be a synthetic construct
FNWDYSLEELREKAKYK

SEQ ID NO: 16 - protein, 18 aa, Artificial Sequence
  Believed to be a synthetic construct
MPIFLDHILNKFWILHYA

SEQ ID NO: 17 - protein, 10 aa, Artificial Sequence
  Believed to be a synthetic construct
LYDVAGSDKY

SEQ ID NO: 18 - protein, 22 aa, Artificial Sequence
  Believed to be a synthetic construct
  DISULFID (1)..(1)
  DISULFID (20)..(20)
CGIQDTNSKKQSDTHLEETCGS

SEQ ID NO: 19 - protein, 11 aa, Artificial Sequence
  Believed to be a synthetic construct
KKRRQRRRPPQ

SEQ ID NO: 20 - protein, 18 aa, Artificial Sequence
  Believed to be a synthetic construct
LGISYGRKKRRQRRRPPQ

SEQ ID NO: 21 - protein, 23 aa, Artificial Sequence
  Believed to be a synthetic construct
FITKALGISYGRKKRRQRRRPPQ

SEQ ID NO: 22 - protein, 16 aa, Artificial Sequence
  Believed to be a synthetic construct
FITKALGISYGRKKRR

SEQ ID NO: 23 - protein, 10 aa, Artificial Sequence
  Believed to be a synthetic construct
GRKKRRQRRR

SEQ ID NO: 24 - protein, 9 aa, Artificial Sequence
  Believed to be a synthetic construct
RKKRRQRRR

SEQ ID NO: 25 - protein, 8 aa, Artificial Sequence
  Believed to be a synthetic construct
RKKRRQRR

SEQ ID NO: 26 - protein, 7 aa, Artificial Sequence
  Believed to be a synthetic construct
RKKRRQR

SEQ ID NO: 27 - protein, 8 aa, Artificial Sequence
  Believed to be a synthetic construct
KKRRQRRR

SEQ ID NO: 28 - protein, 7 aa, Artificial Sequence
  Believed to be a synthetic construct
KRRQRRR

SEQ ID NO: 29 - protein, 9 aa, Artificial Sequence
  Believed to be a synthetic construct
RRRQRRKKR

SEQ ID NO: 30 - protein, 9 aa, Artificial Sequence
  Believed to be a synthetic construct
AKKRRQRRR

SEQ ID NO: 31 - protein, 9 aa, Artificial Sequence
  Believed to be a synthetic construct
RAKRRQRRR

SEQ ID NO: 32 - protein, 9 aa, Artificial Sequence
  Believed to be a synthetic construct
RKARRQRRR

SEQ ID NO: 33 - protein, 9 aa, Artificial Sequence
  Believed to be a synthetic construct
RKKARQRRR

SEQ ID NO: 34 - protein, 9 aa, Homo sapiens
RKKRAQRRR

SEQ ID NO: 35 - protein, 9 aa, Artificial Sequence
  Believed to be a synthetic construct
RKKRRARRR

SEQ ID NO: 36 - protein, 9 aa, Artificial Sequence
  Believed to be a synthetic construct
RKKRRQARR

SEQ ID NO: 37 - protein, 9 aa, Artificial Sequence
  Believed to be a synthetic construct
RKKRRQRAR

SEQ ID NO: 38 - protein, 9 aa, Artificial Sequence
  Believed to be a synthetic construct
RKKRRQRRA

SEQ ID NO: 39 - protein, 13 aa, Artificial Sequence
  Believed to be a synthetic construct
GRKKRRQRRPPQC

SEQ ID NO: 40 - protein, 12 aa, Artificial Sequence
  Believed to be a synthetic construct
GRKKRRQRPPQC

SEQ ID NO: 41 - protein, 11 aa, Artificial Sequence
  Believed to be a synthetic construct
GRKKRRQPPQC

SEQ ID NO: 42 - protein, 11 aa, Artificial Sequence
  Believed to be a synthetic construct
GRKKRRQRRRC

SEQ ID NO: 43 - protein, 14 aa, Artificial Sequence
  Believed to be a synthetic construct
GRKKRRQRARPPQC

SEQ ID NO: 44 - protein, 14 aa, Artificial Sequence
  Believed to be a synthetic construct
GRKKRRQARAPPQC

SEQ ID NO: 45 - protein, 17 aa, Artificial Sequence
  Believed to be a synthetic construct
TRQARRNRRRRWRERQR

SEQ ID NO: 46 - protein, 4 aa, Artificial Sequence
  Believed to be a synthetic construct
RRRR

SEQ ID NO: 47 - protein, 5 aa, Artificial Sequence
  Believed to be a synthetic construct
RRRRR

SEQ ID NO: 48 - protein, 6 aa, Artificial Sequence
  Believed to be a synthetic construct
RRRRRR

SEQ ID NO: 49 - protein, 7 aa, Artificial Sequence
  Believed to be a synthetic construct
RRRRRRR

SEQ ID NO: 50 - protein, 9 aa, Artificial Sequence
  Believed to be a synthetic construct
RRRRRRRRR

SEQ ID NO: 51 - protein, 11 aa, Artificial Sequence
  Believed to be a synthetic construct
RRRRRRRRRRR

SEQ ID NO: 52 - protein, 12 aa, Artificial Sequence
  Believed to be a synthetic construct
RRRRRRRRRRRR

SEQ ID NO: 53 - protein, 16 aa, Artificial Sequence
  Believed to be a synthetic construct
RRRRRRRRRRRRRRRR

SEQ ID NO: 54 - protein, 27 aa, Artificial Sequence
  Believed to be a synthetic construct
GWTLNSAGYLLGKINLKALAALAKKIL

SEQ ID NO: 55 - protein, 27 aa, Artificial Sequence
  Believed to be a synthetic construct
GWTLNSAGYLLGKINLKALAALAKKLL

SEQ ID NO: 56 - protein, 26 aa, Artificial Sequence
  Believed to be a synthetic construct
GWTLNSAGYLLGKFLPLILRKIVTAL

SEQ ID NO: 57 - protein, 27 aa, Artificial Sequence
  Believed to be a synthetic construct
GWTLNPAGYLLGKINLKALAALAKKIL

SEQ ID NO: 58 - protein, 27 aa, Artificial Sequence
  Believed to be a synthetic construct
GWTLNPPGYLLGKINLKALAALAKKIL

SEQ ID NO: 59 - protein, 24 aa, Artificial Sequence
  Believed to be a synthetic construct
LNSAGYLLGKINLKALAALAKKIL

SEQ ID NO: 60 - protein, 18 aa, Artificial Sequence
  Believed to be a synthetic construct
LLGKINLKALAALAKKIL

SEQ ID NO: 61 - protein, 25 aa, Artificial Sequence
  Believed to be a synthetic construct
GWTLNSAGYLLGKLKALAALAKKIL

SEQ ID NO: 62 - protein, 21 aa, Artificial Sequence
  Believed to be a synthetic construct
AGYLLGKINLKALAALAKKIL

SEQ ID NO: 63 - protein, 21 aa, Artificial Sequence
  Believed to be a synthetic construct
GWTLNSKINLKALAALAKKIL

SEQ ID NO: 64 - protein, 21 aa, Artificial Sequence
  Believed to be a synthetic construct
LNSAGYLLGKLKALAALAKIL

SEQ ID NO: 65 - protein, 20 aa, Artificial Sequence
  Believed to be a synthetic construct
LNSAGYLLGKALAALAKKIL

SEQ ID NO: 66 - protein, 19 aa, Artificial Sequence
  Believed to be a synthetic construct
AGYLLGKLKALAALAKKIL

SEQ ID NO: 67 - protein, 19 aa, Artificial Sequence
  Believed to be a synthetic construct
LNSAGYLLGKLKALAALAK

SEQ ID NO: 68 - protein, 27 aa, Artificial Sequence
  Believed to be a synthetic construct
GWTLNSAGYLLGKINLKAPAALAKKIL

SEQ ID NO: 69 - protein, 30 aa, Artificial Sequence
  Believed to be a synthetic construct
GWTLNSAGYLLGPHAVGNHRSFSDKNGLTS

SEQ ID NO: 70 - protein, 14 aa, Artificial Sequence
  Believed to be a synthetic construct
INLKALAALAKKIL

SEQ ID NO: 71 - protein, 18 aa, Artificial Sequence
  Believed to be a synthetic construct
KLALKLALKALKAALKLA

SEQ ID NO: 72 - protein, 18 aa, Artificial Sequence
  Believed to be a synthetic construct
KLALKLALKAWKAALKLA

SEQ ID NO: 73 - protein, 18 aa, Artificial Sequence
  Believed to be a synthetic construct
KLALKAALKAWKAAAKLA

SEQ ID NO: 74 - protein, 18 aa, Artificial Sequence
  Believed to be a synthetic construct
KLALKAAAKAWKAAAKAA

SEQ ID NO: 75 - protein, 18 aa, Artificial Sequence
  Believed to be a synthetic construct
KITLKLAIKAWKLALKAA

SEQ ID NO: 76 - protein, 18 aa, Artificial Sequence
  Believed to be a synthetic construct
KIAAKSIAKIWKSILKIA

SEQ ID NO: 77 - protein, 18 aa, Artificial Sequence
  Believed to be a synthetic construct
KALAKALAKLWKALAKAA

SEQ ID NO: 78 - protein, 18 aa, Artificial Sequence
  Believed to be a synthetic construct
KLALKLALKWAKLALKAA

SEQ ID NO: 79 - protein, 18 aa, Artificial Sequence
  Believed to be a synthetic construct
KLLAKAAKKWLLLALKAA

SEQ ID NO: 80 - protein, 18 aa, Artificial Sequence
  Believed to be a synthetic construct
KLLAKAALKWLLKALKAA

SEQ ID NO: 81 - protein, 18 aa, Artificial Sequence
  Believed to be a synthetic construct
KALKKLLAKWLAAAKALL

SEQ ID NO: 82 - protein, 18 aa, Artificial Sequence
  Believed to be a synthetic construct
KLAAALLKKWKKLAAALL

SEQ ID NO: 83 - protein, 18 aa, Artificial Sequence
  Believed to be a synthetic construct
KALAALLKKWAKLLAALK

SEQ ID NO: 84 - protein, 18 aa, Artificial Sequence
  Believed to be a synthetic construct
KALAALLKKLAKLLAALK

SEQ ID NO: 85 - protein, 16 aa, Artificial Sequence
  Believed to be a synthetic construct
KLALKLALKALKAALK

SEQ ID NO: 86 - protein, 14 aa, Artificial Sequence
  Believed to be a synthetic construct
KLALKALKAALKLA

SEQ ID NO: 87 - protein, 14 aa, Artificial Sequence
  Believed to be a synthetic construct
KLALKLALKALKAA

SEQ ID NO: 88 - protein, 18 aa, Artificial Sequence
  Believed to be a synthetic construct
KLGLKLGLKGLKGGLKLG

SEQ ID NO: 89 - protein, 18 aa, Artificial Sequence
  Believed to be a synthetic construct
KLALKLALKALQAALQLA

SEQ ID NO: 90 - protein, 18 aa, Artificial Sequence
  Believed to be a synthetic construct
KLALQLALQALQAALQLA

SEQ ID NO: 91 - protein, 18 aa, Artificial Sequence
  Believed to be a synthetic construct
QLALQLALQALQAALQLA

SEQ ID NO: 92 - protein, 18 aa, Artificial Sequence
  Believed to be a synthetic construct
ELALELALEALEAALELA

SEQ ID NO: 93 - protein, 18 aa, Artificial Sequence
  Believed to be a synthetic construct
LKTLATALTKLAKTLTTL

SEQ ID NO: 94 - protein, 18 aa, Artificial Sequence
  Believed to be a synthetic construct
LLKTTALLKTTALLKTTA

SEQ ID NO: 95 - protein, 18 aa, Artificial Sequence
  Believed to be a synthetic construct
LKTLTETLKELTKTLTEL

SEQ ID NO: 96 - protein, 18 aa, Artificial Sequence
  Believed to be a synthetic construct
LLKTTELLKTTELLKTTE

SEQ ID NO: 97 - protein, 16 aa, Artificial Sequence
  Believed to be a synthetic construct
RQIKIWFQNRRMKWKK

SEQ ID NO: 98 - protein, 18 aa, Artificial Sequence
  Believed to be a synthetic construct
KALKLKLALALLAKLKLA

SEQ ID NO: 99 - protein, 16 aa, Artificial Sequence
  Believed to be a synthetic construct
RQIKIWFQNRRMKWKK

SEQ ID NO: 100 - protein, 16 aa, Artificial Sequence
  Believed to be a synthetic construct
KKWKMRRNQFWIKIQR

SEQ ID NO: 101 - protein, 16 aa, Artificial Sequence
  Believed to be a synthetic construct
RQIKIWFPNRRMKWKK

SEQ ID NO: 102 - protein, 16 aa, Artificial Sequence
  Believed to be a synthetic construct
RQPKIWFPNRRKPWKK

SEQ ID NO: 103 - protein, 16 aa, Artificial Sequence
  Believed to be a synthetic construct
RQIKIWFQNRRMKWKK

SEQ ID NO: 104 - protein, 14 aa, Artificial Sequence
  Believed to be a synthetic construct
RQIKIWFQNRRMKW

SEQ ID NO: 105 - protein, 13 aa, Artificial Sequence
  Believed to be a synthetic construct
RQIKIWFQNRRMK

SEQ ID NO: 106 - protein, 12 aa, Artificial Sequence
  Believed to be a synthetic construct
RQIKIWFQNRRM

SEQ ID NO: 107 - protein, 11 aa, Artificial Sequence
  Believed to be a synthetic construct
RQIKIWFQNRR

SEQ ID NO: 108 - protein, 10 aa, Artificial Sequence
  Believed to be a synthetic construct
RQIKIWFQNR

SEQ ID NO: 109 - protein, 9 aa, Artificial Sequence
  Believed to be a synthetic construct
RQIKIWFQN

SEQ ID NO: 110 - protein, 8 aa, Artificial Sequence
  Believed to be a synthetic construct
RQIKIWFQ

SEQ ID NO: 111 - protein, 6 aa, Artificial Sequence
  Believed to be a synthetic construct
RQIKIW

SEQ ID NO: 112 - protein, 15 aa, Artificial Sequence
  Believed to be a synthetic construct
QIKIWFQNRRMKWKK

SEQ ID NO: 113 - protein, 14 aa, Artificial Sequence
  Believed to be a synthetic construct
IKIWFQNRRMKWKK

SEQ ID NO: 114 - protein, 13 aa, Artificial Sequence
  Believed to be a synthetic construct
KIWFQNRRMKWKK

SEQ ID NO: 115 - protein, 12 aa, Artificial Sequence
  Believed to be a synthetic construct
IWFQNRRMKWKK

SEQ ID NO: 116 - protein, 11 aa, Artificial Sequence
  Believed to be a synthetic construct
WFQNRRMKWKK

SEQ ID NO: 117 - protein, 10 aa, Artificial Sequence
  Believed to be a synthetic construct
FQNRRMKWKK

SEQ ID NO: 118 - protein, 9 aa, Artificial Sequence
  Believed to be a synthetic construct
QNRRMKWKK

SEQ ID NO: 119 - protein, 8 aa, Artificial Sequence
  Believed to be a synthetic construct
NRRMKWKK

SEQ ID NO: 120 - protein, 7 aa, Artificial Sequence
  Believed to be a synthetic construct
RRMKWKK

SEQ ID NO: 121 - protein, 6 aa, Artificial Sequence
  Believed to be a synthetic construct
RMKWKK

SEQ ID NO: 122 - protein, 16 aa, Artificial Sequence
  Believed to be a synthetic construct
AQIKIWFQNRRMKWKK

SEQ ID NO: 123 - protein, 16 aa, Artificial Sequence
  Believed to be a synthetic construct
RAIKIWFQNRRMKWKK

SEQ ID NO: 124 - protein, 16 aa, Artificial Sequence
  Believed to be a synthetic construct
RQAKIWFQNRRMKWKK

SEQ ID NO: 125 - protein, 16 aa, Artificial Sequence
  Believed to be a synthetic construct
RQIAIWFQNRRMKWKK

SEQ ID NO: 126 - protein, 16 aa, Artificial Sequence
  Believed to be a synthetic construct
RQIKAWFQNRRMKWKK

SEQ ID NO: 127 - protein, 16 aa, Artificial Sequence
  Believed to be a synthetic construct
RQIKIAFQNRRMKWKK

SEQ ID NO: 128 - protein, 16 aa, Artificial Sequence
  Believed to be a synthetic construct
RQIKIWAQNRRMKWKK

SEQ ID NO: 129 - protein, 16 aa, Artificial Sequence
  Believed to be a synthetic construct
RQIKIWFANRRMKWKK

SEQ ID NO: 130 - protein, 16 aa, Artificial Sequence
  Believed to be a synthetic construct
RQIKIWFQARRMKWKK

SEQ ID NO: 131 - protein, 16 aa, Artificial Sequence
  Believed to be a synthetic construct
RQIKIWFQNARMKWKK

SEQ ID NO: 132 - protein, 16 aa, Artificial Sequence
  Believed to be a synthetic construct
RQIKIWFQNRAMKWKK

SEQ ID NO: 133 - protein, 16 aa, Artificial Sequence
  Believed to be a synthetic construct
RQIKIWFQNRRAKWKK

SEQ ID NO: 134 - protein, 16 aa, Artificial Sequence
  Believed to be a synthetic construct
RQIKIWFQNRRMAWKK

SEQ ID NO: 135 - protein, 16 aa, Artificial Sequence
  Believed to be a synthetic construct
RQIKIWFQNRRMKAKK

SEQ ID NO: 136 - protein, 16 aa, Artificial Sequence
  Believed to be a synthetic construct
RQIKIWFQNRRMKWAK

SEQ ID NO: 137 - protein, 16 aa, Artificial Sequence
  Believed to be a synthetic construct
RQIKIWFQNRRMKWKA

SEQ ID NO: 138 - protein, 18 aa, Artificial Sequence
  Believed to be a synthetic construct
CRQIKIWFPNRRMKWKKC

SEQ ID NO: 139 - protein, 16 aa, Artificial Sequence
  Believed to be a synthetic construct
RQIKIWFPNRRMKWKK

SEQ ID NO: 140 - protein, 16 aa, Artificial Sequence
  Believed to be a synthetic construct
RQIKIWFQNRRMKWKK

SEQ ID NO: 141 - protein, 16 aa, Artificial Sequence
  Believed to be a synthetic construct
RQIKIFFQNRRMKFKK

SEQ ID NO: 142 - protein, 16 aa, Artificial Sequence
  Believed to be a synthetic construct
RQIRIWFQNRRMRWRR

SEQ ID NO: 143 - protein, 8 aa, Artificial Sequence
  Believed to be a synthetic construct
RRRRRRRW

SEQ ID NO: 144 - protein, 13 aa, Artificial Sequence
  Believed to be a synthetic construct
GRKKRRQRRRPWQ

SEQ ID NO: 145 - protein, 13 aa, Artificial Sequence
  Believed to be a synthetic construct
GRKKRRQRRRPWQ

SEQ ID NO: 146 - protein, 16 aa, Artificial Sequence
  Believed to be a synthetic construct
RQIRIWFQNRRMRWRR

SEQ ID NO: 147 - protein, 16 aa, Artificial Sequence
  Believed to be a synthetic construct
RRWRRWWRRWWRRWRR

SEQ ID NO: 148 - protein, 16 aa, Artificial Sequence
  Believed to be a synthetic construct
RQIKIWFQNMRRKWKK

SEQ ID NO: 149 - protein, 13 aa, Artificial Sequence
  Believed to be a synthetic construct
KMDCRWRWKCCKK

SEQ ID NO: 150 - protein, 12 aa, Artificial Sequence
  Believed to be a synthetic construct
MDCRWRWKCCKK

SEQ ID NO: 151 - protein, 11 aa, Artificial Sequence
  Believed to be a synthetic construct
DCRWRWKCCKK

SEQ ID NO: 152 - protein, 10 aa, Artificial Sequence
  Believed to be a synthetic construct
CRWRWKCCKK

SEQ ID NO: 153 - protein, 9 aa, Artificial Sequence
  Believed to be a synthetic construct
RWRWKCCKK

SEQ ID NO: 154 - protein, 12 aa, Artificial Sequence
  Believed to be a synthetic construct
KMDCRWRWKCKK

SEQ ID NO: 155 - protein, 11 aa, Artificial Sequence
  Believed to be a synthetic construct
KMDCRWRWKKK

SEQ ID NO: 156 - protein, 10 aa, Artificial Sequence
  Believed to be a synthetic construct
KMDRWRWKKK

SEQ ID NO: 157 - protein, 12 aa, Artificial Sequence
  Believed to be a synthetic construct
KDCRWRWKCCKK

SEQ ID NO: 158 - protein, 11 aa, Artificial Sequence
  Believed to be a synthetic construct
KCRWRWKCCKK

SEQ ID NO: 159 - protein, 10 aa, Artificial Sequence
  Believed to be a synthetic construct
KRWRWKCCKK

SEQ ID NO: 160 - protein, 12 aa, Artificial Sequence
  Believed to be a synthetic construct
  misc_feature (9)..(9): Xaa can be any naturally occurring amino acid
MDCRWRWKXCKK

SEQ ID NO: 161 - protein, 11 aa, Artificial Sequence
  Believed to be a synthetic construct
  misc_feature (8)..(8): Xaa can be any naturally occurring amino acid
DCRWRWKXCKK

SEQ ID NO: 162 - protein, 11 aa, Artificial Sequence
  Believed to be a synthetic construct
  misc_feature (9)..(9): Xaa can be any naturally occurring amino acid
DCRWRWKCXKK

SEQ ID NO: 163 - protein, 10 aa, Artificial Sequence
  Believed to be a synthetic construct
  misc_feature (7)..(7): Xaa can be any naturally occurring amino acid
CRWRWKXCKK

SEQ ID NO: 164 - protein, 10 aa, Artificial Sequence
  Believed to be a synthetic construct
  misc_feature (8)..(8): Xaa can be any naturally occurring amino acid
CRWRWKCXKK

SEQ ID NO: 165 - protein, 9 aa, Artificial Sequence
  Believed to be a synthetic construct
  misc_feature (6)..(6): Xaa can be any naturally occurring amino acid
RWRWKXCKK

SEQ ID NO: 166 - protein, 12 aa, Artificial Sequence
  Believed to be a synthetic construct
  misc_feature (9)..(10): Xaa can be any naturally occurring amino acid
MDCRWRWKXXKK

SEQ ID NO: 167 - protein, 11 aa, Artificial Sequence
  Believed to be a synthetic construct
  misc_feature (8)..(9): Xaa can be any naturally occurring amino acid
DCRWRWKXXKK

SEQ ID NO: 168 - protein, 10 aa, Artificial Sequence
  Believed to be a synthetic construct
  misc_feature (7)..(8): Xaa can be any naturally occurring amino acid
CRWRWKXXKK

SEQ ID NO: 169 - protein, 9 aa, Artificial Sequence
  Believed to be a synthetic construct
  misc_feature (6)..(7): Xaa can be any naturally occurring amino acid
RWRWKXXKK

SEQ ID NO: 170 - protein, 10 aa, Artificial Sequence
  Believed to be a synthetic construct
CRWRWKCSKK

SEQ ID NO: 171 - protein, 10 aa, Artificial Sequence
  Believed to be a synthetic construct
SRWRWKCCKK

SEQ ID NO: 172 - protein, 10 aa, Artificial Sequence
  Believed to be a synthetic construct
SRWRWKCSKK

SEQ ID NO: 173 - protein, 10 aa, Artificial Sequence
  Believed to be a synthetic construct
SRWRWKSCKK

SEQ ID NO: 174 - protein, 10 aa, Artificial Sequence
  Believed to be a synthetic construct
CRWRWKSSKK

SEQ ID NO: 175 - protein, 10 aa, Artificial Sequence
  Believed to be a synthetic construct
SRWRWKSSKK

SEQ ID NO: 176 - protein, 10 aa, Artificial Sequence
  Believed to be a synthetic construct
CRFRWKCCKK

SEQ ID NO: 177 - protein, 10 aa, Artificial Sequence
  Believed to be a synthetic construct
CRWRFKCCKK

SEQ ID NO: 178 - protein, 10 aa, Artificial Sequence
  Believed to be a synthetic construct
CRFRFKCCKK

SEQ ID NO: 179 - protein, 10 aa, Artificial Sequence
  Believed to be a synthetic construct
KCCKWRWRCK

SEQ ID NO: 180 - protein, 11 aa, Artificial Sequence
  Believed to be a synthetic construct
CRWRWKCGCKK

SEQ ID NO: 181 - protein, 14 aa, Artificial Sequence
  Believed to be a synthetic construct
KCGCRWRWKCGCKK

SEQ ID NO: 182 - protein, 8 aa, Artificial Sequence
  Believed to be a synthetic construct
CRWRWKCG

SEQ ID NO: 183 - protein, 13 aa, Artificial Sequence
  Believed to be a synthetic construct
  misc_feature (4)..(4): Xaa can be any naturally occurring amino acid
KMDXRWRWKCCKK

SEQ ID NO: 184 - protein, 13 aa, Artificial Sequence
  Believed to be a synthetic construct
  misc_feature (4)..(4): Xaa can be any naturally occurring amino acid
  misc_feature (10)..(10): Xaa can be any naturally occurring amino acid
KMDXRWRWKXCKK

SEQ ID NO: 185 - protein, 13 aa, Artificial Sequence
  Believed to be a synthetic construct
  misc_feature (4)..(4): Xaa can be any naturally occurring amino acid
  misc_feature (10)..(11): Xaa can be any naturally occurring amino acid
KMDXRWRWKXXKK

SEQ ID NO: 186 - protein, 13 aa, Artificial Sequence
  Believed to be a synthetic construct
  misc_feature (4)..(4): Xaa can be any naturally occurring amino acid
  misc_feature (11)..(11): Xaa can be any naturally occurring amino acid
KMDXRWRWKCXKK

SEQ ID NO: 187 - protein, 12 aa, Artificial Sequence
  Believed to be a synthetic construct
  misc_feature (10)..(10): Xaa can be any naturally occurring amino acid
MDCRWRWKCXKK

SEQ ID NO: 188 - protein, 13 aa, Artificial Sequence
  Believed to be a synthetic construct
KMDCRWRWKCSKK

SEQ ID NO: 189 - protein, 13 aa, Artificial Sequence
  Believed to be a synthetic construct
KMDCRWRWKSCKK

SEQ ID NO: 190 - protein, 13 aa, Artificial Sequence
  Believed to be a synthetic construct
KMDSRWRWKCCKK

SEQ ID NO: 191 - protein, 13 aa, Artificial Sequence
  Believed to be a synthetic construct
KMDCRWRWKSSKK

SEQ ID NO: 192 - protein, 13 aa, Artificial Sequence
  Believed to be a synthetic construct
KMDSRWRWKSSKK

SEQ ID NO: 193 - protein, 13 aa, Artificial Sequence
  Believed to be a synthetic construct
KMDSRWRWKSCKK

SEQ ID NO: 194 - protein, 13 aa, Artificial Sequence
  Believed to be a synthetic construct
KMDSRWRWKCSKK

SEQ ID NO: 195 - protein, 13 aa, Artificial Sequence
  Believed to be a synthetic construct
KMDCRWRPKCCKK

SEQ ID NO: 196 - protein, 13 aa, Artificial Sequence
  Believed to be a synthetic construct
KMDCRPRPKCCKK

SEQ ID NO: 197 - protein, 13 aa, Artificial Sequence
  Believed to be a synthetic construct
  misc_feature (4)..(4): Xaa can be any naturally occurring amino acid
KMDXRPRPKCCKK

SEQ ID NO: 198 - protein, 13 aa, Artificial Sequence
  Believed to be a synthetic construct
  misc_feature (4)..(4): Xaa can be any naturally occurring amino acid
  misc_feature (10)..(10): Xaa can be any naturally occurring amino acid
KMDXRPRPKXCKK

SEQ ID NO: 199 - protein, 13 aa, Artificial Sequence
  Believed to be a synthetic construct
  misc_feature (4)..(4): Xaa can be any naturally occurring amino acid
  misc_feature (11)..(11): Xaa can be any naturally occurring amino acid
KMDXRPRPKCXKK

SEQ ID NO: 200 - protein, 13 aa, Artificial Sequence
  Believed to be a synthetic construct
  misc_feature (10)..(10): Xaa can be any naturally occurring amino acid
KMDCRPRPKXCKK

SEQ ID NO: 201 - protein, 13 aa, Artificial Sequence
  Believed to be a synthetic construct
  misc_feature (11)..(11): Xaa can be any naturally occurring amino acid
KMDCRPRPKCXKK

SEQ ID NO: 202 - protein, 16 aa, Artificial Sequence
  Believed to be a synthetic construct
RQIKIWFQNRRMKWKK

SEQ ID NO: 203 - protein, 16 aa, Artificial Sequence
  Believed to be a synthetic construct
RKKRRRESRKKRRRES

SEQ ID NO: 204 - protein, 17 aa, Artificial Sequence
  Believed to be a synthetic construct
GRPRESGKKRKRKRLKP

SEQ ID NO: 205 - protein, 15 aa, Artificial Sequence
  Believed to be a synthetic construct
GKRKKKGKLGKKRDP

SEQ ID NO: 206 - protein, 17 aa, Artificial Sequence
  Believed to be a synthetic construct
GKRKKKGKLGKKRPRSR

SEQ ID NO: 207 - protein, 18 aa, Artificial Sequence
  Believed to be a synthetic construct
RKKRRRESRRARRSPRHL

SEQ ID NO: 208 - protein, 19 aa, Artificial Sequence
  Believed to be a synthetic construct
SRRARRSPRESGKKRKRKR

SEQ ID NO: 209 - protein, 19 aa, Artificial Sequence
  Believed to be a synthetic construct
VKRGLKLRHVRPRVTRMDV

SEQ ID NO: 210 - protein, 14 aa, Artificial Sequence
  Believed to be a synthetic construct
SRRARRSPRHLGSG

SEQ ID NO: 211 - protein, 16 aa, Artificial Sequence
  Believed to be a synthetic construct
LRRERQSRLRRERQSR

SEQ ID NO: 212 - protein, 22 aa, Artificial Sequence
  Believed to be a synthetic construct
GAYDLRRRERQSRLRRRERQSR

SEQ ID NO: 213 - protein, 13 aa, Artificial Sequence
  Believed to be a synthetic construct
GRKKRRQRRRPPQ

SEQ ID NO: 214 - protein, 5 aa, Artificial Sequence
  Believed to be a synthetic construct
VPMLK

SEQ ID NO: 215 - protein, 5 aa, Artificial Sequence
  Believed to be a synthetic construct
VPTLK

SEQ ID NO: 216 - protein, 5 aa, Artificial Sequence
  Believed to be a synthetic construct
VPALR

SEQ ID NO: 217 - protein, 5 aa, Artificial Sequence
  Believed to be a synthetic construct
VSALK

SEQ ID NO: 218 - protein, 5 aa, Artificial Sequence
  Believed to be a synthetic construct
PMLKE

SEQ ID NO: 219 - protein, 5 aa, Artificial Sequence
  Believed to be a synthetic construct
VPALK

SEQ ID NO: 220 - protein, 5 aa, Artificial Sequence
  Believed to be a synthetic construct
VSLKK

SEQ ID NO: 221 - protein, 5 aa, Artificial Sequence
  Believed to be a synthetic construct
VSGKK

SEQ ID NO: 222 - protein, 5 aa, Artificial Sequence
  Believed to be a synthetic construct
KLPVM

SEQ ID NO: 223 - protein, 5 aa, Artificial Sequence
  Believed to be a synthetic construct
IPMIK

SEQ ID NO: 224 - protein, 5 aa, Artificial Sequence
  Believed to be a synthetic construct
KLGVM

SEQ ID NO: 225 - protein, 5 aa, Artificial Sequence
  Believed to be a synthetic construct
KLPVT

SEQ ID NO: 226 - protein, 5 aa, Artificial Sequence
  Believed to be a synthetic construct
VPMIK

SEQ ID NO: 227 - protein, 5 aa, Artificial Sequence
  Believed to be a synthetic construct
IPALK

SEQ ID NO: 228 - protein, 5 aa, Artificial Sequence
  Believed to be a synthetic construct
IPMLK

SEQ ID NO: 229 - protein, 5 aa, Artificial Sequence
  Believed to be a synthetic construct
VPTLQ

SEQ ID NO: 230 - protein, 5 aa, Artificial Sequence
  Believed to be a synthetic construct
QLPVM

SEQ ID NO: 231 - protein, 5 aa, Artificial Sequence
  Believed to be a synthetic construct
ELPVM

SEQ ID NO: 232 - protein, 5 aa, Artificial Sequence
  Believed to be a synthetic construct
VPTLE

SEQ ID NO: 233 - protein, 20 aa, Artificial Sequence
  Believed to be a synthetic construct
AYRIKPTFRRLKWKYKGKFW

SEQ ID NO: 234 - protein, 20 aa, Artificial Sequence
  Believed to be a synthetic construct
HARIKPTFRRLKWKYKGKFW

SEQ ID NO: 235 - protein, 20 aa, Artificial Sequence
  Believed to be a synthetic construct
HYRIKPTARRLKWKYKGKFW

SEQ ID NO: 236 - protein, 20 aa, Artificial Sequence
  Believed to be a synthetic construct
HYRIKPTFRRLAWKYKGKFW

SEQ ID NO: 237 - protein, 20 aa, Artificial Sequence
  Believed to be a synthetic construct
HYRIKPTFRRLKWKYKGKFA

SEQ ID NO: 238 - protein, 21 aa, Artificial Sequence
  Believed to be a synthetic construct
VNADIKATTVFGGKYVSLTTP

SEQ ID NO: 239 - protein, 22 aa, Artificial Sequence
  Believed to be a synthetic construct
GKYVSLTTPKNPTKRRITPKDV

SEQ ID NO: 240 - protein, 22 aa, Artificial Sequence
  Believed to be a synthetic construct
TKRRITPKDVIDVRSVTTEINT

SEQ ID NO: 241 - protein, 23 aa, Artificial Sequence
  Believed to be a synthetic construct
RSVTTEINTLFQTLTSIAEKVDP

SEQ ID NO: 242 - protein, 26 aa, Artificial Sequence
  Believed to be a synthetic construct
AEKVDPVKLNLTLSAAAEALTGLGDK

SEQ ID NO: 243 - protein, 34 aa, Artificial Sequence
  Believed to be a synthetic construct
GLGDKFGESIVNANTVLDDLNSRMPQSRHDIQQL

SEQ ID NO: 244 - protein, 27 aa, Artificial Sequence
  Believed to be a synthetic construct
GDVYADAAPDLFDFLDSSVTTARTINA

SEQ ID NO: 245 - protein, 32 aa, Artificial Sequence
  Believed to be a synthetic construct
ARTINAQQAELDSALLAAAGFGNTTADVFDRG

SEQ ID NO: 246 - protein, 30 aa, Artificial Sequence
  Believed to be a synthetic construct
ADVFDRGGPYLQRGVADLVPTATLLDTYSP

SEQ ID NO: 247 - protein, 27 aa, Artificial Sequence
  Believed to be a synthetic construct
LDTYSPELFCTIRNFYDADRPDRGAAA

SEQ ID NO: 248 - protein, 22 aa, Artificial Sequence
  Believed to be a synthetic construct
TKRRITPKDVIDVRSVTTEINT

SEQ ID NO: 249 - protein, 22 aa, Artificial Sequence
  Believed to be a synthetic construct
TKRRITPDDVIDVRSVTTEINT

SEQ ID NO: 250 - protein, 22 aa, Artificial Sequence
  Believed to be a synthetic construct
TKRRITPKKVIDVRSVTTEINT

SEQ ID NO: 251 - protein, 22 aa, Artificial Sequence
  Believed to be a synthetic construct
TKRRITPKDVIDVRSVTTKINT

SEQ ID NO: 252 - protein, 13 aa, Artificial Sequence
  Believed to be a synthetic construct
TKRRITPKDVIDV

SEQ ID NO: 253 - protein, 22 aa, Artificial Sequence
  Believed to be a synthetic construct
TKRRITPKDVIDVESVTTEINT

SEQ ID NO: 254 - protein, 22 aa, Artificial Sequence
  Believed to be a synthetic construct
TARRITPKDVIDVRSVTTEINT

SEQ ID NO: 255 - protein, 23 aa, Artificial Sequence
  Believed to be a synthetic construct
TKAARITPKDVIDVRSVTTEINT

SEQ ID NO: 256 - protein, 28 aa, Artificial Sequence
  Believed to be a synthetic construct
HHHHHHTKRRITPKDVIDVRSVTTEINT

SEQ ID NO: 257 - protein, 15 aa, Artificial Sequence
  Believed to be a synthetic construct
KLWMRWYSPTTRRYG

SEQ ID NO: 258 - protein, 15 aa, Artificial Sequence
  Believed to be a synthetic construct
DSLKSYWYLQKFSWR

SEQ ID NO: 259 - protein, 15 aa, Artificial Sequence
  Believed to be a synthetic construct
RTLVNEYKNTLKFSK

SEQ ID NO: 260 - protein, 15 aa, Artificial Sequence
  Believed to be a synthetic construct
IPSRWKDQFWKRWHY

SEQ ID NO: 261 - protein, 15 aa, Artificial Sequence
  Believed to be a synthetic construct
GYGNCRHFKQKPRRD

SEQ ID NO: 262 - protein, 15 aa, Artificial Sequence
  Believed to be a synthetic construct
KNAWKHSSCHHRHQI

SEQ ID NO: 263 - protein, 15 aa, Artificial Sequence
  Believed to be a synthetic construct
RVREWWYTITLKQES

SEQ ID NO: 264 - protein, 15 aa, Artificial Sequence
  Believed to be a synthetic construct
QQHLLIAINGYPRYN

SEQ ID NO: 265 - protein, 15 aa, Artificial Sequence
  Believed to be a synthetic construct
WKCRRQCFRVLHHWN

SEQ ID NO: 266 - protein, 15 aa, Artificial Sequence
  Believed to be a synthetic construct
RLWMRWYSPTTRRYG

SEQ ID NO: 267 - protein, 15 aa, Artificial Sequence
  Believed to be a synthetic construct
KLWMRWYSATTRRYG

SEQ ID NO: 268 - protein, 15 aa, Artificial Sequence
  Believed to be a synthetic construct
KLWMRWYSPWTRRYG

SEQ ID NO: 269 - protein, 15 aa, Artificial Sequence
  Believed to be a synthetic construct
RLWMRWYSPWTRRYG

SEQ ID NO: 270 - protein, 15 aa, Artificial Sequence
  Believed to be a synthetic construct
RLWMRWYSPWTRRWG

SEQ ID NO: 271 - protein, 15 aa, Artificial Sequence
  Unknown source
ALWMRWYSPTTRRYG

SEQ ID NO: 272 - protein, 15 aa, Artificial Sequence
  Believed to be a synthetic construct
RAWMRWYSPTTRRYG

SEQ ID NO: 273 - protein, 15 aa, Artificial Sequence
  Believed to be a synthetic construct
RLAMRWYSPTTRRYG

SEQ ID NO: 274 - protein, 15 aa, Artificial Sequence
  Believed to be a synthetic construct
RLWARWYSPTTRRYG

SEQ ID NO: 275 - protein, 15 aa, Artificial Sequence
  Believed to be a synthetic construct
RLWMAWYSPTTRRYG

SEQ ID NO: 276 - protein, 15 aa, Artificial Sequence
  Believed to be a synthetic construct
RLWMRAYSPTTRRYG

SEQ ID NO: 277 - protein, 15 aa, Artificial Sequence
  Believed to be a synthetic construct
RLWMRWASPTTRRYG

SEQ ID NO: 278 - protein, 15 aa, Artificial Sequence
  Believed to be a synthetic construct
RLWMRWYAPTTRRYG

SEQ ID NO: 279 - protein, 15 aa, Artificial Sequence
  Believed to be a synthetic construct
RLWMRWYSPATRRYG

SEQ ID NO: 280 - protein, 15 aa, Artificial Sequence
  Believed to be a synthetic construct
RLWMRWYSPTARRYG

SEQ ID NO: 281 - protein, 15 aa, Artificial Sequence
  Believed to be a synthetic construct
RLWMRWYSPTTARYG

SEQ ID NO: 282 - protein, 15 aa, Artificial Sequence
  Believed to be a synthetic construct
RLWMRWYSPTTRAYG

SEQ ID NO: 283 - protein, 15 aa, Artificial Sequence
  Believed to be a synthetic construct
RLWMRWYSPTTRRAG

SEQ ID NO: 284 - protein, 15 aa, Artificial Sequence
  Believed to be a synthetic construct
RLWMRWYSPTTRRYA

SEQ ID NO: 285 - protein, 15 aa, Artificial Sequence
  Believed to be a synthetic construct
RLLMRLYSPTTRRYG

SEQ ID NO: 286 - protein, 15 aa, Artificial Sequence
  Believed to be a synthetic construct
RLFMRFYSPTTRRYG

SEQ ID NO: 287 - protein, 15 aa, Artificial Sequence
  Believed to be a synthetic construct
RLIMRIYSPTTRRYG

SEQ ID NO: 288 - protein, 15 aa, Artificial Sequence
  Believed to be a synthetic construct
RLVMRVYSPTTRRYG

SEQ ID NO: 289 - protein, 15 aa, Artificial Sequence
  Believed to be a synthetic construct
RLYMRYYSPTTRRYG

SEQ ID NO: 290 - protein, 12 aa, Artificial Sequence
  Believed to be a synthetic construct
YGRKKKRRQRRR

SEQ ID NO: 291 - protein, 18 aa, Artificial Sequence
  Believed to be a synthetic construct
LLIILRRRIRKQAHAHSK

SEQ ID NO: 292 - protein, 18 aa, Artificial Sequence
  Unknown source
ALIILRRRIRKQAHAHSK

SEQ ID NO: 293 - protein, 18 aa, Artificial Sequence
  Believed to be a synthetic construct
LAIILRRRIRKQAHAHSK

SEQ ID NO: 294 - protein, 18 aa, Artificial Sequence
  Believed to be a synthetic construct
LLAILRRRIRKQAHAHSK

SEQ ID NO: 295 - protein, 18 aa, Artificial Sequence
  Believed to be a synthetic construct
LLIALRRRIRKQAHAHSK

SEQ ID NO: 296 - protein, 18 aa, Artificial Sequence
  Believed to be a synthetic construct
LLIIARRRIRKQAHAHSK

SEQ ID NO: 297 - protein, 18 aa, Artificial Sequence
  Believed to be a synthetic construct
LLIILARRIRKQAHAHSK

SEQ ID NO: 298 - protein, 18 aa, Artificial Sequence
  Believed to be a synthetic construct
LLIILRARIRKQAHAHSK

SEQ ID NO: 299 - protein, 18 aa, Artificial Sequence
  Believed to be a synthetic construct
LLIILRRAIRKQAHAHSK

SEQ ID NO: 300 - protein, 18 aa, Artificial Sequence
  Believed to be a synthetic construct
LLIILRRRARKQAHAHSK

SEQ ID NO: 301 - protein, 19 aa, Artificial Sequence
  Believed to be a synthetic construct
LLIILRRRIARKQAHAHSK

SEQ ID NO: 302 - protein, 18 aa, Artificial Sequence
  Believed to be a synthetic construct
LLIILRRRIRAQAHAHSK

SEQ ID NO: 303 - protein, 18 aa, Artificial Sequence
  Believed to be a synthetic construct
LLIILRRRIRKAAHAHSK

SEQ ID NO: 304 - protein, 18 aa, Artificial Sequence
  Believed to be a synthetic construct
LLIILRRRIRKQAAAHSK

SEQ ID NO: 305 - protein, 18 aa, Artificial Sequence
  Believed to be a synthetic construct
LLIILRRRIRKQAHAASK

SEQ ID NO: 306 - protein, 18 aa, Artificial Sequence
  Believed to be a synthetic construct
LLIILRRRIRKQAHAHAK

SEQ ID NO: 307 - protein, 18 aa, Artificial Sequence
  Believed to be a synthetic construct
LLIILRRRIRKQAHAHSA

SEQ ID NO: 308 - protein, 18 aa, Artificial Sequence
  Believed to be a synthetic construct
KSHAHAQKRIRRRLIILL

SEQ ID NO: 309 - protein, 7 aa, Artificial Sequence
  Believed to be a synthetic construct
RRIRPRP

SEQ ID NO: 310 - protein, 15 aa, Artificial Sequence
  Believed to be a synthetic construct
RRIRPRPPRLPRPRP

SEQ ID NO: 311 - protein, 24 aa, Artificial Sequence
  Believed to be a synthetic construct
RRIRPRPPRLPRPRPRPLPFPRPG

SEQ ID NO: 312 - protein, 17 aa, Artificial Sequence
  Believed to be a synthetic construct
RRIRPRPPRLPRPRPRP

SEQ ID NO: 313 - protein, 20 aa, Artificial Sequence
  Believed to be a synthetic construct
PRPPRLPRPRPRPLPFPRPG

SEQ ID NO: 314 - protein, 18 aa, Artificial Sequence
  Believed to be a synthetic construct
PPRLPRPRPRPLPFPRPG

SEQ ID NO: 315 - protein, 16 aa, Artificial Sequence
  Believed to be a synthetic construct
RLPRPRPRPLPFPRPG

SEQ ID NO: 316 - protein, 14 aa, Artificial Sequence
  Believed to be a synthetic construct
PRPRPRPLPFPRPG

SEQ ID NO: 317 - protein, 12 aa, Artificial Sequence
  Believed to be a synthetic construct
PRPRPLPFPRPG

SEQ ID NO: 318 - protein, 10 aa, Artificial Sequence
  Believed to be a synthetic construct
PRPLPFPRPG

SEQ ID NO: 319 - protein, 9 aa, Artificial Sequence
  Believed to be a synthetic construct
RKKRRQRRR

SEQ ID NO: 320 - protein, 27 aa, Artificial Sequence
  Believed to be a synthetic construct
RQGAARVTSWLGRQLRIAGKRLEGRSK

SEQ ID NO: 321 - protein, 22 aa, Artificial Sequence
  Believed to be a synthetic construct
RVTSWLGRQLRIAGKRLEGRSK

SEQ ID NO: 322 - protein, 16 aa, Artificial Sequence
  Believed to be a synthetic construct
GRQLRIAGKRLEGRSK

SEQ ID NO: 323 - protein, 23 aa, Artificial Sequence
  Believed to be a synthetic construct
RRVTSWLGRQLRIAGKRLEGRSK

SEQ ID NO: 324 - protein, 22 aa, Artificial Sequence
  Believed to be a synthetic construct
RVRSWLGRQLRIAGKRLEGRSK

SEQ ID NO: 325 - protein, 16 aa, Artificial Sequence
  Believed to be a synthetic construct
GRQLRIAGKRLRGRSK

SEQ ID NO: 326 - protein, 16 aa, Artificial Sequence
  Believed to be a synthetic construct
GRQLRIAGRRLRGRSR

SEQ ID NO: 327 - protein, 16 aa, Artificial Sequence
  Believed to be a synthetic construct
GRQLRRAGRRLRGRSR

SEQ ID NO: 328 - protein, 16 aa, Artificial Sequence
  Believed to be a synthetic construct
GRQLRIAGRRLRRRSR

SEQ ID NO: 329 - protein, 16 aa, Artificial Sequence
  Believed to be a synthetic construct
GRQLRRAGRRLRRRSR

SEQ ID NO: 330 - protein, 15 aa, Artificial Sequence
  Believed to be a synthetic construct
RQLRIAGRRLRGRSR

SEQ ID NO: 331 - protein, 29 aa, Artificial Sequence
  Believed to be a synthetic construct
KLIKGRTPIKFGKADCDRPPKHSQNGMGK

SEQ ID NO: 332 - protein, 27 aa, Artificial Sequence
  Believed to be a synthetic construct
KLIKGRTPIKFGKADCDRPPKHSQNGM

SEQ ID NO: 333 - protein, 27 aa, Artificial Sequence
  Believed to be a synthetic construct
KLIKGRTPIKFGKADCDRPPKHSQNGK

SEQ ID NO: 334 - protein, 26 aa, Artificial Sequence
  Believed to be a synthetic construct
KGRTPIKFGKADCDRPPKHSQNGMGK

SEQ ID NO: 335 - protein, 25 aa, Artificial Sequence
  Believed to be a synthetic construct
KLIKGRTPIKFGKADCDRPPKHSGK

SEQ ID NO: 336 - protein, 25 aa, Artificial Sequence
  Believed to be a synthetic construct
KLIKGRTPIKFGKARCRRPPKHSGK

SEQ ID NO: 337 - protein, 13 aa, Artificial Sequence
  Believed to be a synthetic construct
KLIKGRTPIKFGK

SEQ ID NO: 338 - protein, 37 aa, Artificial Sequence
  Believed to be a synthetic construct
KRIPNKKPGKKTTTKPTKKPTIKTTKKDLKPQTTKPK

SEQ ID NO: 339 - protein, 30 aa, Artificial Sequence
  Believed to be a synthetic construct
KRIPNKKPGKKTTTKPTKKPTIKTTKKDLK

SEQ ID NO: 340 - protein, 27 aa, Artificial Sequence
  Believed to be a synthetic construct
KRIPNKKPGKKTTTKPTKKPTIKTTKK

SEQ ID NO: 341 - protein, 23 aa, Artificial Sequence
  Believed to be a synthetic construct
KRIPNKKPGKKTTTKPTKKPTIK

SEQ ID NO: 342 - protein, 19 aa, Artificial Sequence
  Believed to be a synthetic construct
KRIPNKKPGKKTTTKPTKK

SEQ ID NO: 343 - protein, 12 aa, Artificial Sequence
  Believed to be a synthetic construct
KRIPNKKPGKKT

SEQ ID NO: 344 - protein, 11 aa, Artificial Sequence
  Believed to be a synthetic construct
KRIPNKKPGKK

SEQ ID NO: 345 - protein, 10 aa, Artificial Sequence
  Believed to be a synthetic construct
KRIPNKKPKK

SEQ ID NO: 346 - protein, 10 aa, Artificial Sequence
  Believed to be a synthetic construct
RRIPNRRPRR

SEQ ID NO: 347 - protein, 22 aa, Artificial Sequence
  Believed to be a synthetic construct
KKPGKKTTTKPTKKPTIKTTKK

SEQ ID NO: 348 - protein, 14 aa, Artificial Sequence
  Believed to be a synthetic construct
KKPGKKTTTKPTKK

SEQ ID NO: 349 - protein, 10 aa, Artificial Sequence
  Believed to be a synthetic construct
KKPTIKTTKK

SEQ ID NO: 350 - protein, 10 aa, Artificial Sequence
  Believed to be a synthetic construct
KKTTTKPTKK

SEQ ID NO: 351 - protein, 15 aa, Artificial Sequence
  Believed to be a synthetic construct
KSICKTIPSNKPKKK

SEQ ID NO: 352 - protein, 11 aa, Artificial Sequence
  Believed to be a synthetic construct
KTIPSNKPKKK

SEQ ID NO: 353 - protein, 12 aa, Artificial Sequence
  Believed to be a synthetic construct
KPRSKNPPKKPK

SEQ ID NO: 354 - protein, 17 aa, Artificial Sequence
  Believed to be a synthetic construct
DRDDRDDRDDRDDRDDR

SEQ ID NO: 355 - protein, 14 aa, Artificial Sequence
  Believed to be a synthetic construct
ERERERERERERER

SEQ ID NO: 356 - protein, 14 aa, Artificial Sequence
  Believed to be a synthetic construct
WRWRWRWRWRWRWR

SEQ ID NO: 357 - protein, 10 aa, Artificial Sequence
  Believed to be a synthetic construct
DRDRDRDRDR

SEQ ID NO: 358 - protein, 27 aa, Artificial Sequence
  Believed to be a synthetic construct
GALFLGFLGAAGSTMGAWSQPKKKRKV

SEQ ID NO: 359 - protein, 22 aa, Artificial Sequence
  Believed to be a synthetic construct
DRRRRGSRPSGAERRRRRAAAA

SEQ ID NO: 360 - protein, 17 aa, Artificial Sequence
  Believed to be a synthetic construct
DRRRRGSRPSGAERRRR

SEQ ID NO: 361 - protein, 15 aa, Artificial Sequence
  Believed to be a synthetic construct
QTRRRERRAEKQAQW

SEQ ID NO: 362 - protein, 9 aa, Artificial Sequence
  Believed to be a synthetic construct
RRRERRAEK

SEQ ID NO: 363 - protein, 11 aa, Artificial Sequence
  Believed to be a synthetic construct
NRARRNRRRVR

SEQ ID NO: 364 - protein, 10 aa, Artificial Sequence
  Believed to be a synthetic construct
RTRRNRRRVR

SEQ ID NO: 365 - protein, 8 aa, Artificial Sequence
  Believed to be a synthetic construct
RNRSRHRR

SEQ ID NO: 366 - protein, 9 aa, Artificial Sequence
  Believed to be a synthetic construct
KCPSRRPKR

SEQ ID NO: 367 - protein, 16 aa, Artificial Sequence
  Believed to be a synthetic construct
KRPAAIKKAGQAKKKK

SEQ ID NO: 368 - protein, 12 aa, Artificial Sequence
  Believed to be a synthetic construct
TRRSKRRSHRKF

SEQ ID NO: 369 - protein, 17 aa, Artificial Sequence
  Believed to be a synthetic construct
RAGLQFPVGRVHRLLRK

SEQ ID NO: 370 - protein, 22 aa, Artificial Sequence
  Believed to be a synthetic construct
MVRRFLVTLRIRRACGPPRVRV

SEQ ID NO: 371 - protein, 22 aa, Artificial Sequence
  Believed to be a synthetic construct
FVTRGCPRRLVARLIRVMVPRR

SEQ ID NO: 372 - protein, 13 aa, Artificial Sequence
  Believed to be a synthetic construct
VRRFLVTLRIRRA

SEQ ID NO: 373 - protein, 13 aa, Artificial Sequence
  Believed to be a synthetic construct
RVRILARFLRTRV

SEQ ID NO: 374 - protein, 13 aa, Artificial Sequence
  Believed to be a synthetic construct
RVRVFVVHIPRLT

SEQ ID NO: 375 - protein, 13 aa, Artificial Sequence
  Believed to be a synthetic construct
VIRVHFRLPVRTV

SEQ ID NO: 376 - protein, 37 aa, Artificial Sequence
  Believed to be a synthetic construct
MVRRFLVTLRIRRACGPPRVRVFVVHIPRLTGEWAAP

SEQ ID NO: 377 - protein, 37 aa, Artificial Sequence
  Believed to be a synthetic construct
FRVPLRIRPCVVAPRLVMVRHTFGRIARWVAGPLETR

SEQ ID NO: 378 - protein, 21 aa, Artificial Sequence
  Believed to be a synthetic construct
AGYLLGKINLKALAALAKKIL

SEQ ID NO: 379 - protein, 25 aa, Artificial Sequence
  Believed to be a synthetic construct
GTKMIFVGIKKKEERADLIAYLKKA

SEQ ID NO: 380 - protein, 16 aa, Artificial Sequence
  Believed to be a synthetic construct
KKKEERADLIAYLKKA

SEQ ID NO: 381 - protein, 14 aa, Artificial Sequence
  Believed to be a synthetic construct
KMIFVGIKKKEERA

SEQ ID NO: 382 - protein, 10 aa, Artificial Sequence
  Believed to be a synthetic construct
KMIFVGIKKK

SEQ ID NO: 383 - protein, 10 aa, Artificial Sequence
  Believed to be a synthetic construct
EKGKKIFIMK

SEQ ID NO: 384 - protein, 9 aa, Artificial Sequence
  Believed to be a synthetic construct
KGKKIFIMK

SEQ ID NO: 385 - protein, 17 aa, Artificial Sequence
  Believed to be a synthetic construct
RRRRNRTRRNRRRVRGC

SEQ ID NO: 386 - protein, 15 aa, Artificial Sequence
  Believed to be a synthetic construct
TRRQRTRRARRNRGC

SEQ ID NO: 387 - protein, 21 aa, Artificial Sequence
  Believed to be a synthetic construct
KMTRAQRRAAARRNRWTARGC

SEQ ID NO: 388 - protein, 21 aa, Artificial Sequence
  Believed to be a synthetic construct
KLTRAQRRAAARKNKRNTRGC

SEQ ID NO: 389 - protein, 19 aa, Artificial Sequence
  Believed to be a synthetic construct
NAKTRRHERRRKLAIERGC

SEQ ID NO: 390 - protein, 24 aa, Artificial Sequence
  Believed to be a synthetic construct
MDAQTRRRERRAEKQAQWKAANGC

SEQ ID NO: 391 - protein, 20 aa, Artificial Sequence
  Believed to be a synthetic construct
TAKTRYKARRAELIAERRGC

SEQ ID NO: 392 - protein, 14 aa, Artificial Sequence
  Believed to be a synthetic construct
SQMTRQARRLYBGC

SEQ ID NO: 393 - protein, 28 aa, Artificial Sequence
  Believed to be a synthetic construct
KRRIRRERNKMAAAKSRNRRRELTDTGC

SEQ ID NO: 394 - protein, 30 aa, Artificial Sequence
  Believed to be a synthetic construct
RIKAERKRMRNRIAASKSRKRKLERIARGC

SEQ ID NO: 395 - protein, 24 aa, Artificial Sequence
  Believed to be a synthetic construct
KRARNTEAARRSRARKLQRMKQGC

SEQ ID NO: 396 - protein, 22 aa, Artificial Sequence
  Believed to be a synthetic construct
KCFQWQRNMRKVRGPPVSCIKR

SEQ ID NO: 397 - protein, 19 aa, Artificial Sequence
  Believed to be a synthetic construct
KCFQWQRNMRKVRGPPVSC

SEQ ID NO: 398 - protein, 22 aa, Artificial Sequence
  Believed to be a synthetic construct
KCFQWQRNMRKVRGPPVSSIKR

SEQ ID NO: 399 - protein, 13 aa, Artificial Sequence
  Believed to be a synthetic construct
KCFQWQRNMRKVR

SEQ ID NO: 400 - protein, 16 aa, Artificial Sequence
  Believed to be a synthetic construct
FQWQRNMRKVRGPPVS

SEQ ID NO: 401 - protein, 19 aa, Artificial Sequence
  Believed to be a synthetic construct
QWQRNMRKVRGPPVSCIKR

SEQ ID NO: 402 - protein, 10 aa, Artificial Sequence
  Believed to be a synthetic construct
QWQRNMRKVR

SEQ ID NO: 403 - protein, 16 aa, Artificial Sequence
  Believed to be a synthetic construct
RQIKIWFQNRRMKWKK

SEQ ID NO: 404 - protein, 22 aa, Artificial Sequence
  Believed to be a synthetic construct
KCFMWQEMLNKAGVPKLRCARK

SEQ ID NO: 405 - protein, 21 aa, Artificial Sequence
  Believed to be a synthetic construct
KETWWETWWTEWSQPKKKRKV

SEQ ID NO: 406 - protein, 21 aa, Artificial Sequence
  Believed to be a synthetic construct
KETWFETWFTEWSQPKKKRKV

SEQ ID NO: 407 - protein, 15 aa, Artificial Sequence
  Believed to be a synthetic construct
KWFETWFTEWPKKRK

SEQ ID NO: 408 - protein, 20 aa, Artificial Sequence
  Believed to be a synthetic construct
GLWRALWRLLRSLWRLLWRA

SEQ ID NO: 409 - protein, 20 aa, Artificial Sequence
  Believed to be a synthetic construct
GLWWRLWWRLRSWFRLWFRA

SEQ ID NO: 410 - protein, 34 aa, Artificial Sequence
  Believed to be a synthetic construct
DAATATRGRSAASRPTQRPRAPARSASRPRRPVE

SEQ ID NO: 411 - protein, 27 aa, Artificial Sequence
  Believed to be a synthetic construct
GALFLGFLGAAGSTMGAWSQPKKKRKV

SEQ ID NO: 412 - protein, 27 aa, Artificial Sequence
  Believed to be a synthetic construct
GALFLGFLGAAGSTMGAWSQPKSKRKV

SEQ ID NO: 413 - protein, 12 aa, Artificial Sequence
  Believed to be a synthetic construct
PLSSIFSRIGDP

SEQ ID NO: 414 - protein, 12 aa, Artificial Sequence
  Believed to be a synthetic construct
PSSSSSSRIGDP

SEQ ID NO: 415 - protein, 18 aa, Artificial Sequence
  Believed to be a synthetic construct
VELPPPVELPPPVELPPP

SEQ ID NO: 416 - protein, 28 aa, Artificial Sequence
  Believed to be a synthetic construct
ALWMTLLKKVLKAAAKAALNAVLVGANA

SEQ ID NO: 417 - protein, 13 aa, Artificial Sequence
  Believed to be a synthetic construct
ALWKTLLKKVLKA

SEQ ID NO: 418 - protein, 20 aa, Artificial Sequence
  Believed to be a synthetic construct
ALWKTLLKKVLKAPKKKRKV

SEQ ID NO: 419 - protein, 20 aa, Artificial Sequence
  Believed to be a synthetic construct
PKKKRKVALWKTLLKKVLKA

SEQ ID NO: 420 - protein, 20 aa, Artificial Sequence
  Believed to be a synthetic construct
VKRKKKPALWKTLLKKVLKA

SEQ ID NO: 421 - protein, 22 aa, Artificial Sequence
  Believed to be a synthetic construct
RQARRNRRRALWKTLLKKVLKA

SEQ ID NO: 422 - protein, 10 aa, Artificial Sequence
  Believed to be a synthetic construct
RQARRNRRRC

SEQ ID NO: 423 - protein, 14 aa, Artificial Sequence
  Believed to be a synthetic construct
GRKKRRQRRRPPQC

SEQ ID NO: 424 - protein, 13 aa, Artificial Sequence
  Believed to be a synthetic construct
EEEAAGRKRKKRT

SEQ ID NO: 425 - protein, 5 aa, Artificial Sequence
  Believed to be a synthetic construct
EEEAA

SEQ ID NO: 426 - protein, 8 aa, Artificial Sequence
  Believed to be a synthetic construct
EEEAAKKK

SEQ ID NO: 427 - protein, 8 aa, Artificial Sequence
  Believed to be a synthetic construct
GRKRKKRT

SEQ ID NO: 428 - protein, 13 aa, Artificial Sequence
  Believed to be a synthetic construct
FFFAAGRKRKKRT

SEQ ID NO: 429 - protein, 13 aa, Artificial Sequence
  Believed to be a synthetic construct
NNNAAGRKRKKRT

SEQ ID NO: 430 - protein, 13 aa, Artificial Sequence
  Believed to be a synthetic construct
YYYAAGRKRKKRT

SEQ ID NO: 431 - protein, 22 aa, Artificial Sequence
  Believed to be a synthetic construct
MVTVLFRRLRIRRACGPPRVRV

SEQ ID NO: 432 - protein, 16 aa, Artificial Sequence
  Believed to be a synthetic construct
RQIKIWFQNRRMKWKK

SEQ ID NO: 433 - protein, 21 aa, Artificial Sequence
  Believed to be a synthetic construct
AGYLLGKINLKALAALAKKIL

SEQ ID NO: 434 - protein, 10 aa, Artificial Sequence
  Believed to be a synthetic construct
VQRKRQKLMP

SEQ ID NO: 435 - protein, 8 aa, Artificial Sequence
  Believed to be a synthetic construct
SKKKKTKV

SEQ ID NO: 436 - protein, 8 aa, Artificial Sequence
  Believed to be a synthetic construct
GRKRKKRT

SEQ ID NO: 437 - protein, 11 aa, Artificial Sequence
  Believed to be a synthetic construct
GKKKKRKREKL

SEQ ID NO: 438 - protein, 7 aa, Artificial Sequence
  Believed to be a synthetic construct
PKKKRKV

SEQ ID NO: 439 - protein, 8 aa, Artificial Sequence
  Believed to be a synthetic construct
ERKKRRRE

SEQ ID NO: 440 - protein, 7 aa, Artificial Sequence
  Believed to be a synthetic construct
FKKFRKF

SEQ ID NO: 441 - protein, 24 aa, Artificial Sequence
  Believed to be a synthetic construct
LGTYTQDFNKFHTFPQTAIGVGAP

SEQ ID NO: 442 - protein, 24 aa, Artificial Sequence
  Believed to be a synthetic construct
LGTYTQDFNKFHTFPQTAIGVGAP

SEQ ID NO: 443 - protein, 21 aa, Artificial Sequence
  Believed to be a synthetic construct
YTQDFNKFHTFPQTAIGVGAP

SEQ ID NO: 444 - protein, 18 aa, Artificial Sequence
  Believed to be a synthetic construct
DFNKFHTFPQTAIGVGAP

SEQ ID NO: 445 - protein, 15 aa, Artificial Sequence
  Believed to be a synthetic construct
KFHTFPQTAIGVGAP

SEQ ID NO: 446 - protein, 12 aa, Artificial Sequence
  Believed to be a synthetic construct
TFPQTAIGVGAP

SEQ ID NO: 447 - protein, 13 aa, Artificial Sequence
  Believed to be a synthetic construct
GYGRKKRRQRRRG

SEQ ID NO: 448 - protein, 13 aa, Artificial Sequence
  Believed to be a synthetic construct
GYGRKKRRQRRRG

SEQ ID NO: 449 - protein, 13 aa, Artificial Sequence
  Believed to be a synthetic construct
GYGRKKRRQRRRG

SEQ ID NO: 450 - protein, 16 aa, Artificial Sequence
  Believed to be a synthetic construct
RQIKIWFQNRRMKWKK

SEQ ID NO: 451 - protein, 15 aa, Artificial Sequence
  Believed to be a synthetic construct
FLGKKFKKYFLQLLK

SEQ ID NO: 452 - protein, 23 aa, Artificial Sequence
  Believed to be a synthetic construct
FLIFIRVICIVIAKLKANLMCKT

SEQ ID NO: 453 - protein, 17 aa, Artificial Sequence
  Believed to be a synthetic construct
KKAAQIRSQVMTHLRVI

SEQ ID NO: 454 - protein, 16 aa, Artificial Sequence
  Believed to be a synthetic construct
YIVLRRRRKRVNTKRS

SEQ ID NO: 455 - protein, 11 aa, Artificial Sequence
  Believed to be a synthetic construct
RRKLSQQKEKK

SEQ ID NO: 456 - protein, 15 aa, Artificial Sequence
  Believed to be a synthetic construct
VQAILRRNWNQYKIQ

SEQ ID NO: 457 - protein, 16 aa, Artificial Sequence
  Believed to be a synthetic construct
KTVLLRKLLKLLVRKI

SEQ ID NO: 458 - protein, 16 aa, Artificial Sequence
  Believed to be a synthetic construct
LLKKRKVVRLIKFLLK

SEQ ID NO: 459 - protein, 19 aa, Artificial Sequence
  Believed to be a synthetic construct
KLPCRSNTFLNIFRRKKPG

SEQ ID NO: 460 - protein, 16 aa, Artificial Sequence
  Believed to be a synthetic construct
KKICTRKPRFMSAWAQ

SEQ ID NO: 461 - protein, 16 aa, Artificial Sequence
  Believed to be a synthetic construct
RQIKIWFQNRRMKWKK

SEQ ID NO: 462 - protein, 18 aa, Artificial Sequence
  Believed to be a synthetic construct
RGGRLSYSRRRFSTSTGR

SEQ ID NO: 463 - protein, 10 aa, Artificial Sequence
  Believed to be a synthetic construct
RRLSYSRRRF

SEQ ID NO: 464 - protein, 17 aa, Artificial Sequence
  Believed to be a synthetic construct
RGGRLAYLRRRWAVLGR

SEQ ID NO: 465 - protein, 16 aa, Artificial Sequence
  Believed to be a synthetic construct
RQIKIWFQNRRMKWKK

SEQ ID NO: 466 - protein, 28 aa, Artificial Sequence
  Believed to be a synthetic construct
MANLGYWLLALFVTMWTDVGLCKKRPKP

SEQ ID NO: 467 - protein, 28 aa, Artificial Sequence
  Believed to be a synthetic construct
MANLGCWMLVLFVATWSDLGLCKKRPKP

SEQ ID NO: 468 - protein, 30 aa, Artificial Sequence
  Believed to be a synthetic construct
MVKSKIGSWILVLFVAMWSDVGLCKKRPKP

SEQ ID NO: 469 - protein, 38 aa, Artificial Sequence
  Believed to be a synthetic construct
LLGDFFRKSKEKIGKEFKRIVQRIKDFLRNLVPRTESC

SEQ ID NO: 470 - protein, 16 aa, Artificial Sequence
  Believed to be a synthetic construct
RQIKIWFQNRRMKWKK

SEQ ID NO: 471 - protein, 16 aa, Artificial Sequence
  Believed to be a synthetic construct
RVIRVWFQNKRCKDKK

SEQ ID NO: 472 - protein, 23 aa, Artificial Sequence
  Believed to be a synthetic construct
GIGKFLHSAKKWGKAFVGQIMNC

SEQ ID NO: 473 - protein, 24 aa, Artificial Sequence
  Believed to be a synthetic construct
TRSSRAGLQWPVGRVHRLLRKGGC

SEQ ID NO: 474 - protein, 16 aa, Artificial Sequence
  Believed to be a synthetic construct
RHIKIWFQNRRMKWKK

SEQ ID NO: 475 - protein, 9 aa, Artificial Sequence
  Believed to be a synthetic construct
RKKRRQRRR

SEQ ID NO: 476 - protein, 16 aa, Artificial Sequence
  Believed to be a synthetic construct
RQIKIWFQNRRMKWKK

SEQ ID NO: 477 - protein, 60 aa, Artificial Sequence
  Believed to be a synthetic construct
SKRTRQTYTRYQTLELEKEFHFNRYITRRRRIDIANALSLSERQIKIWFQNRRMKSKKDR

SEQ ID NO: 478 - protein, 61 aa, Artificial Sequence
  Believed to be a synthetic construct
EKRPRTAFSSEQLARLKREFNENRYLTTERRRQQLSSELGLNEAQIKIWFQNKRAKIKKST

SEQ ID NO: 479 - protein, 13 aa, Artificial Sequence
  Believed to be a synthetic construct
GRRRRRRRRRPPQ

SEQ ID NO: 480 - protein, 27 aa, Artificial Sequence
  Believed to be a synthetic construct
GALFLGFLGAAGSTMGAWSQPKKKRKV

SEQ ID NO: 481 - protein, 27 aa, Artificial Sequence
  Believed to be a synthetic construct
GALFLAFLAAALSLMGLWSQPKKKRRV

SEQ ID NO: 482 - protein, 10 aa, Artificial Sequence
  Believed to be a synthetic construct
MLLLTRRRST

SEQ ID NO: 483 - protein, 9 aa, Artificial Sequence
  Believed to be a synthetic construct
CGNKRTRGC

SEQ ID NO: 484 - protein, 12 aa, Artificial Sequence
  Believed to be a synthetic construct
TSPLNIHNGQKL

SEQ ID NO: 485 - protein, 16 aa, Artificial Sequence
  Believed to be a synthetic construct
GLRKRLRKFRNKIKEK

SEQ ID NO: 486 - protein, 27 aa, Artificial Sequence
  Believed to be a synthetic construct
GLLEALAELLEGLRKRLRKFRNKIKEK

SEQ ID NO: 487 - protein, 13 aa, Artificial Sequence
  Believed to be a synthetic construct
CVQWSLLRGYQPC

SEQ ID NO: 488 - protein, 6 aa, Artificial Sequence
  Believed to be a synthetic construct
VRLPPP

SEQ ID NO: 489 - protein, 12 aa, Artificial Sequence
  Believed to be a synthetic construct
VRLPPPVRLPPP

SEQ ID NO: 490 - protein, 18 aa, Artificial Sequence
  Believed to be a synthetic construct
VRLPPPVRLPPPVRLPPP

SEQ ID NO: 491 - protein, 6 aa, Artificial Sequence
  Believed to be a synthetic construct
VHLPPP

SEQ ID NO: 492 - protein, 12 aa, Artificial Sequence
  Believed to be a synthetic construct
VHLPPPVHLPPP

SEQ ID NO: 493 - protein, 18 aa, Artificial Sequence
  Believed to be a synthetic construct
VHLPPPVHLPPPVHLPPP

SEQ ID NO: 494 - protein, 6 aa, Artificial Sequence
  Believed to be a synthetic construct
VKLPPP

SEQ ID NO: 495 - protein, 12 aa, Artificial Sequence
  Believed to be a synthetic construct
VKLPPPVKLPPP

SEQ ID NO: 496 - protein, 18 aa, Artificial Sequence
  Believed to be a synthetic construct
VKLPPPVKLPPPVKLPPP

SEQ ID NO: 497 - protein, 16 aa, Artificial Sequence
  Believed to be a synthetic construct
RQIKIWFQNRRMKWKK

SEQ ID NO: 498 - protein, 16 aa, Artificial Sequence
  Believed to be a synthetic construct
RQIKIFFQNRRMKWKK

SEQ ID NO: 499 - protein, 21 aa, Artificial Sequence
  Believed to be a synthetic construct
ASMWERVKSIIKSSLAAASNI

SEQ ID NO: 500 - protein, 21 aa, Artificial Sequence
  Believed to be a synthetic construct
ASMWERVKSIIKSSLAAASNI

SEQ ID NO: 501 - protein, 26 aa, Artificial Sequence
  Believed to be a synthetic construct
DPKGDPKGVTVTVTVTVTGKGDPKPD

SEQ ID NO: 502 - protein, 16 aa, Artificial Sequence
  Believed to be a synthetic construct
CSIPPEVKFNPFVYLI

SEQ ID NO: 503 - protein, 6 aa, Artificial Sequence
  Believed to be a synthetic construct
PFVYLI

SEQ ID NO: 504 - protein, 8 aa, Artificial Sequence
  Believed to be a synthetic construct
NKPILVFY

SEQ ID NO: 505 - protein, 14 aa, Artificial Sequence
  Believed to be a synthetic construct
YKQCHKKGGKKGSG

SEQ ID NO: 506 - protein, 15 aa, Artificial Sequence
  Believed to be a synthetic construct
  misc_feature (10)..(10): Xaa can be any naturally occurring amino acid
YKQCHKKGGXKKGSG

SEQ ID NO: 507 - protein, 14 aa, Artificial Sequence
  Believed to be a synthetic construct
GSGKKGGKKHCQKY

SEQ ID NO: 508 - protein, 14 aa, Artificial Sequence
  Believed to be a synthetic construct
GSGKKGGKKICQKY

SEQ ID NO: 509 - protein, 16 aa, Artificial Sequence
  Believed to be a synthetic construct
YTAIAWVKAFIRKLRK

SEQ ID NO: 510 - protein, 17 aa, Artificial Sequence
  Believed to be a synthetic construct
IAWVKAFIRKLRKGPLG

SEQ ID NO: 511 - protein, 23 aa, Artificial Sequence
  Believed to be a synthetic construct
LIRLWSHLIHIWFQNRRLKWKKK

SEQ ID NO: 512 - protein, 34 aa, Artificial Sequence
  Believed to be a synthetic construct
KKKKKKGGFLGFWRGENGRKTRSAYERMCILKGK

SEQ ID NO: 513 - protein, 14 aa, Artificial Sequence
  Believed to be a synthetic construct
RLSGMNEVLSFRWL

SEQ ID NO: 514 - protein, 13 aa, Artificial Sequence
  Believed to be a synthetic construct
GPFHFYQFLFPPV

SEQ ID NO: 515 - protein, 13 aa, Artificial Sequence
  Believed to be a synthetic construct
GSPWGLQHHPPRT

SEQ ID NO: 516 - protein, 16 aa, Artificial Sequence
  Believed to be a synthetic construct
AAVALLPAVLLALLAP

SEQ ID NO: 517 - protein, 41 aa, Artificial Sequence
  Believed to be a synthetic construct
AAVALLPAVLLALLAPEILLPNNYNAYESYKYPGMFIALSK

SEQ ID NO: 518 - protein, 26 aa, Artificial Sequence
  Believed to be a synthetic construct
AAVALLPAVLLALLAPVQRKRQKLMP

SEQ ID NO: 519 - protein, 30 aa, Artificial Sequence
  Believed to be a synthetic construct
WEAKLAKALAKALAKHLAKALAKALKACEA

SEQ ID NO: 520 - protein, 27 aa, Artificial Sequence
  Believed to be a synthetic construct
MGLGLHLLVLAAALQGAWSQPKKKRKV

SEQ ID NO: 521 - protein, 23 aa, Artificial Sequence
  Believed to be a synthetic construct
MGLGLHLLVLAAALQGAKKKRKV

SEQ ID NO: 522 - protein, 30 aa, Artificial Sequence
  Believed to be a synthetic construct
WEAALAEALAEALAEHLAEALAEALEALAA

SEQ ID NO: 523 - protein, 20 aa, Artificial Sequence
  Believed to be a synthetic construct
GLFEALLELLESLWELLLEA

SEQ ID NO: 524 - protein, 20 aa, Artificial Sequence
  Believed to be a synthetic construct
GLFKALLKLLKSLWKLLLKA

SEQ ID NO: 525 - protein, 20 aa, Artificial Sequence
  Believed to be a synthetic construct
GLFRALLRLLRSLWRLLLRA

SEQ ID NO: 526 - protein, 23 aa, Artificial Sequence
  Believed to be a synthetic construct
CGAYDLRRRERQSRLRRRERQSR

SEQ ID NO: 527 - protein, 17 aa, Artificial Sequence
  Believed to be a synthetic construct
RKKRRRESRKKRRRESC

SEQ ID NO: 528 - protein, 19 aa, Artificial Sequence
  Believed to be a synthetic construct
CVKRGLKLRHVRPRVTRDV

SEQ ID NO: 529 - protein, 17 aa, Artificial Sequence
  Believed to be a synthetic construct
CRQIKIWFQNRRMKWKK

SEQ ID NO: 530 - protein, 11 aa, Artificial Sequence
  Believed to be a synthetic construct
YARAAARQARA

SEQ ID NO: 531 - protein, 15 aa, Artificial Sequence
  Believed to be a synthetic construct
PPKKSAQCLRYKKPE

SEQ ID NO: 532 - protein, 15 aa, Artificial Sequence
  Believed to be a synthetic construct
DPVDTPNPTRRKPGK

SEQ ID NO: 533 - protein, 13 aa, Artificial Sequence
  Believed to be a synthetic construct
KRVSRNKSEKKRR

SEQ ID NO: 534 - protein, 16 aa, Artificial Sequence
  Believed to be a synthetic construct
GRRHHCRSKAKRSRHH

SEQ ID NO: 535 - protein, 16 aa, Artificial Sequence
  Believed to be a synthetic construct
SARHHCRSKAKRSRHH

SEQ ID NO: 536 - protein, 16 aa, Artificial Sequence
  Believed to be a synthetic construct
SRAHHCRSKAKRSRHH

SEQ ID NO: 537 - protein, 16 aa, Artificial Sequence
  Believed to be a synthetic construct
SRRAHCRSKAKRSRHH

SEQ ID NO: 538 - protein, 16 aa, Artificial Sequence
  Believed to be a synthetic construct
SRRHACRSKAKRSRHH

SEQ ID NO: 539 - protein, 16 aa, Artificial Sequence
  Believed to be a synthetic construct
SRRHHARSKAKRSRHH

SEQ ID NO: 540 - protein, 16 aa, Artificial Sequence
  Believed to be a synthetic construct
SRRHHCRAKAKRSRHH

SEQ ID NO: 541 - protein, 16 aa, Artificial Sequence
  Believed to be a synthetic construct
SRRHHCRSAAKRSRHH

SEQ ID NO: 542 - protein, 16 aa, Artificial Sequence
  Believed to be a synthetic construct
SRRHHCRSKAARSRHH

SEQ ID NO: 543 - protein, 16 aa, Artificial Sequence
  Believed to be a synthetic construct
SRRHHCRSKAKASRHH

SEQ ID NO: 544 - protein, 16 aa, Artificial Sequence
  Believed to be a synthetic construct
SRRHHCRSKAKRARHH

SEQ ID NO: 545 - protein, 16 aa, Artificial Sequence
  Believed to be a synthetic construct
SRRHHCRSKAKRSAHH

SEQ ID NO: 546 - protein, 13 aa, Artificial Sequence
  Believed to be a synthetic construct
RRHHCRSKAKRSR

SEQ ID NO: 547 - protein, 12 aa, Artificial Sequence
  Believed to be a synthetic construct
GRKGKHKRKKLP

SEQ ID NO: 548 - protein, 10 aa, Artificial Sequence
  Believed to be a synthetic construct
GKKKKKKKKK

SEQ ID NO: 549 - protein, 18 aa, Artificial Sequence
  Believed to be a synthetic construct
GKRVAKRKLIEQNRERRR

SEQ ID NO: 550 - protein, 18 aa, Artificial Sequence
  Believed to be a synthetic construct
GRKLKKKKNEKEDKRPRT

SEQ ID NO: 551 - protein, 17 aa, Artificial Sequence
  Believed to be a synthetic construct
GKKTNLFSALIKKKKTA

SEQ ID NO: 552 - protein, 18 aa, Artificial Sequence
  Believed to be a synthetic construct
GRRERNKMAAAKCRNRRR

SEQ ID NO: 553 - protein, 18 aa, Artificial Sequence
  Believed to be a synthetic construct
GKRARNTEAARRSRARKL

SEQ ID NO: 554 - protein, 14 aa, Artificial Sequence
  Believed to be a synthetic construct
GRRRRATAKYRTAH

SEQ ID NO: 555 - protein, 15 aa, Artificial Sequence
  Believed to be a synthetic construct
GKRRRRATAKYRSAH

SEQ ID NO: 556 - protein, 12 aa, Artificial Sequence
  Believed to be a synthetic construct
GRRRRKRLSHRT

SEQ ID NO: 557 - protein, 10 aa, Artificial Sequence
  Believed to be a synthetic construct
GRRRRRERNK

SEQ ID NO: 558 - protein, 16 aa, Artificial Sequence
  Believed to be a synthetic construct
GKHRHERGHHRDRRER

SEQ ID NO: 559 - protein, 17 aa, Artificial Sequence
  Believed to be a synthetic construct
GKKKRKLSNRESAKRSR

SEQ ID NO: 560 - protein, 10 aa, Artificial Sequence
  Believed to be a synthetic construct
MIIYRDLISH

SEQ ID NO: 561 - protein, 9 aa, Artificial Sequence
  Believed to be a synthetic construct
MIIYRDLIS

SEQ ID NO: 562 - protein, 8 aa, Artificial Sequence
  Believed to be a synthetic construct
MIIYRDLI

SEQ ID NO: 563 - protein, 9 aa, Artificial Sequence
  Believed to be a synthetic construct
IIYRDLISH

SEQ ID NO: 564 - protein, 7 aa, Artificial Sequence
  Believed to be a synthetic construct
MIIYRDL

SEQ ID NO: 565 - protein, 6 aa, Artificial Sequence
  Believed to be a synthetic construct
MIIYRD

SEQ ID NO: 566 - protein, 8 aa, Artificial Sequence
  Believed to be a synthetic construct
IYRDLISH

SEQ ID NO: 567 - protein, 9 aa, Artificial Sequence
  Believed to be a synthetic construct
AIIYRDLIS

SEQ ID NO: 568 - protein, 9 aa, Artificial Sequence
  Believed to be a synthetic construct
MAIYRDLIS

SEQ ID NO: 569 - protein, 9 aa, Artificial Sequence
  Believed to be a synthetic construct
MIAYRDLIS

SEQ ID NO: 570 - protein, 9 aa, Artificial Sequence
  Believed to be a synthetic construct
MIIARDLIS

SEQ ID NO: 571 - protein, 9 aa, Artificial Sequence
  Believed to be a synthetic construct
MIIYADLIS

SEQ ID NO: 572 - protein, 9 aa, Artificial Sequence
  Believed to be a synthetic construct
MIIYRALIS

SEQ ID NO: 573 - protein, 9 aa, Artificial Sequence
  Believed to be a synthetic construct
MIIYRDAIS

SEQ ID NO: 574 - protein, 9 aa, Artificial Sequence
  Believed to be a synthetic construct
MIIYRDLAS

SEQ ID NO: 575 - protein, 9 aa, Artificial Sequence
  Believed to be a synthetic construct
MIIYRDLIA

SEQ ID NO: 576 - protein, 11 aa, Artificial Sequence
  Believed to be a synthetic construct
MIIYRDLISKK

SEQ ID NO: 577 - protein, 10 aa, Artificial Sequence
  Believed to be a synthetic construct
MIIYRDKKSH

SEQ ID NO: 578 - protein, 10 aa, Artificial Sequence
  Believed to be a synthetic construct
MIIFRDLISH

SEQ ID NO: 579 - protein, 10 aa, Artificial Sequence
  Believed to be a synthetic construct
MIISRDLISH

SEQ ID NO: 580 - protein, 10 aa, Artificial Sequence
  Believed to be a synthetic construct
QIISRDLISH

SEQ ID NO: 581 - protein, 10 aa, Artificial Sequence
  Believed to be a synthetic construct
CIISRDLISH

SEQ ID NO: 582 - protein, 12 aa, Artificial Sequence
  Believed to be a synthetic construct
MIIYRALISHKK

SEQ ID NO: 583 - protein, 12 aa, Artificial Sequence
  Believed to be a synthetic construct
MIIYRIAASHKK

SEQ ID NO: 584 - protein, 10 aa, Artificial Sequence
  Believed to be a synthetic construct
MIIRRDLISE

SEQ ID NO: 585 - protein, 10 aa, Artificial Sequence
  Believed to be a synthetic construct
MIIYRAEISH

SEQ ID NO: 586 - protein, 10 aa, Artificial Sequence
  Believed to be a synthetic construct
MIIYARRAEE

SEQ ID NO: 587 - protein, 12 aa, Artificial Sequence
  Believed to be a synthetic construct
MIIFRIAASHKK

SEQ ID NO: 588 - protein, 12 aa, Artificial Sequence
  Believed to be a synthetic construct
MIIFRALISHKK

SEQ ID NO: 589 - protein, 12 aa, Artificial Sequence
  Believed to be a synthetic construct
MIIFRAAASHKK

SEQ ID NO: 590 - protein, 12 aa, Artificial Sequence
  Believed to be a synthetic construct
FIIFRIAASHKK

SEQ ID NO: 591 - protein, 12 aa, Artificial Sequence
  Believed to be a synthetic construct
LIIFRIAASHKK

SEQ ID NO: 592 - protein, 12 aa, Artificial Sequence
  Believed to be a synthetic construct
WIIFRIAASHKK

SEQ ID NO: 593 - protein, 12 aa, Artificial Sequence
  Believed to be a synthetic construct
WIIFRAAASHKK

SEQ ID NO: 594 - protein, 12 aa, Artificial Sequence
  Believed to be a synthetic construct
WIIFRALISHKK

SEQ ID NO: 595 - protein, 12 aa, Artificial Sequence
  Believed to be a synthetic construct
MIIFRIAAYHKK

SEQ ID NO: 596 - protein, 12 aa, Artificial Sequence
  Believed to be a synthetic construct
WIIFRIAAYHKK

SEQ ID NO: 597 - protein, 12 aa, Artificial Sequence
  Believed to be a synthetic construct
MIIFRIAATHKK

SEQ ID NO: 598 - protein, 12 aa, Artificial Sequence
  Believed to be a synthetic construct
WIIFRIAATHKK

SEQ ID NO: 599 - protein, 12 aa, Artificial Sequence
  Believed to be a synthetic construct
MIIFKIAASHKK

SEQ ID NO: 600 - protein, 12 aa, Artificial Sequence
  Believed to be a synthetic construct
WIIFKIAASHKK

SEQ ID NO: 601 - protein, 12 aa, Artificial Sequence
  Believed to be a synthetic construct
MIIFAIAASHKK

SEQ ID NO: 602 - protein, 12 aa, Artificial Sequence
  Believed to be a synthetic construct
LIIFRILISHKK

SEQ ID NO: 603 - protein, 12 aa, Artificial Sequence
  Believed to be a synthetic construct
MIIFRILISHKK

SEQ ID NO: 604 - protein, 12 aa, Artificial Sequence
  Believed to be a synthetic construct
LIIFRILISHRR

SEQ ID NO: 605 - protein, 12 aa, Artificial Sequence
  Believed to be a synthetic construct
LIIFRILISHHH

SEQ ID NO: 606 - protein, 11 aa, Artificial Sequence
  Believed to be a synthetic construct
LIIFRILISHK

SEQ ID NO: 607 - protein, 11 aa, Artificial Sequence
  Believed to be a synthetic construct
LIIFRILISHR

SEQ ID NO: 608 - protein, 10 aa, Artificial Sequence
  Believed to be a synthetic construct
LIIFRILISH

SEQ ID NO: 609 - protein, 12 aa, Artificial Sequence
  Believed to be a synthetic construct
LIIFAIAASHKK

SEQ ID NO: 610 - protein, 12 aa, Artificial Sequence
  Believed to be a synthetic construct
LIIFAILISHKK

SEQ ID NO: 611 - protein, 20 aa, Artificial Sequence
  Believed to be a synthetic construct
RILQQLLFIHFRIGCRHSRI

SEQ ID NO: 612 - protein, 17 aa, Artificial Sequence
  Believed to be a synthetic construct
RILQQLLFIHFRIGCRH

SEQ ID NO: 613 - protein, 15 aa, Artificial Sequence
  Believed to be a synthetic construct
RILQQLLFIHFRIGC

SEQ ID NO: 614 - protein, 10 aa, Artificial Sequence
  Believed to be a synthetic construct
RIFIHFRIGC

SEQ ID NO: 615 - protein, 8 aa, Artificial Sequence
  Believed to be a synthetic construct
RIFIRIGC

SEQ ID NO: 616 - protein, 11 aa, Artificial Sequence
  Believed to be a synthetic construct
RILQQLLFIHF

SEQ ID NO: 617 - protein, 6 aa, Artificial Sequence
  Believed to be a synthetic construct
RIFIGC

SEQ ID NO: 618 - protein, 6 aa, Artificial Sequence
  Believed to be a synthetic construct
FIRIGC

SEQ ID NO: 619 - protein, 22 aa, Artificial Sequence
  Believed to be a synthetic construct
DTWAGVEAIIRILQQLLFIHFR

SEQ ID NO: 620 - protein, 5 aa, Artificial Sequence
  Believed to be a synthetic construct
IGCRH

SEQ ID NO: 621 - protein, 16 aa, Artificial Sequence
  Believed to be a synthetic construct
RQIKIWFQNRRMKWKK

SEQ ID NO: 622 - protein, 23 aa, Artificial Sequence
  Believed to be a synthetic construct
GYGRKKRRGRRRTHRLPRRRRRR

SEQ ID NO: 623 - protein, 12 aa, Artificial Sequence
  Believed to be a synthetic construct
KRIIQRILSRNS

SEQ ID NO: 624 - protein, 12 aa, Artificial Sequence
  Believed to be a synthetic construct
KRIHPRLTRSIR

SEQ ID NO: 625 - protein, 12 aa, Artificial Sequence
  Believed to be a synthetic construct
PPRLRKRRQLNM

SEQ ID NO: 626 - protein, 12 aa, Artificial Sequence
  Believed to be a synthetic construct
PIRRRKKLRRLK

SEQ ID NO: 627 - protein, 12 aa, Artificial Sequence
  Believed to be a synthetic construct
RRQRRTSKLMKR

SEQ ID NO: 628 - protein, 12 aa, Artificial Sequence
  Believed to be a synthetic construct
MHKRPTTPSRKM

SEQ ID NO: 629 - protein, 12 aa, Artificial Sequence
  Believed to be a synthetic construct
RQRSRRRPLNIR

SEQ ID NO: 630 - protein, 12 aa, Artificial Sequence
  Believed to be a synthetic construct
RIRMIQNLIKKT

SEQ ID NO: 631 - protein, 12 aa, Artificial Sequence
  Believed to be a synthetic construct
SRRKRQRSNMRI

SEQ ID NO: 632 - protein, 12 aa, Artificial Sequence
  Believed to be a synthetic construct
QRIRKSKISRTL

SEQ ID NO: 633 - protein, 12 aa, Artificial Sequence
  Believed to be a synthetic construct
PSKRLLHNNLRR

SEQ ID NO: 634 - protein, 12 aa, Artificial Sequence
  Believed to be a synthetic construct
HRHIRRQSLIML

SEQ ID NO: 635 - protein, 12 aa, Artificial Sequence
  Believed to be a synthetic construct
PQNRLQIRRHSK

SEQ ID NO: 636 - protein, 12 aa, Artificial Sequence
  Believed to be a synthetic construct
PPHNRIQRRLNM

SEQ ID NO: 637 - protein, 12 aa, Artificial Sequence
  Believed to be a synthetic construct
SMLKRNHSTSNR

SEQ ID NO: 638 - protein, 12 aa, Artificial Sequence
  Believed to be a synthetic construct
GSRHPSLIIPRQ

SEQ ID NO: 639 - protein, 12 aa, Artificial Sequence
  Believed to be a synthetic construct
SPMQKTMNLPPM

SEQ ID NO: 640 - protein, 12 aa, Artificial Sequence
  Believed to be a synthetic construct
NKRILIRIMTRP

SEQ ID NO: 641 - protein, 12 aa, Artificial Sequence
  Believed to be a synthetic construct
HGWZIHGLLHRA

SEQ ID NO: 642 - protein, 11 aa, Artificial Sequence
  Believed to be a synthetic construct
AVPAKKRZKSV

SEQ ID NO: 643 - protein, 11 aa, Artificial Sequence
  Believed to be a synthetic construct
PNTRVRPDVSF

SEQ ID NO: 644 - protein, 12 aa, Artificial Sequence
  Believed to be a synthetic construct
LTRNYEAWVPTP

SEQ ID NO: 645 - protein, 13 aa, Artificial Sequence
  Believed to be a synthetic construct
SAETVESCLAKSH

SEQ ID NO: 646 - protein, 12 aa, Artificial Sequence
  Believed to be a synthetic construct
YSHIATLPFTPT

SEQ ID NO: 647 - protein, 12 aa, Artificial Sequence
  Believed to be a synthetic construct
SYIQRTPSTTLP

SEQ ID NO: 648 - protein, 12 aa, Artificial Sequence
  Believed to be a synthetic construct
AVPAENALNNPF

SEQ ID NO: 649 - protein, 12 aa, Artificial Sequence
  Believed to be a synthetic construct
SFHQFARATLAS

SEQ ID NO: 650 - protein, 12 aa, Artificial Sequence
  Believed to be a synthetic construct
QSPTDFTFPNPL

SEQ ID NO: 651 - protein, 12 aa, Artificial Sequence
  Believed to be a synthetic construct
HFAAWGGWSLVH

SEQ ID NO: 652 - protein, 12 aa, Artificial Sequence
  Believed to be a synthetic construct
HIQLSPFSQSWR

SEQ ID NO: 653 - protein, 12 aa, Artificial Sequence
  Believed to be a synthetic construct
LTMPSDLQPVLW

SEQ ID NO: 654 - protein, 12 aa, Artificial Sequence
  Believed to be a synthetic construct
FQPYDHPAEVSY

SEQ ID NO: 655 - protein, 12 aa, Artificial Sequence
  Believed to be a synthetic construct
FDPFFWKYSPRD

SEQ ID NO: 656 - protein, 12 aa, Artificial Sequence
  Believed to be a synthetic construct
FAPWDTASFMLG

SEQ ID NO: 657 - protein, 12 aa, Artificial Sequence
  Believed to be a synthetic construct
FTYKNFFWLPEL

SEQ ID NO: 658 - protein, 12 aa, Artificial Sequence
  Believed to be a synthetic construct
SATGAPWKMWVR

SEQ ID NO: 659 - protein, 12 aa, Artificial Sequence
  Believed to be a synthetic construct
SLGWMLPFSPPF

SEQ ID NO: 660 - protein, 12 aa, Artificial Sequence
  Believed to be a synthetic construct
SHAFTWPTYLQL

SEQ ID NO: 661 - protein, 12 aa, Artificial Sequence
  Believed to be a synthetic construct
SHNWLPLWPLRP

SEQ ID NO: 662 - protein, 12 aa, Artificial Sequence
  Believed to be a synthetic construct
SWLPYPWHVPSS

SEQ ID NO: 663 - protein, 12 aa, Artificial Sequence
  Believed to be a synthetic construct
SWWTPWHVHSES

SEQ ID NO: 664 - protein, 12 aa, Artificial Sequence
  Believed to be a synthetic construct
SWAQHLSLPPVL

SEQ ID NO: 665 - protein, 12 aa, Artificial Sequence
  Believed to be a synthetic construct
SSSIFPPWLSFF

SEQ ID NO: 666 - protein, 12 aa, Artificial Sequence
  Believed to be a synthetic construct
LNVPPSWFLSQR

SEQ ID NO: 667 - protein, 12 aa, Artificial Sequence
  Believed to be a synthetic construct
LDITPFLSLTLP

SEQ ID NO: 668 - protein, 12 aa, Artificial Sequence
  Believed to be a synthetic construct
LPHPVLHMGPLR

SEQ ID NO: 669 - protein, 12 aa, Artificial Sequence
  Believed to be a synthetic construct
VSKQPYYMWNGN

SEQ ID NO: 670 - protein, 12 aa, Artificial Sequence
  Believed to be a synthetic construct
NYTTYKSHFQDR

SEQ ID NO: 671 - protein, 12 aa, Artificial Sequence
  Believed to be a synthetic construct
AIPNNQLGFPFK

SEQ ID NO: 672 - protein, 12 aa, Artificial Sequence
  Believed to be a synthetic construct
NIENSTLATPLS

SEQ ID NO: 673 - protein, 12 aa, Artificial Sequence
  Believed to be a synthetic construct
YPYDANHTRSPT

SEQ ID NO: 674 - protein, 12 aa, Artificial Sequence
  Believed to be a synthetic construct
DPATNPGPHFPR

SEQ ID NO: 675 - protein, 12 aa, Artificial Sequence
  Believed to be a synthetic construct
TLPSPLALLTVH

SEQ ID NO: 676 - protein, 12 aa, Artificial Sequence
  Believed to be a synthetic construct
HPGSPFPPEHRP

SEQ ID NO: 677 - protein, 12 aa, Artificial Sequence
  Believed to be a synthetic construct
TSHTDAPPARSP

SEQ ID NO: 678 - protein, 12 aa, Artificial Sequence
  Believed to be a synthetic construct
MTPSSLSTLPWP

SEQ ID NO: 679 - protein, 12 aa, Artificial Sequence
  Believed to be a synthetic construct
VLGQSGYLMPMR

SEQ ID NO: 680 - protein, 12 aa, Artificial Sequence
  Believed to be a synthetic construct
QPIIITSPYLPS

SEQ ID NO: 681 - protein, 12 aa, Artificial Sequence
  Believed to be a synthetic construct
TPKTMTQTYDFS

SEQ ID NO: 682 - protein, 12 aa, Artificial Sequence
  Believed to be a synthetic construct
NSGTMQSASRAT

SEQ ID NO: 683 - protein, 12 aa, Artificial Sequence
  Believed to be a synthetic construct
QAASRVENYMHR

SEQ ID NO: 684 - protein, 12 aa, Artificial Sequence
  Believed to be a synthetic construct
HQHKPPPLTNNW

SEQ ID NO: 685 - protein, 12 aa, Artificial Sequence
  Believed to be a synthetic construct
SNPWDSLLSVST

SEQ ID NO: 686 - protein, 12 aa, Artificial Sequence
  Believed to be a synthetic construct
KTIEAHPPYYAS

SEQ ID NO: 687 - protein, 12 aa, Artificial Sequence
  Believed to be a synthetic construct
EPDNWSLDFPRR

SEQ ID NO: 688 - protein, 12 aa, Artificial Sequence
  Believed to be a synthetic construct
HQHKPPPLTNNW

SEQ ID NO: 689 - protein, 20 aa, Artificial Sequence
  Believed to be a synthetic construct
GLWRALWRLLRSLWRLLWKA

SEQ ID NO: 690 - protein, 21 aa, Artificial Sequence
  Believed to be a synthetic construct
GLWRALWRALWRSLWKLKRKV

SEQ ID NO: 691 - protein, 20 aa, Artificial Sequence
  Believed to be a synthetic construct
GLWRALWRALRSLWKLKRKV

SEQ ID NO: 692 - protein, 20 aa, Artificial Sequence
  Believed to be a synthetic construct
GLWRALWRGLRSLWKLKRKV

SEQ ID NO: 693 - protein, 20 aa, Artificial Sequence
  Believed to be a synthetic construct
GLWRALWRGLRSLWKKKRKV

SEQ ID NO: 694 - protein, 21 aa, Artificial Sequence
  Believed to be a synthetic construct
GLWRALWRALWRSLWKLKWKV

SEQ ID NO: 695 - protein, 21 aa, Artificial Sequence
  Believed to be a synthetic construct
GLWRALWRALWRSLWKSKRKV

SEQ ID NO: 696 - protein, 21 aa, Artificial Sequence
  Believed to be a synthetic construct
GLWRALWRALWRSLWKKKRKV

SEQ ID NO: 697 - protein, 21 aa, Artificial Sequence
  Believed to be a synthetic construct
GLWRALWRALWRSLWKLKRKV

SEQ ID NO: 698 - protein, 27 aa, Artificial Sequence
  Believed to be a synthetic construct
GLWRALWRLLRSLWRLLWSQPKKKRKV

SEQ ID NO: 699 - protein, 11 aa, Artificial Sequence
  Believed to be a synthetic construct
YARAARRAARR

SEQ ID NO: 700 - protein, 11 aa, Artificial Sequence
  Believed to be a synthetic construct
PARAARRAARR

SEQ ID NO: 701 - protein, 11 aa, Artificial Sequence
  Believed to be a synthetic construct
YPRAARRAARR

SEQ ID NO: 702 - protein, 11 aa, Artificial Sequence
  Believed to be a synthetic construct
YRRAARRAARA

SEQ ID NO: 703 - protein, 11 aa, Artificial Sequence
  Believed to be a synthetic construct
YGRAARRAARR

SEQ ID NO: 704 - protein, 11 aa, Artificial Sequence
  Believed to be a synthetic construct
YAREARRAARR

SEQ ID NO: 705 - protein, 11 aa, Artificial Sequence
  Believed to be a synthetic construct
YEREARRAARR

SEQ ID NO: 706 - protein, 11 aa, Artificial Sequence
  Believed to be a synthetic construct
YKRAARRAARR

SEQ ID NO: 707 - protein, 11 aa, Artificial Sequence
  Believed to be a synthetic construct
YARKARRAARR

SEQ ID NO: 708 - protein, 11 aa, Artificial Sequence
  Believed to be a synthetic construct
YKRKARRAARR

SEQ ID NO: 709 - protein, 11 aa, Artificial Sequence
  Believed to be a synthetic construct
YGRRARRAARR

SEQ ID NO: 710 - protein, 11 aa, Artificial Sequence
  Believed to be a synthetic construct
YGRRARRRARR

SEQ ID NO: 711 - protein, 11 aa, Artificial Sequence
  Believed to be a synthetic construct
YGRRARRRRRR

SEQ ID NO: 712 - protein, 11 aa, Artificial Sequence
  Believed to be a synthetic construct
YGRRRRRRRRR

SEQ ID NO: 713 - protein, 11 aa, Artificial Sequence
  Believed to be a synthetic construct
YRRRRRRRRRR

SEQ ID NO: 714 - protein, 16 aa, Artificial Sequence
  Believed to be a synthetic construct
GKINLKALAALAKKIL

SEQ ID NO: 715 - protein, 39 aa, Artificial Sequence
  Believed to be a synthetic construct
GRKKRRQRRRPPQGRKKRRQRRRPPQGRKKRRQRRRPPQ

SEQ ID NO: 716 - protein, 16 aa, Artificial Sequence
  Believed to be a synthetic construct
RVIRVWFQNKRCKDKK

SEQ ID NO: 717 - protein, 22 aa, Artificial Sequence
  Believed to be a synthetic construct
GEQIAQLIAGYIDIILKKKKSK

SEQ ID NO: 718 - protein, 14 aa, Artificial Sequence
  Believed to be a synthetic construct
GRKKRRQRRRPPQC

SEQ ID NO: 719 - protein, 28 aa, Artificial Sequence
  Believed to be a synthetic construct
AAVALLPAVLLALLAPRKKRRQRRRPPQ

SEQ ID NO: 720 - protein, 29 aa, Artificial Sequence
  Believed to be a synthetic construct
AAVALLPAVLLALLAPRKKRRQRRRPPQC

SEQ ID NO: 721 - protein, 28 aa, Artificial Sequence
  Believed to be a synthetic construct
AAVALLPAVLLALLAPRKKRRQRRRPPQ

SEQ ID NO: 722 - protein, 29 aa, Artificial Sequence
  Believed to be a synthetic construct
RKKRRQRRRPPQCAAVALLPAVLLALLAP

SEQ ID NO: 723 - protein, 29 aa, Artificial Sequence
  Believed to be a synthetic construct
RRRQRRKRGGDIMGEWGNEIFGAIAGFLG

SEQ ID NO: 724 - protein, 25 aa, Artificial Sequence
  Believed to be a synthetic construct
YGRKKRRQRRRGCYGRKKRRQRRRG

SEQ ID NO: 725 - protein, 13 aa, Artificial Sequence
  Believed to be a synthetic construct
GRKKRRQRRRPPQ

SEQ ID NO: 726 - protein, 22 aa, Artificial Sequence
  Believed to be a synthetic construct
AAVALLPAVLLALLAPRRRRRR

SEQ ID NO: 727 - protein, 16 aa, Artificial Sequence
  Believed to be a synthetic construct
RLWRALPRVLRRLLRP

SEQ ID NO: 728 - protein, 28 aa, Artificial Sequence
  Believed to be a synthetic construct
AAVALLPAVLLALLAPSGASGLDKRDYV

SEQ ID NO: 729 - protein, 33 aa, Artificial Sequence
  Believed to be a synthetic construct
LLETLLKPFQCRICMRNFSTRQARRNHRRRHRR

SEQ ID NO: 730 - protein, 27 aa, Artificial Sequence
  Believed to be a synthetic construct
AAVACRICMRNFSTRQARRNHRRRHRR

SEQ ID NO: 731 - protein, 16 aa, Artificial Sequence
  Believed to be a synthetic construct
RQIKIWFQNRRMKWKK

SEQ ID NO: 732 - protein, 35 aa, Artificial Sequence
  Believed to be a synthetic construct
RQIKIWFQNRRMKWKKDIMGEWGNEIFGAIAGFLG

SEQ ID NO: 733 - protein, 37 aa, Artificial Sequence
  Believed to be a synthetic construct
SGRGKQGGKARAKAKTRSSRAGLQFPVGRVHRLLRKG

SEQ ID NO: 734 - protein, 38 aa, Artificial Sequence
  Believed to be a synthetic construct
SGRGKQGGKARAKAKTRSSRAGLQFPVGRVHRLLRKGC

SEQ ID NO: 735 - protein, 25 aa, Artificial Sequence
  Believed to be a synthetic construct
KKDGKKRKRSRKESYSVYVYKVLKQ

SEQ ID NO: 736 - protein, 36 aa, Artificial Sequence
  Believed to be a synthetic construct
KGSKKAVTKAQKKDGKKRKRSRKESYSVYVYKVLKQ

SEQ ID NO: 737 - protein, 27 aa, Artificial Sequence
  Believed to be a synthetic construct
GWTLNSAGYLLGKINLKALAALAKKIL

SEQ ID NO: 738 - protein, 18 aa, Artificial Sequence
  Believed to be a synthetic construct
KLALKLALKALKAALKLA

SEQ ID NO: 739 - protein, 21 aa, Artificial Sequence
  Believed to be a synthetic construct
KETWWETWWTEWSQPKKKRKV

SEQ ID NO: 740 - protein, 28 aa, Artificial Sequence
  Believed to be a synthetic construct
KETWWETWWTEWSQPGRKKRRQRRRPPQ

SEQ ID NO: 741 - protein, 15 aa, Artificial Sequence
  Believed to be a synthetic construct
RVIRWFQNKRCKDKK

SEQ ID NO: 742 - protein, 18 aa, Artificial Sequence
  Believed to be a synthetic construct
LGLLLRHLRHHSNLLANI

SEQ ID NO: 743 - protein, 16 aa, Artificial Sequence
  Believed to be a synthetic construct
KLWSAWPSLWSSLWKP

SEQ ID NO: 744 - protein, 22 aa, Artificial Sequence
  Believed to be a synthetic construct
GLGSLLKKAGKKLKQPKSKRKV

SEQ ID NO: 745 - protein, 4 aa, Artificial Sequence
  Believed to be a synthetic construct
YRFK

SEQ ID NO: 746 - protein, 13 aa, Artificial Sequence
  Believed to be a synthetic construct
YRFKYRFKYRLFK

SEQ ID NO: 747 - protein, 10 aa, Artificial Sequence
  Believed to be a synthetic construct
WRFKKSKRKV

SEQ ID NO: 748 - protein, 20 aa, Artificial Sequence
  Believed to be a synthetic construct
WRFKAAVALLPAVLLALLAP

SEQ ID NO: 749 - protein, 8 aa, Artificial Sequence
  Believed to be a synthetic construct
WRFKWRFK

SEQ ID NO: 750 - protein, 12 aa, Artificial Sequence
  Believed to be a synthetic construct
WRFKWRFKWRFK

SEQ ID NO: 751 - protein, 36 aa, Artificial Sequence; identical to SEQ ID NO: 736
  Believed to be a synthetic construct

SEQ ID NO: 752 - protein, 36 aa, Artificial Sequence
  Believed to be a synthetic construct
RGSRRAVTRAQRRDGRRRRRSRRESYSVYVYRVLRQ

SEQ ID NO: 753 - protein, 15 aa, Artificial Sequence
  Believed to be a synthetic construct
RVIRWFQNKRSKDKK

SEQ ID NO: 754 - protein, 27 aa, Artificial Sequence
  Believed to be a synthetic construct
GWTLNSAGYLLGKINLKALAALAKKIL

SEQ ID NO: 755 - protein, 28 aa, Artificial Sequence
  Believed to be a synthetic construct
AAVALLPAVLLALLAPRKKRRQRRRPPQ

SEQ ID NO: 756 - protein, 5 aa, Artificial Sequence
  Believed to be a synthetic construct
CWKKK

SEQ ID NO: 757 - protein, 10 aa, Artificial Sequence
  Believed to be a synthetic construct
CWKKKKKKKK

SEQ ID NO: 758 - protein, 15 aa, Artificial Sequence
  Believed to be a synthetic construct
CWKKKKKKKKKKKKK

SEQ ID NO: 759 - protein, 20 aa, Artificial Sequence
  Believed to be a synthetic construct
CWKKKKKKKKKKKKKKKKKK

SEQ ID NO: 760 - protein, 19 aa, Artificial Sequence
  Believed to be a synthetic construct
KKKKKKKKKKKKKKKKKKK

SEQ ID NO: 761 - protein, 12 aa, Artificial Sequence
  Believed to be a synthetic construct
APWHLSSQYSRT

SEQ ID NO: 762 - protein, 26 aa, Artificial Sequence
  Believed to be a synthetic construct
AAVALLPAVLLALLAKKNNLKDCGLF

SEQ ID NO: 763 - protein, 26 aa, Artificial Sequence
  Believed to be a synthetic construct
AAVALLPAVLLALLAKKNNLKECGLY

SEQ ID NO: 764 - protein, 43 aa, Artificial Sequence
  Believed to be a synthetic construct
AAVALLPAVLLALLAVTDQLGEDFFAVDLEAFLQEFGLLPEKE

SEQ ID NO: 765 - protein, 16 aa, Artificial Sequence
  Believed to be a synthetic construct
AAVALLPAVLLALLAK

SEQ ID NO: 766 - protein, 26 aa, Artificial Sequence
  Believed to be a synthetic construct
AHALCLTERQIKIWFQNRRMKWKKEN

SEQ ID NO: 767 - protein, 26 aa, Artificial Sequence
  Believed to be a synthetic construct
AHALCPPERQIKIWFQNRRMKWKKEN

SEQ ID NO: 768 - protein, 26 aa, Artificial Sequence
  Believed to be a synthetic construct
AYALCLTERQIKIWFANRRMKWKKEN

SEQ ID NO: 769 - protein, 34 aa, Artificial Sequence
  Believed to be a synthetic construct
GGVCPKILKKCRRDSDCPGACICRGNGYCGSGSD

SEQ ID NO: 770 - protein, 34 aa, Artificial Sequence
  Believed to be a synthetic construct
GGVCPKILAACRRDSDCPGACICRGNGYCGSGSD

SEQ ID NO: 771 - protein, 34 aa, Artificial Sequence
  Believed to be a synthetic construct
GGVCPAILKKCRRDSDCPGACICRGNGYCGSGSD

SEQ ID NO: 772 - protein, 34 aa, Artificial Sequence
  Believed to be a synthetic construct
GGVCPKILAKCRRDSDCPGACICRGNGYCGSGSD

SEQ ID NO: 773 - protein, 34 aa, Artificial Sequence
  Believed to be a synthetic construct
GGVCPKILKACRRDSDCPGACICRGNGYCGSGSD

SEQ ID NO: 774 - protein, 29 aa, Artificial Sequence
  Believed to be a synthetic construct
GLPVCGETCVGGTCNTPGCKCSWPVCTRN

SEQ ID NO: 775 - protein, 29 aa, Artificial Sequence
  Believed to be a synthetic construct
GLPVCGETCVGGTCNTPGCTCSWPKCTRN

SEQ ID NO: 776 - protein, 14 aa, Artificial Sequence
  Believed to be a synthetic construct
GRCTKSIPPICFPD

SEQ ID NO: 777 - protein, 16 aa, Artificial Sequence
  Believed to be a synthetic construct
RQIKIWFQNRRMKWKK

SEQ ID NO: 778 - protein, 33 aa, Artificial Sequence
  Believed to be a synthetic construct
RQIKIWFQNRRMKWKKTYADFIASGRTGRRNAI

SEQ ID NO: 779 - protein, 13 aa, Artificial Sequence
  Believed to be a synthetic construct
GRKKRRQRRRPPQ

SEQ ID NO: 780 - protein, 30 aa, Artificial Sequence
  Believed to be a synthetic construct
GRKKRRQRRRPPQTYADFIASGRTGRRNAI

SEQ ID NO: 781 - protein, 21 aa, Artificial Sequence
  Believed to be a synthetic construct
AGYLLGKINLKALAALAKKIL

SEQ ID NO: 782 - protein, 38 aa, Artificial Sequence
  Believed to be a synthetic construct
AGYLLGKINLKALAALAKKILTYADFIASGRTGRRNAI

SEQ ID NO: 783 - protein, 28 aa, Artificial Sequence
  Believed to be a synthetic construct
RRRRRRRRRRRTYADFIASGRTGRRNAI

SEQ ID NO: 784 - protein, 16 aa, Artificial Sequence
  Believed to be a synthetic construct
RQIKIWFQNRRMKWKK

SEQ ID NO: 785 - protein, 22 aa, Artificial Sequence
  Believed to be a synthetic construct
KCFQWQRNMRKVRGPPVSCIKR

SEQ ID NO: 786 - protein, 20 aa, Artificial Sequence
  Believed to be a synthetic construct
KLALKLALKALKAALKLAGC

SEQ ID NO: 787 - protein, 35 aa, Artificial Sequence
  Believed to be a synthetic construct
GGGARKKAAKAARKKAAKAARKKAAKAARKKAAKA

SEQ ID NO: 788 - protein, 14 aa, Artificial Sequence
  Believed to be a synthetic construct
GRKKRRQRRRPPQC

SEQ ID NO: 789 - protein, 19 aa, Artificial Sequence
  Believed to be a synthetic construct
TRQARRNRRRRWRERQRGC

SEQ ID NO: 790 - protein, 17 aa, Artificial Sequence
  Believed to be a synthetic construct
RRRRNRTRRNRRRVRGC

SEQ ID NO: 791 - protein, 21 aa, Artificial Sequence
  Believed to be a synthetic construct
KMTRAQRRAAARRNRWTARGC

SEQ ID NO: 792 - protein, 15 aa, Artificial Sequence
  Believed to be a synthetic construct
TRRQRTRRARRNRGC

SEQ ID NO: 793 - protein, 30 aa, Artificial Sequence; identical to SEQ ID NO: 394
  Believed to be a synthetic construct

SEQ ID NO: 794 - protein, 28 aa, Artificial Sequence
  Believed to be a synthetic construct
KRRIRRERNKMAAAKSRNRRRELTDTGC

SEQ ID NO: 795 - protein, 23 aa, Artificial Sequence
  Believed to be a synthetic construct
WLRRIKAWLRRIKALNRQLGVAA

SEQ ID NO: 796 - protein, 12 aa, Artificial Sequence
  Believed to be a synthetic construct
GRKKRRQRRRPP

SEQ ID NO: 797 - protein, 34 aa, Artificial Sequence
  Believed to be a synthetic construct
AKVKDEPQRRSARLSAKPAPPKPEPKPKKAPAKK

SEQ ID NO: 798 - protein, 5 aa, Unknown
  May be a covalent enzyme-substrate intermediate with saccharide distortion in a mutant T4 lysozyme
  misc_feature (2)..(5): Xaa can be any naturally occurring amino acid
AXXXX

SEQ ID NO: 799 - protein, 9 aa, Unknown
  Perhaps associated with human immunodeficiency virus
TPYDINQML

SEQ ID NO: 800 - protein, 8 aa, Unknown
  perhaps associated with human immunodeficiency virus
VPLRPMTY

SEQ ID NO: 801 - protein, 9 aa, Plasmodium falciparum
KPIVQYDNF

SEQ ID NO: 802 - protein, 16 aa, Unknown
  Perhaps associated with hepatitus C virus
GSVVIVGRIVLSGKPA

SEQ ID NO: 803 - protein, 21 aa, Unknown
  Perhaps associated with hepatitus C virus
KGSVVIVGRIVLSGKPAIIPK

SEQ ID NO: 804 - protein, 36 aa, Unknown
  May be a thrombin inhibited by aeruginosin298-A from a blue-green alga
TFGSGEADCGLRPLFEKKSLEDKTERELLESYIDGR

SEQ ID NO: 805 - protein, 10 aa, Unknown
  May be a thrombin inhibited by aeruginosin298-A from a blue-green alga
  misc_feature (9)..(9): Xaa can be any naturally occurring amino acid
DFEEIPEEXL

SEQ ID NO: 806 - protein, 4 aa, Unknown
  May be a thrombin inhibited by aeruginosin298-A from a blue-green alga
  misc_feature (1)..(1): Xaa can be any naturally occurring amino acid
  misc_feature (3)..(4): Xaa can be any naturally occurring amino acid
XLXX

SEQ ID NO: 807 - protein, 31 aa, Unknown
  May be related to a complex of troponin C with a fragment of troponin I
EEKRNRAITARRQHLKSVMLQIAATELEKEE

SEQ ID NO: 808 - protein, 27 aa, Homo sapiens [sequence omitted]

SEQ ID NO: 809 - protein, 5 aa, Unknown
  May be from a bovine source
TPGVY

SEQ ID NO: 810 - protein, 33 aa, Unknown
  May be a hirulog 3-thrombin complex
SGEADCGLRPLFEKKSLEDKTERELLESYIDGR

SEQ ID NO: 811 - protein, 20 aa, Unknown
  May be a hirulog 3-thrombin complex
  misc_feature (1)..(1): Xaa can be any naturally occurring amino acid
  misc_feature (3)..(3): Xaa can be any naturally occurring amino acid
  misc_feature (12)..(12): Xaa can be any naturally occurring amino acid
XPXGGGGGNGDXEEIPEEYL

SEQ ID NO: 812 - protein, 27 aa, Unknown [sequence omitted]

SEQ ID NO: 813 - protein, 6 aa, Unknown
  May be a bungarotoxin/nicotinic receptor peptide complex
KHWVYY

SEQ ID NO: 814 - protein, 28 aa, Unknown
  May be a complex of thrombin with a p1 moiety
EADCGLRPLFEKKSLEDKTERELLESYI

SEQ ID NO: 815 - protein, 10 aa, Homo sapiens
  misc_feature (9)..(9): Xaa can be any naturally occurring amino acid
DFEEIPEEXL

SEQ ID NO: 816 - protein, 36 aa, Homo sapiens; identical to SEQ ID NO: 804

SEQ ID NO: 817 - protein, 36 aa, Homo sapiens; identical to SEQ ID NO: 804

SEQ ID NO: 818 - protein, 10 aa, Unknown
  May be from a bovine source
CGVPAIQPVL

SEQ ID NO: 819 - protein, 8 aa, Unknown
  May be from human immunodeficiency virus
GGRKKYKL

SEQ ID NO: 820 - protein, 8 aa, Unknown
  May be from human immunodeficiency virus
GGKKKYQL

SEQ ID NO: 821 - protein, 8 aa, Unknown
  May be from human immunodeficiency virus
GGKKKYKL

SEQ ID NO: 822 - protein, 8 aa, Unknown
  May be from human immunodeficiency virus
GGKKKYRL

SEQ ID NO: 823 - protein, 8 aa, Unknown
  May be from human immunodeficiency virus
GGKKRYKL

SEQ ID NO: 824 - protein, 28 aa, Homo sapiens
EADCGLRPLFEKKSLEDKTERELLESYI

SEQ ID NO: 825 - protein, 10 aa, Homo sapiens
  misc_feature (9)..(9): Xaa can be any naturally occurring amino acid
DFEEIPEEXL

SEQ ID NO: 826 - protein, 13 aa, Homo sapiens
SDWRFLRGYHQYA

SEQ ID NO: 827 - protein, 14 aa, Homo sapiens
GSDWRFLRGYHQYA

SEQ ID NO: 828 - protein, 33 aa, Unknown
  May be from Homo sapiens
GTIVRFNRPFLMIIVPTDTQNIFFMSKVTNPKQ

SEQ ID NO: 829 - protein, 20 aa, Artificial Sequence
  Believed to be a synthetic construct
CGIQDTNSKKQSDTHLEETC

(19) United States
(12) Patent Application Publication
(10) Pub. No.: US 2023/0248837 A1
(43) Pub. Date: Aug. 10, 2023

(54) Technology for Modulating Targeting Chimeras Induced by Cell-Penetrating Peptide and Application Thereof

(71) Applicant: Miao LIU, Rizhao City (CN)

(72) Inventors: Miao LIU, Shenzhen City (CN); Radhakrishnan SRIDHAR, Shenzhen City (CN)

(21) Appl. No.: 17/853,839

(22) Filed: Jun. 29, 2022

Related U.S. Application Data
(60) Provisional application No. 63/217,115, filed on Jun. 30, 2021.

(51) Int. Cl. A61K 47/62; A61K 47/54

(52) U.S. Cl. CPC A61K 47/62; A61K 47/54

(57) ABSTRACT

The invention provides a modulating targeting chimera molecule induced by a cell-penetrating peptide comprising at least one cell-penetrating peptide module, at least one targeting peptide module and at least one small molecule ligand module connected with each other, wherein the targeting peptide module is a peptide sequence that can bind to a targeted protein. The characteristics and advantages of the invention are as follows: in the modulating targeting chimera molecule induced by the cell-penetrating peptide provided by the invention, a modulating design is adopted, each sequence or small molecule compound module with different functions can be replaced and superimposed as required, and cyclization or secondary microprotein structural modification can be performed on all peptide modules. Under this design idea, the application effect and scope of targeted drugs are greatly enhanced.

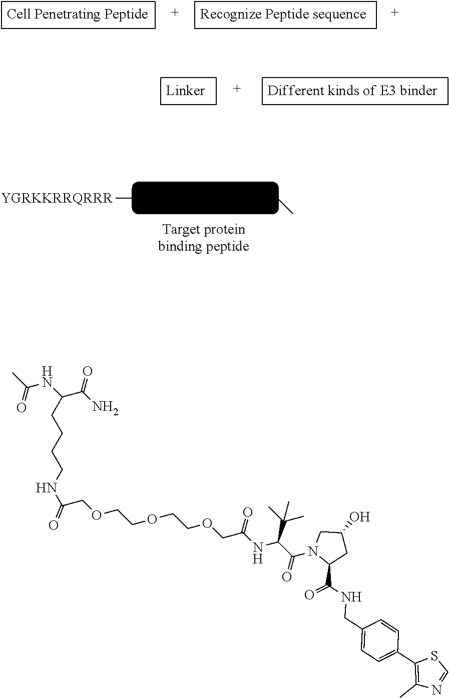

### CROSS REFERENCE TO RELATED APPLICATION(S)

This application claims the benefit of U.S. Provisional Patent Application No. 63/217,115, filed on Jun. 30, 2021, incorporated herein by reference as if fully set forth herein.

### REFERENCE TO AN ELECTRONIC SEQUENCE LISTING

The contents of the electronic sequence listing (DOP2121000006US Sequence Listing_S25.txt; Size: 228,742 bytes; and Date of Creation: Apr. 28, 2023) is incorporated herein by reference in its entirety.

### FIELD

The invention relates to the field of bioengineering, in particular to the technology for a modulating targeted chimera induced by a cell-penetrating peptide and application thereof.

### BACKGROUND

Technology for protein degraders has become popular in the world in recent years. The earliest and most popular technology is the technology of Proteolysis targeting Chimera (PROTAC). Structurally, PROTAC comprises three components: a small molecule E3 ubiquitin ligase ligand and a small molecule target protein ligand, as well as a specially designed “Linker” structure through which the two active ligands are connected and finally the active form of “PROTAC” of the small molecule triplet is formed with a structure as follows: a small molecule ligand (for targeting a target protein)+linker+small molecule ligand (for binding to E3 ligase). The target protein binds to the small molecule target protein ligand, and the E3 ligase ligand also binds to its ligand. A ubiquitin label is added to the target protein by the E3 ligase, and then after multiple rounds of ubiquitination, there are multiple ubiquitin labels. The target protein after polyubiquitination will be recognized and degraded by the proteasome.

The PROTAC technology has been successfully applied to the induced degradation of a variety of pathological proteins. In nature, a special recognition signal is required for E3 ubiquitin ligase to recruit and ubiquitinate its target protein. The appearance of the PROTAC technology enables E3 to ubiquitinate any protein. In this technology, a dual-function molecule is designed, with one end of which can bind to a target protein while the other end binds to E3 ligase, combining the two into a polymer. E3 then ubiquitinates the target protein and guides it into the degradation pathway. The most attractive aspect of the targeted protein degradation is that it can target those protein targets that are traditionally considered non-pharmaceutical, which may account for more than 80% of the human proteome. Since the targeted protein degradation strategy can selectively degrade proteins by binding to almost any site on the protein rather than the active site, theoretically this strategy can be used for any protein.

However, in actual research or operation, it is very difficult to screen out the small molecule ligand component of the above small molecule “triplet” PROTAC that binds to the target protein. Due to the inability to find suitable ligands for many targets, targeted drugs of which cannot be developed. Moreover, in nature, many protein targets are naturally not suitable for small molecule binding, which may lead to the failure of drug research and development of small molecule PROTAC.

Existing therapeutic drugs are mainly concentrated in two categories: small molecules and biologics. However, due to the limitations of their own biophysical properties, these two types of therapeutic drugs cannot effectively cover all these confirmed important molecular targets. Peptide drugs are another class of targeted molecules that have attracted widespread attention and interest. Similar to biological macromolecules, peptide molecules also have high binding ability and selectivity for targets, and have less off-target effects than small molecule drugs. The metabolites of peptides in the body are amino acids, which minimizes toxicity. Compared with small molecule drugs, peptide drugs have incomparable advantages, which are mainly reflected in the ease of modification, the specificity of target recognition, and the wide targeting range of peptide molecules.

### SUMMARY

An object of the present invention is to provide a technology for modulating targeting chimeras induced by a cell-penetrating peptide that can target a target protein thereby effectively degrading the target protein, and applications thereof, that is, a technology for Cell-penetrating-peptide Induced Targeting Chimera (CePPiTAC) for use in degrading the target protein.

The object of the present invention described above can be realized by using the following technical solutions:

The first object of the present invention is to provide a modulating targeting chimera molecule induced by a cell-penetrating peptide, comprising at least one cell-penetrating peptide module, at least one targeting peptide module, one (or no) small molecule Linker module and at least one small molecule ligand module connected with each other, wherein the targeting peptide module is a peptide sequence that can bind to a targeted protein.

Optionally, the modulating targeting chimera molecule induced by the cell-penetrating peptide described above further comprises at least one Linker module, wherein the targeting peptide module is chimeric with the small molecule ligand module through the Linker module.

Optionally, the modulating targeting chimera molecule induced by the cell-penetrating peptide described above, wherein the cell-penetrating peptide module is connected to the free end of the targeting peptide module and used to guide the targeting chimera molecule for penetrating the cell membrane.

Optionally, the modulating targeting chimera molecule induced by the cell-penetrating peptide described above, wherein the small molecule ligand module is a small molecule E3 ligand that can bind to E3 ligase, preferably, the protease degrader adapted to the small molecule E3 ligand is one or more of CRBN (Cereblon protein), VHL (von Hippel-Lindau) and IAP (Inhibitor of apoptosis proteins).

Optionally, the modulating targeting chimera molecule induced by the cell-penetrating peptide described above, wherein the cell-penetrating peptide module has an amino acid sequence of any one of SEQ ID NO: 1-SEQ ID NO: 3.

Optionally, the modulating targeting chimera molecule induced by the cell-penetrating peptide described above, wherein the targeting peptide module has an amino acid sequence of any one or more of SEQ ID NO: 4-SEQ ID NO: 17.

Optionally, the modulating targeting chimera molecule induced by the cell-penetrating peptide described above, wherein the Linker module is a small molecule compound with a structural formula shown in formula I:

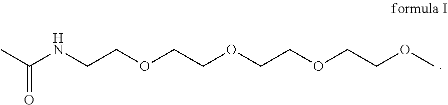 [SMILES: COCCOCCOCCOCCNC(C)=O]

Optionally, the modulating targeting chimera molecule induced by the cell-penetrating peptide described above, wherein:

when the adapted protease degrader is CRBN, the structural formula of the small molecule ligand module is shown in formula II:

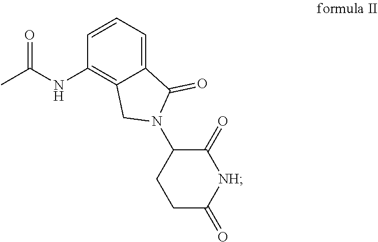 [SMILES: CC(=O)Nc1cccc2c1CN(C1CCC(=O)NC1=O)C2=O]

when the adapted protease degrader is VHL, the structural formula of the small molecule ligand module is shown in formula III:

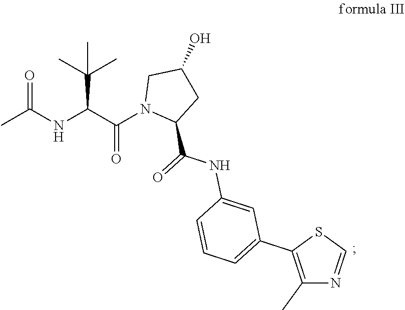 [SMILES: CC(=O)N[C@H](C(=O)N1C[C@H](O)C[C@H]1C(=O)Nc1cccc(-c2scnc2C)c1)C(C)(C)C]

and

when the adapted protease degrader is TAP, the structural formula of the small molecule ligand module is as shown in formula IV:

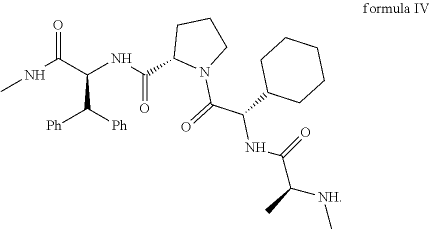

Optionally, the modulating targeting chimera molecule induced by the cell-penetrating peptide described above has a structure of any one or more of the following structures:

1) the cell-penetrating peptide of SEQ ID NO: 1+the targeting peptide of SEQ ID NO: 4+the Linker of formula I+the small molecule ligand of formula II;

2) the cell-penetrating peptide of SEQ ID NO: 2+the targeting peptide of SEQ ID NO: 5+the Linker of formula I+the small molecule ligand of formula III;

3) the cell-penetrating peptide of SEQ ID NO: 1+the targeting peptide of SEQ ID NO: 5+the Linker of formula I+the small molecule ligand of formula IV;

4) the cell-penetrating peptide of SEQ ID NO: 1+the targeting peptide of SEQ ID NO: 6+the Linker of formula I+the small molecule ligand of formula II;

5) the cell-penetrating peptide of SEQ ID NO: 1+the targeting peptide of SEQ ID NO: 7+the Linker of formula I+the small molecule ligand of formula III;

6) the cell-penetrating peptide of SEQ ID NO: 1+the targeting peptide of SEQ ID NO: 8+the Linker of formula I+the small molecule ligand of formula II;

7) the cell-penetrating peptide of SEQ ID NO: 1+the targeting peptide of SEQ ID NO: 9+the Linker of formula I+the small molecule ligand of formula III;

8) the cell-penetrating peptide of SEQ ID NO: 1+the targeting peptide of SEQ ID NO: 10+the Linker of formula I+the small molecule ligand of formula II;

9) the cell-penetrating peptide of SEQ ID NO: 1+the targeting peptide of SEQ ID NO: 11+the Linker of formula I+the small molecule ligand of formula IV;

10) the cell-penetrating peptide of SEQ ID NO: 1+the targeting peptide of SEQ ID NO: 12+the Linker of formula I+the small molecule ligand of formula III;

11) the cell-penetrating peptide of SEQ ID NO: 1+the targeting peptide of SEQ ID NO: 13+the Linker of formula I+the small molecule ligand of formula III;

12) the cell-penetrating peptide of SEQ ID NO: 3+the targeting peptide of SEQ ID NO: 14+the Linker of formula I+the small molecule ligand of formula IV;

13) the cell-penetrating peptide of SEQ ID NO: 1+the targeting peptide of SEQ ID NO: 15+the Linker of formula I+the small molecule ligand of formula II;

14) the cell-penetrating peptide of SEQ ID NO: 1+the targeting peptide of SEQ ID NO: 16+the Linker of formula I+the small molecule ligand of formula II;

15) the cell-penetrating peptide of SEQ ID NO: 2+the targeting peptide of SEQ ID NO: 17+the Linker of formula I+the small molecule ligand of formula IV;

16) the cell-penetrating peptide of SEQ ID NO: 3+the targeting peptide of SEQ ID NO: 14+the Linker of formula I+(dual E3 ligands: the small molecule ligand of formula II+the small molecule ligand of formula III); and

17) the cell-penetrating peptide of SEQ ID NO: 1+(dual targets: the targeting peptide of SEQ ID NO: 4+the targeting peptide of SEQ ID NO: 5)+the Linker of formula I+the small molecule ligand of formula II.

Optionally, the modulating targeting chimera molecule induced by the cell-penetrating peptide described above, wherein the targeting peptide module further comprises a modified stapled peptide sequence or circular peptide sequence, and the stapled peptide sequence or circular peptide sequence has a function of cell penetration. In this case, the modulating targeting chimera molecule induced by the cell-penetrating peptide can be achieved without the cell-penetrating peptide.

Optionally, the modulating targeting chimera molecule induced by the cell-penetrating peptide described above, wherein the stapled peptide has a structural formula shown in formula V:

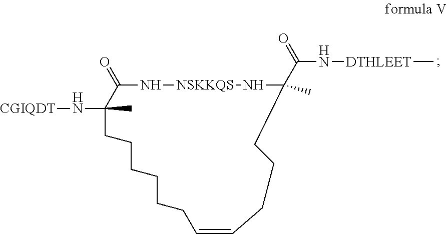

and

the cyclic peptide has a structural formula shown in formula VI:

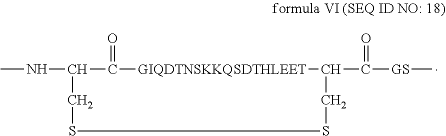

Optionally, the modulating targeting chimera molecule induced by the cell-penetrating peptide described above, wherein the modulating targeting chimera molecule induced by the cell-penetrating peptide containing the stapled peptide has the structure as follows: the stapled peptide of formula V+the Linker of formula I+the small molecule ligand of formula II.

Optionally, the modulating targeting chimera molecule induced by the cell-penetrating peptide described above, wherein the modulating targeting chimera molecule induced by the cell-penetrating peptide containing the circular peptide has the structure as follows: the circular peptide of formula VI+the Linker of formula I+the small molecule ligand of formula II.

The second object of the present invention is to provide a use of the modulating targeting chimera molecule induced by the cell-penetrating peptide described above in preparing a product for degrading a targeted protein or a product for degrading a targeted protein with a mutant amino acid position.

Optionally, the use described above, wherein the degraded targeted protein comprises one or more of the novel coronavirus S protein HR2, novel coronavirus N protein, novel coronavirus M protein, novel coronavirus E protein, novel coronavirus Orf6 protein, LAG-3 protein, Her2 protein, SHP-2 protein, STAT5B protein, MUC16 protein, CTLA-4 protein, PCSK9 protein, PD-1 protein, PD-L1 protein and KRAS protein with G12V mutation.

Based on the above technical description, the core idea of the present invention is to connect multiple freely transformable “module” sequences or small molecular compounds to form “modulating” targeting chimera molecules with strong targeting ability, good cell-penetration effect and high degradation efficiency.

In the technical solution of the present invention, the most basic composition is a cell-penetrating peptide module, a targeting peptide module and a small molecule ligand module, which are connected to each other. The further composition can be a cell-penetrating peptide module, a targeting peptide module, a Linker module and a small molecule ligand module, thereby forming the basic structure of the cell-penetrating peptide-targeting peptide-Linker-small molecule ligand.

The basic structure of the cell-penetrating peptide-targeting peptide-small molecule ligand can direct the small molecule ligand to the target protein. Although some chimeras under this basic structure can exert targeted therapeutic properties, they have certain defects. For example, the connection of the three is not stable and easy to fall off.

The cell-penetrating peptide-targeting peptide-Linker-small molecule ligand is an upgraded structure of the above basic structure, which overcomes the defects of poor cell penetration performance of targeting peptides and unstable direct connection between targeting peptides and small molecule ligands. Through penetrating the membrane by the cell-penetrating peptide, the targeting peptide can be directed to the target protein, so as to achieve the effect of targeted penetration. Using the Linker to connect the two can effectively reduce the probability of falling off; meanwhile, it overcomes defects of both poor cell penetration and unstable connection, solving the technical effect of targeted therapy almost perfectly and improving the targeting efficiency. The most important technical point is that, by using the cell-penetrating peptide+targeting peptide in this optimal structure for “replacing” the targeting proteins in the existing PROTAC technology, the selectivity of targeted peptides to targeted proteins can be greatly expanded, and almost all known target proteins can be targeted and degraded through a linked small molecule ligand (E3).

Penetration into cells through cell membranes is a prerequisite for the functioning of many biological macromolecules whose targets are in the cell. However, the biological barrier function of the biofilm prevents many macromolecules from entering the cell, thus limiting the application of these substances in the field of therapy to a great extent. Therefore, how to guide these substances to penetrate the cell membrane is an urgent problem to be solved. As an intermediate product of protein hydrolysis, peptide has poor ability for cell penetration. In recent years, with the development of technology, it has gradually been discovered that the transactivator (TAT) in human immunodeficiency virus (HIV) can effectively pass through the cell membrane and enter the cell, followed by a variety of proteins capable of penetrating cell membranes were discovered and named as cell-penetrating peptides (CPPs). In general, cell-penetrating peptides are usually peptide molecules of no more than 30 amino acids that can independently pass through the cell membrane independent of specific membrane receptors. Used as a tool for intracellular transport of bioactive molecules, these cell-penetrating peptides have characteristics of low toxicity, convenience and effectiveness compared with other iontophoresis and nano carriers, and play a more and more important role in drug development. Even some drugs containing CPPs have passed FDA's clinical trials.

In the Cell-penetrating-peptide Induced Targeting Chimera (CePPiTAC) technology of the present invention, the peptide that can bind to the target protein “replaces” the component of the small molecule target protein ligand in the common “triplet” PROTAC structure, by which the Linker and the small molecule E3 ligand are connected, and a sequence of a cell-penetrating peptide is added to form a structure as: the cell-penetrating peptide (cell-penetrating peptide)+peptide (targeting the target)+Linker+E3 ligand. The Linker can be removed if necessary, allowing the peptide containing the cell-penetrating peptide (targeting the target) to be directly linked to the E3 ligand.

The drug synthesized by the present invention is a complex of peptides and small molecules, a small molecule Linker can be connected therebetween or be removed. At the non-small molecular junction of the peptide, a cell-penetrating peptide sequence can be added, which can carry the peptide-small molecule complex (CePPiTAC complex) into the cell. Meanwhile, the component of the peptide targeting a target protein can bind to the target protein, while the small molecule E3 ligand at the other end of the Linker can bind to E3 ligase and initiate E3 ubiquitinase reaction to ubiquitinate the target protein, so that the 26S protease in the cell can recognize the target protein for degradation.

The features and advantages of the present invention are as follows: the modulating targeting chimera molecule induced by the cell-penetrating peptide provided by the present invention can penetrate the cell membrane and target all targeted proteins through interconnected cell-penetrating peptides, targeting peptides and small molecule ligands, in which the connected small molecule ligands can bind to the immobilized ligase and initiate a ubiquitinase reaction, thereby ubiquitinating the target protein, so that intracellular proteases can target and recognize the target protein for degradation, thus the targeted drugs can be screened out more extensively. Since the peptide module is used to bind the target protein, in theory, all target proteins can be targeted, which cannot be achieved by other degrader technologies in the past. In addition, since in this technology, ubiquitination is achieved by combining high-efficiency small molecule E3 ligands, it is much more efficient than other peptide-based PROTAC/degraders that use peptide ligands. In most cases, degradation of target proteins can be achieved at the nmol level in cell experiments.

In this chimera molecule structure, a Linker can also be added, and the addition of the Linker can further solidify the connection between the targeting peptide and the small molecule ligand.

Meanwhile, in the targeting chimera molecule provided by the present invention, a modulating design is adopted, and each sequence or small molecule compound module with different functions can be replaced and superimposed as needed. Under this design idea, the application effect and scope of targeted drugs are greatly enhanced.

Targets that can be developed for the small molecule triplet PROTAC are Limited, while Peptide-Based PROTAC/degrader has low degradation efficiency, and the target can only be degraded on the cell at the umol level. However, under this technology, all the targets can be targeted, while high-efficiency degradation (cellular degradation) can also be achieved at the nmol level, truly achieving the purpose of “drugs for all diseases”. Meanwhile, the present invention also has subversive significance to the previous technical concepts. Taking virus-related proteins as an example, the relevant targets for viruses were mainly related proteins that infect human cells (such as the S protein of novel coronavirus), as well as enzymes needed for virus synthesis, but there were few targets to choose. However, in this technology, the peptide sequence is used to bind to the inactive site of the target, which can target all proteins of all viruses for degradation, and is also effective in overcoming drug resistance caused by virus mutation, which greatly improves the possibility and convenience of successful research and development of drugs for virus. In addition, under this technology, a single mutation of a certain target can be degraded, while the wild-type homologous protein without such a mutation will not be affected or be less affected, which cannot be realized for other degrader technologies such as PROTAC.

### BRIEF DESCRIPTION OF DRAWINGS

In order to illustrate the technical solutions in the embodiments of the present invention more clearly, the following briefly introduces the accompanying drawings used in the description of the embodiments. Obviously, the accompanying drawings in the following description are only some embodiments of the present invention. For those of ordinary skill in the art, other drawings can also be obtained from these drawings without creative effort.

FIG. 1 shows a conventional design of the modulating targeting chimera molecule induced by the cell-penetrating peptide (targeting degrader) described in Example 2 of the present invention, i.e., a universal pattern of the cell-penetrating peptide-targeting peptide-Linker-small molecule ligand.

FIG. 2 shows the modulating targeting chimera molecule induced by the cell-penetrating peptide described in Example 3 of the present invention, which is specially designed for one or a certain class of refractory pathogenic proteins, and can combine the targeting peptide with two or multiple different E3 ligase conjugates to efficiently degrade target pathogenic proteins.

FIG. 3 shows the modulating targeting chimera molecule induced by the cell-penetrating peptide described in Example 4 of the present invention, which can effectively target multiple targets related to the formation of protein-protein complexes of pathogenic proteins, thereby achieving the efficacy of targeting pathogenic proteins and effectively inhibiting the entire pathogenic pathway as well as completely inhibiting a specific disease by degrading multiple targets.

FIG. 4 shows a process flow chart of the solid-phase synthesis of the peptide in Example 5, wherein the synthetic product peptide is marked as 1.

FIG. 5 shows the synthesis reaction of the synthetic compound of lenalidomide and succinic anhydride in Example 5, wherein lenalidomide is marked as 2, succinic anhydride as 3, and the synthetic product as 4.

FIG. 6 shows a process flow chart of the solid-phase synthesis of the LEN-binding peptide in Example 5, wherein the synthesis product (mixture of diastereomers) is marked as 5.

FIG. 7A-FIG. 7C show the modulating targeting chimera molecule induced by the cell-penetrating peptide for degrading novel coronavirus S protein HR2 in an example of the present invention and the effect verification thereof, in which FIG. 7A is a structural diagram of the modulating targeting chimera molecule induced by the cell-penetrating peptide, FIG. 7B is the verification of the degradation effect on the protein, and FIG. 7C shows the verification by a protease inhibitor MG132 that the effect is indeed produced by the protease degrader.

FIG. 8A-FIG. 8C show the modulating targeting chimera molecule induced by the cell-penetrating peptide for degrading novel coronavirus N protein in an example of the present invention and the effect verification thereof, in which FIG. 8A is a structural diagram of the modulating targeting chimera molecule induced by the cell-penetrating peptide, FIG. 8B is the verification of the degradation effect on the protein, and FIG. 8C shows the verification by a protease inhibitor MG132 that the effect is indeed produced by the protease degrader.

FIG. 9A-FIG. 9C show the modulating targeting chimera molecule induced by the cell-penetrating peptide for degrading novel coronavirus M protein in an example of the present invention and the effect verification thereof, in which FIG. 9A is a structural diagram of the modulating targeting chimera molecule induced by the cell-penetrating peptide, FIG. 9B is the verification of the degradation effect on the protein, and FIG. 9C shows the verification by a protease inhibitor MG132 that the effect is indeed produced by the protease degrader.

FIG. 10A-FIG. 10C show the modulating targeting chimera molecule induced by the cell-penetrating peptide for degrading novel coronavirus E protein in an example of the present invention and the effect verification thereof, in which FIG. 10A is a structural diagram of the modulating targeting chimera molecule induced by the cell-penetrating peptide, FIG. 10B is the verification of the degradation effect on the protein, and FIG. 10C shows the verification by a protease inhibitor MG132 that the effect is indeed produced by the protease degrader.

FIG. 11A-FIG. 11C show the modulating targeting chimera molecule induced by the cell-penetrating peptide for degrading novel coronavirus Orf6 protein in an example of the present invention and the effect verification thereof, in which FIG. 11A is a structural diagram of the modulating targeting chimera molecule induced by the cell-penetrating peptide, FIG. 11B is the verification of the degradation effect on the protein, and FIG. 11C shows the verification by a protease inhibitor MG132 that the effect is indeed produced by the protease degrader.

FIG. 12A-FIG. 12C show the modulating targeting chimera molecule induced by the cell-penetrating peptide for degrading Lag-3 protein in an example of the present invention and the effect verification thereof, in which FIG. 12A is a structural diagram of the modulating targeting chimera molecule induced by the cell-penetrating peptide, FIG. 12B is the verification of the degradation effect on the protein, and FIG. 12C shows the verification by a protease inhibitor MG132 that the effect is indeed produced by the protease degrader.

FIG. 13A-FIG. 13C show the modulating targeting chimera molecule induced by the cell-penetrating peptide for degrading Her2 protein in an example of the present invention and the effect verification thereof, in which FIG. 13A is a structural diagram of the modulating targeting chimera molecule induced by the cell-penetrating peptide, FIG. 13B is the verification of the degradation effect on the protein, and FIG. 13C shows the verification by a protease inhibitor MG132 that the effect is indeed produced by the protease degrader.

FIG. 14A-FIG. 14C show the modulating targeting chimera molecule induced by the cell-penetrating peptide for degrading SHP-2 protein in an example of the present invention and the effect verification thereof, in which FIG. 14A is a structural diagram of the modulating targeting chimera molecule induced by the cell-penetrating peptide, FIG. 14B is the verification of the degradation effect on the protein, and FIG. 14C shows the verification by a protease inhibitor MG132 that the effect is indeed produced by the protease degrader.

FIG. 15A-FIG. 15C show the modulating targeting chimera molecule induced by the cell-penetrating peptide for degrading STAT5B protein in an example of the present invention and the effect verification thereof, in which FIG. 15A is a structural diagram of the modulating targeting chimera molecule induced by the cell-penetrating peptide, FIG. 15B is the verification of the degradation effect on the protein, and FIG. 15C shows the verification by a protease inhibitor MG132 that the effect is indeed produced by the protease degrader.

FIG. 16A-FIG. 16C show the modulating targeting chimera molecule induced by the cell-penetrating peptide for degrading MUC16 protein in an example of the present invention and the effect verification thereof, in which FIG. 16A is a structural diagram of the modulating targeting chimera molecule induced by the cell-penetrating peptide, FIG. 16B is the verification of the degradation effect on the protein, and FIG. 16C shows the verification by a protease inhibitor MG132 that the effect is indeed produced by the protease degrader.

FIG. 17A-FIG. 17B show the modulating targeting chimera molecule induced by the cell-penetrating peptide for degrading CTLA-4 protein in an example of the present invention and the effect verification thereof, in which FIG. 17A is a structural diagram of the modulating targeting chimera molecule induced by the cell-penetrating peptide, and FIG. 17B is the verification of the degradation effect on the protein shows the verification.

FIG. 18A-FIG. 18B show the modulating targeting chimera molecule induced by the cell-penetrating peptide for degrading PCSK9 protein in an example of the present invention and the effect verification thereof, in which FIG. 18A is a structural diagram of the modulating targeting chimera molecule induced by the cell-penetrating peptide, and FIG. 18B shows the verification by a protease inhibitor MG132 that the effect is indeed produced by the protease degrader.

FIG. 19A-FIG. 19B show the modulating targeting chimera molecule induced by the cell-penetrating peptide for degrading PD-1 protein in an example of the present invention and the effect verification thereof, in which FIG. 19A is a structural diagram of the modulating targeting chimera molecule induced by the cell-penetrating peptide, and FIG. 19B shows the verification by a protease inhibitor MG132 that the effect is indeed produced by the protease degrader.

FIG. 20A-FIG. 20B show the modulating targeting chimera molecule induced by the cell-penetrating peptide for degrading PD-L1 protein in an example of the present invention and the effect verification thereof, in which FIG. 20A is a structural diagram of the modulating targeting chimera molecule induced by the cell-penetrating peptide, and FIG. 20B shows the verification by a protease inhibitor MG132 that the effect is indeed produced by the protease degrader.

FIG. 21A-FIG. 21C show the modulating targeting chimera molecule induced by the cell-penetrating peptide for precisely targeting and degrading KRAS protein with G12V mutation in an example of the present invention and the effect verification thereof, in which FIG. 21A is the structure diagram of the modulating targeting chimera molecule induced by the cell-penetrating peptide, FIG. 21B is the verification of the degradation effect on KRAS protein with G12V mutation, and FIG. 21C is the verification of the degradation effect on the non-mutated KRAS protein (wild type).

FIG. 22A-FIG. 22B show the modulating dual-E3 ligand targeting chimera molecule induced by the cell-penetrating peptide for degrading PCSK9 protein in an example of the present invention and the effect verification thereof, in which FIG. 22A is the structure diagram of the modulating dual-E3 ligand (CRBN+VHL) targeting chimera molecule induced by the cell-penetrating peptide FIG. 22B shows the verification by a protease inhibitor MG132 that the effect is indeed produced by the protease degrader and the overall dosage of the drug is less.

FIG. 23A-FIG. 23B show the modulating dual-target targeting chimera molecule induced by the cell-penetrating peptide for simultaneously degrading HR2 protein and N protein in an example of the present invention and the effect verification thereof, in which FIG. 23A is the structure diagram of the modulating dual target (targets of the novel coronavirus HR2+the novel coronavirus N protein) targeting chimera molecule induced by the cell-penetrating peptide, and FIG. 23B shows the verification by a protease inhibitor MG132 that the effect is indeed produced by the protease degrader.

FIG. 24A-FIG. 24B show the modulating stapled peptide-modified targeting chimera molecule induced by the cell-penetrating peptide for degrading PD-L1 protein in an example of the present invention and the effect verification thereof, in which FIG. 24A is the structure diagram of the modulating stapled peptide-modified targeting chimera molecule induced by the cell-penetrating peptide, and FIG. 24B is the verification of the degradation effect on the protein.

FIG. 25A-FIG. 25B show the modulating cyclic peptide-modified targeting chimera molecule induced by the cell-penetrating peptide for degrading PD-L1 protein in an example of the present invention and the effect verification thereof, in which FIG. 25A is the structure diagram of the modulating cyclic peptide-modified targeting chimera molecule induced by the cell-penetrating peptide, and FIG. 25B is the verification of the degradation effect on the protein.

FIG. 26A-FIG. 26C show a graph showing the validation of the staining of cell penetration of the modulating targeting chimera molecule induced by the cell-penetrating peptide in FIGS. 12A-14C.

FIG. 27A-FIG. 27B show a graph showing the validation of the staining of cell penetration of the modulating targeting chimera molecule induced by the cell-penetrating peptide in FIGS. 15A-C and FIGS. 16A-C.

FIG. 28A-FIG. 28B show a graph showing the validation of the staining of cell penetration of the modulating targeting chimera molecule induced by the cell-penetrating peptide in FIGS. 17A-C and FIGS. 18A-C.

FIG. 29A-FIG. 29B show a graph showing the validation of the staining of cell penetration of the modulating targeting chimera molecule induced by the cell-penetrating peptide in FIGS. 19A-C and FIGS. 20A-C.

FIGS. 30-32 show a structural representation of a stapled peptide+small molecule ligand chimera. Among them, terminal A of FIG. 30 is connected to terminal A of FIG. 31, and terminal B of FIG. 31 is connected to terminal B of FIG. 32. The combination of FIGS. 30-32 shows the structure of a chimera molecule compound containing a stapled peptide.

FIGS. 33-35 show another structural representation of a cyclic peptide+small molecule ligand chimera. Among them, terminal A of FIG. 33 is connected to terminal A of FIG. 34, terminal B of FIG. 33 is connected to terminal B of FIG. 34, and terminal D of FIG. 34 is connected to terminal D of FIG. 35. The combination of FIGS. 33-35 shows the structure of a chimera molecule compound containing a cyclic peptide.

### DETAILED DESCRIPTION

The technical solutions in the examples of the present invention will be clearly and completely described below with reference to the accompanying drawings in the examples of the present invention. Obviously, the described examples are only a part of the examples of the present invention, but not all of the examples. Based on the examples of the present invention, all other examples obtained by those of ordinary skill in the art without creative efforts shall fall within the protection scope of the present invention.

### Example 1

A modulating targeting chimera molecule induced by a cell-penetrating peptide comprises at least one cell-penetrating peptide module, at least one targeting peptide module and at least one small molecule ligand module connected with each other, wherein the targeting peptide module is a peptide sequence that can bind to a targeted protein.

The chimera molecule formed by the cell-penetrating peptide+targeting peptide+small molecule ligand can effectively target the small molecule ligand to the pathogenic target protein through the targeting peptide, so as to achieve the pertinence and specificity of drug treatment. In the field of treatment of various diseases (especially tumor diseases), it can exert more stable and broader effects.

There is one cell-penetrating peptide module, one, two, three or even more targeting peptide modules, and one, two, three or even more small molecule ligand modules.

The modulating targeting chimera molecule induced by the cell-penetrating peptide further comprises at least one Linker module for chimerizing the targeting peptide module and small molecule ligand module. Linker modules can be one, two, three or even more.

The cell-penetrating peptide module is connected to the free end of the targeting peptide module and used to guide the targeting chimera molecule for penetrating the cell membrane.

The small molecule ligand module is a small molecule E3 ligand that can bind to E3 ligase.

Preferably, the protease degrader adapted to the small molecule E3 ligand is one or more of CRBN, VHL, and IAP.

The cell-penetrating peptide module has an amino acid sequence of any one of SEQ ID NO: 1-SEQ ID NO: 3:

(SEQ ID NO: 1)
YGRKKRRQRRR;
(SEQ ID NO: 2)
RRRRRRRR;
and
(SEQ ID NO: 3)
RQIKIWFQNRRMKWK.

The targeting peptide module has an amino acid sequence of any one or more of SEQ ID NO: 4-SEQ ID NO: 17:

(SEQ ID NO: 4)
SAIGKIQDSLSSTAS;
(SEQ ID NO: 5)
PQEESEEEVEEP;
(SEQ ID NO: 6)
GGKGLGKacGGA;
(SEQ ID NO: 7)
DTMVGWDKDARTK;
(SEQ ID NO: 8)
FNGARSFIDI;
(SEQ ID NO: 9)
WARLWNYLYR;
(SEQ ID NO: 10)
RSFIDIGSGT;
(SEQ ID NO: 11)
KAVDG(p)YVKPQI;
(SEQ ID NO: 12)
WIDPVNGDTE;
(SEQ ID NO: 13)
ARHPSWYRPFEGCG;
(SEQ ID NO: 14)
MESFPGWNLV(homoR)IGLLR;
(SEQ ID NO: 15)
FNWDYSLEELREKAKYK;
(SEQ ID NO: 16)
MPIFLDHILNKFWILHYA;
and
(SEQ ID NO: 17)
LYDVAGSDKY.

The Linker module is a small molecule compound with a structural formula shown in formula I:

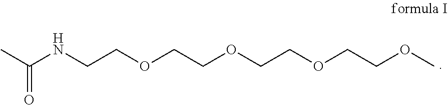 [SMILES: COCCOCCOCCOCCNC(C)=O]

The modulating targeting chimera molecule induced by the cell-penetrating peptide, wherein:

when the adapted protease degrader is CRBN, the structural formula of the small molecule ligand module is shown in formula II:

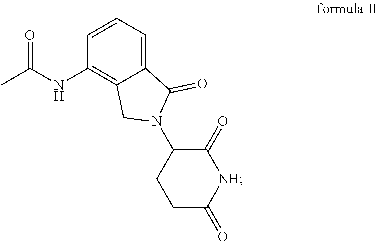 [SMILES: CC(=O)Nc1cccc2c1CN(C1CCC(=O)NC1=O)C2=O]

when the adapted protease degrader is VHL, the structural formula of the small molecule ligand module is shown in formula III:

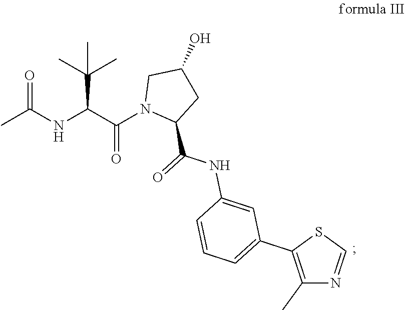 [SMILES: CC(=O)N[C@H](C(=O)N1C[C@H](O)C[C@H]1C(=O)Nc1cccc(-c2scnc2C)c1)C(C)(C)C]

and

when the adapted protease degrader is TAP, the structural formula of the small molecule ligand module is as shown in formula IV:

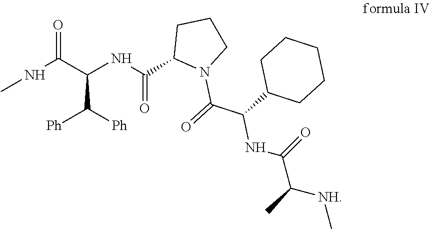

The modulating targeting chimera molecule induced by the cell-penetrating peptide has a structure of any one or more of the following structures:

1) the cell-penetrating peptide of SEQ ID NO: 1+the targeting peptide of SEQ ID NO: 4+the Linker of formula I+the small molecule ligand of formula II;

2) the cell-penetrating peptide of SEQ ID NO: 2+the targeting peptide of SEQ ID NO: 5+the Linker of formula I+the small molecule ligand of formula III;

3) the cell-penetrating peptide of SEQ ID NO: 1+the targeting peptide of SEQ ID NO: 5+the Linker of formula I+the small molecule ligand of formula IV;

4) the cell-penetrating peptide of SEQ ID NO: 1+the targeting peptide of SEQ ID NO: 6+the Linker of formula I+the small molecule ligand of formula II;

5) the cell-penetrating peptide of SEQ ID NO: 1+the targeting peptide of SEQ ID NO: 7+the Linker of formula I+the small molecule ligand of formula III;

6) the cell-penetrating peptide of SEQ ID NO: 1+the targeting peptide of SEQ ID NO: 8+the Linker of formula I+the small molecule ligand of formula II;

7) the cell-penetrating peptide of SEQ ID NO: 1+the targeting peptide of SEQ ID NO: 9+the Linker of formula I+the small molecule ligand of formula III;

8) the cell-penetrating peptide of SEQ ID NO: 1+the targeting peptide of SEQ ID NO: 10+the Linker of formula I+the small molecule ligand of formula II;

9) the cell-penetrating peptide of SEQ ID NO: 1+the targeting peptide of SEQ ID NO: 11+the Linker of formula I+the small molecule ligand of formula IV;

10) the cell-penetrating peptide of SEQ ID NO: 1+the targeting peptide of SEQ ID NO: 12+the Linker of formula I+the small molecule ligand of formula III;

11) the cell-penetrating peptide of SEQ ID NO: 1+the targeting peptide of SEQ ID NO: 13+the Linker of formula I+the small molecule ligand of formula III;

12) the cell-penetrating peptide of SEQ ID NO: 3+the targeting peptide of SEQ ID NO: 14+the Linker of formula I+the small molecule ligand of formula IV;

13) the cell-penetrating peptide of SEQ ID NO: 1+the targeting peptide of SEQ ID NO: 15+the Linker of formula I+the small molecule ligand of formula II;

14) the cell-penetrating peptide of SEQ ID NO: 1+the targeting peptide of SEQ ID NO: 16+the Linker of formula I+the small molecule ligand of formula II;

15) the cell-penetrating peptide of SEQ ID NO: 2+the targeting peptide of SEQ ID NO: 17+the Linker of formula I+the small molecule ligand of formula IV;

16) the cell-penetrating peptide of SEQ ID NO: 3+the targeting peptide of SEQ ID NO: 14+the Linker of formula I+(dual E3 ligands: the small molecule ligand of formula II+the small molecule ligand of formula III); and

17) the cell-penetrating peptide of SEQ ID NO: 1+(dual targets: the targeting peptide of SEQ ID NO: 4+the targeting peptide of SEQ ID NO: 5)+the Linker of formula I+the small molecule ligand of formula II.

The targeting peptide module in the modulating targeting chimera molecule induced by the cell-penetrating peptide further comprises a modified stapled peptide sequence or circular peptide sequence.

The stapled peptide has the structural formula shown in formula V:

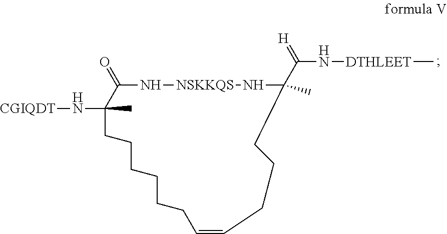

and

the cyclic peptide has the structural formula shown in formula VI:

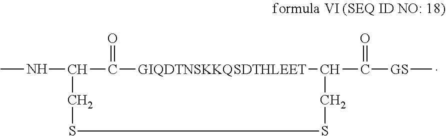

The modulating targeting chimera molecule induced by the cell-penetrating peptide containing the stapled peptide has the structure as follows: the stapled peptide of formula V+the Linker of formula I+the small molecule ligand of formula II.

The modulating targeting chimera molecule induced by the cell-penetrating peptide containing the circular peptide has the structure as follows: the circular peptide of formula VI+the Linker of formula I+the small molecule ligand of formula II.

### Example 2

The modulating targeting chimera molecule induced by the cell-penetrating peptide of Example 1 can be used in preparing a product for degrading a targeted protein or a product for degrading a targeted protein with a mutant amino acid position.

The degraded targeted protein comprises one or more of the novel coronavirus S protein HR2, novel coronavirus N protein, novel coronavirus M protein, novel coronavirus E protein, novel coronavirus Orf6 protein, LAG-3 protein, Her2 protein, SHP-2 protein, STAT5B protein, MUC16 protein, CTLA-4 protein, PCSK9 protein, PD-1 protein, PD-L1 protein and KRAS protein with G12V mutation.

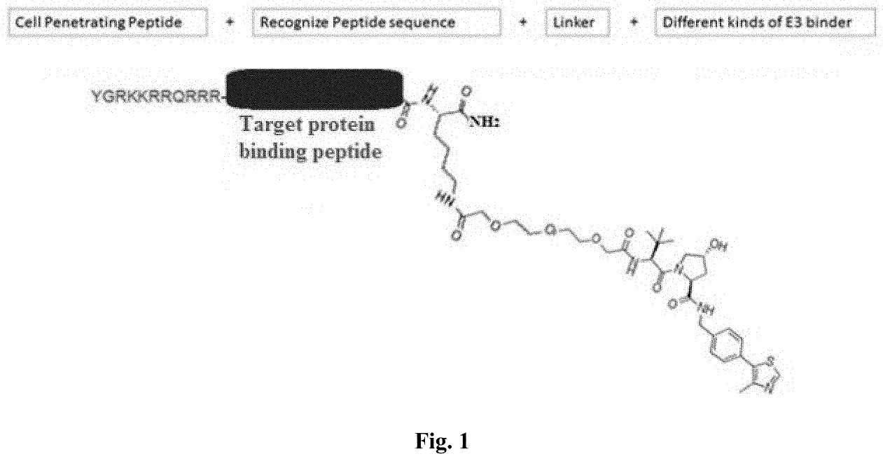

Until now, targeting, inhibiting, and medicating proteins involved in protein-protein interactions has been nearly impossible with the help of inhibitor molecules. Targeting harmful/pathogenic proteins using proteosomal degradation mechanisms is a promising therapeutic approach. With the help of the ubiquitin-proteasome system (UPS), the target protein-protein interaction (PPI) interacts with the target protein via key peptide sequences to selectively degrade the “non-pharmaceutical” target protein. The inventors have devised a method for this bifunctional peptide-based degrader, which targets and degrades target proteins involved in PPIs. The inventors achieved the degradation of the expected target protein with the help of the Linker by binding the peptide with high affinity and selective interaction with the target protein to the E3 ligase. In order to achieve cell permeability of peptide degraders, the inventors further combined peptide degrader sequences with cell permeable peptides (cell-penetrating peptides). The design of the targeting degrader in general is shown in FIG. 1 below.

The inventors have found that peptide degraders were designed to degrade >15000 targets involved in protein-protein interactions. With the help of corresponding targeting ligands/peptides, these ligands could be coupled to about 1100 linkers (of which about 300 PEG-type linkers), with about 65 ligands for E3 ligase binding. In order to improve the permeability of cells, the inventors further combined the peptide PROTAC technology with about 800 cell-penetrating peptides.

SMILES (Simplified molecular input line entry specification) is a specification that clearly describes the molecular structure with ASCII strings; InChI Key is the abbreviation of International Chemical Identifier, and InCHI code is the unique identification code for the chemical structure of each compound given by the International Union of Pure and Applied Chemistry (IUPAC), and its only corresponding compound can be easily found in the PubMed ChemCompound database (https://www.ncbi.nlm.nih.gov/pccompound) via the InChI key.

Table 1 shows selections of the Linker module, including but not limited to the molecular structures represented by SMILES and the compounds corresponding to the InChI key, as shown in Table 1 below for details.

| Smiles | InChI Key |
|---|---|
| CCOCCOC | CAQYAZNFWDDMIT-UHFFFAOYSA-N |
| CCOCCOCCOCCOC | JRRDISHSXWGFRF-UHFFFAOYSA-N |
| CCNC(═O)COCCOCC | QETLNMPRYFLOQP-UHFFFAOYSA-N |
| CCNC(═O)COCCOCCOCCOCC | PEHBLNOEQICMNP-UHFFFAOYSA-N |
| CCOCCOCC═O | VAKGZFIDFWQAJM-UHFFFAOYSA-N |
| CCOCCOCCOCCOCC═O | FMXVEMRCLXJCIA-UHFFFAOYSA-N |
| CCOCCOCCOCCOCCOC | YZWVMKLQNYGKLJ-UHFFFAOYSA-N |
| CCCCCOCCOC | OJTBQXZLANYDLF-UHFFFAOYSA-N |
| CCOCCOCCOC | CNJRPYFBORAQAU-UHFFFAOYSA-N |
| COCCOCCOCC═O | IGENRCKJLAQXEW-UHFFFAOYSA-N |
| CCOCCOCC | LZDKZFUFMNSQCJ-UHFFFAOYSA-N |
| CCOCCOCCOCCOCC | KIAMPLQEZAMORJ-UHFFFAOYSA-N |
| CCOCC | RTZKZFJDLAIYFH-UHFFFAOYSA-N |
| CCOCCOCCOCC | RRQYJINTUHWNHW-UHFFFAOYSA-N |
| CCCCC | OFBQJSOFQDEBGM-UHFFFAOYSA-N |
| CCCCCOCC | VDMXPMYSWFDBJB-UHFFFAOYSA-N |
| CCOCC═O | IAHZBRPNDIVNNR-UHFFFAOYSA-N |
| CCOCCOCC═O | VAKGZFIDFWQAJM-UHFFFAOYSA-N |
| CCOCCOCCOCC═O | VIGPEKUIVIPDAG-UHFFFAOYSA-N |
| CCCCC═O | HGBOYTHUEUWSSQ-UHFFFAOYSA-N |
| CCOCCCCC═O | BCIRIYVFTVIZSE-UHFFFAOYSA-N |
| O═CCO | WGCNASOHLSPBMP-UHFFFAOYSA-N |
| CCCC | IJDNQMDRQITEOD-UHFFFAOYSA-N |
| CC | OTMSDBZUPAUEDD-UHFFFAOYSA-N |
| CCC | ATUOYWHBWRKTHZ-UHFFFAOYSA-N |
| CCCCCC | VLKZOEOYAKHREP-UHFFFAOYSA-N |
| CCCCCCC | IMNFDUFMRHMDMM-UHFFFAOYSA-N |
| C#CCCC | IBXNCJKFFQIKKY-UHFFFAOYSA-N |
| C#CCC | KDKYADYSIPSCCQ-UHFFFAOYSA-N |
| C#CCOCCOCCOCC | NIFIKPHRYNDRRF-UHFFFAOYSA-N |
| C#CCCCCCC | UMIPWJGWASORKV-UHFFFAOYSA-N |
| CCCCCCCC | TVMXDCGIABBOFY-UHFFFAOYSA-N |
| CCCCCCCCC | BKIMMITUMNQMOS-UHFFFAOYSA-N |
| CCCCCOC | DBUJFULDVAZULB-UHFFFAOYSA-N |
| CCCN1CCC(CC)CC1 | JKHJGIRRPGGIIV-UHFFFAOYSA-N |
| CCCN1CCN(CC)CC1 | ZJAQKWSLYRNXDR-UHFFFAOYSA-N |
| CCCC#CC1═CN(CC)N═C1 | BDURCJQVOLLHBO-UHFFFAOYSA-N |
| CCC#CC1═CN(C)N═C1 | YFZOFEJRYGXPSN-UHFFFAOYSA-N |
| CCCCC1═CN(C)N═C1 | QTCJYVJPWUREDL-UHFFFAOYSA-N |
| CCCCCCCCCC | DIOQZVSQGTUSAI-UHFFFAOYSA-N |
| CCCCCCCCCCC | RSJKGSCJYJTIGS-UHFFFAOYSA-N |
| C#CC1═CC═C(CCCCCC)N═C1 | JVXALQWTVBJACI-UHFFFAOYSA-N |
| C#CC1═CC═C(N2CCN(CCC)CC2)N═C1 | KUIOGLKGGGGIAM-UHFFFAOYSA-N |
| C#CC1═CC═C(N2CCN(CCCC)CC2)N═C1 | FCWKVTCLYUQKNS-UHFFFAOYSA-N |
| C#CC1═CC═C(N2CCN(CC)CC2)N═C1 | APRHSTGCOUVFGK-UHFFFAOYSA-N |
| C#CC1═CC═C(N2CCN(C)CC2)N═C1 | OILUJPMEHSBBQL-UHFFFAOYSA-N |
| C#CC1═CC═C(N2CCNCC2)N═C1 | IMSQYOVMMQROAO-UHFFFAOYSA-N |
| C#CC1CCN(C2CCNCC2)CC1 | BRIAZNMAVALSCM-UHFFFAOYSA-N |
| CCCCCCCCCNC═O | HEIIJVVALRPNFV-UHFFFAOYSA-N |
| CCCCCCCCCCNC═O | ZNCRMMYZWDNTCE-UHFFFAOYSA-N |
| CCCCNC(═O)CO | WFYNRXPMFUYIDC-UHFFFAOYSA-N |
| CCCCNC(C)═O | GYLDXXLJMRTVSS-UHFFFAOYSA-N |
| CCCCCCCO | BBMCTIGTTCKYKF-UHFFFAOYSA-N |
| CCCOCC | NVJUHMXYKCUMQA-UHFFFAOYSA-N |
| CCCCCNC═O | UBKOTQBYKQFINX-UHFFFAOYSA-N |
| NCCN1C═C(CO)N═N1 | QBRSPHHAFDKHBS-UHFFFAOYSA-N |
| NCCN1C═C(COCCO)N═N1 | MLEHCIIJTRPTGA-UHFFFAOYSA-N |
| NCCN1C═C(COCCOCCO)N═N1 | VYVPEKOLZKXSGT-UHFFFAOYSA-N |
| NCCN1C═C(COCCOCCOCCO)N═N1 | XNRQJYPYBNBWJK-UHFFFAOYSA-N |
| NCCN1C═C(COCCOCCOCCOCCO)N═N1 | CMBXUPPKXOSUED-UHFFFAOYSA-N |
| COCC1═CN(CCN)N═N1 | XUQZBMLYINPFHS-UHFFFAOYSA-N |
| COCCOCC1═CN(CCN)N═N1 | QPYGOKLPMSIQDN-UHFFFAOYSA-N |
| COCCOCCOCC1═CN(CCN)N═N1 | LDNUXLMZQAVQMX-UHFFFAOYSA-N |
| COCCOCCOCCOCC1═CN(CCN)N═N1 | VDWXNHBISFHPAI-UHFFFAOYSA-N |
| COCCOCCOCCOCCOCC1═CN(CCN)N═N1 | TVAHVEKMLUNMOI-UHFFFAOYSA-N |
| CO | OKKJLVBELUTLKV-UHFFFAOYSA-N |
| CCOCCOCCOCCNC(═O)CC | DQKOWFBYHIENAP-UHFFFAOYSA-N |
| CCOCCOCCOCCOCCNC(C)═O | ATHNQMOBOHHVGV-UHFFFAOYSA-N |
| CCCC1═CN(CCOCCOCCOCCOCC)N═N1 | FCKRLESEKMOAAY-UHFFFAOYSA-N |
| CCCC1═CN(CCOCCOCCOCCOCCNC(C)═O)N═N1 | LEIGAYMEZRVQMF-UHFFFAOYSA-N |
| CCCCOCCOCCOCC | VXVGKMGIPAWMJC-UHFFFAOYSA-N |
| CCCCCCCCCCCOCCCC | BFTNUQIEWVRTCA-UHFFFAOYSA-N |
| CCCCCCOCCCCCCOCCCCC | QGMIIQRSLFEZNE-UHFFFAOYSA-N |
| CCCCCCCCCOCCCCOCCCC | ZRZNGCGNHBPEEW-UHFFFAOYSA-N |
| CCCCCCOCCOCCOCCOCCOCCOCCCCC | GBTJHLJNCMMVEE-UHFFFAOYSA-N |
| CCCCCCOCCCCOCCCCOCCCCOCCCCC | CCRDSFBIFBOEPO-UHFFFAOYSA-N |
| CCCOCCOCC | MZBACIJSSOHXQA-UHFFFAOYSA-N |
| CCCOCCCOCC | ZFHCEEOJBANLST-UHFFFAOYSA-N |
| CCCCOCCCCOC | FBPKXEFBJHBHJN-UHFFFAOYSA-N |
| CCCCOCCCC | DURPTKYDGMDSBL-UHFFFAOYSA-N |
| CCCOCCOCCC | HQSLKNLISLWZQH-UHFFFAOYSA-N |
| CCCOCCCOCCC | PZYMDANKTMTEIY-UHFFFAOYSA-N |
| CCCCNC(C)═O | GYLDXXLJMRTVSS-UHFFFAOYSA-N |
| CCCCCCCCNC(C)═O | GLJKLMQZANYKBO-UHFFFAOYSA-N |
| CCCCCC═O | JARKCYVAAOWBJS-UHFFFAOYSA-N |
| CCOCCOCCO | XXJWXESWEXIICW-UHFFFAOYSA-N |
| CCCOCCOCCOCCCNC(C)═O | FCOSMYXPOFLRFY-UHFFFAOYSA-N |
| CCCCCN | DPBLXKKOBLCELK-UHFFFAOYSA-N |
| CCOC | XOBKSJJDNFUZPF-UHFFFAOYSA-N |
| CC(═O)NCCOCCOCC(═O)NCCOCCO | CBFGQJLCZYDUKF-UHFFFAOYSA-N |
| CC(═O)NCCOCCO | DJDAFXBIBNKCBR-UHFFFAOYSA-N |
| CC(═O)NCCOCCOCC(═O)NCCOCCOCC(═O)NCCOCCO | OWOUJOSDJHEXFQ-UHFFFAOYSA-N |
| NCCOCCOCCOCCN | NIQFAJBKEHPUAM-UHFFFAOYSA-N |
| COCCCCOCCCOC1═CC═C(N)C═C1 | SINCTDJNRNXKAO-UHFFFAOYSA-N |
| CCO | LFQSCWFLJHTTHZ-UHFFFAOYSA-N |
| CCOC | XOBKSJJDNFUZPF-UHFFFAOYSA-N |
| CCOCCO | ZNQVEEAIQZEUHB-UHFFFAOYSA-N |
| CCOCCOC | CAQYAZNFWDDMIT-UHFFFAOYSA-N |
| CCOCCOCCO | XXJWXESWEXIICW-UHFFFAOYSA-N |
| CCOCCOCCOCCO | WFSMVVDJSNMRAR-UHFFFAOYSA-N |
| CCOCCOCCOCCOC | JRRDISHSXWGFRF-UHFFFAOYSA-N |
| CCOCCOCCOCCOCCO | GTAKOUPXIUWZIA-UHFFFAOYSA-N |
| CCOCCOCCOCCOCCOC | YZWVMKLQNYGKLJ-UHFFFAOYSA-N |
| CCOCCOCCOCCOCCOCCO | NJRFAMBTWHGSDE-UHFFFAOYSA-N |
| CCOCCOCCOCCOCCOCCOC | PJXDGFJDVVVXCY-UHFFFAOYSA-N |
| OCCOCCOCCOCCOCCO | JLFNLZLINWHATN-UHFFFAOYSA-N |
| OCCOCCOCCOCCOCCOCCO | IIRDTKBZINWQAW-UHFFFAOYSA-N |
| CCOCCOCCOCCOCCOCCNC(C)═O | CUAFPSUETCCBKZ-UHFFFAOYSA-N |
| CCNC(C)═O | PMDCZENCAXMSOU-UHFFFAOYSA-N |
| CCN1C═C(C)N═N1 | MJIOJWXRXDWUBV-UHFFFAOYSA-N |
| CCOCCN1C═C(C)N═N1 | ZSUBJILEBOCSPA-UHFFFAOYSA-N |
| CCOCCOCCN1C═C(C)N═N1 | IUKAVXDFGYPRJN-UHFFFAOYSA-N |
| CCOCCOCCOCCN1C═C(C)N═N1 | XYQHQTXKPLBAMW-UHFFFAOYSA-N |
| COCCOCCOCCN | OKUWOEKJQRUMBW-UHFFFAOYSA-N |
| CCCCCCCCN | IOQPZZOEVPZRBK-UHFFFAOYSA-N |
| CCOCCOCCN | KURRHYKFNUZCSJ-UHFFFAOYSA-N |
| CCOCCOCCOC1═CC═C(N)C═C1 | ZPPAIDISWIOLFL-UHFFFAOYSA-N |
| CCOCCOCCOC1═CC═CC(N)═C1 | JALUOFLXLMYSLF-UHFFFAOYSA-N |
| NCC═O | LYIIBVSRGJSHAV-UHFFFAOYSA-N |
| O═CCCC(═O)NCC═O | GZVFPGVIQBINMS-UHFFFAOYSA-N |
| O═CCCCC(═O)NCC═O | PIJCAVNFNFGLTJ-UHFFFAOYSA-N |
| CCOCCOCCOCCNC(C)═O | JOVFTSYTPCENPI-UHFFFAOYSA-N |
| CCOCCOCCOCCNC(═O)CO | HQTQBLVLNRHRPZ-UHFFFAOYSA-N |
| CCOCCOCCN1C═C(C═O)N═N1 | ZNAOSTXJYFIKBZ-UHFFFAOYSA-N |
| CCOCCOCCOCCN1C═C(C═O)N═N1 | YUYIWPLVJSWWGQ-UHFFFAOYSA-N |
| CCOCCOCCOCCOCCN1C═C(C═O)N═N1 | YESZEWIOSFYVAA-UHFFFAOYSA-N |
| CCCC1═CN(CCOCCOCC)N═N1 | BQZDDUUCYNHBBL-UHFFFAOYSA-N |
| CCOCCOCCOCCN1N═NC═C1C═O | ZBCVVBUUELQFGX-UHFFFAOYSA-N |
| CCOCCOCCOCCOCCN1N═NC═C1C═O | OFARQSZAAFNSEE-UHFFFAOYSA-N |
| CC(═O)NCCOCCOCCN1C═C(CCCC═O)N═N1 | NGHQHEBDKFKOGF-UHFFFAOYSA-N |
| CC(═O)NCCOCCOCCOCCN1C═CN═N1 | HAQDEBRMWUNDNK-UHFFFAOYSA-N |
| CC(═O)NCCOCCOCCOCCN1C═C(C═O)N═N1 | JIGCWMDYYHPXEW-UHFFFAOYSA-N |
| CC(═O)NCCOCCOCCOCCOCCN1C═C(C═O)N═N1 | MADSWKVPPGQVEW-UHFFFAOYSA-N |
| CCCCCCCCCCCN1C═C(C═O)N═N1 | JGTUSDGGOIALAA-UHFFFAOYSA-N |
| CCCCCN1C═C(CCCC═O)N═N1 | WBYOUGPMCNNHAC-UHFFFAOYSA-N |
| CCCCCN1C═C(C═O)N═N1 | SLJXYIVRAZQICO-UHFFFAOYSA-N |
| CCCCCCCC═O | NUJGJRNETVAIRJ-UHFFFAOYSA-N |
| COCCOCCOCCO | JLGLQAWTXXGVEM-UHFFFAOYSA-N |
| COCCCOCCCCCO | XPPFZKCGEIHABQ-UHFFFAOYSA-N |
| COCCCCOC1═CC═CC═C1 | JZDHXBGNBZJRFK-UHFFFAOYSA-N |
| CCC(═O)NCCCOCCOCCO | ZSOUNYZAIRTFCF-UHFFFAOYSA-N |
| CCCC═O | ZTQSAGDEMFDKMZ-UHFFFAOYSA-N |
| CCCCCC═O | JARKCYVAAOWBJS-UHFFFAOYSA-N |
| CCCOC | VNKYTQGIUYNRMY-UHFFFAOYSA-N |
| CCCOCC | NVJUHMXYKCUMQA-UHFFFAOYSA-N |
| CCCOCCC(═O)NCCO | ZWZRKLRMHPVSCL-UHFFFAOYSA-N |
| CCOCCOCCC(═O)NCCO | CGZNNRDCSGZCCN-UHFFFAOYSA-N |
| CCOCCOCCOCCOCCC(═O)NCCO | SESQQSBFFNCJEC-UHFFFAOYSA-N |
| CCCCCOCCOCC | UOECJVYWINLCEV-UHFFFAOYSA-N |
| CCCCCOCCCCOCCCC | BEGITWDDPUIZSL-UHFFFAOYSA-N |
| O═CCCCNC(═O)CO | LNMQGYJLRFKBBM-UHFFFAOYSA-N |
| CCCCCCOCCOCC═O | MDCZWRMRSILCQG-UHFFFAOYSA-N |
| CCCCOCCCCOCC═O | GAIQORAOHTYFOZ-UHFFFAOYSA-N |
| CCCOCCCOCCC═O | DDVUDFQFSHPIKB-UHFFFAOYSA-N |
| CCCOCCCOCCC(═O)NCCO | NCLTXMWFZMRLQT-UHFFFAOYSA-N |
| CCCCCCC═O | FXHGMKSSBGDXIY-UHFFFAOYSA-N |
| CCCCCCCC═O | NUJGJRNETVAIRJ-UHFFFAOYSA-N |
| CCCCCCCCCC═O | KSMVZQYAVGTKIV-UHFFFAOYSA-N |
| CC1═CN(CCCCCC═O)N═N1 | GBVUXEUIQYTUOX-UHFFFAOYSA-N |
| CC1═CN(CCCCCCC═O)N═N1 | PEWQKAAXGLGGNO-UHFFFAOYSA-N |
| CC1═CN(CCCCCCCC═O)N═N1 | CMCUANCCXFQPIK-UHFFFAOYSA-N |
| CC1═CN(CCCCCCCCCC═O)N═N1 | WKBRZAASKLMMQP-UHFFFAOYSA-N |
| CN(C═O)CCCN(C)C1═CC═C(CCCO)C═C1 | UKHJUYQLLMDXPO-UHFFFAOYSA-N |
| CCCOCCCCOCCC | JNOSBGUMRLGKJN-UHFFFAOYSA-N |
| CCCOCCOCCOCCC | BOGFHOWTVGAYFK-UHFFFAOYSA-N |
| OCCOCCOCCO | ZIBGPFATKBEMQZ-UHFFFAOYSA-N |
| O═CNCCOCCOCCNC═O | XIXYICXGUYBZDP-UHFFFAOYSA-N |
| CCCCCO | AMQJEAYHLZJPGS-UHFFFAOYSA-N |
| O═CCCCNC(═O)CCC═O | SKLRXSCPVXBXCO-UHFFFAOYSA-N |
| O═CCCCCCNC(═O)CCC(═O)NCCCC═O | LKWKWZFHIXKOEG-UHFFFAOYSA-N |
| O═CCCCCCNC(═O)CCC═O | HQPRYQBUTVJJOR-UHFFFAOYSA-N |
| CC1═CN(CCCC═O)N═N1 | LIZRSROCAZLHER-UHFFFAOYSA-N |
| CCCCCCN | BMVXCPBXGZKUPN-UHFFFAOYSA-N |
| CCCCN | HQABUPZFAYXKJW-UHFFFAOYSA-N |
| CCOCCN | BPGIOCZAQDIBPI-UHFFFAOYSA-N |
| CCCOCCCN | UTOXFQVLOTVLSD-UHFFFAOYSA-N |
| CCCCOCCN | BFBKUYFMLNOLOQ-UHFFFAOYSA-N |
| CCCCCCOCCOCCOCCCCCC═O | KADLFJHSNYQITD-UHFFFAOYSA-N |
| CCCCCCOCCCCCOCCCCCC═O | FBKPSQUKFDEYMJ-UHFFFAOYSA-N |
| CCCCCCOCCOCCOCCOCCOCCOCC═O | YSWVYWOXYLIZEF-UHFFFAOYSA-N |
| CCCCCCOCCOC | RVDZRFYFUCWKPB-UHFFFAOYSA-N |
| CCCCCCOCCOCCOCCCCC | HQOQXJPHOPMXDY-UHFFFAOYSA-N |
| CCCCCCOCCCCCOCCCCC | KDUUQQCQQVVSBB-UHFFFAOYSA-N |
| CCCCCCOCCOCCOCCOCCOCCOC | CNLOXGUTRGRTFZ-UHFFFAOYSA-N |
| COCCCOCCN | FELLHWJAUQSSNB-UHFFFAOYSA-N |
| NCCCCNC(═O)CO | AKNMVWRGOGXKKH-UHFFFAOYSA-N |
| O═CCCC(═O)NCCCCNC(═O)CO | TZEKEZIWKZSWOI-UHFFFAOYSA-N |
| O═CCCC(═O)NCCCOCCOCCOCCCNC(═O)CO | FHOBUEQAJHGQQA-UHFFFAOYSA-N |
| COCCOCCOCCOCCN | DQTQYVYXIOQYGN-UHFFFAOYSA-N |
| COCCOCCOCCNC(C)═O | IOEHUBKJBRVLHW-UHFFFAOYSA-N |
| NCCCCCCCCNC(═O)CO | YRHIKLITYIBTQJ-UHFFFAOYSA-N |
| NCCOCCOCCOCCOCCOCCOCCOCCOCCOCCNC(═O)CO | GGPNIVAICDTPRA-UHFFFAOYSA-N |
| CCN | QUSNBJAOOMFDIB-UHFFFAOYSA-N |
| NCCO | HZAXFHJVJLSVMW-UHFFFAOYSA-N |
| NCCCCCCCCO | WDCOJSGXSPGNFK-UHFFFAOYSA-N |
| CCCN | WGYKZJWCGVVSQN-UHFFFAOYSA-N |
| C#CC1CN(C2CCNCC2)C1 | KJXOBLQLPXCXFC-UHFFFAOYSA-N |
| C#CC1CCN(C2CNC2)CC1 | NPMKYYQTCFIXHK-UHFFFAOYSA-N |
| C#CC1CCNCC1 | FWOORBMXLUBSEV-UHFFFAOYSA-N |
| C1CC(C2CNC2)CCN1 | BXGJTOWQMBDJGT-UHFFFAOYSA-N |
| C1CN(C2CCNCC2)C1 | DDOFLXMWWKLMSZ-UHFFFAOYSA-N |
| C1CCNCC1 | NQRYJNQNLNOLGT-UHFFFAOYSA-N |
| C1CNCCN1 | GLUUGHFHXGJENI-UHFFFAOYSA-N |
| CCCNC(C)═O | IHPHPGLJYCDONF-UHFFFAOYSA-N |
| CCOCCOCCN1C═C(C)N═N1 | IUKAVXDFGYPRJN-UHFFFAOYSA-N |
| CCOCCOCCOCCN1C═C(C)N═N1 | XYQHQTXKPLBAMW-UHFFFAOYSA-N |
| CCOCCOCCOCCOCCN1C═C(C)N═N1 | YHUVOJYLWJUSCL-UHFFFAOYSA-N |
| CCOCCNC(C)═O | VNVZKKJQVMBZNN-UHFFFAOYSA-N |
| CCOCCOCCNC(C)═O | XENYNHWLGLZAAS-UHFFFAOYSA-N |
| CCOCCOCCOCCOCCNC(C)═O | ATHNQMOBOHHVGV-UHFFFAOYSA-N |
| CCOCCN1C═C(C)N═N1 | ZSUBJILEBOCSPA-UHFFFAOYSA-N |
| CCCCCNC(C)═O | PTBCMKWBUAWWMQ-UHFFFAOYSA-N |
| CCCCCN1C═C(C)N═N1 | NGPORLBABXGWLK-UHFFFAOYSA-N |
| CCCCCCN1C═C(C)N═N1 | WQWGJNYVTILRSK-UHFFFAOYSA-N |
| CCOCCN1C═C(C═O)N═N1 | BUTDPEQHKNRYSQ-UHFFFAOYSA-N |
| CCOCCN1C═C(CC═O)N═N1 | MLQKFJRBGXZNPJ-UHFFFAOYSA-N |
| CCCN1C═C(CCC═O)N═N1 | COBLXHJSQAZDEI-UHFFFAOYSA-N |
| CCN1C═C(CCCC═O)N═N1 | UEEFSVRERBYXAS-UHFFFAOYSA-N |
| C#CCCCCN1C═C(C)N═N1 | GCTIRBGRXALNIU-UHFFFAOYSA-N |
| C#CCCCCCN1C═C(C)N═N1 | WZKZKIJGBGNPDU-UHFFFAOYSA-N |
| CCOCCOCCOCCOCCOCC | HYDWALOBQJFOMS-UHFFFAOYSA-N |
| CCOCCOCCOCCOCCOCCOCC | IXFAFGFZFQHRLB-UHFFFAOYSA-N |
| CC(═O)NCCOCCN1C═C(C)N═N1 | QPHBYCZOFUQLRX-UHFFFAOYSA-N |
| CC(═O)NCCOCCOCCN1C═C(C)N═N1 | MCKJFZJLSFEXGY-UHFFFAOYSA-N |
| CC(═O)NCCOCCOCCOCCN1C═C(C)N═N1 | XFNSODNPEBFOCR-UHFFFAOYSA-N |
| CCC(═O)NCCOCCN1C═C(C)N═N1 | XWAFIJZKAUTKLW-UHFFFAOYSA-N |
| CCC(═O)NCCOCCOCCN1C═C(C)N═N1 | AITLBNIGQBURHS-UHFFFAOYSA-N |
| CCC(═O)NCCOCCOCCOCCN1C═C(C)N═N1 | QZSYXQTZEQRRLH-UHFFFAOYSA-N |
| CCC(═O)NCCOCCOCCOCCOCCN1C═C(C)N═N1 | CJTNAYUHLNOBKI-UHFFFAOYSA-N |
| CC1═CN(CCOCCN)N═N1 | QHVJBCXMEZEORX-UHFFFAOYSA-N |
| CC1═CN(CCOCCOCCN)N═N1 | FOAMGHKADZQZHG-UHFFFAOYSA-N |
| CC1═CN(CCOCCOCCOCCN)N═N1 | LXUHWBGKFLKKKF-UHFFFAOYSA-N |
| CC1═CN(CCOCCOCCOCCOCCN)N═N1 | SBUPHHQGPCNIGC-UHFFFAOYSA-N |
| CCC(═O)NCCOCCOCCOC | URKSSMOBHOURQG-UHFFFAOYSA-N |
| CCC(═O)NCCOCCOCCOCCOC | VHRWXKFRKRROGZ-UHFFFAOYSA-N |
| COCCOCCOCCNC(═O)CO | QALSNBOYMNJLAA-UHFFFAOYSA-N |
| COCCOCCOCCOCCNC(═O)CO | QIHZDIJVVMWURU-UHFFFAOYSA-N |
| COCCOCCOCCNC═O | WBXRGXRTHMHNQH-UHFFFAOYSA-N |
| COCCOCCOCCOCCNC═O | RVVXLQFQZBAHEU-UHFFFAOYSA-N |
| CNCCOCCOCCOC | XOTTZADIXWMSNG-UHFFFAOYSA-N |
| CNCCOCCOCCOCCOC | OCYLGYGLYZJUGM-UHFFFAOYSA-N |
| COCCOCCOCCNC(C)═O | IOEHUBKJBRVLHW-UHFFFAOYSA-N |
| COCCOCCOCCOCCNC(C)═O | YKUZVFLJHLWLIY-UHFFFAOYSA-N |
| O═CCOCCOCCOCCNC═O | BCTUXHHLONLIIU-UHFFFAOYSA-N |
| O═CCOCCOCCOCCOCCNC═O | WRZPPQPGUIFVIT-UHFFFAOYSA-N |
| CNCCOCCOCCOCC═O | PXLLLGLWNQABNB-UHFFFAOYSA-N |
| CNCCOCCOCCOCCOCC═O | JBSBLUUBPFMTTH-UHFFFAOYSA-N |
| CC(═O)NCCOCCOCCOCC═O | HQUQFJSZJHNHQH-UHFFFAOYSA-N |
| CC(═O)NCCOCCOCCOCCOCC═O | ZZKRGZDUJWNBDH-UHFFFAOYSA-N |
| CCC(═O)NCCOCCOCCOCC═O | UFYKRLZOLKHWOC-UHFFFAOYSA-N |
| CCC(═O)NCCOCCOCCOCCOCC═O | YQFLFBLMNIYTOX-UHFFFAOYSA-N |
| O═CCOCCOCCOCCNC(═O)CO | RWBADFLVOPAZFF-UHFFFAOYSA-N |
| O═CCOCCOCCOCCOCCNC(═O)CO | LYRYAVFGHCODDF-UHFFFAOYSA-N |
| O═CNCCOCCOCCOCCO | APICKUFCNMZSLX-UHFFFAOYSA-N |
| O═CNCCOCCOCCOCCOCCOCCO | NJLZVMKIWZVWFY-UHFFFAOYSA-N |
| CNCCOCCOCCOCCOCCOCCO | RDQINYCFOSDCHI-UHFFFAOYSA-N |
| CNCCOCCOCCOCCO | JBLOFIJEJSEJCB-UHFFFAOYSA-N |
| CC(═O)NCCOCCOCCOCCO | SLDVGISICCQBOZ-UHFFFAOYSA-N |
| CC(═O)NCCOCCOCCOCCOCCOCCO | UTWLXLUBBXONPL-UHFFFAOYSA-N |
| CCC(═O)NCCOCCOCCOCCO | YWVQPILJDJGJBG-UHFFFAOYSA-N |
| CCC(═O)NCCOCCOCCOCCOCCOCCO | KUSFIFBDJWJPFO-UHFFFAOYSA-N |
| O═C(CO)NCCOCCOCCOCCOCCOCCO | XYMRJXHYEQNOFC-UHFFFAOYSA-N |
| CCCCCOCCOCCOCCCCCC═O | ZWMDTRIMMQOZQV-UHFFFAOYSA-N |
| CCOCCOCCOCCOCCOCCC═O | XYLCAEDVEIYMPO-UHFFFAOYSA-N |
| CCOCCOCCOCCOCCC═O | QNYDWCLUZIEJKP-UHFFFAOYSA-N |
| CCOCCOCCOCCC═O | AJMGGIZNAPWJIH-UHFFFAOYSA-N |
| CCOCCOCCC═O | HMJYMPSQSRSWMR-UHFFFAOYSA-N |
| COCCOCC═O | KEAGYJMKALOSDP-UHFFFAOYSA-N |
| CCCCCCCCCCCCCCC═O | XGQJZNCFDLXSIJ-UHFFFAOYSA-N |
| CCCCCCCCCCCCC═O | BGEHHAVMRVXCGR-UHFFFAOYSA-N |
| CCCCCCCCC═O | GYHFUZHODSMOHU-UHFFFAOYSA-N |
| CCCCCCC═O | FXHGMKSSBGDXIY-UHFFFAOYSA-N |
| CCC═O | NBBJYMSMWIIQGU-UHFFFAOYSA-N |
| CCCN1C═C(CCCC═O)N═N1 | IXEAGSXAQJLWLN-UHFFFAOYSA-N |
| CCCC(═O)NCC═O | ODOUTTTVJWVZSZ-UHFFFAOYSA-N |
| CCCCCCCCC(═O)NCCCCC═O | MYFQXLLLRBVRSP-UHFFFAOYSA-N |
| CCCCCCCCC(═O)NCCCCC | JGFASXASDJWOCB-UHFFFAOYSA-N |
| C#CCCCC | CGHIBGNXEGJPQZ-UHFFFAOYSA-N |
| C#CCCCCC | YVXHZKKCZYLQOP-UHFFFAOYSA-N |
| C#CCCCCCCCC | ILLHQJIJCRNRCJ-UHFFFAOYSA-N |
| C#CCCCCO | GOQJMMHTSOQIEI-UHFFFAOYSA-N |
| C#CCCCCN | ONUHRYKLJYSRMY-UHFFFAOYSA-N |
| C#CCCCN1C═CC═N1 | OJFUNMFSXWAZPG-UHFFFAOYSA-N |
| C#CC1═CN(CCC)N═C1 | IIIOFGJLACNCFE-UHFFFAOYSA-N |
| C#CCCC1CCNCC1 | CGXLFBWBODOMPY-UHFFFAOYSA-N |
| C#CC1CCN(CC)CC1 | BINSJKUZTYWDEH-UHFFFAOYSA-N |
| C#CC1CCN(C(C)═O)CC1 | PBMXIHNKFXZVGP-UHFFFAOYSA-N |
| CCCCCCNC(═O)CCCCCN | MLWOJDWSEFRSSW-UHFFFAOYSA-N |
| CCCCCCCCCC═O | KSMVZQYAVGTKIV-UHFFFAOYSA-N |
| CCOCCOCCNC(C)═O | XENYNHWLGLZAAS-UHFFFAOYSA-N |
| C#CCCC═O | VWYVHZFRBJJWSM-UHFFFAOYSA-N |
| C#CCCCC═O | JIBLCOIURXDOGU-UHFFFAOYSA-N |
| C#CCCCCC═O | UIRZQOFYQKICOZ-UHFFFAOYSA-N |
| C#CCCC(═O)NCC═O | WEMNOUZFMRIBSN-UHFFFAOYSA-N |
| C#CCCCC(═O)NCC═O | GMAQFMCBFYVZOJ-UHFFFAOYSA-N |
| C#CCCCCC(═O)NCC═O | YCFWTMPORMEAAJ-UHFFFAOYSA-N |
| C#CC | MWWATHDPGQKSAR-UHFFFAOYSA-N |
| CC═O | IKHGUXGNUITLKF-UHFFFAOYSA-N |
| CCCCC═O | HGBOYTHUEUWSSQ-UHFFFAOYSA-N |
| CC(═O)NCCC═O | ARJPPNFIEQKVBB-UHFFFAOYSA-N |
| O═CCCCCCCCCCCC═O | SZCGBFUWBCDIEA-UHFFFAOYSA-N |
| NCCCCCCCCCCCC═O | GDWQVDJDAUJLON-UHFFFAOYSA-N |
| CCCCCCCCCCCCN | JRBPAEWTRLWTQC-UHFFFAOYSA-N |
| CCCCCCCCCCC═O | KMPQYAYAQWNLME-UHFFFAOYSA-N |
| NCCCCCCCCCC═O | XMIVZZVWXHLGDM-UHFFFAOYSA-N |
| NCCCCCCCC═O | GKOPBHPTLGFKOR-UHFFFAOYSA-N |
| NCCCCCC═O | CCYXEHOXJOKCCJ-UHFFFAOYSA-N |
| CCOCCC═O | RKSGQXSDRYHVTM-UHFFFAOYSA-N |
| CCOCCNC(═O)CC | IMOYXTIJNKLJOW-UHFFFAOYSA-N |
| CCOCCOCCOCCOCCOCCNC(═O)CC | PMPWGAMCIWMUBB-UHFFFAOYSA-N |
| CCCCCCNC(═O)CC | WVNFFHLXDXOSGO-UHFFFAOYSA-N |
| CCC(═O)NCCCCCCCCO | JQRQVXPEZIPWQF-UHFFFAOYSA-N |
| O═CCCCCCNC(═O)CO | KGQYSVDXYQVIEV-UHFFFAOYSA-N |
| CCNC(═O)CCC═O | OTIBNEGBGVSWFM-UHFFFAOYSA-N |
| CCCCNC(═O)CCC═O | ARICYFIDJVFXKT-UHFFFAOYSA-N |
| CCCCCCNC(═O)CCC═O | VCAFBVUKPAUEEG-UHFFFAOYSA-N |
| O═CCCC(═O)NCCNC(═O)CO | OQAZTSBOTAAJRR-UHFFFAOYSA-N |
| O═CCCC(═O)NCCCCCCNC(═O)CO | QJIAPTKTDZEFAD-UHFFFAOYSA-N |
| O═CCOCCOCCNC(═O)CO | VNCLZPDQVSNYFJ-UHFFFAOYSA-N |
| COCCCOCC═O | XHQZGALFKOIZRE-UHFFFAOYSA-N |
| CCOCCOCCOCCOCC═O | FMXVEMRCLXJCIA-UHFFFAOYSA-N |
| CCOCCOCCNC═O | RNWZJESYGONOHW-UHFFFAOYSA-N |
| CCOCCOCCNC═S | QFMUBEXVTGHSBI-UHFFFAOYSA-N |
| CCOCCOCC1═CN(CCCC═O)N═N1 | IWWAUXMBDMJSSV-UHFFFAOYSA-N |
| CCOCCOCCOCC1═CN(CCCC═O)N═N1 | OPVXSAVGINBJEB-UHFFFAOYSA-N |
| CCOCCOCC1═CN(CCCCCCC═O)N═N1 | DOYCDXHJCFENHY-UHFFFAOYSA-N |
| CCOCCOCCOCC1═CN(CCCCCCC═O)N═N1 | SFSUBKPMRDKRBN-UHFFFAOYSA-N |
| CCCCCCCN1C═C(COCCOCC)N═N1 | VNPKSTFQNRZCDX-UHFFFAOYSA-N |
| CCCCCCCN1C═C(COCCOCCOCC)N═N1 | ZZXCHRNDDZQTMP-UHFFFAOYSA-N |
| OCCN1C═CN═N1 | PKHVUMMJMYKRNO-UHFFFAOYSA-N |
| OCCCCCN1C═CN═N1 | PTYAAEDDIDZYTP-UHFFFAOYSA-N |
| OCCCCCCN1C═CN═N1 | MKLKVRBLQFXYPA-UHFFFAOYSA-N |
| OCCCCCCCCN1C═CN═N1 | GSYUZMZQCSLBPA-UHFFFAOYSA-N |
| NCCCCCC═O | CCYXEHOXJOKCCJ-UHFFFAOYSA-N |
| NC(═O)CCC(═O)NCCCCCC-O | MRWKUXULCQWHDJ-UHFFFAOYSA-N |
| NCCOCCOCCOCCC═O | PGRFVPHGKLDGEE-UHFFFAOYSA-N |
| NCCOCCOCCOCCOCCOCCOCCOCCC═O | WQVLRRLIBUBEEL-UHFFFAOYSA-N |
| CCOCCNC(═O)CON | IKZNZUUOZXXBSK-UHFFFAOYSA-N |
| CCOCCOCCNC(═O)CON | MVBWGWFEBZTDRL-UHFFFAOYSA-N |
| CCOCCOCCOCCNC(═O)CON | CGVIAZZERQTZME-UHFFFAOYSA-N |
| CCCCCCNC(C)═O | SYIOXNGUUYGIIF-UHFFFAOYSA-N |
| CCCCCCNC(═O)CO | SCPUJVKFJZGFHO-UHFFFAOYSA-N |
| CCCNC(═O)CO | AVMYPXOTVDWXBQ-UHFFFAOYSA-N |
| CCNC(═O)CO | HWVOWKVXWMUGMS-UHFFFAOYSA-N |
| CCOCCOCCOCCNC═O | PMJXDYYSGBCCKN-UHFFFAOYSA-N |
| CCOCCOCCOCCOCCNC═O | USNKTLYDYCQPIY-UHFFFAOYSA-N |
| O═CCCCN1C═C(COCCOCCOCC═O)N═N1 | PIERMBSUWGQIIM-UHFFFAOYSA-N |
| CC1═CN(CCCCC═O)N═N1 | BDVLFPPECGWUGW-UHFFFAOYSA-N |
| CCOCCN1C═C(CCCC(N)═O)N═N1 | HHFSCJJMVBUCCJ-UHFFFAOYSA-N |
| CCOCCOCCN1C═C(CCCC(N)═O)N═N1 | YUKGCMCMOXBDMR-UHFFFAOYSA-N |
| CCOCCOCCOCCN1C═C(CCCC(N)═O)N═N1 | VKADYKRAOVRMLK-UHFFFAOYSA-N |
| CCOCCOCCOCCOCCN1C═C(CCCC(N)═O)N═N1 | SGKUSCWMMYTJMU-UHFFFAOYSA-N |
| CCCCCNC(═O)CO | BOELJSPAIXVQPX-UHFFFAOYSA-N |
| CCCCCCCCCCCNC(═O)CO | SPCBFTQUZSEEMK-UHFFFAOYSA-N |
| NCCOCCOCCOCC(═O)NCCCO | OMPNHKUEEONSGM-UHFFFAOYSA-N |
| NCCOCCOCCNC(═O)CON | NVASRKBDKFQDEH-UHFFFAOYSA-N |
| NCCOCCOCCOCC-O | CFXYEARFECEFDH-UHFFFAOYSA-N |
| CCNC(═O)CC | ABMDIECEEGFXNC-UHFFFAOYSA-N |
| CCCNC(═O)CC | YUMCRXLLWKQDJY-UHFFFAOYSA-N |
| CCCCNC(═O)CC | XQZDWKBCGAJXLC-UHFFFAOYSA-N |
| CCCCCNC(═O)CC | IVHSRZSQMQJLKE-UHFFFAOYSA-N |
| CCOCCOCCOCC═O | VIGPEKUIVIPDAG-UHFFFAOYSA-N |
| CC(═O)NCCOCCOCCOCCC═O | SVZLKBWUDJMXGH-UHFFFAOYSA-N |
| NCCCCC═O | SZBGXBOFCGNPEU-UHFFFAOYSA-N |
| NCCCCCCC═O | UGLZOVSJRNQLCS-UHFFFAOYSA-N |
| NCCCCCCCCCCC═O | XSVQSLVVCYRXCL-UHFFFAOYSA-N |
| CCCOCCC═O | OPNGQDQTKIMUGS-UHFFFAOYSA-N |
| O═CCCCCCCCCCO | DCZAVTGNGIDZGP-UHFFFAOYSA-N |
| CCCCCCCCCO | ZWRUINPWMLAQRD-UHFFFAOYSA-N |
| O═CCOCCOCCOCCOCCO | YLRRZGLZQJPGIB-UHFFFAOYSA-N |
| COCCOCCOCCOCCO | ZNYRFEPBTVGZDN-UHFFFAOYSA-N |
| O═CCOCCOCCOCCOCCOCCOCCO | CTLLATPOKUEFSQ-UHFFFAOYSA-N |
| COCCOCCOCCOCCOCCOCCO | FHHGCKHKTAJLOM-UHFFFAOYSA-N |
| CCOCCOCCOCCOCCOCCOCC═O | RRFBZXWBPXSEKK-UHFFFAOYSA-N |
| CCOCCOCCOCCOCCOCCOC | PJXDGFJDVVVXCY-UHFFFAOYSA-N |
| CC(═O)NCCCC═O | DDSLGZOYEPKPSJ-UHFFFAOYSA-N |
| CC(═O)NCCCCC═O | CMKXYMURJCKXEU-UHFFFAOYSA-N |
| CC(═O)NCCCCCC═O | LKNUIOZKLGQZEF-UHFFFAOYSA-N |
| CC(═O)NCCCCCCC═O | UGBYBQRARHYUPP-UHFFFAOYSA-N |
| CC(═O)NCCCCCCCC═O | MCEDGEZMTOLISW-UHFFFAOYSA-N |
| CC1═CN(CCOCC═O)N═N1 | LTPBTBKYFLGBGS-UHFFFAOYSA-N |
| CC1═CN(CCOCCOCC═O)N═N1 | QIHDIUBRPOAASD-UHFFFAOYSA-N |
| CC1═CN(CCOCCOCCOCC═O)N═N1 | BROMBAVESWKBCM-UHFFFAOYSA-N |
| CC1═CN(CCOCCOCCOCCOCC═O)N═N1 | IYNSOQCJNWTULH-UHFFFAOYSA-N |
| CCC1═CC═C(C)C═C1 | JRLPEMVDPFPYPJ-UHFFFAOYSA-N |
| CCOC1═CC═C(C)C═C1 | WSWPHHNIHLTAHB-UHFFFAOYSA-N |
| CCCCCCNC(C)═O | SYIOXNGUUYGIIF-UHFFFAOYSA-N |
| CCCC(═O)NCCCCO | NLLUMRGWPIHEMT-UHFFFAOYSA-N |
| CC(═O)NCCOCCOCCC═O | QFBWCXMYMIWRBS-UHFFFAOYSA-N |
| O═CCCCCNC(═O)CCCC═O | IQCMDZNBHIBZSX-UHFFFAOYSA-N |
| CC(═O)NCCOCCOCCOCCOCCC═O | ITOYTQVWBLQPSD-UHFFFAOYSA-N |
| CC(═O)NCCOCCOCCN | MLKVGKULFLDWJW-UHFFFAOYSA-N |
| C#CCOCCOCCOCC | NIFIKPHRYNDRRF-UHFFFAOYSA-N |
| C#CCCCCCCCCCC | ZVDBUOGYYYNMQI-UHFFFAOYSA-N |
| C#CCOCCOCCOCCO | CXJWUJYYDLYCCQ-UHFFFAOYSA-N |
| C#CCCCCCCCCCCO | XNRAUTMOUDUPET-UHFFFAOYSA-N |
| C#CCOCCOCCOCCC═O | SYPOHBRJZXHGFH-UHFFFAOYSA-N |
| C#CCCCCCCCCCCC═O | ZEFRCWJAFLYMSB-UHFFFAOYSA-N |
| C#CCN1CCC2(CC1)CCN(C1═NC═CC═N1)CC2 | OESPBQYPZBPQTF-UHFFFAOYSA-N |
| COCCOCCOCCNC(═O)CON | NQKDEZXAUUNANW-UHFFFAOYSA-N |
| COC1═CC═C(/C═N/NC═O)C═C1 | BUMDTJVYJRVRIX-UXBLZVDNSA-N |
| CCCOC1═CC═C(/C═N/NC═O)C═C1 | DDZUHXZWWDNROQ-XYOKQWHBSA-N |
| CCCCOC1═CC═C(/C═N/NC═O)C═C1 | QJOREBJFYYGLGD-UKTHLTGXSA-N |
| CCCCCOC1═CC═C(/C═N/NC═O)C═C1 | HBPCKSDVQXWCJB-GXDHUFHOSA-N |
| CCCCCCOC1═CC═C(/C═N/NC═O)C═C1 | KDAOUWVFTUQSOM-RVDMUPIBSA-N |
| CCCCCCCOC1═CC═C(/C═N/NC═O)C═C1 | OCZUZWSFEPFMNM-FOWTUZBSSA-N |
| COCCOC1═CC═C(/C═N/NC═O)C═C1 | WANQARQNRHYIGG-XYOKQWHBSA-N |
| COCCOCCOC1═CC═C(/C═N/NC═O)C═C1 | FMQPRUKKTCBPEK-GXDHUFHOSA-N |
| COCCOCCOCCOC1═CC═C(/C═N/NC═O)C═C1 | VJVIQOAIZCSWDV-FOWTUZBSSA-N |
| COCCOCCOCCOCCOC1═CC═C(/C═N/NC═O)C═C1 | BCEMPQXHJBHMKK-NBVRZTHBSA-N |
| COCCOCCOCCOCCOCCOC1═CC═C(/C═N/NC═O)C═C1 | ADBUPJVGDAVGTN-CAPFRKAQSA-N |
| COCCOCCOCCOCCOCCOCCOC1═CC═C(/C═N/NC═O)C═C1 | PGFFJCINGISICS-RELWKKBWSA-N |
| C#CCNC(═O)COC1═CC═C(/C═N/NC═O)C═C1 | GAFQWLJNNYFHQQ-OVCLIPMQSA-N |
| C#CCNC(═O)CCCOC1═CC═C(/C═N/NC═O)C═C1 | KKKLEMASLHHQPZ-GZTJUZNOSA-N |
| C#CCNC(═O)CCCCOC1═CC═C(/C═N/NC═O)C═C1 | YHKQAGDXSKKCLI-LDADJPATSA-N |
| C#CCNC(═O)CCCCCOC1═CC═C(/C═N/NC═O)C═C1 | SZOXJFMKFJKTCM-CPNJWEJPSA-N |
| C#CCNC(═O)CCCCCCOC1═CC═C(/C═N/NC═O)C═C1 | WZYZJSCZZKFJQK-XSFVSMFZSA-N |
| C#CCNC(═O)CCCCCCCOC1═CC═C(/C═N/NC═O)C═C1 | OXSFPFIZYKCSHT-RCCKNPSSSA-N |
| C#CCNC(═O)COCCOC1═CC═C(/C═N/NC═O)C═C1 | FNJIVVPTPZXBJN-LICLKQGHSA-N |
| C#CCNC(═O)COCCOCCOC1═CC═C(/C═N/NC═O)C═C1 | CTYKIWOGVVKSIP-XDHOZWIPSA-N |
| C#CCNC(═O)COCCOCCOCCOC1═CC═C(/C═N/NC═O)C═C1 | OPDCBWXZBZLRBM-KGENOOAVSA-N |
| C#CCNC(═O)COCCOCCOCCOCCOC1═CC═C(/C═N/NC═O)C═C1 | GROBTEUFCXRUNR-XQNSMLJCSA-N |
| C#CCNC(═O)COCCOCCOCCOCCOCCOC1═CC═C(/C═N/NC═O) | RHXYDIXBWDSPPO-XIEYBQDHSA-N |
| C═C1 | |
| C#CCNC(═O)COCCOCCOCCOCCOCCOCCOC1═CC═C(/C═N/NC | RKWZEGMUMOWRTL-NHFJDJAPSA-N |
| ═O)C═C1 | |
| COC1═CC═C(/C═N/N)C═C1 | YGPAJUBACUQPDB-UXBLZVDNSA-N |
| CCCOC1═CC═C(/C═N/N)C═C1 | OXYFCXREAYFPDZ-XYOKQWHBSA-N |
| CCCCOC1═CC═C(/C═N/N)C═C1 | MFWGQLZQFZJLJT-UKTHLTGXSA-N |
| CCCCCOC1═CC═C(/C═N/N)C═C1 | OTPACXZHXCCNAU-GXDHUFHOSA-N |
| CCCCCCOC1═CC═C(/C═N/N)C═C1 | PULLXMCFWFSODK-RVDMUPIBSA-N |
| CCCCCCCOC1═CC═C(/C═N/N)C═C1 | DPZIXPPVZMYWGQ-FOWTUZBSSA-N |
| COCCOC1═CC═C(C═N/N)C═C1 | LUDQJGRVDULCNY-XYOKQWHBSA-N |
| COCCOCCOC1═CC═C(/C═N/N)C═C1 | ODGUMAOJSGSGLV-GXDHUFHOSA-N |
| COCCOCCOCCOC1═CC═C(/C═N/N)C═C1 | XDVVVPJBIVJSHY-FOWTUZBSSA-N |
| COCCOCCOCCOCCOC1═CC═C(/C═N/N)C═C1 | JLHHDMPTKJQBKK-NBVRZTHBSA-N |
| COCCOCCOCCOCCOCCOC1═CC═C(/C═N/N)C═C1 | TXHJCKIISNHBJE-CAPFRKAQSA-N |
| COCCOCCOCCOCCOCCOCCOC1═CC═C(/C═N/N)C═C1 | FCIFBZLVTZMJGJ-RELWKKBWSA-N |
| CCCOC1═CC═C(CCNC═O)C═C1 | VGYCUVHCRSKUCP-UHFFFAOYSA-N |
| CCCCOC1═CC═C(CCNC═O)C═C1 | RXIJOEWSBXIZIM-UHFFFAOYSA-N |
| CCCCCOC1═CC═C(CCNC═O)C═C1 | WFJXHPFHAUFGOO-UHFFFAOYSA-N |
| CCNC(═O)COCCOCCOCC | SNIYVBISNQQNQP-UHFFFAOYSA-N |
| CCCCCCOCCOCCOC | SLXZPRDVXSNULE-UHFFFAOYSA-N |
| CCCCCCOCCOCCOCCOC | WECDVJWNQLMVAZ-UHFFFAOYSA-N |
| CCCCCCOCCCCCCOC | GICBTTLLBOJUPJ-UHFFFAOYSA-N |
| CCOC1═CC═C(OC)C═C1 | FTFNFGIOGXKJSP-UHFFFAOYSA-N |
| CCNC═O | KERBAAIBDHEFDD-UHFFFAOYSA-N |
| CCCNC═O | SUUDTPGCUKBECW-UHFFFAOYSA-N |
| CCCCNC═O | QQGNLKJAIVSNCO-UHFFFAOYSA-N |
| CCOCCOCCNC(═O)CCN | AMHKPKUSSYLBPW-UHFFFAOYSA-N |
| CCOCCOCCOCCOCCNC(═O)CCN | IGWZYCHVGYXPSZ-UHFFFAOYSA-N |
| NCCCC1═CN(COCCOCCOCCC═O)N═N1 | ZIHXQJDNOBPNNV-UHFFFAOYSA-N |
| NCCOCCOCCOCCC1═CN(COCCOCCOCCC═O)N═N1 | KQVVWIORIYTOAJ-UHFFFAOYSA-N |
| NCCOCCOCCOCCO | ANOJXMUSDYSKET-UHFFFAOYSA-N |
| NCCOCCOCCOCCOCCO | DEOUHEFHTMMUCM-UHFFFAOYSA-N |
| NCCOCCOCCOCCOCCOCCO | ICUIZKMGHRMMDZ-UHFFFAOYSA-N |
| CCOCCOCCOCCN | WWJVRDMJNJTOBL-UHFFFAOYSA-N |
| CCCCCCCCCCCCN | JRBPAEWTRLWTQC-UHFFFAOYSA-N |
| CCCN(C)CCCN | SMGLLZNMFIPPIT-UHFFFAOYSA-N |
| CCN1CCN(CCN)CC1 | SHUQIGHJQMJUHB-UHFFFAOYSA-N |
| CCCN1CCN(CCCN)CC1 | DJOXCABHIQUIJR-UHFFFAOYSA-N |
| CCCCN1CCN(CCCCN)CC1 | FTOWGLPSVKBMII-UHFFFAOYSA-N |
| CCOCCN1CCN(CCOCCN)CC1 | DSSQVCBOGPFZBU-UHFFFAOYSA-N |
| C | VNWKTOKETHGBQD-UHFFFAOYSA-N |
| COCCO | XNWFRZJHXBZDAG-UHFFFAOYSA-N |
| CCNC(═O)COCCOCCOC | KOZUMTCLSXRRSF-UHFFFAOYSA-N |
| CCNC(═O)COCCOCCOCCOC | GPYHKDJMEODULP-UHFFFAOYSA-N |
| CCNC(═O)COCCOCCOCCOCCOC | LGHRRVYRCMBQFR-UHFFFAOYSA-N |
| CCNC(═O)COCCOCCOCCOCCOCCOC | DXJUBWSTMHZRES-UHFFFAOYSA-N |
| CCNC(═O)COCCOCCOCCOCCOCCOCCOCCOCCOC | VHOVKYPYCOBJOC-UHFFFAOYSA-N |
| CCNC(═O)COCCCCCCOC | KYNRZXOWMLTXAP-UHFFFAOYSA-N |
| CCNC(═O)COCCCCCOCCOCCCCCOC | MVYNEDGDADJOKN-UHFFFAOYSA-N |
| CCNC(═O)COCCOCCOCC | SNIYVBISNQQNQP-UHFFFAOYSA-N |
| CCNC(═O)COCCOCCOCCOCCOCC | ZZGOPRXTBIHCQJ-UHFFFAOYSA-N |
| CCCCC(═O)NCC | ZOQTYYYRQHZQAR-UHFFFAOYSA-N |
| CCOCCOCCN1C═C(COCC)N═N1 | QOLOWJNCZRRAHU-UHFFFAOYSA-N |
| CCOCCOCCN1C═C(CCCC═O)N═N1 | ITZCDTIFEKCUFV-UHFFFAOYSA-N |
| CCOCCOCCOCCO | WFSMVVDJSNMRAR-UHFFFAOYSA-N |
| CCN1CCN(C2═NC═C(C═O)C═N2)CC1 | GNGGNAVEVANVSB-UHFFFAOYSA-N |
| CCCN1CCN(C2═CN═C(C═O)C═N2)CC1 | YYDOCAUSPSENMY-UHFFFAOYSA-N |
| CCOCCOCCOCCOCCOCCOCCC═O | YAMDEEMOVZPPRF-UHFFFAOYSA-N |
| CCOCCOCCOCCOCCOCCOCCOCCC═O | BOJVKVNRZUHKQQ-UHFFFAOYSA-N |
| CCOCCOCCOCCOCCOCCOCCOCCOCCOCCC═O | GFMWTQARKWSFOE-UHFFFAOYSA-N |
| O═CCCOCCOCCC(═O)NCCCO | FHHWBMOQOXPJTQ-UHFFFAOYSA-N |
| O═CCCOCCOCCOCCC(═O)NCCCO | BPHLQRMSGMPZBJ-UHFFFAOYSA-N |
| O═CCCOCCOCCOCCOCCOCCC(═O)NCCCO | XQFFPESYTIAUBO-UHFFFAOYSA-N |
| O═CCCOCCOCCOCCC(═O)NCCO | IUYGDSIFHWLZOJ-UHFFFAOYSA-N |
| O═CCCOCCOCCOCCOCCOCCOCCC(═O)NCCO | URXUTUCXMCFYQP-UHFFFAOYSA-N |
| O═CCCOCCOCCOCCOCCOCCOCCC(═O)NCCCO | VOWVHARCXATYBQ-UHFFFAOYSA-N |
| O═CCCOCCOCCOCCOCCOCCOCCOCCOCCOCCC(═O)NCCCO | XILVHWQJUGZJDA-UHFFFAOYSA-N |
| O═CCCOCCOCCOCCOCCOCCN1C═C(CO)N═N1 | FSOCHXPSNNQMBS-UHFFFAOYSA-N |
| COCCOCCOCCOC | YFNKIDBQEZZDLK-UHFFFAOYSA-N |
| COCCOCCOCCOCCOC | ZUHZGEOKBKGPSW-UHFFFAOYSA-N |
| COCCOCCOCCOCCOCCOC | DMDPGPKXQDIQQG-UHFFFAOYSA-N |
| CC(═O)NCCOCCOCCOCCOCCOCCN1C═C(C)N═N1 | UUBGNOYJKQHNOQ-UHFFFAOYSA-N |
| CCCCC1═CN(CCOCC)N═N1 | JZZCXCHBVFODNW-UHFFFAOYSA-N |
| CCCCC1═CN(CCOCCOCC)N═N1 | BINIRCXGDOCLLP-UHFFFAOYSA-N |
| CCCCC1═CN(CCOCCOCCOCC)N═N1 | SGOFPUDMPULTSZ-UHFFFAOYSA-N |
| CCCCC1═CN(CCOCCOCCOCCOCC)N═N1 | QQVHZLHVTFONSZ-UHFFFAOYSA-N |
| CCCCCCCCCCNC(C)═O | DXFXMYYJKFIEGI-UHFFFAOYSA-N |
| CCCCCCCCCCNC(═O)CO | AOLDPOOTBNCOFB-UHFFFAOYSA-N |
| CCOCCOCCN | KURRHYKFNUZCSJ-UHFFFAOYSA-N |
| COCCOCCOCCN | OKUWOEKJQRUMBW-UHFFFAOYSA-N |
| CCOCCOCCOC | CNJRPYFBORAQAU-UHFFFAOYSA-N |
| NCCC═O | PCXDJQZLDDHMGX-UHFFFAOYSA-N |
| NCCOCC═O | JKAJSVFYWNIQEL-UHFFFAOYSA-N |
| NCCOCCOCC═O | QZKNUSUQYMYGNK-UHFFFAOYSA-N |
| NCCOCCOCCOCCOCC═O | OISXLZLHNVGYLK-UHFFFAOYSA-N |
| CCCCOCCOCCOCCOCCN | DQNHOVOCLXFUNU-UHFFFAOYSA-N |
| CCOCCOCCOCCOCCN | LYKXJZZKGTXDRB-UHFFFAOYSA-N |
| O═CCOCCOCCOCCO | UYUVFRYLZGZLIO-UHFFFAOYSA-N |
| O═CCOCCOCCOCCOCC-O | KUDYUUPKMBPKIY-UHFFFAOYSA-N |
| CCCCNC(═O)COCCOCCOCCOCC-O | FCJGGDHUABZCPJ-UHFFFAOYSA-N |
| O═CCOCCOCCOCCOC1═CC═C(O)C═C1 | ISVAGPFLGNBSTE-UHFFFAOYSA-N |
| NCCOCCOCCOCCNC═O | OYZLQBKGSLNULP-UHFFFAOYSA-N |
| CCCOCCOCCOCC | PXQCQQARRAYIFS-UHFFFAOYSA-N |
| CCOC1═CC═C(OCCO)C═C1 | OKNKODGWSKWZIY-UHFFFAOYSA-N |
| CCOC1═CC═CC(OCCO)═C1 | QRYGDQPYLYVBTJ-UHFFFAOYSA-N |
| CCOC1═CC═CC═C1OCCO | KOFVDOFNEZNSKF-UHFFFAOYSA-N |
| CCCCC(═O)NC | XKEKKGKDCHCOSA-UHFFFAOYSA-N |
| CCCC(═O)NC | OLLZXQIFCRIRMH-UHFFFAOYSA-N |
| CCCCCC(═O)NC | RSPBQSYWXAROOO-UHFFFAOYSA-N |
| CCOCCC(═O)NC | WUEOXQAHIGLVQF-UHFFFAOYSA-N |
| CCCCCCC(═O)NC | PRCOHDSOXCGBAX-UHFFFAOYSA-N |
| CCCCCCCC(═O)NC | XDXKSZZAKNNKSG-UHFFFAOYSA-N |
| CCOCCOCCC(═O)NC | GHLLCTDCHRCASG-UHFFFAOYSA-N |
| CCOCCOCCOCCC(═O)NC | MLKNWKNNXGTPDS-UHFFFAOYSA-N |
| CCOCCOCCOCCOCCC(═O)NC | RVTMVIKCTXIPNF-UHFFFAOYSA-N |
| CCOCCOCCOCCOCCOCCC(═O)NC | KRBFLYOMQQTPTJ-UHFFFAOYSA-N |
| CCOCCN1C═C(CCCO)N═N1 | IWSQPTMFHBEAHK-UHFFFAOYSA-N |
| CCOCCN1C═C(CCCCO)N═N1 | DFTQGVZBCBLMFW-UHFFFAOYSA-N |
| CCOCCN1C═C(CCCCCO)N═N1 | HMEOFVRQQVDJFG-UHFFFAOYSA-N |
| CCOCCOCCN1C═C(CCCO)N═N1 | UNSWNVRCIMZFJJ-UHFFFAOYSA-N |
| CCOCCOCCN1C═C(CCCCO)N═N1 | PBBILCHMBMDMDY-UHFFFAOYSA-N |
| CCOCCOCCN1C═C(CCCCCO)N═N1 | ADGKYXDPFMCPHG-UHFFFAOYSA-N |
| CCOCCOCCOCCN1C═C(CCCO)N═N1 | PCHVTBIANZSUSG-UHFFFAOYSA-N |
| CCOCCOCCOCCN1C═C(CCCCO)N═N1 | VNTVPWWEIQTGQV-UHFFFAOYSA-N |
| CCOCCOCCOCCN1C═C(CCCCCO)N═N1 | XCXTYYXCSPSSTJ-UHFFFAOYSA-N |
| CCOCCOCCOCCOCCN1C═C(CCCO)N═N1 | DMKCTBXHKFRVQM-UHFFFAOYSA-N |
| CCOCCOCCOCCOCCN1C═C(CCCCO)N═N1 | QFNQVKWRDXBDSY-UHFFFAOYSA-N |
| CCOCCOCCOCCOCCN1C═C(CCCCCO)N═N1 | ZJBHOQJQDRHVFV-UHFFFAOYSA-N |
| CCOCCOCCOCCOCCOCCN1C═C(CCCO)N═N1 | OIVJUZPUZLKRGR-UHFFFAOYSA-N |
| CCOCCOCCOCCOCCOCCN1C═C(CCCCO)N═N1 | YEDVCDHMPLYQAJ-UHFFFAOYSA-N |
| CCOCCOCCOCCOCCOCCN1C═C(CCCCCO)N═N1 | RRFJMZCCJGNQLS-UHFFFAOYSA-N |
| CCCCCCCN1C═C(CO)N═N1 | PRZSQBMDQIOHLF-UHFFFAOYSA-N |
| CCCCCCN1C═C(CCCO)N═N1 | DYLGTNGZRWFHFJ-UHFFFAOYSA-N |
| CCCCCCCN1C═C(CCCO)N═N1 | QORVYNWMNGCIRL-UHFFFAOYSA-N |
| CCCCCCCN1C═C(CCCCO)N═N1 | DKQTWKGFGGXQAP-UHFFFAOYSA-N |
| CCCCCCCN1C═C(CCCCCO)N═N1 | LWAOBXABFHXDDO-UHFFFAOYSA-N |
| CCCCCCCN1C═C(CCCCCCO)N═N1 | OMTVQLKNGOTPFB-UHFFFAOYSA-N |
| CCCCCCCN1C═C(CCCCCCCO)N═N1 | JEBYVCNLWPYZBG-UHFFFAOYSA-N |
| C#CCOCCN1C═C(CCCO)N═N1 | BILFLRBABZUDQJ-UHFFFAOYSA-N |
| C#CCOCCCN1C═C(CCCO)N═N1 | SSJAZVKHKXJSGT-UHFFFAOYSA-N |
| C#CCOCCCCN1C═C(CCCO)N═N1 | FRJJREJZRCAOBR-UHFFFAOYSA-N |
| C#CCOCCCCCN1C═C(CCCO)N═N1 | HKHUPMKFFKWYMC-UHFFFAOYSA-N |
| C#CCOCCCCCCN1C═C(CCCO)N═N1 | MOOPHKQJFPDRPG-UHFFFAOYSA-N |
| C#CCOCCCCCCCN1C═C(CCCO)N═N1 | GOKFRTINPSATAP-UHFFFAOYSA-N |
| CCCOCCN1C═C(CCCO)N═N1 | YHKMGSCTIRYPBB-UHFFFAOYSA-N |
| CCCOCCCN1C═C(CCCO)N═N1 | DRKVUMKIVFWZCO-UHFFFAOYSA-N |
| CCCOCCCCN1C═C(CCCO)N═N1 | GVAJHRBFQACNFO-UHFFFAOYSA-N |
| CCCOCCCCCN1C═C(CCCO)N═N1 | YESSXODTLDJENJ-UHFFFAOYSA-N |
| CCCOCCCCCCN1C═C(CCCO)N═N1 | COOMZBGRZHIZTC-UHFFFAOYSA-N |
| CCCOCCCCCCCN1C═C(CCCO)N═N1 | QJHHQISDRBGOMA-UHFFFAOYSA-N |
| CCCCCCNCCC | WBLXZSQLBOFHAB-UHFFFAOYSA-N |
| CCCCCCCCCCNCCC | LBTAXVIGKZQJMU-UHFFFAOYSA-N |
| CCCNCCOCCOCCOC | VEMCFHQRDLCNDG-UHFFFAOYSA-N |
| CCCNCCOCC | IKQYXWYZNWOCCV-UHFFFAOYSA-N |
| CCCNCC | XCVNDBIXFPGMIW-UHFFFAOYSA-N |
| CC1═CN(CCO)N═N1 | NRWUEYNWZWYICS-UHFFFAOYSA-N |
| CC1═CN(CCCO)N═N1 | KRUUJTZGQCNXHD-UHFFFAOYSA-N |
| CC1═CN(CCCCO)N═N1 | JKMQIHWCNUFEPC-UHFFFAOYSA-N |
| CC1═CN(CCCCCO)N═N1 | IUUPUASDOPLFAW-UHFFFAOYSA-N |
| CC1═CN(CCCCCCO)N═N1 | HOZOHESWDGGHPU-UHFFFAOYSA-N |
| CCC1═CN(CCO)N═N1 | BMUHMPXOOYMXBI-UHFFFAOYSA-N |
| CCC1═CN(CCCO)N═N1 | MWMJCIBSEHTNII-UHFFFAOYSA-N |
| CCC1═CN(CCCCO)N═N1 | SSIHTHZHHHWLPH-UHFFFAOYSA-N |
| CCCC1═CN(CCO)N═N1 | CSMMGTBVKLOOHC-UHFFFAOYSA-N |
| CCCC1═CN(CCCO)N═N1 | JVMBNSCDMIGKPE-UHFFFAOYSA-N |
| CCCC1═CN(CCCCO)N═N1 | RUISCQUSWXRDES-UHFFFAOYSA-N |
| CCCCC1═CN(CCO)N═N1 | KOCRUAQUOXFDII-UHFFFAOYSA-N |
| CCCCC1═CN(CCCO)N═N1 | JCXDQYGIIXAYDR-UHFFFAOYSA-N |
| CCCCC1═CN(CCCCO)N═N1 | JSRCAHMNEWDCGG-UHFFFAOYSA-N |
| CCCCC1═CN(CCCCCO)N═N1 | LOEAYKUOBVRMTQ-UHFFFAOYSA-N |
| CCCCC1═CN(CCCCCCO)N═N1 | XPCNPRBLXZKINK-UHFFFAOYSA-N |
| CCCCC1═CN(CCCCCCCO)N═N1 | BMPLYQYIDNRXTH-UHFFFAOYSA-N |
| CCCCC1═CN(CCCCCCCCO)N═N1 | CVVFEFBEGZUQCN-UHFFFAOYSA-N |
| CCCCC1═CN(CCCCCCCCCO)N═N1 | JDQVNUOGRXCASB-UHFFFAOYSA-N |
| CCCCC1═CN(CCCCCCCCCCO)N═N1 | YUGPODYNJVNIKL-UHFFFAOYSA-N |
| CCCCC1═CN(CCCCCCCCCCCO)N═N1 | LSFLKGDYYTXSAV-UHFFFAOYSA-N |
| CCCCC1═CN(CCCCCCCCCCCCO)N═N1 | QOFRJYTZYRGQMG-UHFFFAOYSA-N |
| CCCCC1═CN(CCOCCO)N═N1 | PXMOEILMQODSNH-UHFFFAOYSA-N |
| CCCCC1═CN(CCOCCOCCO)N═N1 | GTLVDKUPJMRXML-UHFFFAOYSA-N |
| CC(═O)NCCCCNC(═O)CO | YEFZOXRWQYKCTR-UHFFFAOYSA-N |
| CC(═O)NCCCCCCCCNC(═O)CO | PXSMGLZBYVTOHW-UHFFFAOYSA-N |
| CC(═O)NCCCOCCOCCOCCCNC(═O)CO | PYXQPJNDXZWANA-UHFFFAOYSA-N |
| CC(═O)NCCCCCCO | VJPODIJDERZHMG-UHFFFAOYSA-N |
| CC(═O)N1CCC(C2CCN(C(═O)CO)CC2)CC1 | KUBRWDVJAZPXJP-UHFFFAOYSA-N |
| COCCOCCNC(C)═O | LOQWIGQMEJJWNU-UHFFFAOYSA-N |
| CCNC(═O)CCOCC | YEGOEFQOEQJSRZ-UHFFFAOYSA-N |
| CCNC(═O)CCOCCOCCO | GIUBFYPQILEXBY-UHFFFAOYSA-N |
| CCNC(═O)CCOCCOCCOCCOCCO | DATGOUIAIUXPPU-UHFFFAOYSA-N |
| CCNC(═O)CCOCCOCCOCCOCCOCCOCCOCCOCCO | ZISVZBSOFTWHBM-UHFFFAOYSA-N |
| CCNC(═O)CCCCCO | QHLUIQTVNISOIX-UHFFFAOYSA-N |
| CCNC(═O)CCCCCCCO | XLRRTYKEUHTHAB-UHFFFAOYSA-N |
| CCNC(═O)CCCCCCCCO | MWKCVHREYBCVST-UHFFFAOYSA-N |
| O═CCOCCN1C═C(CNC═O)N═N1 | FAZNERCMJWLWII-UHFFFAOYSA-N |
| O═CCOCCOCCN1C═C(CNC═O)N═N1 | BVDRXENQISFSCE-UHFFFAOYSA-N |
| O═CCOCCOCCOCCN1C═C(CNC═O)N═N1 | WERPWBACJUTJFQ-UHFFFAOYSA-N |
| O═CCOCCOCCOCCOCCN1C═C(CNC═O)N═N1 | XGFNLYULIBUKHN-UHFFFAOYSA-N |
| CCCOCCOCCOCCCNC═O | YCNHUBWRMHEXTJ-UHFFFAOYSA-N |
| O═CNCCOCCOCCOCCNC(═O)CO | KFXXYDXPDPSTKQ-UHFFFAOYSA-N |
| O═CNCCOCCCOCCCOCCNC(═O)CO | OWJZTKVATBQRQW-UHFFFAOYSA-N |
| O═CNCCOCCOCCNC(═O)CO | QIMRSKVQPPNDRK-UHFFFAOYSA-N |
| O═CNCCCCCCNC(═O)CO | WGDQNVYGYCISGI-UHFFFAOYSA-N |
| CCOCCOCCOCCNC(C)═O | JOVFTSYTPCENPI-UHFFFAOYSA-N |
| O═CCCC(═O)NCCCCCCCCNC(═O)CO | LXFPQXZBCPFWNJ-UHFFFAOYSA-N |
| CCCNC(═O)CCOCCOCC | NEWWMLJVESJAAF-UHFFFAOYSA-N |
| NCCNC(═O)CCCC═O | CEUGEDINLFNUSC-UHFFFAOYSA-N |
| O═CCCCC(═O)N1CCNCC1 | SLIHUUCJUNRANB-UHFFFAOYSA-N |
| NCCCCNC(═O)CCCC═O | RHVPBISPAJWJGG-UHFFFAOYSA-N |
| NCCCNC(═O)CCCC═O | XRFMLSFNHDXOHL-UHFFFAOYSA-N |
| NCCCCCNC(═O)CCCC═O | XJUKTVLMFRDALL-UHFFFAOYSA-N |
| O═CCCC(═O)N1CCNCC1 | DHPNOXGPTQKXSO-UHFFFAOYSA-N |
| NCCNC(═O)CCC═O | KJBWSGQVSDIYTF-UHFFFAOYSA-N |
| NCCNC(═O)COCC═O | FXQPSFDYDXNJOE-UHFFFAOYSA-N |
| O═CCCCN1C═C(COCCOCCO)N═N1 | CWABEEFATYGLMY-UHFFFAOYSA-N |
| O═CCCCN1C═C(COCCOCCOCCO)N═N1 | YHEGHZHPIWGXTC-UHFFFAOYSA-N |
| O═CCOCCOCCO | UKSDOCCELISUDS-UHFFFAOYSA-N |
| O═CCOCCOCCOCCOCCO | YLRRZGLZQJPGIB-UHFFFAOYSA-N |
| O═CCCCO | PIAOXUVIBAKVSP-UHFFFAOYSA-N |
| O═CCCCCO | CNRGMQRNYAIBTN-UHFFFAOYSA-N |
| O═CCCCCCO | FPFTWHJPEMPAGE-UHFFFAOYSA-N |
| O═CCCCCCCO | JOXWSBFBXNGDFD-UHFFFAOYSA-N |
| CCCCC1═CN(CCOCCOCCOCCOCCOCC)N═N1 | GSUYJWNWMQZZGH-UHFFFAOYSA-N |
| CCCCC(═O)NCCOCCOCCOCC | SKVWTCBCFAUHJU-UHFFFAOYSA-N |
| CCCCCC(═O)NCCOCCOCCOCC | NSRZCJUJFTZOLR-UHFFFAOYSA-N |
| CCCCC1═CN(CCOCCOCCOCCC)N═N1 | NDFAKQBEFLGODK-UHFFFAOYSA-N |
| CCCCC1═CN(CCOCCOCCOCCOCCC)N═N1 | HVTUOIAYHRVEIG-UHFFFAOYSA-N |
| CCOCCOCCOCCOCCOCCOCCOCC | AETIPHBKCCVQJU-UHFFFAOYSA-N |
| NCCCOCCOCCOCCCNC(═O)CO | LPMUYWFVSIBQPT-UHFFFAOYSA-N |
| NCCCCCCO | SUTWPJHCRAITLU-UHFFFAOYSA-N |
| OCCN1C═C(CNCC2═CC═CC═C2)N═N1 | GKPQACDDXDBDBA-UHFFFAOYSA-N |
| OCCCN1C═C(CNCC2═CC═CC═C2)N═N1 | NPQOERSJJRPCOH-UHFFFAOYSA-N |
| OCCCCN1C═C(CNCC2═CC═CC═C2)N═N1 | HXELQKPWCCNTSR-UHFFFAOYSA-N |
| OCCCCCN1C═C(CNCC2═CC═CC═C2)N═N1 | XHYXPJQPXQMZHW-UHFFFAOYSA-N |
| OCCCCCCN1C═C(CNCC2═CC═CC═C2)N═N1 | QMVZZMVMANFZMQ-UHFFFAOYSA-N |
| OCCCCCCCN1C═C(CNCC2═CC═CC═C2)N═N1 | SMRNNCAVAQDLKA-UHFFFAOYSA-N |
| CCCCCCCCCCCC | SNRUBQQJIBEYMU-UHFFFAOYSA-N |
| CCCCCCCCCCCC═O | HFJRKMMYBMWEAD-UHFFFAOYSA-N |
| CCCOCCOCCOCCOCCOCCOCCOCCOCC | PNZYVXVXQOENSX-UHFFFAOYSA-N |
| CCOCCOCCNC(═O)CC | QKYXHWUMMQJDMD-UHFFFAOYSA-N |
| CCCNC1═CC═C(OCCOCC)C═C1 | WKDQTXUJGGIDFP-UHFFFAOYSA-N |
| CCCNC1═CC═C(OCCOCCOCC)C═C1 | CQYNWZNEZRJJIC-UHFFFAOYSA-N |
| CCCN1C═C(CNC2═CC═C(OCCOCC)C═C2)N═N1 | CVYQFNNRJNFSIS-UHFFFAOYSA-N |
| CCCN1C═C(CNC2═CC═C(OCCOCCOCC)C═C2)N═N1 | YXNMWXZHLHSOFQ-UHFFFAOYSA-N |
| CCCCCCNC═O | NHTXRWUMLXSOGJ-UHFFFAOYSA-N |
| O═CNCCOCCOCCO | UZVKVPOLCGMMJN-UHFFFAOYSA-N |
| CCOCCOCCOCCOCCOCC═O | CWLPBNKOWOZCQP-UHFFFAOYSA-N |
| CCCCCCCN | WJYIASZWHGOTOU-UHFFFAOYSA-N |
| CCCCCCOCCOCC | CMZCBYJFESJOFV-UHFFFAOYSA-N |
| CCCCCCOCCOCCOCCOCCO | VUEUVIPIBVJLCY-UHFFFAOYSA-N |
| COCCOCCOCCOCCOCCO | SLNYBUIEAMRFSZ-UHFFFAOYSA-N |
| CCCCOCCOCCOCCOCCO | MXVMODFDROLTFD-UHFFFAOYSA-N |
| CC(C)CCCCCOCCOCCOCCO | FVNOIEMNRONTEK-UHFFFAOYSA-N |
| COCCOCCOCCOCCO | ZNYRFEPBTVGZDN-UHFFFAOYSA-N |
| COCCOCCOCCO | JLGLQAWTXXGVEM-UHFFFAOYSA-N |
| CCC1═CN(CCOCCOCCOCCOCCOCCO)N═N1 | IRFBBMPVJLKBGZ-UHFFFAOYSA-N |
| CCC1═CN(CCOCCOCCOCCO)N═N1 | OWGQNMNFCUTSGC-UHFFFAOYSA-N |
| CCC1═CN(CCOCCOCCO)N═N1 | XEUGWMQNMWTUKF-UHFFFAOYSA-N |
| CCC1═CN(CCOCCO)N═N1 | LQQLIFHJYQZQOT-UHFFFAOYSA-N |
| CCC1═CN(CCOCCOCCOCCOC(═O)NCCO)N═N1 | MFATVXPNYNAXJA-UHFFFAOYSA-N |
| CCC1═CN(CCOCCOCCOC(═O)NCCO)N═N1 | JKLJYBMEECRFBU-UHFFFAOYSA-N |
| CCC1═CN(CCOCCOC(═O)NCCO)N═N1 | POYTYACNRWCRMP-UHFFFAOYSA-N |
| CCC1═CN(CCOCCOCCOCCOCCOCCOC(═O)NCCO)N═N1 | ZPMPGBDPRYIRPZ-UHFFFAOYSA-N |
| CCCCCCCCNC(═O)CO | PWGOKBGNJNLIDS-UHFFFAOYSA-N |
| CCCOCCOCCOCCCNC(═O)CO | BBDDYLXZIGBXQL-UHFFFAOYSA-N |
| CCCCCCCCNC(═O)C1═CC═C(C)C═C1 | YLOQYJXWAPFPMJ-UHFFFAOYSA-N |
| CCCCCCNC═O | NHTXRWUMLXSOGJ-UHFFFAOYSA-N |
| CCCCCCCCNC═O | ZBWPKQRQZDZVSF-UHFFFAOYSA-N |
| COCCOCCOCCN1C═C(CC═O)N═N1 | UDQPFKUVGNTGHJ-UHFFFAOYSA-N |
| CN(CC═O)CCNC(═O)CO | XWRCNXZASLPYBQ-UHFFFAOYSA-N |
| O═CCNCCOCCOCCOCCNC(═O)CO | HBFMQYKFJKCGBG-UHFFFAOYSA-N |
| O═CCNCCCCNC(═O)CO | LYCLRJBFYCMHGF-UHFFFAOYSA-N |
| CC(═O)NCCN(C)CC═O | RDDOXWPLJUWKHY-UHFFFAOYSA-N |
| CCCCCOCCCOC | SRNPGWZFBBKXEF-UHFFFAOYSA-N |
| COCCCOCCN1CCCCC1 | GJFTZLWOCFLINT-UHFFFAOYSA-N |
| COCCCCCO | OMNKOGMRWWOOFR-UHFFFAOYSA-N |
| COCCCCCN | DMRHQYSRQGGRCK-UHFFFAOYSA-N |
| COCCC1═CC═CC═C1 | CQLYXIUHVFRXLT-UHFFFAOYSA-N |
| COCC1═CC═CC═C1 | GQKZBCPTCWJTAS-UHFFFAOYSA-N |
| CCOCCOCCOCCNC(═O)CC | DQKOWFBYHIENAP-UHFFFAOYSA-N |
| CCCOCCCCCO | YAWHDVYIZZNQLG-UHFFFAOYSA-N |
| CCCCCOC1═CC═CC═C1 | HPUOAJPGWQQRNT-UHFFFAOYSA-N |
| CCOCCOCCOCCOCCOC1═CC═C(N)C═C1 | RLLXXZFEMFANLF-UHFFFAOYSA-N |
| CCOCCOC1═CC═C([C@H](C)N)C═C1 | VDYRRXUKQOWKRN-JTQLQIEISA-N |
| CCOCCN1CCCCC1CNC═O | PYZSMHZTVRAXMP-UHFFFAOYSA-N |
| CCNCCOCCN | VQUWJUIPMCCLSD-UHFFFAOYSA-N |
| CCCOCCOCCN(C)C═O | HSUUAHXXMSJDIC-UHFFFAOYSA-N |
| CCCOCCOCCN | UPYSGFWVMLQLGJ-UHFFFAOYSA-N |
| NCCOCCOCCN1CCCCC1 | SYZCJSVGJSTZHP-UHFFFAOYSA-N |
| CCOCCN1CCOC(CNC)C1 | BBGHDSWTIVBDJG-UHFFFAOYSA-N |
| CCCOCCOCCN(C)C | ZLIFDEDLYLCCGA-UHFFFAOYSA-N |
| CCCOCCN(C)C | DARJCOYDJSZUAC-UHFFFAOYSA-N |
| CN1CCN(CCOCCOCCO)CC1 | ORNJSISNUBXZAT-UHFFFAOYSA-N |
| CN1CCN(CCOCCOCCOCCO)CC1 | AHDKSJCZGIBPOH-UHFFFAOYSA-N |
| CN1CCN(CC2CCNCC2)CC1 | MIRBDUREIHMEMK-UHFFFAOYSA-N |
| CCOCCO | ZNQVEEAIQZEUHB-UHFFFAOYSA-N |
| CN1CCN(CC2CCC(O)CC2)CC1 | ARPHSRLZPCLRNY-UHFFFAOYSA-N |
| COCCCOCCO | YYIOSNBPOLUPST-UHFFFAOYSA-N |
| COCCCOCCCO | QCAHUFWKIQLBNB-UHFFFAOYSA-N |
| COCCOCCC(F)(F)CCO | YSHKLLQWEMCYRM-UHFFFAOYSA-N |
| COCCOCCCCCO | NTXQMKIQPXFSAA-UHFFFAOYSA-N |
| COCCOCCOCCOCCN(C)C(═O)CCN | DEPUURDOWPFPKV-UHFFFAOYSA-N |
| COCCOCCOCCOCCOCCN(C)C(═O)CCN | QSEIYFPGGDEASH-UHFFFAOYSA-N |
| CCOCCOCCOCCN(C)C(═O)CCN | JAPQVSRYVVVHLP-UHFFFAOYSA-N |
| CN(CCOCCOCCOCCOCCO)C(═O)CCN | FWQUOUJESIJPDG-UHFFFAOYSA-N |
| CCNC(═O)CC═O | CNSQBBYLYQXGGP-UHFFFAOYSA-N |
| CCCNC(═O)CC═O | DWERFNSKTADUGD-UHFFFAOYSA-N |
| CCCCNC(═O)CC═O | PVWAVSAIGBNVEN-UHFFFAOYSA-N |
| CCCCCNC(═O)CC═O | ITVQKZMUSBWQBQ-UHFFFAOYSA-N |
| CCCCCCNC(═O)CC═O | AUSHRFGMUIXPMZ-UHFFFAOYSA-N |
| CCCCCCCNC(═O)CC═O | ZILMYZIAGQKVFP-UHFFFAOYSA-N |
| CCCCCCCCNC(═O)CC-O | SNNNCFJLPODPQU-UHFFFAOYSA-N |
| CCCCCCCCCNC(═O)CC═O | QWMZZSBBFSTZQM-UHFFFAOYSA-N |
| CCCCCCCCCCNC(═O)CC═O | TWAZOSXOKOSESD-UHFFFAOYSA-N |
| CCCCCCCCCCCNC(═O)CC═O | BVGSZJINMRFREK-UHFFFAOYSA-N |
| CCCCCCCCCCNC(═O)CCC═O | MTRKRZOEMQQXBU-UHFFFAOYSA-N |
| CCCCCCCCCCCNC(═O)CCC═O | AMXCHGPKQNMCCQ-UHFFFAOYSA-N |
| CCCCCCCCCCCNC(═O)CCCC═O | GKYGBGKPALXWAX-UHFFFAOYSA-N |
| COCCOCCOCCNC(═O)CCC═O | FWZIDJPEAIKIRE-UHFFFAOYSA-N |
| CCOCCOCCOCCNC(═O)CCC═O | LGBVNQAEIUSEGZ-UHFFFAOYSA-N |
| CCOCCOCCOCCOCCNC(═O)CCC═O | LVWSUIJNUPKSOQ-UHFFFAOYSA-N |
| CCCCCCCCCCN1CCN(CC═O)CC1 | NEDMKGHXZIHEOA-UHFFFAOYSA-N |
| CCCCCCCCCCCN1CCN(CC═O)CC1 | NGHBSVFOXOQHEK-UHFFFAOYSA-N |
| CCCCCCCCN1CCN(C(═O)CCC═O)CC1 | QKQKJQVNDQREQU-UHFFFAOYSA-N |
| CCCCCCCCCN1CCN(C(═O)CCC═O)CC1 | FCGQTZDAHVECBX-UHFFFAOYSA-N |
| CCCCCCCOC1═CC═C(NC(═O)CCC═O)C═C1 | QNXNMFGSQVNMIC-UHFFFAOYSA-N |
| CCCCN1CCN(CCCCNC(═O)CCC═O)CC1 | GPIIUCXHISRUGD-UHFFFAOYSA-N |
| CCOCCOCCN1C═C(C(N)═O)N═N1 | VIPMOOADDACLIH-UHFFFAOYSA-N |
| CCOC1═CC═C(/N═N/C2═CC═C(C═O)C═C2)C═C1 | YDVIPWQIWQDCOL-WUKNDPDISA-N |
| CCCOC1═CC═C(/N═N/C2═CC═C(C═O)C═C2)C═C1 | BNAYKHHBJGUGEU-ISLYRVAYSA-N |
| CCCCOC1═CC═C(/N═N/C2═CC═C(C═O)C═C2)C═C1 | FWBXPKPOECYWPJ-VHEBQXMUSA-N |
| CCCCCOC1═CC═C(/N═N/C2═CC═C(C═O)C═C2)C═C1 | SHUJZLJCZICRKA-FMQUCBEESA-N |
| CCCCCCOC1═CC═C(/N═N/C2═CC═C(C═O)C═C2)C═C1 | WSUPCNHPNYJESO-QZQOTICOSA-N |
| O═CC1═CC(F)═C(/N═N/C2═C(F)C═CC═C2F)C(F)═C1 | PUDWNQLFAIUHLK-VHEBQXMUSA-N |
| COC1═CC(N═N)═CC(OC)═C1OCC(═O)NCCN | RNABUUZQSVDBHF-UHFFFAOYSA-N |
| COC1═CC(N═N)═CC(OC)═C1OCC(═O)NCCCN | WKQAPJVXMOPDAF-UHFFFAOYSA-N |
| COC1═CC(N═N)═CC(OC)═C1OCC(═O)NCCCCN | JHZPHMRQFKRVGR-UHFFFAOYSA-N |
| COC1═CC(N═N)═CC(OC)═C1OCC(═O)NCCCCCN | IRWQAIBTQBIVLP-UHFFFAOYSA-N |
| COC1═CC(N═N)═CC(OC)═C1OCC(═O)NCCCCCCN | XSDKMNILSJTBHK-UHFFFAOYSA-N |
| N═NC1═CC═C(OCC(═O)NCCN)C═C1 | XHJWSVKSIDOIIN-UHFFFAOYSA-N |
| N═NC1═CC═C(OCC(═O)NCCCCN)C═C1 | COEAFSFRZAIQIX-UHFFFAOYSA-N |
| N═NC1═CC═C(OCC(═O)NCCCCCCN)C═C1 | ZSZVDUURTJZCJL-UHFFFAOYSA-N |
| NC1═CC═C(/N═N/C2═CC═C(NC(═O)CO)C═C2)C═C1 | DMSNNDSZXSHGHL-ISLYRVAYSA-N |
| NCC1═CC═C(/N═N/C2═CC═C(NC(═O)CO)C═C2)C═C1 | VWXHUHBUMINWMB-VHEBQXMUSA-N |
| NCCC1═CC═C(/N═N/C2═CC═C(NC(═O)CO)C═C2)C═C1 | DVMAQNQLRXSAKX-FMQUCBEESA-N |
| NC1═CC═C2/N═N\C3═CC═C(NC(═O)CO)C═C3CCC2═C1 | JXQSKOZOEWNFBG-VXPUYCOJSA-N |
| COC1═CC(N═N)═CC(OC)═C1OCC(═O)NCCCNC(═O)CO | AEPAXJWFDYSIPS-UHFFFAOYSA-N |
| COC1═CC(N═N)═CC(OC)═C1OCC(═O)NCCCCNC(═O)CO | CGFSXBHORDYMJF-UHFFFAOYSA-N |
| COC1═CC(N═N)═CC(OC)═C1OCC(═O)NCCCCCNC(═O)CO | OWNVBRBRJYRFNI-UHFFFAOYSA-N |
| COC1═CC(N═N)═CC(OC)═C1OCC(═O)NCCCCCCNC(═O)CO | GGJJUCQIRZLBFH-UHFFFAOYSA-N |
| N═NC1═CC═C(OCC(═O)NCCNC(═O)CO)C═C1 | LASZCECNYCYBSH-UHFFFAOYSA-N |
| O═C(CO)NC1═CC═C(/N═N/C2═CC═C(NC(═O)CO)C═C2)C═C1 | YPEPEJYZIWQRAF-FMQUCBEESA-N |
| CCOCCOC[C@@H](N)COCCOCCOC | DTGQDSGUKRYWTK-GFCCVEGCSA-N |
| CCOC[C@@H](N)COCCOCCOC | NVMIASBFGXTRPT-SNVBAGLBSA-N |
| CCOCC═O | IAHZBRPNDIVNNR-UHFFFAOYSA-N |
| CC(═O)NCCOCCOCCOCCNC(═O)CO | NILXGSCJCWYLAE-UHFFFAOYSA-N |
| OCCOCCOCCOCCO | UWHCKJMYHZGTIT-UHFFFAOYSA-N |
| COCCCOCCCCO | GBDXTMUABRNWDV-UHFFFAOYSA-N |
| COCC(F)(F)C(F)(F)C(F)(F)COCCCO | ISUXYHDVHYAOMT-UHFFFAOYSA-N |
| COCCCOC1═CC═CC═C1 | BNBJSIGRGDMPEZ-UHFFFAOYSA-N |
| COCC(F)(F)COC1═CC═CC═C1 | BMXMKJYJQLCFHV-UHFFFAOYSA-N |
| COCCCOCC(F)(F)C(F)(F)CO | FFHMJFWIMUIENX-UHFFFAOYSA-N |
| COCCOCCO | SBASXUCJHJRPEV-UHFFFAOYSA-N |
| COCCOCCOCCOCCOCCO | SLNYBUIEAMRFSZ-UHFFFAOYSA-N |
| O[C@H]1C[C@H](OCCCN2CCNCC2)C1 | SELCIEUYJJJDSH-XYPYZODXSA-N |
| CN1CCN(CCN2CCC(O[C@H]3C[C@H](O)C3)CC2)CC1 | LCUANHCLPFXWJR-KOMQPUFPSA-N |
| O═CC1CNC1 | ZWNNNSPVWRKCKH-UHFFFAOYSA-N |
| CCCCOCCCOC | SYQZZZDASGVPDV-UHFFFAOYSA-N |
| COCCCOCCCCOC1═CC═CC═C1 | LIGUZNOMUTYXPY-UHFFFAOYSA-N |
| NCCOCCOCCOCCNC(═O)C1═CC═CC═C1 | ZLCYMSYYWWTFHW-UHFFFAOYSA-N |
| NCCCNC(═O)C1═CC═CC═C1 | AOGPUGLWMPUQQZ-UHFFFAOYSA-N |
| CCOCCN1C═C(CCCC═O)N═N1 | ZYVQFJBCPSQGOQ-UHFFFAOYSA-N |
| CCOCCOCCOCCN1C═C(CCCC═O)N═N1 | MEJIFLPNQOXNNA-UHFFFAOYSA-N |
| CCOCCOCCOCCOCCN1C═C(CCCC═O)N═N1 | QVHKCQSQAQUGIM-UHFFFAOYSA-N |
| CCN1C═C(CCCC(N)═O)N═N1 | LTHPNMAUFSSHHQ-UHFFFAOYSA-N |
| CCOCCOCCOCCNC(═O)C1═CC═CC═C1 | JMCBUBRKTCPJBA-UHFFFAOYSA-N |
| CCCNC(═O)C1═CC═CC═C1 | DYZWXBMTHNHXML-UHFFFAOYSA-N |
| CCCCNC═O | QQGNLKJAIVSNCO-UHFFFAOYSA-N |
| CCOCCOCCNC═O | RNWZJESYGONOHW-UHFFFAOYSA-N |
| CCCCC1═CN(CCCC(═O)NCCOCCOCC)N═N1 | HQCOXSCMVZOVSI-UHFFFAOYSA-N |
| CCCCC1═CN(CCCC(═O)NCCOCCOCCNC═O)N═N1 | FCVBQIQYHKPARL-UHFFFAOYSA-N |
| CCCCCCNC(═O)CCCN1C═C(CCCC)N═N1 | SNHQLNFPKZVGOE-UHFFFAOYSA-N |
| CCCCCNC(═O)CCCN1C═C(CCCC)N═N1 | NHRQHDZERZZKFP-UHFFFAOYSA-N |
| CCNC(═O)CCCN1C═C(CC)N═N1 | OYGQPLAUVYHOPP-UHFFFAOYSA-N |
| CCCNC(═O)CCCN1C═C(CC)N═N1 | CAFZGVRRJDAZCY-UHFFFAOYSA-N |
| CCCNC(═O)CCCN1C═C(CCC)N═N1 | BQRMKTHSKNFIIL-UHFFFAOYSA-N |
| CCCCNC(═O)CCCN1C═C(CC)N═N1 | PGUUDHXNTZZKJH-UHFFFAOYSA-N |
| CCCCC1═CN(CCCC(═O)NCC)N═N1 | TVPMNLVGUJLSQE-UHFFFAOYSA-N |
| CCCCNC(═O)CCCN1C═C(CCC)N═N1 | PTBBPVRGVZZYEN-UHFFFAOYSA-N |
| CCCCC1═CN(CCCC(═O)NCCC)N═N1 | JEWBJEOLZTVPMD-UHFFFAOYSA-N |
| CCCCNC(═O)CCCN1C═C(CCCC)N═N1 | YJNOKWKENGTWAC-UHFFFAOYSA-N |
| CCCCNC(═O)[C@H](CCN1C═C(CCCC)N═N1)NC(═O)OC(C)(C)C | FGCATKFDSJLLTN-INIZCTEOSA-N |
| CCCCNC(═O)[C@@H]([NH3+])CCN1C═C(CCCC)N═N1[C]-] | UDEGIFBSQKBXHC-ZOWNYOTGSA-N |
| CCOCCOCCOCCOCCOCCOCCOCCOCCO | CUDPPTPIUWYGFI-UHFFFAOYSA-N |
| COCCOCCN | QWCGXANSAOXRFE-UHFFFAOYSA-N |
| CCOCCOCCOCCC═O | AJMGGIZNAPWJIH-UHFFFAOYSA-N |
| CCCCOCC | PZHIWRCQKBBTOW-UHFFFAOYSA-N |
| CCCNC(═O)CCCNC(═O)CO | SGKPVKWQAWXALE-UHFFFAOYSA-N |
| CCCNC(═O)CCCCCNC(═O)CO | MCLRTDKSPHAETG-UHFFFAOYSA-N |
| CCCCCNC(═O)CCCNC(═O)CO | GXNDLRLGVFAISP-UHFFFAOYSA-N |
| CCCCCNC(═O)CCCCCNC(═O)CO | KVEPXMCPBZBSGG-UHFFFAOYSA-N |
| CCCCCNC(═O)CCCNC(C)═O | RNRZMNLOCNEALK-UHFFFAOYSA-N |
| CCCCCNC(═O)CCCCCNC(C)═O | NVVTUVYYIDNSAW-UHFFFAOYSA-N |
| CCCCC(═O)NCC | ZOQTYYYRQHZQAR-UHFFFAOYSA-N |
| CCCCCCC(═O)NCC | KATPGOFDKJMROY-UHFFFAOYSA-N |
| CCCCCCCCC(═O)NCC | ACGGEROLCNXKHZ-UHFFFAOYSA-N |
| CCCCCCCCCC(═O)NCC | VXUAXBGDCYWTME-UHFFFAOYSA-N |
| CCCCCCCCCCC(═O)NCC | QFZHIMKZKRGXQF-UHFFFAOYSA-N |
| CCCCCCCCCCCC(═O)NCC | FEQGPEABBFYLNO-UHFFFAOYSA-N |
| CCCCCCCCCCCCC(═O)NCC | KNIHPOVOZDEDNP-UHFFFAOYSA-N |
| CCCCCCCCCCC(═O)NCCCC | BUXGVKFCLMHGNI-UHFFFAOYSA-N |
| CCCCCCCCCCC(═O)NCCCCCC | ISHXKOLGCAOECB-UHFFFAOYSA-N |
| CCCCCCCCCCC(═O)NCCOCC | CCJAYVXOVAERAG-UHFFFAOYSA-N |
| CCCCCCCCCCC(═O)NCCOCCOCC | XBVSIDRSXMQSMH-UHFFFAOYSA-N |
| CCCCCCCCCCC(═O)NCCCOCCOCCOCCC | WELVBSIIGXHCCX-UHFFFAOYSA-N |
| CCCCCCCCCCC(═O)NCCN | AVSPYMHLHOTOSQ-UHFFFAOYSA-N |
| CCCCCCCCCCC(═O)NC | MUZVJRFZJAXVBM-UHFFFAOYSA-N |
| CCCCCCCCCCC(═O)NCCC | JAEHFCHZFQZJAC-UHFFFAOYSA-N |
| CCCCCCCCCCC(═O)NCCCCC | LOTNCCWXPMLJSC-UHFFFAOYSA-N |
| CCC(═O)NCCOCCOC | GAWZLRJLUGPRSN-UHFFFAOYSA-N |
| CCOCCOCCOCCOCCNC(═O)CC | VIPWWMOAIBUCIE-UHFFFAOYSA-N |
| CCC(═O)N1CCC(CN2CCNCC2)CC1 | BSMXDYCSRGWMMS-UHFFFAOYSA-N |
| CCC(═O)N1CCC(CCN2CCNCC2)CC1 | RHOGJCWLCZMMFS-UHFFFAOYSA-N |
| CCCNC | GVWISOJSERXQBM-UHFFFAOYSA-N |
| CCCNCCC | WEHWNAOGRSTTBQ-UHFFFAOYSA-N |
| CCCCNCCC | CWYZDPHNAGSFQB-UHFFFAOYSA-N |
| CCCCCNCCC | GFAQQAUTKWCQHA-UHFFFAOYSA-N |
| CCCCCCCNCCC | SENZORBCFNGGDX-UHFFFAOYSA-N |
| CCCCCCCCNCCC | BBFLXVFMAOURDU-UHFFFAOYSA-N |
| CCCCCCCCCNCCC | FLSRMWBZKWEQHA-UHFFFAOYSA-N |
| CCCNCCOC | UDZCEFCJEGGQOJ-UHFFFAOYSA-N |
| CCCNCCOCCOC | WKFOEWLRIULZTA-UHFFFAOYSA-N |
| CCCNCCOCCOCC | AJWFKZWPBABFEU-UHFFFAOYSA-N |
| CCCNCCOCCOCCOCC | JOMYWNOLDXWEFK-UHFFFAOYSA-N |
| CCCNCCOCCOCCOCCOCC | PTEMPWOAYXZHTI-UHFFFAOYSA-N |
| CCCCCCCCCCCNCCC | QRPSACOFSBZHOT-UHFFFAOYSA-N |
| CCCCCCCCCCN(C)CCC | ZBYKVOSVXQAAPR-UHFFFAOYSA-N |
| CCCCCCCCCNCCCC | ZJFJKMQYBGYDFS-UHFFFAOYSA-N |
| CCCCCCCCCCNCCCC | QBQJQKTUTURRNX-UHFFFAOYSA-N |
| CCCCCCCCCCCNCCCC | BNCIDZMEGPXEPT-UHFFFAOYSA-N |
| CCCNCCOCCOCCOCCOCCOCC | FSJYFPZQCHZQAS-UHFFFAOYSA-N |
| CCCNCCCN | OWKYZAGJTTTXOK-UHFFFAOYSA-N |
| CCCNCCCCN | GHQFRBNLAGNQOE-UHFFFAOYSA-N |
| CCCNCCCCCN | TXZYEZDYAHKAPO-UHFFFAOYSA-N |
| CCCNCCCCCCN | GJPGEOGGZHPOMJ-UHFFFAOYSA-N |
| CCCNCCCCCCCN | ITHQCIDSGFBWIV-UHFFFAOYSA-N |
| CCCNCCCCCCCCN | JYAUAGKHEUJRDI-UHFFFAOYSA-N |
| CCCNCCOCCN | FOVBRQXVOFMZRK-UHFFFAOYSA-N |
| CCCNCCOCCOCCN | QEBHIHPWUXFIBD-UHFFFAOYSA-N |
| CCCNCCOCCOCCOCCN | QAPCIMTVCRODAQ-UHFFFAOYSA-N |
| CCCNCCOCCOCCOCCOCCN | IPOLTFPETRYHCV-UHFFFAOYSA-N |
| CCCNCCOCCOCCOCCOCCOCCN | LFEBEQHOJOMUMQ-UHFFFAOYSA-N |
| CCCCO | LRHPLDYGYMQRHN-UHFFFAOYSA-N |
| CCCCCCO | ZSIAUFGUXNUGDI-UHFFFAOYSA-N |
| CCCCCCCCO | KBPLFHHGFOOTCA-UHFFFAOYSA-N |
| CCCCCCCCCO | ZWRUINPWMLAQRD-UHFFFAOYSA-N |
| CCCCCCCCCCO | MWKFXSUHUHTGQN-UHFFFAOYSA-N |
| CCCCCCCCCCCO | KJIOQYGWTQBHNH-UHFFFAOYSA-N |
| CCCCCCCCCCCCO | LQZZUXJYWNFBMV-UHFFFAOYSA-N |
| COCC═O | YSEFYOVWKJXNCH-UHFFFAOYSA-N |
| COCCOCCOCCOCCOCC═O | PERZZOPFAPJECS-UHFFFAOYSA-N |
| CCOC1═CC(N)═CC(CCOC)═C1 | COXWDCOKOBYDPU-UHFFFAOYSA-N |
| CCOC1═CC(O)═CC(CCOC)═C1 | GASBROUVWUECLM-UHFFFAOYSA-N |
| COCCC1═CC(N)═CC(OCCN)═C1 | WAOPHHHWVCMEPH-UHFFFAOYSA-N |
| COC[C@@HJ1CNC[C@H](COCCN)C1 | QWLRGHWNIKYJDV-VHSXEESVSA-N |
| C#CCOCCOCCC1═CC(N)═CC(CC)═C1 | SVPJJVOCPCNJHP-UHFFFAOYSA-N |
| COCC#CC#CCN | FUTGOKPVWKUISE-UHFFFAOYSA-N |
| CCOCCN1C═C(CNC═O)N═N1 | FNHHKAJWLIMXHV-UHFFFAOYSA-N |
| CCOCCOCCN1C═C(CNC═O)N═N1 | ZXPQDLSPFYTOOR-UHFFFAOYSA-N |
| CCOCCOCCOCCN1C═C(CNC═O)N═N1 | GNMPCOFWUCPVKW-UHFFFAOYSA-N |
| CCOCCN1C═C(C(N)═O)N═N1 | XZHHUAZGHGSJIZ-UHFFFAOYSA-N |
| CCOCCOCCOCCN1C═C(C(N)═O)N═N1 | PWSIHVFBUUGICA-UHFFFAOYSA-N |
| CCOCCOCCOCCOCCN1C═C(C(N)═O)N═N1 | HQJBCKRSISCTDZ-UHFFFAOYSA-N |
| CCCCCCCCN1C═C(C(N)═O)N═N1 | KNBCNGDFMRNFCZ-UHFFFAOYSA-N |
| C#CCOCCOCCN1C═C(C(N)═O)N═N1 | WKHXKTDFEDKHQI-UHFFFAOYSA-N |
| C#CCOCCCCCN1C═C(C(N)═O)N═N1 | BTOWMKGPVFDZKD-UHFFFAOYSA-N |
| C#CCCCCCCCN1C═C(C(N)═O)N═N1 | RDSFRMGWNWIKKB-UHFFFAOYSA-N |
| CCCOCCOCCN1C═C(C(N)═O)N═N1 | WSABLGQCUKFRJE-UHFFFAOYSA-N |
| CCCOCCCCCN1C═C(C(N)═O)N═N1 | ZDKKHZAGQOKSJW-UHFFFAOYSA-N |
| CCCCCCCCCN1C═C(C(N)═O)N═N1 | PRMPITDVHITUCQ-UHFFFAOYSA-N |
| CCOCCOCCOCCOCCOCCOCCOCCOCCOCC | NFSLVRIGWIGOHW-UHFFFAOYSA-N |
| CCOCCOCCOCCOCCOCCN1C═C(C)N═N1 | YMSCFAMQHZLDKV-UHFFFAOYSA-N |
| O═CCOCCN1C═CN═N1 | YLUPPLUVICDFAJ-UHFFFAOYSA-N |
| O═CCOCCOCCN1C═CN═N1 | HRQDZVPLACCNBU-UHFFFAOYSA-N |
| O═CCOCCOCCOCCN1C═CN═N1 | UJEFTVBPSIVESU-UHFFFAOYSA-N |
| NC(═O)COCCOCCNC(═O)CO | UUJPZVBGKZKXNK-UHFFFAOYSA-N |
| CCO | LFQSCWFLJHTTHZ-UHFFFAOYSA-N |
| CCCO | BDERNNFJNOPAEC-UHFFFAOYSA-N |
| NC1CCNCC1 | BCIIMDOZSUCSEN-UHFFFAOYSA-N |
| C1CCNCC1 | NQRYJNQNLNOLGT-UHFFFAOYSA-N |
| CCCCCNC(C)═O | PTBCMKWBUAWWMQ-UHFFFAOYSA-N |
| CCCNC(C)═O | IHPHPGLJYCDONF-UHFFFAOYSA-N |
| O═CC1═CC═C(N2CCN(CCCCCCCCCO)CC2)C═C1 | WEDHJFMVBWTLMV-UHFFFAOYSA-N |
| CCCCCCCCCN1CCN(C2═CC═C(C═O)C═C2)CC1 | BMGOQQWDWPYXNE-UHFFFAOYSA-N |
| CCCCCCCCN1CCN(C2═CC═C(C═O)C═C2)CC1 | CPPJYZUYWLBIJT-UHFFFAOYSA-N |
| CCCCCCCN1CCN(C2═CC═C(C═O)C═C2)CC1 | XPQVLCZXSDSCTG-UHFFFAOYSA-N |
| CCCCN1CCN(C2═NC═C(C═O)C═N2)CC1 | XEOODTHFOLADBO-UHFFFAOYSA-N |
| CCCN1CCN(C2═NC═C(C═O)C═N2)CC1 | KXRMMGIROWBXFF-UHFFFAOYSA-N |
| CCCCCN1CCN(C2═NC═C(C═O)C═N2)CC1 | NWSGZKHTZAVIBX-UHFFFAOYSA-N |
| CCCCCCN1CCN(C2═NC═C(C═O)C═N2)CC1 | XETWPJMWIJNKCM-UHFFFAOYSA-N |
| CCCCCCCN1CCN(C2═NC═C(C═O)C═N2)CC1 | NJXXSMMCJGIVTH-UHFFFAOYSA-N |
| NCCCCCCN1CCN(C2═NC═C(C═O)C═N2)CC1 | BDWYHUOENGKEKO-UHFFFAOYSA-N |
| NCCCCCN1CCN(C2═NC═C(C═O)C═N2)CC1 | OAZULUUEWYPAOB-UHFFFAOYSA-N |
| NCCCCN1CCN(C2═NC═C(C═O)C═N2)CC1 | DHXPLHKSBHUODO-UHFFFAOYSA-N |
| NCCCN1CCN(C2═NC═C(C═O)C═N2)CC1 | CIZRTCZAAFEONT-UHFFFAOYSA-N |
| NCCCCCCCN1CCN(C2═NC═C(C═O)C═N2)CC1 | DWJXSLJOPAAYER-UHFFFAOYSA-N |
| O═CC1═CC═C(C#CC2CCN(C3CCNCC3)CC2)C═C1 | QLCXDGXPMMSVBD-UHFFFAOYSA-N |
| NCCCCCCCCN1CCN(C2═CC═C(C═O)C═C2)CC1 | LBWZUSHLKKMMNK-UHFFFAOYSA-N |
| NCCCCCCCCCN1CCN(C2═CC═C(C═O)C═C2)CC1 | ZBFWEXOQCDXMIV-UHFFFAOYSA-N |
| NCCCCCN1CCN(C2═CC═C(C═O)C═C2)CC1 | OAZBIRCKDIGQTB-UHFFFAOYSA-N |
| NCCCCCCN1CCN(C2═CC═C(C═O)C═C2)CC1 | GWYZXSMPWHWNIO-UHFFFAOYSA-N |
| NCCCCCCCN1CCN(C2═CC═C(C═O)C═C2)CC1 | XHFSKXOTCBJIPY-UHFFFAOYSA-N |
| COCCN1CCN(C2═CC═C(C═O)C═C2)CC1 | NXCINCOIPOCMDN-UHFFFAOYSA-N |
| CN1CCN(C2═CC═C(C═O)C═C2)CC1 | PFODEVGLOVUVHS-UHFFFAOYSA-N |
| CCN1CCN(C2═CC═C(C═O)C═C2)CC1 | UXVDOPUAJVRFDG-UHFFFAOYSA-N |
| COCCN1CCN(C2═CC═C(C═O)N═N2)CC1 | KTJJPXYKTRJPKM-UHFFFAOYSA-N |
| COCCN1CCN(C2═CC═C(C═O)C═N2)CC1 | DDXDJLGMOJZWPH-UHFFFAOYSA-N |
| COCCN1CCN(C2═CN═C(C═O)C═N2)CC1 | AVQYUGJIJNRSFW-UHFFFAOYSA-N |
| COCCN1CCN(C2═NC═C(C═O)C═N2)CC1 | ACNPUZJXSFRZGH-UHFFFAOYSA-N |
| COCCN1CCN(C2═CC═C(C═O)N═C2)CC1 | NWEJJQZGEDNEAQ-UHFFFAOYSA-N |
| C#CCOCC | ADJMUEKUQLFLQP-UHFFFAOYSA-N |
| CCOCCOCCOCCOCCO | GTAKOUPXIUWZIA-UHFFFAOYSA-N |
| CCCCCCCCN1C═C(CO)N═N1 | IEKALTKLFGFMSM-UHFFFAOYSA-N |
| CCOCCOCCN1C═C(CO)N═N1 | ANXFOEJMIUVYKR-UHFFFAOYSA-N |
| CCOCCOCCOCCN1C═C(CO)N═N1 | JQDWUIXCABEAEW-UHFFFAOYSA-N |
| CC(═O)NCCNC(═O)CO | JWGSAIZRDOFOEH-UHFFFAOYSA-N |
| CC(═O)NCCCNC(═O)CO | CHIKLXYQCFDKRA-UHFFFAOYSA-N |
| CC(═O)NCCCCCNC(═O)CO | JLRWBEZBLSVYBD-UHFFFAOYSA-N |
| CC(═O)NCCCCCCNC(═O)CO | KJPXHCILCUXKMA-UHFFFAOYSA-N |
| CC(═O)NCCOCCNC(═O)CO | FXKBINPJWPVOTP-UHFFFAOYSA-N |
| NCCCCCO | LQGKDMHENBFVRC-UHFFFAOYSA-N |
| NCCNC(═O)CO | IHQDUEDKOPTHNY-UHFFFAOYSA-N |
| NCCCNC(═O)CO | AYRTYYKMBLVODF-UHFFFAOYSA-N |
| NCCCCCNC(═O)CO | SFBZTMMQUMHXQN-UHFFFAOYSA-N |
| NCCCCCCNC(═O)CO | LYPXVPQYJHRDFB-UHFFFAOYSA-N |
| NCCCCCCCNC(═O)CO | NXUCFNATDPJBIC-UHFFFAOYSA-N |
| C#CCCC(N)═O | KNEPBZMJMJUNQX-UHFFFAOYSA-N |
| C#CCCCC(N)═O | DRXWSHCGSIJSID-UHFFFAOYSA-N |
| C#CCCCCC(N)═O | WEGGYBNXYQHSQO-UHFFFAOYSA-N |
| NCCOCCO | GIAFURWZWWWBQT-UHFFFAOYSA-N |
| NCCOCCOCCO | ASDQMECUMYIVBG-UHFFFAOYSA-N |
| CCCCCCCCNC(═O)CC | RCNSZOSXOKUSCL-UHFFFAOYSA-N |
| CCC(═O)NCCCCNCC═O | XGRNWTWHTZDCIG-UHFFFAOYSA-N |
| CCC(═O)NCCCCCCNCC═O | QVQAHLBIFVURJF-UHFFFAOYSA-N |
| CCC(═O)NCCCCCCCCNCC═O | GYNAJAVMRWINBN-UHFFFAOYSA-N |
| CCC(═O)NCCOCCNCC═O | VSGVZXJYXPORPB-UHFFFAOYSA-N |
| CCC(═O)NCCOCCOCCNCC═O | OIJWZFOMOBKPDT-UHFFFAOYSA-N |
| CCC(═O)NCCOCCOCCOCCNCC═O | QUDIUNZQAQSCGY-UHFFFAOYSA-N |
| CCC(═O)NCCCCNCCC═O | BFLFIMOTAZXOOR-UHFFFAOYSA-N |
| CCC(═O)NCCCCCCNCCC═O | ZRHHQLIPOIANNN-UHFFFAOYSA-N |
| CCC(═O)NCCOCCNCCC═O | NPQYVPWHMDNIIO-UHFFFAOYSA-N |
| CCC(═O)NCCOCCOCCNCCC═O | YXOZGMDVCSMFEO-UHFFFAOYSA-N |
| CCC(═O)NCCCCNC(═O)CO | BMKGRZVHBMIQOK-UHFFFAOYSA-N |
| CCC(═O)NCCCCCCNC(═O)CO | MKVKXGSQRHLSLH-UHFFFAOYSA-N |
| CCC(═O)NCCCCCCCCNC(═O)CO | ZEGWMZYHPJAPDX-UHFFFAOYSA-N |
| CCC(═O)NCCOCCNC(═O)CO | QAEYEPVQGPGHDZ-UHFFFAOYSA-N |
| CCC(═O)NCCOCCOCCNC(═O)CO | FLRGLMYGMGIDDO-UHFFFAOYSA-N |
| CCC(═O)NCCOCCOCCOCCNC(═O)CO | LNVXGEATCIPVSB-UHFFFAOYSA-N |
| CCOCCOCCN(C)CC═O | NCFBSCAHPYQLNT-UHFFFAOYSA-N |
| CCOCCOCCOCCN(C)CC═O | SYGRXDVWYZCFJZ-UHFFFAOYSA-N |
| CCOCCOCCOCCOCCN(C)CC═O | TYIOYOVEQMGKPQ-UHFFFAOYSA-N |
| NCCOCCOCCOCCOCCC-O | ORBOSPTWQGKEDA-UHFFFAOYSA-N |
| CCOCCNC(═O)CO | BOQCRSNIDAXGBH-UHFFFAOYSA-N |
| CCOCCOCCNC(═O)CO | YSJMRGUNPOPSRN-UHFFFAOYSA-N |
| CCOCCOCCOCCOCCNC(═O)CO | NUHSPVQXOVPPHQ-UHFFFAOYSA-N |
| CCOCCOCC(N)═O | NHCPQLWQFVVDHX-UHFFFAOYSA-N |
| CCOCCOCCOCC(N)═O | NNBNUYZWLQIAQC-UHFFFAOYSA-N |
| CCOCCOCCOCCOCC(N)═O | HORIVGWLKLJAPF-UHFFFAOYSA-N |
| CCC(N)═O | QLNJFJADRCOGBJ-UHFFFAOYSA-N |
| CCCCC(N)═O | IPWFJLQDVFKJDU-UHFFFAOYSA-N |
| CCCCCCC(N)═O | AEDIXYWIVPYNBI-UHFFFAOYSA-N |
| CCCCCCCCC(N)═O | GHLZUHZBBNDWHW-UHFFFAOYSA-N |
| CCOCCOCCOCCCCC═O | TVTONMGAFMBIEE-UHFFFAOYSA-N |
| CCCOCCCCOCCCNC(═O)CCC═O | LYEKRVSKERKZTQ-UHFFFAOYSA-N |
| CCCCCCOCCCCCCCCCC═O | MREZGIJDDKLJAB-UHFFFAOYSA-N |
| CCCCCCOCCCCCCOCCCCCC═O | SGYAHJQVKILNRH-UHFFFAOYSA-N |
| CCCCCCCCOCCCCOCCCCCC═O | ILSJXDDMHYRCAR-UHFFFAOYSA-N |
| CCCCCCOCCCCCCCCCCCCOCCCCCC═O | HFKSJAJZQDJXQR-UHFFFAOYSA-N |
| CCCCCCCCCCOCCOCCOCCOCCCCCC═O | AZIKOAQQSVTNGC-UHFFFAOYSA-N |
| CCOCCOCCOCCN | WWJVRDMJNJTOBL-UHFFFAOYSA-N |
| NCCOCCOCCNC(═O)CO | YSTUQSIFXBNAOT-UHFFFAOYSA-N |
| NCCOCCNC(═O)CO | FTBLOENAESLBFM-UHFFFAOYSA-N |
| CCOCCOCCOCCOCCOCCOCCOCCOCCC═O | NSIIVBKTTWZVBU-UHFFFAOYSA-N |
| CCCOCCOCCOCCCNC(═O)CCC═O | KAZVYKPEGHVBSS-UHFFFAOYSA-N |
| CCCNC(═O)CCOCCOCCOCC | NBKPZJBBKGPIJV-UHFFFAOYSA-N |
| CCCNC(═O)CCOCCOCCOCCOCC | DFLDBRXTRJJFKC-UHFFFAOYSA-N |
| CCCNC(═O)CCOCCOCCOCCOCCOCC | UDSYHQHLBBGNDT-UHFFFAOYSA-N |
| CCCNC(═O)CCOCCOCCOCCOCCOCCOCC | REPCTFORHOGVEE-UHFFFAOYSA-N |
| CCCNC(═O)CCOCCOCCOCCOCCOCCNC(═O)CCOCCOCCOCC | DDCZNDUMQFIGCE-UHFFFAOYSA-N |
| OCCOCC | |
| CCCCCCNC(═O)CC | WVNFFHLXDXOSGO-UHFFFAOYSA-N |
| CCOCCOCCOCCOCCC(═O)N1CCCCC1 | GORQSGKROVUXHB-UHFFFAOYSA-N |
| CCOCCOCCOCCC(═O)N1CCCCC1 | IOKWKYZOEBVEBU-UHFFFAOYSA-N |
| CCOCCOCCOCCC(═O)N1CC(C)C1 | CUZHEHSKFZQZPO-UHFFFAOYSA-N |
| CCCCCCCCCC(═O)N1CCN(CC2═CC═C(C)C═C2)CC1 | XZIZADNFZVHVRR-UHFFFAOYSA-N |
| CCCCCCCCCC(═O)N1CCNCC1 | CJFPGCKUAUNMHO-UHFFFAOYSA-N |
| CCCOCCCCOCCCNC(C)═O | QXBRTKUNDWWBCM-UHFFFAOYSA-N |
| CCOCC1═CN(CCOCCOCCC═O)N═N1 | VRVRIXZCPFKIQB-UHFFFAOYSA-N |
| C#CCOCCOCCOCCOCCC═O | NEBHYQIAMLUJDI-UHFFFAOYSA-N |
| OCCOCCO | MTHSVFCYNBDYFN-UHFFFAOYSA-N |
| OCC1═CN═C(CO)C═N1 | HTXOIHGJECPJIU-UHFFFAOYSA-N |
| CCOCCN1C═C(COCC)N═N1 | GRLLJNJWRWKWSX-UHFFFAOYSA-N |
| CCOCCOCCOCCN1C═C(COCC)N═N1 | YBYSAXFBXHPGBP-UHFFFAOYSA-N |
| CN1CCNCC1 | PVOAHINGSUIXLS-UHFFFAOYSA-N |
| CCCO | BDERNNFJNOPAEC-UHFFFAOYSA-N |
| CC(═O)NCCCN(C)C(C)C | KDDWELLQASOHNW-UHFFFAOYSA-N |
| CC(═O)NCCCCN(C)C(C)C | LVBHCIMSCQFXKA-UHFFFAOYSA-N |
| CC(═O)NCCOCCN(C)C(C)C | BXCKIGATIPFOMS-UHFFFAOYSA-N |
| CC(═O)NCCOCCOCCN(C)C(C)C | WDKNYGIKNAZDNY-UHFFFAOYSA-N |
| CC(═O)NCCOCCOCCOCCN(C)C(C)C | MFJGKGROGOLTMK-UHFFFAOYSA-N |
| CC(═O)NCCN(C)C(C)C | WFGDITSPCFYUOZ-UHFFFAOYSA-N |
| CCCCN(C)C(C)C | KKBDINYFLTTZAQ-UHFFFAOYSA-N |
| CCCN(C)C(C)C | OYQDUCLFZSKBCZ-UHFFFAOYSA-N |
| CC(C)N(C)CCCNC(═O)CO | GECLTMRIXDTICE-UHFFFAOYSA-N |
| CC(═O)NCCCN(C)C | OHLICMMXIJECIN-UHFFFAOYSA-N |
| CCCN(C)CCCNC(C)═O | JANJMBPFCYDLQZ-UHFFFAOYSA-N |
| CC(═O)NCCOCCN(C)C | NCCAWFFMEPWOOH-UHFFFAOYSA-N |
| CCCN(C)CCOCCNC(C)═O | ZFWHIVXUTMRFOV-UHFFFAOYSA-N |
| CC(═O)NCCCN(C)C(C)(C)CCC#N | LJNAQLYOVRMQER-UHFFFAOYSA-N |
| CN(C)CCOCCNC(═O)CN | IELFBMABZWXVAF-UHFFFAOYSA-N |
| CCCN(C)CCOCCNC(═O)CN | WYTCOWNFAQMJJZ-UHFFFAOYSA-N |
| CC(C)N(C)CCOCCNC(═O)CN | OOMRMVJYDVKEIA-UHFFFAOYSA-N |
| CCCNC(═O)CC | YUMCRXLLWKQDJY-UHFFFAOYSA-N |
| NCCCCCCCC═O | GKOPBHPTLGFKOR-UHFFFAOYSA-N |
| NCCCCCCCCCCC═O | XSVQSLVVCYRXCL-UHFFFAOYSA-N |
| NCCCCCCCCCCCC═O | GDWQVDJDAUJLON-UHFFFAOYSA-N |
| CCCCNC(═O)CO | WFYNRXPMFUYIDC-UHFFFAOYSA-N |
| CNC(═O)CCCC═O | MGPSVKVFOOVOFZ-UHFFFAOYSA-N |
| O═CCN1CCC(CNC═O)CC1 | AYWVMRKDYWNHKQ-UHFFFAOYSA-N |
| O═CCCCC═O | SXRSQZLOMIGNAQ-UHFFFAOYSA-N |
| CC1CCN(C═O)CC1 | WCKITLGQGJSRBV-UHFFFAOYSA-N |
| NCCCCCC(N)═O | ZLHYDRXTDZFRDZ-UHFFFAOYSA-N |
| NCCOCCOCC(N)═O | CDFONQZJZPNQML-UHFFFAOYSA-N |
| CCCCCCCCNC(C)═O | GLJKLMQZANYKBO-UHFFFAOYSA-N |
| C1CC(CCN2CCNCC2)CCN1 | MXTMUSBXYDAXEK-UHFFFAOYSA-N |
| C1CC(OCCN2CCNCC2)CCN1 | JXCKBJKSCSVLCY-UHFFFAOYSA-N |
| OCCCCCN1CCNCC1 | JQOBQNAVHUEQPB-UHFFFAOYSA-N |
| C1CC(CN2CCN(CC3CCNCC3)CC2)CCN1 | CXPVURPCWVRDAG-UHFFFAOYSA-N |
| C1CC(CCN2CCC3(CCNCC3)CC2)CCN1 | DILBUBUHQKMCKO-UHFFFAOYSA-N |
| C1CC(CCN2CCC3(CCNCC3)CC2)CCN1 | DILBUBUHQKMCKO-UHFFFAOYSA-N |
| C1CC(CN2CCOC3(CCNCC3)CC2)CCN1 | BFCQBZFXQYTGJH-UHFFFAOYSA-N |
| C1CC(CCN2CCOC3(CCNCC3)CC2)CCN1 | YMEPHYOPKJJPFD-UHFFFAOYSA-N |
| C1CC2(CCN1)CC(CN1CC3(CCNCC3)C1)C2 | KMDFIPVKOHKDMJ-UHFFFAOYSA-N |
| C1CC(CN2CCNCC2)CCN1 | INUYEZQAQSEPMT-UHFFFAOYSA-N |
| OC1CCC2(CC1)CCN(CC1CCNCC1)CC2 | WBRXCBGYGQLNQG-UHFFFAOYSA-N |
| CCOC1═CC═C(CC)C═C1 | BQBROHBMIBOPFU-UHFFFAOYSA-N |
| CCCOC1═CC═C(C)C═C1 | QLTKVDKXTKKJOX-UHFFFAOYSA-N |
| CCCC1═CC═C(C)C═C1 | JXFVMNFKABWTHD-UHFFFAOYSA-N |
| CCC1═CC═C(C)C(F)═C1 | DYKMICWOBLSRIW-UHFFFAOYSA-N |
| NCCCNC(═O)CCCCCCNC(═O)CO | GXRUEVSAWFUYNL-UHFFFAOYSA-N |
| CCCCCCCC(═O)NCCCN | AZGZCTUTODQNFW-UHFFFAOYSA-N |
| CCCCCCCNC(═O)CO | BPFGOQULJGPNTD-UHFFFAOYSA-N |
| OCCCCCN1C═C(CN2CCNCC2)N═N1 | MTIMZAHMBOLSHZ-UHFFFAOYSA-N |
| OCCN1CCNCC1 | WFCSWCVEJLETKA-UHFFFAOYSA-N |
| OCCCN1CCNCC1 | LWEOFVINMVZGAS-UHFFFAOYSA-N |
| OCCCCCCN1CCNCC1 | RMIIQABTVWHYKA-UHFFFAOYSA-N |
| CCCCN1CCNCC1 | YKSVXVKIYYQWBB-UHFFFAOYSA-N |
| CCCCCN1CCNCC1 | MJWWNBHUIIRNDZ-UHFFFAOYSA-N |
| CCCCCN1C═C(CN2CCNCC2)N═N1 | SPLUTQOUWLEIRU-UHFFFAOYSA-N |
| CCOCCNC(═O)CCOCCOCC | RRHXXGGYPBLUBP-UHFFFAOYSA-N |
| CCCN1CCN(CCNC(═O)CCOCCOCC)CC1 | FVQKRLOBYKWDEY-UHFFFAOYSA-N |
| O═CNCCCCCCCCNC(═O)CO | MLFHWPIWTNJODL-UHFFFAOYSA-N |
| CCOCCOCCOCCOCCC(═O)NC1═CC═CC(C)═C1 | WRKJGBIKJXWNON-UHFFFAOYSA-N |
| O═CCCCCCCCNC(═O)CO | WIQGTRQGYZGWML-UHFFFAOYSA-N |
| CCCCO | LRHPLDYGYMQRHN-UHFFFAOYSA-N |
| CCOCCOCCNC(═O)CN | PHICTDCYZJZWBO-UHFFFAOYSA-N |
| CC(═O)NCCOCCOCCOCCC═O | SVZLKBWUDJMXGH-UHFFFAOYSA-N |
| CCOCCNC(C)═O | VNVZKKJQVMBZNN-UHFFFAOYSA-N |
| CCCOCCOCCOCCNC(═O)CO | DXHOONGGAMPJPN-UHFFFAOYSA-N |
| CCCNC(═O)CCCCCCNC(═O)CO | WPFSARBSQPVWDD-UHFFFAOYSA-N |
| O═CCCCCCCNC(═O)CO | FBJRVOACVHVTEO-UHFFFAOYSA-N |
| O═C(CO)NCCCCCCC(═O)NCCO | CDVOMNZSLUETHP-UHFFFAOYSA-N |
| CCOCCOCCOCCNC(═O)CN | DCKAJMRUSXPRRX-UHFFFAOYSA-N |
| CCOCCNC(═O)CN | JRKNJYMVYPWZND-UHFFFAOYSA-N |
| CCOCCOCCC(N)═O | KMFYAANKBFXERF-UHFFFAOYSA-N |
| CCOCCOCCOCCOCCC(N)═O | FZZPLPGZROHDIE-UHFFFAOYSA-N |
| CC(N)═O | DLFVBJFMPXGRIB-UHFFFAOYSA-N |
| CCCC(N)═O | DNSISZSEWVHGLH-UHFFFAOYSA-N |
| CCCCCC(N)═O | ALBYIUDWACNRRB-UHFFFAOYSA-N |
| C#CCCCCCCC | OSSQSXOTMIGBCF-UHFFFAOYSA-N |
| CCOCCNC(═O)CCC═O | HQNNHAYYQCEVPP-UHFFFAOYSA-N |
| CCOCCOCCNC(═O)CCC═O | KDAIXUIQPNUHIR-UHFFFAOYSA-N |
| NCCOCCNC(═O)CCC═O | VEEHVJQSYMNZHW-UHFFFAOYSA-N |
| NCCOCCOCCNC(═O)CCC═O | KROVULALCQJERG-UHFFFAOYSA-N |
| NCCOCCOCCOCCNC(═O)CCC═O | DPXRNKZXXHGWBJ-UHFFFAOYSA-N |
| NCCCOCCOCCOCCCNC(═O)CCC═O | RKNUFOYHPXSCQV-UHFFFAOYSA-N |
| CCCCCCCCNC(═O)CCC═O | DBDJJJHMJGHNTO-UHFFFAOYSA-N |
| CCCCCCCCCNC(═O)CCC═O | JFOFEDMCDUEZAL-UHFFFAOYSA-N |
| CCCCCCCCCCCCNC(═O)CCC═O | AYKSXGYVGBZFIU-UHFFFAOYSA-N |
| CCCCCCCCCCCCCNC(═O)CCC═O | RUQGAESQCNEDIH-UHFFFAOYSA-N |
| CCCOCCOCCOCCCNC(═O)CO | BBDDYLXZIGBXQL-UHFFFAOYSA-N |

Table 2 shows selections of another part of the Linker module, namely PEG type linker, including but not limited to the compounds indicated by “name” therein and corresponding to the EnamineStore ID, see Table 2 below for details. Among them, EnamineStore is the compound database (web site as https://www.enaminestore.com/search).

| ID | Name |
|---|---|
| EN300-25338 | 2-bromo-4-(2-hydroxyethoxy)benzaldehyde |
| EN300-83836 | 2-(benzyloxy)ethan-1-ol |
| EN300-24604 | N-[3-(benzyloxy)propyl]-2-chloroacetamide |
| EN300-30793 | 1-bromo-2-(2-bromoethoxy)ethane |
| EN300-42107 | 3-(2-hydroxyethoxy)-4-methoxybenzoic acid |
| EN300-01038 | 2-hydroxy-4-propoxybenzaldehyde |
| EN300-41170 | 4-(4-cyano-2-methoxyphenoxy)butanoic acid |
| EN300-45144 | 2-[4-(aminomethyl)-2-methoxyphenoxy]ethan-1-ol |
| EN300-53108 | 2-(4-amino-3-methylphenoxy)ethan-1-ol |
| EN300-59306 | 2-[4-(aminomethyl)phenoxy]ethan-1-ol hydrochloride |
| EN300-69417 | 2-[4-(3-cyanopropoxy)phenyl]acetic acid |
| EN300-77411 | 2-[4-(2-aminoethoxy)phenyl]acetonitrile hydrochloride |
| EN300-91593 | methyl 3-(2-aminophenoxy)propanoate hydrochloride |
| EN300-104088 | 2-(piperidin-4-yloxy)ethan-1-ol |
| EN300-108904 | 2-(prop-2-yn-1-yloxy)ethan-1-ol |
| EN300-105595 | 3-(piperidin-4-yloxy)propan-1-ol |
| EN300-106301 | methyl 4-[(4-aminonaphthalen-1-yl)oxy]butanoate |
| EN300-108866 | methyl[2-(prop-2-yn-1-yloxy)ethyl]amine hydrochloride |
| EN300-75227 | 4-(2-hydroxyethoxy)benzoic acid |
| EN300-60360 | methyl 3-(4-aminophenoxy)propanoate |
| EN300-53019 | 2-hydroxypropane-1,2,3-tricarboxylic acid; dimethyl |
| | ({2-[(2-methylphenyl)(phenyl)methoxy]ethyl})amine |
| EN300-45326 | 4-(3-aminophenoxy)butanenitrile |
| EN300-109303 | 1-chloro-3-(2-hydroxyethoxy)propan-2-ol |
| EN300-95947 | 4-(2-hydroxyethoxy)phenol |
| EN300-84087 | 2-[2-(aminomethyl)phenoxy]ethan-1-ol |
| EN300-117907 | 2-(tert-butoxy)ethan-1-ol |
| EN300-125535 | 2,2,2-trifluoro-N-[2-(2-hydroxyethoxy)ethyl]acetamide |
| EN300-129658 | 2-(3-aminopropoxy)ethan-1-ol |
| EN300-133893 | 2-(2-aminoethoxy)acetic acid hydrochloride |
| EN300-135058 | [(3-chloropropoxy)methyl]benzene |
| EN300-131583 | 4-(but-3-yn-1-yloxy)benzoic acid |
| EN300-118560 | 2-[2-(2-aminoethoxy)ethoxy]ethan-1-amine |
| EN300-138168 | 3-(2-carboxyethoxy)propanoic acid |
| EN300-130529 | 2-(4-amino-2-methylphenoxy)ethan-l-ol |
| EN300-140872 | tert-butyl N-[1-cyano-3-(propan-2-yloxy)propyl]carbamate |
| EN300-154609 | [4-(but-3-yn-1-yloxy)phenyl]methanamine |
| EN300-153872 | sodium 2-(2-hydroxyethoxy)acetate |
| EN300-58533 | 2-[4-(2-aminoethyl)phenoxy]ethan-1-ol |
| EN300-156651 | [5-(2-methoxyethoxy)piperidin-3-yl]methanol |
| EN300-173622 | methyl 4-butoxy-2-hydroxybenzoate |
| EN300-184469 | 2-(3-amino-5-methylphenoxy)ethan-1-ol hydrochloride |
| EN300-187795 | 2-[(2-{2-[(2-hydroxyethyl)sulfanyl]ethoxy}ethyl)sulfanyl]ethan-1-ol |
| EN300-188510 | 2-{[2-(2-hydroxyethoxy)ethyl]sulfanyl}ethan-1-ol |
| EN300-119687 | tert-butyl N-[2-(2-hydroxyethoxy)ethyl]carbamate |
| EN300-115110 | 3-(benzyloxy)propan-1-ol |
| EN300-200831 | 2-[2-(methylamino)ethoxy]ethan-1-ol |
| EN300-201578 | 2-[2-(ethylamino)ethoxy]ethan-1-ol |
| EN300-201781 | 2-{[4-(aminomethyl)oxan-4-yl]oxy}ethan-1-ol |
| EN300-207948 | 3-(2-aminoethoxy)propan-1-ol hydrochloride |
| EN300-208106 | 5-(tert-butoxy)pentanoic acid |
| EN300-205023 | tert-butyl N-[2-(2-bromoethoxy)ethyl]carbamate |
| EN300-208387 | 4-(tert-butoxy)butanoic acid |
| EN300-202642 | tert-butyl N-{[4-(2-aminoethoxy)oxan-4-yl]methyl}carbamate |
| EN300-209494 | 3-(4-aminophenoxy)propan-1-ol |
| EN300-215454 | 2-(3-propoxypropanamido)pentanedioic acid |
| EN300-202971 | tert-butyl N-{2-[2-(2-hydroxyethoxy)ethoxy]ethyl}carbamate |
| EN300-214094 | 2-[(1-amino-2-methylpropan-2-yl)oxy]ethan-1-ol |
| EN300-222271 | 3-(2-amino-4-methanesulfonylphenoxy)propan-1-ol |
| EN300-222272 | 2-(2-amino-4-methanesulfonylphenoxy)ethan-1-ol |
| EN300-219006 | N-[2-(2-bromoethoxy)ethyl]acetamide |
| EN300-217925 | N-[2-(2-hydroxyethoxy)ethyl]acetamide |
| EN300-234923 | 2-(3-aminophenoxy)ethan-1-ol |
| EN300-227945 | 4-(2-hydroxyethoxy)-3-methoxybenzaldehyde |
| EN300-244237 | ethyl 2-(2-aminoethoxy)acetate hydrochloride |
| EN300-164412 | 2-[2-(aminomethyl]phenoxy]ethan-1-amine dihydrochloride |
| EN300-74512 | 5-(3-cyanophenoxy)pentanoic acid |
| EN300-75370 | 3-(4-aminophenoxy)propanoic acid hydrochloride |
| EN300-116457 | 2-(2-bromoethoxy)ethan-1-ol |
| EN300-253397 | [2-(2-aminoethoxy)ethyl](ethyl)amine |
| EN300-258081 | 2-(azetidin-3-yloxy)ethan-1-ol |
| EN300-257374 | 4-(4-chlorobutoxy)butan-1-ol |
| EN300-256898 | 2-[(4-aminophenyl)methoxy]ethan-1-ol |
| EN300-257274 | 2-(4-hydroxybutoxy)benzaldehyde |
| EN300-266067 | (1s,3s)-3-[2-(tert-butoxy)ethoxy]cyclobutan-1-ol |
| EN300-264061 | propyl (3S)-3-(aminomethyl)-5-methylhexanoate hydrochloride |
| EN300-298869 | 2-{[3-(aminomethyl)oxolan-3-yl]oxy}ethan-1-ol |
| EN300-142192 | 2-(2-azidoethoxy)ethan-1-ol |
| EN300-297883 | 2-[2-(3-aminopropoxy)ethoxy]ethan-1-ol |
| EN300-298653 | 2-(2-iodoethoxy)ethan-1-ol |
| EN300-299042 | tert-butyl N-{3-[2-(2-hydroxyethoxy)ethoxy]propyl}carbamate |
| EN300-312631 | 3-fluoro-4-(2-hydroxyethoxy)benzaldehyde |
| EN300-315819 | tert-butyl N-methyl-N-{2-[2-(methylamino)ethoxy]ethyl}carbamate |
| EN300-315965 | 2-({2-[2-(2-{[(tert-butoxy)carbonyl]amino}ethoxy)ethoxy]ethyl}amino)acetic acid |
| EN300-312945 | 1-bromo-3-(3-bromopropoxy)propane |
| EN300-317293 | tert-butyl N-[3-(2-aminoethoxy)propyl]carbamate |
| EN300-332506 | 1-bromo-4-(4-bromobutoxy)butane |
| EN300-315510 | 4-amino-3-(2-hydroxyethoxy)benzoic acid |
| EN300-345187 | 5-(5-amino-2-methylphenoxy)pentan-1-ol |
| EN300-365537 | 3-{[2-(2,2-dimethylpropanamido)acetyl]oxy}propanoic acid |
| EN300-370460 | 2-[3-(2-aminoethoxy)phenyl]acetic acid hydrochloride |
| EN300-19706 | 1-chloro-2-[2-(2-chloroethoxy)ethoxy]ethane |
| EN300-19916 | 2-[2-(2-hydroxyethoxy)ethoxy]ethan-1-ol |
| EN300-378267 | 2-{2-[(2-aminoethyl)(methyl)amino]ethoxy}ethan-1-ol |
| EN300-398865 | 2-{[1-(aminomethyl)-4-(difluoromethyl)cyclohexyl]oxy}ethan-1-ol hydrochloride |
| EN300-686899 | 3-(prop-2-yn-1-yloxy)propan-1-ol |
| EN300-247320 | tert-butyl 2-[2-(2-aminoethoxy)ethoxy]acetate |
| EN300-1067029 | 3-[2-(tert-butoxy)ethoxy]propanoic acid |
| EN300-814287 | 2-(2-chloroethoxy)acetyl chloride |
| EN300-104458 | 2-(2-chloroethoxy)acetic acid |
| EN300-171481 | 2-[1-(aminomethyl)cyclobutoxy]ethan-1-ol |
| EN300-20351 | 2-[2-(carboxymethoxy)ethoxy]acetic acid |
| EN300-1091618 | 2-{[1-(aminomethyl)cyclohexyl]oxy}ethan-1-ol |
| EN300-1178083 | 2-{[4-(aminomethyl)thian-4-yl]oxy}ethan-1-ol |
| EN300-192866 | {[2-(2-chloroethoxy)ethoxy]methyl}benzene |
| EN300-1293808 | 2-{[3-(aminomethyl)thiolan-3-yl]oxy}ethan-1-ol |
| EN300-52813 | 2-[4-amino-3-(trifluoromethyl)phenoxy]ethan-1-ol |
| EN300-1588524 | 2-[2-(aminooxy)ethoxy]ethan-1-ol |
| EN300-1658972 | O-[2-(prop-2-yn-1-yloxy)ethyl]hydroxylamine |
| EN300-1609001 | 3-(aminomethyl)-3-(2-hydroxyethoxy)-llambda6-thiolane-1,1-dione hydrochloride |
| EN300-1700498 | 2-(3-aminopropoxy)acetic acid hydrochloride |
| EN300-1696667 | 3-hydroxy-4-propoxybenzaldehyde |
| EN300-1603980 | 4-amino-2-{2-[2-(2-methoxyethoxy)ethoxy]ethyl}butanoic acid |
| EN300-1704639 | 2-[2-(benzyloxy)ethyl]propane-1,3-diol |
| EN300-1700367 | rac-2-{[(2R,6R)-6-methylpiperidin-2-yl]methoxy}ethan-1-ol |
| EN300-1719373 | methyl 3-(2-aminoethoxy)propanoate hydrochloride |
| EN300-298890 | butyl 2-(aminooxy)acetate |
| EN300-384462 | 4-(benzyloxy)-2,2-dimethylbutan-1-ol |
| EN300-7440871 | 12-bromo-2,2,3,3-tetramethyl-4,7,10-trioxa-3-siladodecane |
| EN300-207151 | 1-[2-(2-aminoethoxy)ethoxyl-2-(2-azidoethoxy)ethane |
| EN300-207147 | 2-[2-(2-azidoethoxy)ethoxy]ethan-1-ol |
| EN300-74137 | 2-(4-aminophenoxy)ethan-1-ol |
| EN300-6948257 | 2-(but-3-yn-1-yloxy)acetic acid |
| EN300-6963624 | 2-[2-(prop-2-yn-1-yloxy)ethoxy]acetic acid |
| EN300-305336 | 1-phenyl-2,5,8,11-tetraoxatridecan-13-ol |
| EN300-27187326 | sodium 2-[6-(4-ethynylphenoxy)hexyl]oxirane-2-carboxylate |
| EN300-7403031 | 3,6,9,12,15,18,21,24-octaoxahexacosane-1,26-diol |
| EN300-7492463 | sodium 2-(2-hydroxyethoxy)ethane-1-sulfonate |
| EN300-7472919 | tert-butyl N-{[4-(2-aminoethoxy)oxan-4-yl]methyl}carbamate hydrochloride |
| EN300-6474931 | 2-(2-aminoethoxy)phenol hydrochloride |
| EN300-7493941 | 4-[4-(hydroxymethyl)-2-methoxy-5-nitrophenoxy]butanoic acid |
| EN300-26698161 | methyl 4-[(1,3-dihydroxypropan-2-yl)oxy]butanoate |
| EN300-6986641 | 3-[2-(2-aminoethoxy)ethoxy]prop-1-yne |
| EN300-7462425 | 2-{2-[2-(prop-2-yn-1-yloxy)ethoxy]ethoxy}acetic acid |
| EN300-6493675 | tert-butyl N-(2-{2-[2-(2-hydroxyethoxy)ethoxy]ethoxy}ethyl)carbamate |
| EN300-174976 | 3-(tert-butoxy)propan-1-ol |
| EN300-7468960 | 2-[4-(prop-2-yn-1-yloxy)butoxy]acetic acid |
| EN300-2008265 | N-{2-[2-(2-azidoethoxy)ethoxy]ethyl}-2-iodoacetamide |
| EN300-1264318 | tert-butyl 4-(2-hydroxyethoxy)piperidine-1-carboxylate |
| EN300-6476878 | 2-[2-(piperazin-1-yl)ethoxy]ethan-1-ol |
| EN300-2009274 | 3-(2-hydroxyethoxy)-4-methoxybenzaldehyde |
| EN300-7541483 | 3-[(oxiran-2-yl)methoxy]propan-1-ol |
| EN300-7541876 | 2-aminoethyl 2-cyanoacetate hydrochloride |
| EN300-137387 | 2-[2-(2-aminoethoxy)ethoxy]ethan-1-ol |
| EN300-226150 | 2-{2-[(2-aminoethyl)amino]ethoxy}ethan-1-ol |
| EN300-6482855 | 2-[(1-amino-3,3-dimethylbutan-2-yl)oxy]ethan-1-ol hydrochloride |
| EN300-6496816 | tert-butyl N-[2-(2-aminoethoxy)ethyl]carbamate hydrochloride |
| EN300-6497222 | 2-[2-(aminomethyl)phenoxy]ethan-1-ol hydrochloride |
| EN300-6498752 | 4-(2-aminoethoxy)-3-methoxyphenol hydrochloride |
| EN300-247319 | tert-butyl 2-[2-(2-aminoethoxy)ethoxy]acetate hydrochloride |
| EN300-6728975 | 2-(2-aminophenoxy)ethan-1-ol hydrochloride |
| EN300-7016274 | 3-(3-chloropropoxy)propan-1-ol |
| EN300-6494414 | 2-(2-hydroxyethoxy)ethyl formate |
| EN300-6746213 | 2-(2-bromoethoxy)ethan-1-amine hydrobromide |
| EN300-6734328 | 2-(2-chloroethoxy)-2-methylpropanoic acid |
| EN300-6746825 | 5-(2-hydroxyethoxy)pentan-1-ol |
| EN300-384559 | 4-[(2-hydroxyethoxy)methyl]piperidin-4-ol hydrochloride |
| EN300-6738369 | 2-(3-bromopropoxy)ethan-1-amine hydrobromide |
| EN300-192867 | 2-[2-(benzyloxy)ethoxy]ethan-1-ol |
| EN300-7431084 | 3-(3-aminopropoxy)propan-1-ol hydrochloride |
| EN300-317133 | 2-{2-[2-(benzyloxy)ethoxy]ethoxy}ethan-1-ol |
| EN300-7354186 | 2-[(piperidin-4-yl)methoxy]ethan-1-ol hydrochloride |
| EN300-6730172 | tert-butyl N-[2-(3-aminopropoxy)ethyl]carbamate |
| EN300-6748987 | 2-[(2-hydroxyethoxy)carbonyl]benzoic acid |
| EN300-6746824 | 3-(2-hydroxyethoxy)-2-methylpropan-1-ol |
| EN300-1472331 | 2-{2-[4-(2-{[2-(2-aminoethoxy)ethyl]amino}ethyl)piperazin-1-yl]ethoxy}ethan-1-amine |
| EN300-1655786 | [2-(2-chloroethoxy)ethyl]phosphonic acid |
| EN300-6764893 | 2-(pyrrolidin-3-yloxy)ethan-1-ol hydrochloride |
| EN300-315500 | 4-amino-3-(3-hydroxypropoxy)benzoic acid |
| EN300-19202 | 1-chloro-2-(2-chloroethoxy)ethane |
| EN300-244444 | 2-{2-[2-(2-azidoethoxy)ethoxy]ethoxy}ethan-1-ol |
| EN300-7438426 | tert-butyl[2-(2-iodoethoxy)ethoxy]dimethylsilane |
| EN300-7441287 | {1-[2-(benzyloxy)ethyl]piperidin-4-yl}methanol hydrochloride |
| EN300-7360457 | 3-(2-aminoethoxy)benzoic acid hydrochloride |
| EN300-263879 | 2,2,3,3-tetramethyl-4,7,10-trioxa-3-siladodecan-12-ol |
| EN300-7460414 | tert-butyl N-[2-(2-aminoethoxy)phenyl]carbamate hydrochloride |
| EN300-7459316 | O-{6-[3,5-bis(chloromethyl)phenoxy]hexyl}hydroxylamine; trifluoroacetic acid |
| EN300-761318 | 2-(pent-4-yn-1-yloxy)acetic acid |
| EN300-7353728 | 3-[2-(2-chloroethoxy)ethoxy]propan-1-ol |
| EN300-7411332 | 1-iodo-2-(2-iodoethoxy)ethane |
| EN300-7417011 | 2-{2-[2-(prop-2-yn-1-yloxy)ethoxy]ethoxy}ethan-1-ol |
| EN300-7435275 | ({2-[2-(2-bromoethoxy)ethoxy]ethoxy}methyl)benzene |
| EN300-7465452 | 5-(2-aminoethoxy)-2-fluorobenzoic acid hydrochloride |
| EN300-1694009 | chloro[2-(prop-2-yn-1-yloxy)ethoxy]methanone |
| EN300-1294256 | 2-[(1-amino-3-methylbutan-2-yl)oxy]ethan-1-ol |
| EN300-7456815 | 4-(2-{[4-(tert-butoxy)-4-oxobutanoyl]oxy}ethoxy)-4-oxobutanoic acid |
| EN300-7443005 | 12-iodo-2,2,3,3-tetramethyl-4,7,10-trioxa-3-siladodecane |
| EN300-19499 | 2-{2-[2-(2-hydroxyethoxy)ethoxy]ethoxy}ethan-1-ol |
| EN300-16700 | 2-(2-hydroxyethoxy)phenol |
| EN300-134537 | 2-[2-(2-chloroethoxy)ethoxy]ethan-1-ol |
| EN300-7427656 | 14-amino-3,6,9,12-tetraoxatetradecan-1-ol hydrochloride |
| EN300-157830 | 2-(piperidin-3-yloxy)ethan-1-ol hydrochloride |
| EN300-245588 | tert-butyl 3-(2-hydroxyethoxy)propanoate |
| EN300-344268 | [2-(2-chloroethoxy)ethyl](methyl)amine hydrochloride |
| EN300-7471137 | sodium 5-(2-aminoethoxy)-2-chlorobenzoate |
| EN300-7440874 | 2-[4-(bezyloxy)butoxy]ethan-1-ol |
| EN300-1587973 | 2-{[1-(aminomethyl)cyclopentyl]oxy}ethan-1-ol hydrochloride |
| EN300-1589200 | 2-{2-[2-(2-aminoethoxy)ethoxy]ethoxy}ethan-1-ol |
| EN300-7463738 | 2-{2-[2-(2-chloroethoxy)ethoxy]ethoxy}ethan-1-ol |
| EN300-6477608 | 2-[2-(2-hydroxyethoxy)ethoxy]acetic acid |
| EN300-7549881 | 5-(2-aminoethoxy)-2-methylbenzoic acid hydrochloride |
| EN300-7549176 | 3-(3-aminopropoxy)benzoic acid hydrochloride |
| EN300-7549473 | 3-(2-aminoethoxy)-4-methylbenzoic acid hydrochloride |
| EN300-7563216 | 2-{2-[4-(azetidin-3-yl)piperazin-1-yl]ethoxy}ethan-1-ol dihydrochloride |
| EN300-7562783 | lithium(1+) 3-(3-hydroxypropoxy)propanoate |
| EN300-7549954 | 3-(2-aminoethoxy)-2-methylbenzoic acid hydrochloride |
| EN300-24791881 | 3-(3-azidopropoxy)propan-1-ol |
| EN300-7500523 | 2-{2-[2-(2-hydroxyethoxy)ethoxy]ethoxy}acetic acid |
| EN300-26619695 | 2-(2-aminoethoxy)-3-methoxyphenol hydrochloride |
| EN300-7348271 | 2-[2-(prop-2-yn-1-yloxy)ethoxy]ethan-1-ol |
| EN300-7545089 | 3-[2-(3-chloropropoxy)ethoxy]propan-1-ol |
| EN300-7440875 | tert-butyl[4-(2-iodoethoxy)butoxy]dimethylsilane |
| EN300-22991138 | 4-[3-(4-azidobutoxy)propoxy]butan-1-ol |
| EN300-105617 | 4-(benzyloxy)butan-1-ol |
| EN300-26860934 | 2-(methylamino)ethyl 2-cyanoacetate hydrochloride |
| EN300-26675060 | 3-[2-(3-aminopropoxy)ethoxy]propan-1-ol hydrochloride |
| EN300-7563169 | 4-(4-azidobutoxy)butan-1-ol |
| EN300-7461359 | 4-[3-(4-hydroxybutoxy)propoxy]butan-1-ol |
| EN300-6490355 | 4,7,10,13-tetraoxahexadec-15-ynoic acid |
| EN300-22991137 | 3-[2-(3-azidopropoxy)ethoxy]propan-1-ol |
| EN300-6493668 | tert-butyl 3-[2-(2-aminoethoxy)ethoxylpropanoate |
| EN300-22991140 | 4-[3-(4-hydroxybutoxy)propoxy]butanoic acid |
| EN300-26286168 | 1-(2-aminoethoxy)-2-(2-azidoethoxy)ethane hydrochloride |
| EN300-23254233 | 3-(2-{2-[2-(3-formyl-2-hydroxyphenoxy)ethoxy]ethoxy]ethoxy)-2-hydroxybenzaldehyde |
| EN300-227949 | 3-ethoxy-4-(2-hydroxyethoxy)benzaldehyde |
| EN300-22991136 | 4-[3-(4-chlorobutoxy)propoxy]butan-1-ol |
| EN300-7472215 | 3-{2-[2-(2-aminoethoxy)ethoxy]ethoxy}prop-1-yne hydrochloride |
| EN300-7373222 | 4,7,10,13,16-pentaoxanonadec-18-ynoic acid |
| EN300-7086397 | 4-(prop-2-yn-1-yloxy)butan-1-ol |
| EN300-6491579 | 3,6,9,12-tetraoxapentadec-14-yn-1-amine |
| EN300-7441482 | 3-hydroxy-4-(2-methoxyethoxy)benzaldehyde |
| EN300-53715 | 2-(2-amino-5-fluorophenoxy)ethan-1-ol |
| EN300-109693 | 2-[(oxiran-2-yl)methoxy]ethan-1-ol |
| EN300-20950 | 2-(2-aminoethoxy)ethan-1-ol |
| EN300-7420955 | 1-azido-2-[2-(2-bromoethoxy)ethoxy]ethane |
| EN300-298437 | 2-{[1-(aminomethyl)-4,4-difluorocyclohexyl]oxy}ethan-1-ol |
| EN300-7624053 | 3-(prop-2-yn-1-yloxy)propan-1-amine hydrochloride |
| EN300-26673732 | methyl 3-[2-(2-aminoethoxy)ethoxy]propanoate hydrochloride |
| EN300-343270 | methyl 4-amino-3-(2-hydroxyethoxy)benzoate hydrochloride |
| EN300-300233 | 3-{[2-(tert-butoxy)ethyl]amino}propanoic acid |
| EN300-315506 | 4-amino-3-(4-hydroxybutoxy)benzoic acid |
| EN300-145574 | 3-(4-aminophenoxy)propanoic acid |
| EN300-1719224 | 3-(2-aminoethoxy)propanoic acid hydrochloride |
| EN300-746601 | 4-(2-aminoethoxy)benzoic acid hydrochloride |
| EN300-213748 | [(3-bromopropoxy)methyl]benzene |
| EN300-108377 | [(2-bromoethoxy)methyl]benzene |
| EN300-317578 | tert-butyl N-{2-[2-(methylamino)ethoxy]ethyl}carbamate |
| EN300-77716 | methyl 4-[4-(aminomethyl)phenoxy]butanoate hydrochloride |
| EN300-1709680 | 2-(2-azidoethoxy)ethan-1-amine hydrochloride |
| EN300-315817 | tert-butyl N-[2-(2-aminoethoxy)ethyl]-N-methylcarbamate |
| EN300-208938 | 3-(2-{[(tert-butoxy)carbonyl]amino}ethoxy)propanoic acid |
| EN300-74705 | 2-(2-{[(tert-butoxy)carbonyl]amino}ethoxy)acetic acid |
| EN300-74728 | methyl 4-(4-aminophenoxy)butanoate hydrochloride |
| EN300-157099 | methyl 2-(2-aminoethoxy)acetate hydrochloride |
| EN300-1704345 | tert-butyl N-{2-[2-(2-aminoethoxy)ethoxy]ethyl}carbamate |
| EN300-209079 | {2-[(3-carboxypropanoyl)oxy]ethyl}trimethylazanium chloride |
| EN300-1721470 | 5-(2-hydroxyethoxy)pentanoic acid |
| EN300-22054521 | 3-[2-(3-hydroxypropoxy)ethoxy]propanoic acid |
| EN300-736822 | 2-acetamidoethyl (3S)-3-(aminomethyl)-5-methylhexanoate hydrochloride |
| EN300-1165943 | disodium 2-[2-(carboxymethoxy)ethoxy]acetate |
| EN300-1266169 | bis((2E)-but-2-enedioic acid); 3-ethyl 5-methyl 2,6-bis[(2-aminoethoxy)methyl]- |
| | 4-(2-chlorophenyl)-1,4-dihydropyridine-3,5-dicarboxylate |
| EN300-6736929 | 2-(3-chloropropoxy)ethan-1-amine hydrochloride |
| EN300-383885 | 3-{2-[2-(3-aminopropoxy)ethoxy]ethoxy}propan-1-amine |
| EN300-7426195 | 4-(2-hydroxyethoxy)butan-1-ol |
| EN300-7378103 | 2-(2-{[2-(2-hydroxyethoxy)ethyl]amino}ethoxy)ethan-1-ol |
| EN300-1723415 | 2-(2,5-dioxopyrrolidin-1-yl)ethyl (3S)-3-(aminomethyl)-5-methylhexanoate hydrochloride |
| EN300-19318 | 2-(2-hydroxyethoxy)ethan-1-ol |
| EN300-54211 | 2-[2-({5-[2-(2-hydroxyethoxy)ethoxy]naphthalen-1-yl}oxy)ethoxy]ethan-1-ol |
| EN300-258766 | 3,6,9,12-tetraoxatetradecane-1,14-diol |
| EN300-298141 | 2-methoxyethyl (3S)-3-(aminomethyl)-5-methylhexanoate; trifluoroacetic acid |
| EN300-106544 | 4-[(4-aminonaphthalen-1-yl)oxy]butanenitrile |
| EN300-306385 | [2-(2-aminoethoxy)ethyl](methyl)amine |
| EN300-71579 | propyl 3-aminopropanoate hydrochloride |
| EN300-246911 | 2-(propan-2-yloxy)ethyl 3-(aminomethyl)-5-methylhexanoate; trifluoroacetic acid |
| EN300-252444 | 2-{[2-(2-hydroxyethoxy)ethyl]amino}ethan-1-ol |
| EN300-252599 | 2-{2-[bis(2-hydroxyethyl)amino]ethoxy}ethan-1-ol |
| EN300-316362 | 2-(2-aminoethoxy)ethan-1-amine |
| EN300-10061 | 2-(2-chloroacetamido)ethyl 2-chloroacetate |
| EN300-222334 | tert-butyl N-(2-{[2-(2-hydroxyethoxy)ethyl]amino}ethyl)carbamate |
| EN300-7414647 | hexyl 5-amino-4-oxopentanoate hydrochloride |
| EN300-6976741 | 2-(but-3-yn-1-yloxy)propanoic acid |
| EN300-134148 | 2-(prop-2-yn-1-yloxy)ethan-1-amine hydrochloride |
| EN300-203531 | 3-(2-aminoethoxy)propan-1-ol |
| EN300-52467 | 2-(2-chloroethoxy)ethan-1-ol |
| EN300-231011 | 2-(2-aminophenoxy)ethan-1-ol |
| EN300-6493684 | 2-{2-[2-(2-{[(tert-butoxy)carbonyl]amino}ethoxy)ethoxy]ethoxy}acetic acid |
| EN300-6493687 | 3-[2-(2-{[(tert-butoxy)carbonyl]amino}ethoxy)ethoxy]propanoic acid |
| EN300-6493658 | tert-butyl N-(2-{2-[2-(2-azidoethoxy)ethoxy]ethoxy}ethyl)carbamate |
| EN300-6493693 | tert-butyl N-(2-{2-[2-(2-bromoethoxy)ethoxy]ethoxy}ethyl)carbamate |
| EN300-27736133 | 2-[(1-amino-2-methylpropan-2-yl)oxy]ethan-1-ol hydrochloride |
| EN300-104201 | 3-(2-hydroxyethoxy)propan-1-ol |
| EN300-7425446 | 2-(2-aminoethoxy)-6-methoxyphenol hydrochloride |
| EN300-27702276 | lithium(1+) 5-(2-hydroxyethoxy)pentanoate |
| EN300-226100 | 3,6,9,12,15-pentaoxaheptadecane-1,17-diol |
| EN300-118233 | 3-(prop-2-yn-1-yloxy)propanoic acid |
| EN300-27719669 | tert-butyl N-[2-(2-aminoethoxy)-2-methylpropyl]carbamate hydrochloride |
| EN300-42227 | 2-(5-amino-2-methoxyphenoxy)ethan-1-ol |
| EN300-8332618 | tert-butyl 2-(3-hydroxypropoxy)acetate |
| EN300-1590283 | 3,6,9,12,15,18,21-heptaoxatricosane-1,23-diol |
| EN300-1706889 | 2-{2-[2-(2-bromoethoxy)ethoxy]ethoxy}ethan-1-ol |
| EN300-27780250 | 2-{2-[2-(2-aminoethoxy)ethoxy]ethoxy}ethan-1-ol hydrochloride |
| EN300-6493670 | tert-butyl 3-{2-[2-(2-aminoethoxy)ethoxy]ethoxy}propanoate |
| EN300-7398805 | 2-(2-aminoethoxy)ethan-1-amine dihydrochloride |
| EN300-27721120 | tert-butyl N-15-(prop-2-yn-1-yloxy)pentyl]carbamate |
| EN300-7406235 | 14-azido-3,6,9,12-tetraoxatetradecan-1-ol |
| EN300-316286 | tert-butyl N-(2-{2-[2-(2-aminoethoxy)ethoxy]ethoxy}ethyl)carbamate |
| EN300-27082270 | 1-chloro-3-(2-chloroethoxy)propan-2-one |
| EN300-7364909 | 2-(4-aminophenoxy)ethan-1-ol hydrochloride |
| EN300-7421448 | tert-butyl 3-{2-[2-(2-hydroxyethoxy)ethoxy]ethoxy}propanoate |

Table 3 shows selections of the ligand module for E3 ligase binding, including but not limited to the molecular structures represented by SMILES and the compounds corresponding to the InChI key, as shown in Table 3 below for details.

| Sm1les | InChI Key |
|---|---|
| CN[C@@H](C)C(═O)N[C@H](C(═O)N1CC2═CC═CC═C2C[C@H]1C(═O)N[C@@H]1CC | ATYRKWQJUXHQEF- |
| CC2═CC═CC═C21)C(C)(C)C | QNMIOERPSA-N |
| NC1═CC═CC2═C1C(═O)N(C1CCC(═O)NC1═O)C2═O | UVSMNLNDYGZFPF- |
| | UHFFFAOYSA-N |
| O═C1CCC(N2C(═O)C3═CC═CC═C3C2═O)C(═O)N1 | UEJJHQNACJXSKW- |
| | UHFFFAOYSA-N |
| NC1═CC═CC2═C1CN(C1CCC(═O)NC1═O)C2═O | GOTYRUGSSMKFNF- |
| | UHFFFAOYSA-N |
| CC(═O)N[C@H](C(═O)N1C[C@H](O)[C@@H](F)[C@H]1C(═O)NCC1═CC═C(C2═C(C) | MNNVXLLCYGGFOQ- |
| N═CS2)C═C1)C(C)(C)C | YOUFYPILSA-N |
| NC1═CC═CC2═C1C(═O)N(C1CCC(═O)NC1═O)N═N2 | DXZBHVQOULDEPN- |
| | UHFFFAOYSA-N |
| CC(═O)N[C@@H](CC1═CC═CC═C1)C(═O)N[C@H](C(═O)N1C[C@H](O)C[C@H]1C | ALYDGEQICGMVIP- |
| (═O)NCC1═CC═C(C2═C(C)N═CS2)C═C1)C(C)(C)C | UYIZUTNXSA-N |
| NS(═O)(═O)C1═CC═C(S(═O)(═O)NC2═CC═CC3═C2[NH]C═C3Cl)C═C1 | SETFNECMODOHTO- |
| | UHFFFAOYSA-N |
| COC1═CC═C(C2═NC(C3═CC═C(C1)C═C3)C(C3═CC═C(C1)C═C3)N2C(═O)N2CCNC | BDUHCSBCVGXTJM- |
| (═O)C2)C(OC(C)C)═C1 | UHFFFAOYSA-N |
| COC1═CC(C(═O)O)═CC═C1NC(═O)[C@@H]1N[C@@H](CC(C)(C)C)[C@](C#N) | TVTXCJFHQKSQQM- |
| (C2═CC═C(C1)C═C2F)[C@H]1C1═CC═CC(C1)═C1F | LJQIRTBHSA-N |
| CCOC1═CC(C(C)(C)C)═CC═C1C1═N[C@@](C)(C2═CC═C(C1)C═C2)[C@@](C) | QBGKPEROWUKSBK- |
| (C2═CC═C(C1)C═C2)N1C(═O)N1CCN(CCCS(O(═O)═O)CC1 | QPPIDDCLSA-N |
| CC(═O)N[C@H](C(═O)N1C[C@H](O)C[C@H]1C(═O)NCC1═CC═C(C2═C(C)N═CS2) | GFVIEZBZIUKYOG- |
| C═C1)C(C)(C)C | SVFBPWRDSA-N |
| CC1═C(C2═CC═C(CNC(═O)[C@@H]3C[C@@H](O)CN3C(═O)[C@@H](NC(═O)C3 | NDVQUNZCNAMROD- |
| (C#N)CC3)C(C)(C)C)C═C2)SC═N1 | RZUBCFFCSA-N |
| CC1═C(C2═CC═C(CNC(═O)[C@@H]3C[C@@H](O)CN3C(═O)[C@@H](NC(═O)C3(F)C | GFNCBUDQFXZVNN- |
| C3)C(C)(C)C)C═C2)SC═N1 | SVFBPWRDSA-N |
| CC(═O)N[C@H](C(═O)N1C[C@H](O)C[C@H]1C(═O)N[C@@H](C)C1═CC═C(C2═C(C) | JAHUHEDUDMTTTF- |
| N═CS2)C═C1)C(C)(C)C | COWZOJLOSA-N |
| CC(═O)N[C@HJ(C(═O)N1C[C@H](O)[C@H](F)[C@H]1C(═O)NCC1═CC═C(C2═C(C) | MNNVXLLCYGGFOQ- |
| N═CS2)C═C1)C(C)(C)C | VNYTWHDVSA-N |
| CC(═O)N[C@H](C(═O)N1C[C@H](O)C[C@H]1C(═O)N[C@@H](C)C1═CC═C(C1)C═C1) | QORQDSURGXKKLU- |
| C(C)(C)C | PSMGESJCSA-N |
| CC(═O)N[C@H](C(═O)N1C[C@H](O)C[C@H]1C(═O)N[C@@H](C)C1═CC═C(C#N) | OXUULQVWMAOYDZ- |
| C═C1)C(C)(C)C | NUDXDXSLSA-N |
| C#CC1═CC═C([C@H](C)NC(═O)[C@@H]2C[C@@H](O)CN2C(═O)[C@@H](NC(C)═O) | WHTWICJCWCIKHM- |
| C(C)(C)C)C═C1 | WEFJBSGNSA-N |
| CC(═O)N[C@H](C(═O)N1C[C@H](O)C[C@H]1C(═O)N[C@@H](C)C1═CC═C(C2CC2) | CFQQOSHEPDRZSL- |
| C═C1)C(C)(C)C | MYDCNYLUSA-N |
| CC(═O)N[C@H](C(═O)N1C[C@H](O)C[C@H]1C(═O)N[C@@H](C)C1═CC═C(C(C)C) | FPHVSWUJMFIEOJ- |
| C═C1)C(C)(C)C | PSMQTCRGSA-N |
| CC(═O)N[C@H](C(═O)N1C[C@H](O)C[C@H]1C(═O)N[C@@H](C)C1═CC═C(C(C)(C)C) | HWRVUOLZBZRHRB- |
| C═C1)C(C)(C)C | PSMQTCRGSA-N |
| CNC(═O)C[C@H](NC(═O)[C@@H]lC[C!i@H](O)CN1C(═O)[C@@H](NC(C)═O)C(C)(C) | DHJHMRSRAVXNPQ- |
| C)C1═CC═C(C2═C(C)N═CS2)C═C1 | MDAIXWLXSA-N |
| CNC(═O)C[C@H](NC(═O)(C@@H]1C[C@@H)(O)CN1C(═O)[C@@H](C1═CC(C)═NO1) | BUFJKORGWVCZBG- |
| C(C)C)C1═CC═C(C2═C(C)N═CS2)C═C1 | HXKBJWFLSA-N |
| CNC(═O)C[C@H](NC(═O)[C@@H]1C[C@@H](O)CN1C(═O)[C@@H](NC(═O)C1(F) | PTPPMFQIIPBSRV- |
| CC1)C(C)(C)C)C1═CC═C(C2═C(C)N═CS2)C═C1 | MDAIXWLXSA-N |
| CNC(═O)C[C@H](NC(═O)[C@@H]1C[C@@H](O)CN1C(═O)[C@@H](NC(═O)C1(C#N)C | TXOUIGUFBKWBLO- |
| C1)C(C)(C)C)C1═CC═C(C2═C(C)N═CS2)C═C1 | HXKBJWFLSA-N |
| CNC(═O)C[C@@H1(NC(═O)[C@@H]1C[C@@H)(O)CN1C(═O)[C@@H](NC(═O)C1(F)C | PTPPMFQIIPBSRV- |
| C1)C(C)(C)C)C1═CC═C(C2═C(C)N═CS2)C═C1 | IUBSTNSRSA-N |
| CNC(═O)C[C@H](NC(═O)[C@@H][C@@H](O)CN1C(═O)[C@H](NC(═O)C1(F)CC1)C | PTPPMFQIIPBSRV- |
| (C)(C)C)C1═CC═C(C2═C(C)N═CS2)C═C1 | XBJMDHIQSA-N |
| CC1═C(C2═CC═C(CNC(═O)[C@@H]3C[C@@H](O)CN3C(═O)[C@H](C(C)C)N3CC4═CC | HEDFFPYRFJKXQP- |
| ═CC═C4C3═O)C═C2)SC═N1 | VJTSUQJLSA-N |
| C#CC1═CC═C([C@H](CC(═O)NC)NC(═O)[C@@H]2C[C@@H](O)CN2C(═O)[C@@H] | RILDEXHTOOMQCV- |
| (C2═CC(C)═NO2)C(C)C)C═C1 | MDAIXWLXSA-N |
| CNC(═O)C[C@H1(NC(═O)[C@@H]1C[C@@H](O)CN1C(═O)[C@@H1(C1═CC(C)═NO1) | GEWZUXIXBYUKKN- |
| C(C)C)C1═CC═C(C#N)C═C1 | QTDGGUCWSA-N |
| CNC(═O)C[C@HJ(NC(═O)[C@@H]1C[C@@H](O)CN1C(═O)[C@@H](C1═CC(C)═NO1) | DZVFTEQNLPBNLU- |
| C(C)C)C1═CC═C(Br)C═C1 | MDPIYQRISA-N |
| CNC(═O)C[C@H](NC(═O)[C@@H]1C[C@@H](O)CN1C(═O)[C@@H](C1═CC(C)═NO1) | AVTUCSSCVXIPKR- |
| C(C)C)C1═CC═C(C1)C═C1 | MDPIYQRISA-N |
| CNC(═O)C[C@H](NC(═O)[C@@H]1C[C@@H](O)CN1C(═O)[C@@H](C1═CC(C)═NO1) | QOVORLCCBWSWNE- |
| C(C)C)C1═CC═C(F)C═C1 | MDPIYQRISA-N |
| CNC(═O)C[C@H](NC(═O)[C@@H]1C[C@@H](O)CN1C(═O)[C@@H1(C1═CC(C)═NO1) | YRJWYFLCJKATIJ- |
| C(C)C)C1═CC═CC═C1 | MDPIYORISA-N |
| CNC(═O)C[C@HJ(NC(═O)[C@@H]1C[C@@H](O)CN1C(═O)[C@@H](NC(C)═O)C(C)C) | UEIQRCHIEZGBTN- |
| C1═CC═CC═C1 | LXZJYRNTSA-N |
| COC1═CC═C(C2═N[C@H](C3═CC═C(C1)C═C3)[C@H](C3═CC═C(C1)C═C3)N2C(═O) | BDUHCSBCVGXTJM- |
| N2CCNC(═O)C2)C(OC(C)C)═C1 | IZLXSDGUSA-N |
| CC(C)C[C@H](NC(═O)[C@@H](O)[C@H](N)CC1═CC═CC═C1)C(═O)O | VGGGPCQERPFHOB- |
| | RDBSUJKOSA-N |
| CN[C@@H](C)C(═O)N[C@H](C(═O)N1C[C@@H](N)C[C@H]1C(═O)NC1═C(F)C═CC═C1F) | QESAPZFIIYNQSO- |
| C(C)(C)C | WXRXAMBDSA-N |
| CN[C@@H](C)C(═O)N[C@H](C(═O)N1CCC[C@H]1C(═O)N[C@@H]1CCCC2═CC═CC═C21) | NJUHOYLCAHNNLJ- |
| C1CCCCC1 | QODFHFCMSA-N |
| COC1═CC═C2C(═C1)CCCN2C(═O)CC1 | XJPUWRWIBSSPSL- |
| | UHFFFAOYSA-N |
| O═C1C═C(C2═CC═CC═C2)OC2═CC═C3C═CC═CC3═C12 | OUGIDAPQYNCXRA- |
| | UHFFFAOYSA-N |
| O═C1C═C(C2═CC═CC═C2)OC2═C1C═CC1═CC═CC═C12 | VFMMPHCGEFXGIP- |
| | UHFFFAOYSA-N |
| COC(═O)C1═CSC(C(═O)C2═C[NH]C3═CC═CC═C23)═N1 | KDDXOGDIPZSCTM- |
| | UHFFFAOYSA-N |
| CN[C@@H](C)C(═O)N[C@H](C(═O)N1CCC[C@H]1C1═NC(C(═O)C2═CC═C(F)C═C2)═C | JIOIGDQISXGQSO- |
| S1)C1CCN(C)CC1 | SSKFGXFMSA-N |
| COC1═CC═C(C2═N[C@@H](C3═CC═C(C1)C═C3)[C@@H](C3═CC═C(C1)C═C3)N2C | BDUHCSBCVGXTJM- |
| (═O)N2CCNC(═O)C2)C(OC(C)C)═C1 | WUFINQPMSA-N |
| CN[C@@H](C)C(═O)N[C@H](C(═O)N1CCC[C@H]1C(═O)N[C@H](C(═O)OC)C | UUPZYAHONNHULX- |
| (C1═CC═CC═C1)C1═CC═CC═C1)C1CCCCC1 | CJBSCAABSA-N |
| CN[C@@H](C)C(═O)N[C@H](C(═O)N1CCC[C@H]1C(═O)N[C@H](C(═O)OC)C | UUPZYAHONNHULX- |
| (C1═CC═CC═C1)C1═CC═CC═C1)C1CCCCC1 | CJBSCAABSA-N |
| CN[C@@H](C)C(═O)N[C@H](C(═O)N1CCC[C@H]1C1═NC(C(═O)C2═CC═CC | LCQFGFLQFLFDST- |
| (OC)═C2)═CS1)C1CCCCC1 | RTFZILSDSA-N |
| CN[C@@H](C)C(═O)N[C@H](C(═O)N1CCC[C@H]1C1═NC(C(═O)C2═CC═CC | LCQFGFLQFLFDST- |
| (OC)═C2)═CS1)C1CCCCC1 | RTFZILSDSA-N |
| CN[C@@H](C)C(═O)N[C@H](C(═O)N1CCC[C@H]1C1═NC(C(═O)C2═CC═CC | LCQFGFLQFLFDST- |
| (OC)═C2)═CS1)C1CCCCC1 | RTFZILSDSA-N |
| CN[C@@H](QC(═O)N[C@H](C(═O)N1C[C@@H](OC2═CC═CC═C2)C[C@H]1C(═O) | GUOVHJVNYPRNQS- |
| N[C@@H]1CCCC2═CC═CC═C21)C1CCCCC1 | SHUILBLCSA-N |
| CN[C@@H](C)C(═O)N[C@H](C(═O)N1C[C@H](OC2═CC═CC═C2)C[C@H]1C(═O) | GUOVHJVNYPRNQS- |
| N[C@@H]1CCCC2═CC═CC═C21)C1CCCCC1 | XIKYZZKVSA-N |
| CN[C@@H](C)C(═O)N[C@H](C(═O)N[C@H]1C[C@H]2CC[C@@H]1N(CCC1═CC═CC═C1) | GBQXNBCXOOCMBG- |
| C2)C1CCCCC1 | CFYMNZCRSA-N |
| CN[C@@H](C)C(═O)N[C@H](C(═O)N[C@H]1C[C@H]2CC[C@@H]1N(CCC1═CC═CC═C1) | GBQXNBCXOOCMBG- |
| C2)C1CCCCC1 | CFYMNZCRSA-N |
| CN[C@@H](C)C(═O)N[C@H](C(═O)N1CCC[C@H]1C1═NC(C2═CC═C(F) | OXVDUVSHCWCOFU- |
| C3═CC═CC═C23)═CS1)C1CCCCC1 | ATANMQQVSA-N |
| CN[C@@H](C)C(═O)N[C@HJ(C(═O)N1CCC[C@H]1C1═NC2═C(C3═CC═CC═C3)N═CC═C2 | DAXYGNXUBMFNHC- |
| S1)C1CCCCC1 | XZOYJPPVSA-N |
| CN[C@@H](C)C(═O)N[C@H](C(═O)N1CCC[C@H]1C1═CN═CC(C2═CC═C(F)C(C(═O) | ORPIGORNDCHEIA- |
| O)═C2)═C1)C1CCCCC1 | HDBFHEOPSA-N |
| CN[C@@H](C)C(═O)N[C@H](C(═O)N1CCC[C@H]1C1═CN═CC(N2C═CC3═C(C(═O)O)C | FFGHOKFUXXWKFH- |
| ═CC═C32)═C1)C(C)C | OWSXEPHWSA-N |
| CN[C@@H](C)C(═O)N[C@H](C(═O)N1CCC[C@H]1C(═O)NC1═C(C2═CC═CC═C2)N═NS1) | WZRFLSDVFPIXOV- |
| C1CCCCC1 | LRQRDZAKSA-N |
| CN[C@@H](C)C(═O)N[C@H](C(═O)N1CCC[C@H]1C(═O)NC1═C(C2═CC═CC═C2)N═NS1) | WZRFLSDVFPIXOV- |
| C1CCCCC1 | LRQRDZAKSA-N |
| CN[C@@H](C)C(═O)N[C@H](C(═O)N1CCC[C@H]1C(═O)NC1═C(C2═CC═CC═C2)N═NS1) | WZRFLSDVFPIXOV- |
| C1CCCCC1 | LRQRDZAKSA-N |
| CN[C@@H](OC(═O)N[C@H](C(═O)N1CCC[C@H]1C1═CN═CC(C(═O)C2═CC═C(F) | PLYJYKAWZXEBFC- |
| C═C2)═C1)C1CCCCC1 | WDNCENIBSA-N |
| NC1═CC═C2C(═O)N(C3CCC(═O)NC3═O)C(═O)C2═C1 | IICWMVJMJVXCLY- |
| | UHFFFAOYSA-N |
| CN[C@@H](C)C(═O)N[C@H]1CCO[C@H]2CC(C)(C)[C@@H](C(═O)N[C@@H]3CCC | PBGOFGSVVXGJCA- |
| C4═CC═CC═C43)N2C1═O | KDJJVYBXSA-N |
| O═C1CCC(N2C(═O)C3═CC═CC4═CC═CC(═C34)C2═O)C(═O)N1 | BERVIROBWDIAQO- |
| | UHFFFAOYSA-N |
| COC1═CC═C(OC2═CC═C(N(CC3═CC═CC═C3)C(═O)CC1)C═C2)C═C1 | DPADEQNOMBTITM- |
| | UHFFFAOYSA-N |
| CN1CCN(C2═CC═C(NC3CCC(═O)NC3═O)C═C2)CC1 | GRQMMULSBXXEST- |
| | UHFFFAOYSA-N |
| O═C1CCC(N2C(═O)OC3═CC═CC═C32)C(═O)N1 | WCKFQKVNTHAOGC- |
| | UHFFFAOYSA-N |
| NC1═CC═CC2═C1CN([C@H]1CCC(═O)NC1═O)C2═O | GOTYRUGSSMKFNF- |
| | JTQLQIEISA-N |
| COC(═O)C[C@H]1[C@12(C)C3═C(C)[C@H](C4═COC═C4)C[C@H]3O[C@@H]2 | JZIQWNPPBKFOPT- |
| [C@@H]2OC(═O)[C@]3(C)C═CC(═O)[C@@]1(C)[C@@H]23 | LSYMHUITSA-N |
| COC1═CC═CC2═C1C(═O)N(C1CCC(═O)NC1═O)C2 | WQBYRVHGTFSBTA- |
| | UHFFFAOYSA-N |
| O═C(CC1)NC(C(═O)NCC1═CC═CC═C1)C1═CC═C(C1)C(C1)═C1 | IARWWDNCNPMRCK- |
| | UHFFFAOYSA-N |
| O═C(CC1)NC(C(═O)NCC1═CC═CC═C1)C1═CC═C(C1)C═C1 | OOWBZXBMBGHPDI- |
| | UHFFFAOYSA-N |

Table 4 shows selections of the cell-penetrating peptide module, including but not limited to the sequences indicated by “cell-penetrating peptide sequence”, as shown in Table 4 below for details.

| Cell-penetrating peptide sequence | Cell-penetrating peptide sequence |
|---|---|
| KKRRQRRRPPQ (SEQ ID NO: 19) | akvkdepqrrsarlsakpappkpepkpkkapakk |
| LGISYGRKKRRQRRRPPQ (SEQ ID NO: | PLSSIFSRIGDP (SEQ ID NO: 413) |
| 20) | |
| FITKALGISYGRKKRRQRRRPPQ (SEQ | PSSSSSSRIGDP (SEQ ID NO: 414) |
| ID NO: 21) | |
| FITKALGISYGRKKRR (SEQ ID NO: 22) | vrlpppvrlpppvrlppp |
| GRKKRRQRRR (SEQ ID NO: 23) | VELPPPVELPPPVELPPP (SEQ ID NO: 415) |
| RKKRRQRRR (SEQ ID NO: 24) | ALWMTLLKKVLKAAAKAALNAVLVGANA (SEQ ID NO: |
| | 416) |
| RKKRRQRR (SEQ ID NO: 25) | ALWKTLLKKVLKA (SEQ ID NO: 417) |
| RKKRRQR (SEQ ID NO: 26) | ALWKTLLKKVLKAPKKKRKV (SEQ ID NO: 418) |
| KKRRQRRR (SEQ ID NO: 27) | PKKKRKVALWKTLLKKVLKA (SEQ ID NO: 419) |
| KRRQRRR (SEQ ID NO: 28) | VKRKKKPALWKTLLKKVLKA (SEQ ID NO: 420) |
| rkkrrqrrr | RQARRNRRRALWKTLLKKVLKA (SEQ ID NO: 421) |
| RRRQRRKKR (SEQ ID NO: 29) | RQARRNRRRC (SEQ ID NO: 422) |
| rrrqrrkkr | GRKKRRQRRRPPQC (SEQ ID NO: 423) |
| AKKRRQRRR (SEQ ID NO: 30) | EEEAAGRKRKKRT (SEQ ID NO: 424) |
| RAKRRQRRR (SEQ ID NO: 31) | EEE |
| RKARRQRRR (SEQ ID NO: 32) | EEEAA (SEQ ID NO: 425) |
| RKKARQRRR (SEQ ID NO: 33) | EEEAAKKK (SEQ ID NO: 426) |
| RKKRAQRRR (SEQ ID NO: 34) | GRKRKKRT (SEQ ID NO: 427) |
| RKKRRARRR (SEQ ID NO: 35) | FFFAAGRKRKKRT (SEQ ID NO: 428) |
| RKKRRQARR (SEQ ID NO: 36) | NNNAAGRKRKKRT (SEQ ID NO: 429) |
| RKKRRQRAR (SEQ ID NO: 37) | YYYAAGRKRKKRT (SEQ ID NO: 430) |
| RKKRRQRRA (SEQ ID NO: 38) | MVTVLFRRLRIRRACGPPRVRV (SEQ ID NO: 431) |
| GRKKRRQRRPPQC (SEQ ID NO: 39) | RQIKIWFQNRRMKWKK (SEQ ID NO: 432) |
| GRKKRRQRPPQC (SEQ ID NO: 40) | AGYLLGKINLKALAALAKKIL (SEQ ID NO: 433) |
| GRKKRRQPPQC (SEQ ID NO: 41) | VQRKRQKLMP (SEQ ID NO: 434) |
| GRKKRRQRRRC (SEQ ID NO: 42) | SKKKKTKV (SEQ ID NO: 435) |
| GRKKRRQRARPPQC (SEQ ID NO: 43) | GRKRKKRT (SEQ ID NO: 436) |
| GRKKRRQARAPPQC (SEQ ID NO: 44) | GKKKKRKREKL (SEQ ID NO: 437) |
| TRQARRNRRRRWRERQR (SEQ ID NO: 45) | PKKKRKV (SEQ ID NO: 438) |
| RRRR (SEQ ID NO: 46) | ERKKRRRE (SEQ ID NO: 439) |
| RRRRR (SEQ ID NO: 47) | FKKFRKF (SEQ ID NO: 440) |
| RRRRRR (SEQ ID NO: 48) | LGTYTQDFNKFHTFPQTAIGVGAP (SEQ ID NO: 441) |
| RRRRRRR (SEQ ID NO: 49) | LGTYTQDFNKFHTFPQTAIGVGAP (SEQ ID NO: 442) |
| RRRRRRRR (SEQ ID NO: 2) | YTQDFNKFHTFPQTAIGVGAP (SEQ ID NO: 443) |
| RRRRRRRRR (SEQ ID NO: 50) | DFNKFHTFPQTAIGVGAP (SEQ ID NO: 444) |
| RRRRRRRRRRR (SEQ ID NO: 51) | KFHTFPQTAIGVGAP (SEQ ID NO: 445) |
| RRRRRRRRRRRR (SEQ ID NO: 52) | TFPQTAIGVGAP (SEQ ID NO: 446) |
| RRRRRRRRRRRRRRRR (SEQ ID NO: 53) | GYGRKKRRQRRRG (SEQ ID NO: 447) |
| rrrrr | GYGRKKRRQRRRG (SEQ ID NO: 448) |
| rrrrrr | GYGRKKRRQRRRG (SEQ ID NO: 449) |
| rrrrrrr | RQIKIWFQNRRMKWKK (SEQ ID NO: 450) |
| rrrrrrrr | RQIKIWFQNRRMKWKK (SEQ ID NO: 450) |
| rrrrrrrrr | RQIKIWFQNRRMKWKK (SEQ ID NO: 450) |
| GWTLNSAGYLLGKINLKALAALAKKIL | FLGKKFKKYFLQLLK (SEQ ID NO: 451) |
| (SEQ ID NO: 54) | |
| GWTLNSAGYLLGKINLKALAALAKKLL | FLIFIR VICIVIAKLKANLMCKT (SEQ ID NO: 452) |
| (SEQ ID NO: 55) | |
| GWTLNSAGYLLGKFLPLILRKIVTAL | KKAAQIRSQVMTHLRVI (SEQ ID NO: 453) |
| (SEQ ID NO: 56) | |
| GWTLNPAGYLLGKINLKALAALAKKIL | YIVLRRRRKRVNTKRS (SEQ ID NO: 454) |
| (SEQ ID NO: 57) | |
| GWTLNPPGYLLGKINLKALAALAKKIL | RRKLSQQKEKK (SEQ ID NO: 455) |
| (SEQ ID NO: 58) | |
| LNSAGYLLGKINLKALAALAKKIL (SEQ | VQAILRRNWNQYKIQ (SEQ ID NO: 456) |
| ID NO: 59) | |
| LLGKINLKALAALAKKIL (SEQ ID NO: | KTVLLRKLLKLLVRKI (SEQ ID NO: 457) |
| 60) | |
| GWTLNSAGYLLGKLKALAALAKKIL | LLKKRKVVRLIKFLLK (SEQ ID NO: 458) |
| (SEQ ID NO: 61) | |
| AGYLLGKINLKALAALAKKIL (SEQ ID | KLPCRSNTFLNIFRRKKPG (SEQ ID NO: 459) |
| NO: 62) | |
| GWTLNSKINLKALAALAKKIL (SEQ ID | KKICTRKPRFMSAWAQ (SEQ ID NO: 460) |
| NO: 63) | |
| LNSAGYLLGKLKALAALAKIL (SEQ ID | RQIKIWFQNRRMKWKK (SEQ ID NO: 461) |
| NO: 64) | |
| LNSAGYLLGKALAALAKKIL (SEQ ID | RGGRLSYSRRRFSTSTGR (SEQ ID NO: 462) |
| NO: 65) | |
| AGYLLGKLKALAALAKKIL (SEQ ID | rggrlsysrrrfststgr |
| NO: 66) | |
| LNSAGYLLGKLKALAALAK (SEQ ID | RRLSYSRRRF (SEQ ID NO: 463) |
| NO: 67) | |
| GWTLNSAGYLLGKINLKAPAALAKKIL | rrlsysrrrf |
| (SEQ ID NO: 68) | |
| GWTLNSAGYLLGPHAVGNHRSFSDKN | RGGRLAYLRRRWAVLGR (SEQ ID NO: 464) |
| GLTS (SEQ ID NO: 69) | |
| INLKALAALAKKIL (SEQ ID NO: 70) | RQIKIWFQNRRMKWKK (SEQ ID NO: 465) |
| KLALKLALKALKAALKLA (SEQ ID NO: | MANLGYWLLALFVTMWTDVGLCKKRPKP (SEQ ID NO: |
| 71) | 466) |
| KLALKLALKAWKAALKLA (SEQ ID | MANLGCWMLVLFVATWSDLGLCKKRPKP (SEQ ID NO: |
| NO: 72) | 467) |
| KLALKAALKAWKAAAKLA (SEQ ID | MVKSKIGSWILVLFVAMWSDVGLCKKRPKP (SEQ ID NO: |
| NO: 73) | 468) |
| KLALKAAAKAWKAAAKAA (SEQ ID | LLGDFFRKSKEKIGKEFKRIVQRIKDFLRNLVPRTESC (SEQ |
| NO: 74) | ID NO: 469) |
| KITLKLAIKAWKLALKAA (SEQ ID NO: | RQIKIWFQNRRMKWKK (SEQ ID NO: 470) |
| 75) | |
| KIAAKSIAKIWKSILKIA (SEQ ID NO: | RVIRVWFQNKRCKDKK (SEQ ID NO: 471) |
| 76) | |
| KALAKALAKLWKALAKAA (SEQ ID | GIGKFLHSAKKWGKAFVGQIMNC (SEQ ID NO: 472) |
| NO: 77) | |
| KLALKLALKWAKLALKAA (SEQ ID | TRSSRAGLQWPVGRVHRLLRKGGC (SEQ ID NO: 473) |
| NO: 78) | |
| KLLAKAAKKWLLLALKAA (SEQ ID | YGRKKRRQRRR (SEQ ID NO: 1) |
| NO: 79) | |
| KLLAKAALKWLLKALKAA (SEQ ID | RHIKIWFQNRRMKWKK (SEQ ID NO: 474) |
| NO: 80) | |
| KALKKLLAKWLAAAKALL (SEQ ID | RKKRRQRRR (SEQ ID NO: 475) |
| NO: 81) | |
| KLAAALLKKWKKLAAALL (SEQ ID | RQIKIWFQNRRMKWKK (SEQ ID NO: 476) |
| NO: 82) | |
| KALAALLKKWAKLLAALK (SEQ ID | SKRTRQTYTRYQTLELEKEFHFNRYITRRRRIDIANALSLSE |
| NO: 83) | RQIKIWFQNRRMKSKKDR (SEQ ID NO: 477) |
| KALAALLKKLAKLLAALK (SEQ ID NO: | EKRPRTAFSSEQLARLKREFNENRYLTTERRRQQLSSELGL |
| 84) | NEAQIKIWFQNKRAKIKKST (SEQ ID NO: 478) |
| KLALKLALKALKAALK (SEQ ID NO: 85) | GRRRRRRRRRPPQ (SEQ ID NO: 479) |
| KLALKALKAALKLA (SEQ ID NO: 86) | GALFLGFLGAAGSTMGAWSQPKKKRKV (SEQ ID NO: 480) |
| KLALKLALKALKAA (SEQ ID NO: 87) | GALFLAFLAAALSLMGLWSQPKKKRRV (SEQ ID NO: 481) |
| KLGLKLGLKGLKGGLKLG (SEQ ID NO: 88) | MLLLTRRRST (SEQ ID NO: 482) |
| KLALKLALKALQAALQLA (SEQ ID NO: 89) | CGNKRTRGC (SEQ ID NO: 483) |
| KLALQLALQALQAALQLA (SEQ ID NO: 90) | TSPLNIHNGQKL (SEQ ID NO: 484) |
| QLALQLALQALQAALQLA (SEQ ID NO: 91) | GLRKRLRKFRNKIKEK (SEQ ID NO: 485) |
| ELALELALEALEAALELA (SEQ ID NO: 92) | GLLEALAELLEGLRKRLRKFRNKIKEK (SEQ ID NO: 486) |
| LKTLATALTKLAKTLTTL (SEQ ID NO: 93) | CVQWSLLRGYQPC (SEQ ID NO: 487) |
| LLKTTALLKTTALLKTTA (SEQ ID NO: 94) | VRLPPP (SEQ ID NO: 488) |
| LKTLTETLKELTKTLTEL (SEQ ID NO: 95) | VRLPPPVRLPPP (SEQ ID NO: 489) |
| LLKTTELLKTTELLKTTE (SEQ ID NO: 96) | VRLPPPVRLPPPVRLPPP (SEQ ID NO: 490) |
| RQIKIWFQNRRMKWKK (SEQ ID NO: 97) | VHLPPP (SEQ ID NO: 491) |
| klalklalkalkaalkla | VHLPPPVHLPPP (SEQ ID NO: 492) |
| KALKLKLALALLAKLKLA (SEQ ID NO: 98) | VHLPPPVHLPPPVHLPPP (SEQ ID NO: 493) |
| RQIKIWFQNRRMKWKK (SEQ ID NO: 99) | VKLPPP (SEQ ID NO: 494) |
| KKWKMRRNQFWIKIQR (SEQ ID NO: 100) | VKLPPPVKLPPP (SEQ ID NO: 495) |
| rqikiwfqnrrmkwkk | VKLPPPVKLPPPVKLPPP (SEQ ID NO: 496) |
| RQIKIWFPNRRMKWKK (SEQ ID NO: 101) | RQIKIWFQNRRMKWKK (SEQ ID NO: 497) |
| RQPKIWFPNRRKPWKK (SEQ ID NO: 102) | RQIKIFFQNRRMKWKK (SEQ ID NO: 498) |
| RQIKIWFQNRRMKWKK (SEQ ID NO: 103) | ASMWERVKSIIKSSLAAASNI (SEQ ID NO: 499) |
| RQIKIWFQNRRMKWK (SEQ ID NO: 3) | ASMWERVKSIIKSSLAAASNI (SEQ ID NO: 500) |
| RQIKIWFQNRRMKW (SEQ ID NO: 104) | DPKGDPKGVTVTVTVTVTGKGDPKPD (SEQ ID NO: 501) |
| RQIKIWFQNRRMK (SEQ ID NO: 105) | CSIPPEVKFNPFVYLI (SEQ ID NO: 502) |
| RQIKIWFQNRRM (SEQ ID NO: 106) | csippevkfnpfvyli |
| RQIKIWFQNRR (SEQ ID NO: 107) | PFVYLI (SEQ ID NO: 503) |
| RQIKIWFQNR (SEQ ID NO: 108) | NKPILVFY (SEQ ID NO: 504) |
| RQIKIWFQN (SEQ ID NO: 109) | YKQCHKKGGKKGSG (SEQ ID NO: 505) |
| RQIKIWFQ (SEQ ID NO: 110) | YKQCHKKGGXKKGSG (SEQ ID NO: 506) |
| RQIKIW (SEQ ID NO: 111) | GSGKKGGKKHCQKY (SEQ ID NO: 507) |
| QIKIWFQNRRMKWKK (SEQ ID NO: 112) | GSGKKGGKKICQKY (SEQ ID NO: 508) |
| IKIWFQNRRMKWKK (SEQ ID NO: 113) | YTAIAWVKAFIRKLRK (SEQ ID NO: 509) |
| KIWFQNRRMKWKK (SEQ ID NO: 114) | IAWVKAFIRKLRKGPLG (SEQ ID NO: 510) |
| IWFQNRRMKWKK (SEQ ID NO: 115) | LIRLWSHLIHIWFQNRRLKWKKK (SEQ ID NO: 511) |
| WFQNRRMKWKK (SEQ ID NO: 116) | KKKKKKGGFLGFWRGENGRKTRSAYERMCILKGK (SEQ |
| | ID NO: 512) |
| FQNRRMKWKK (SEQ ID NO: 117) | RLSGMNEVLSFRWL (SEQ ID NO: 513) |
| QNRRMKWKK (SEQ ID NO: 118) | GPFHFYQFLFPPV (SEQ ID NO: 514) |
| NRRMKWKK (SEQ ID NO: 119) | GSPWGLQHHPPRT (SEQ ID NO: 515) |
| RRMKWKK (SEQ ID NO: 120) | AAVALLPAVLLALLAP (SEQ ID NO: 516) |
| RMKWKK (SEQ ID NO: 121) | AAVALLPAVLLALLAPEILLPNNYNAYESYKYPGMFIALSK |
| | (SEQ ID NO: 517) |
| AQIKIWFQNRRMKWKK (SEQ ID NO: 122) | AAVALLPAVLLALLAPVQRKRQKLMP (SEQ ID NO: 518) |
| RAIKIWFQNRRMKWKK (SEQ ID NO: 123) | WEAKLAKALAKALAKHLAKALAKALKACEA (SEQ ID |
| | NO: 519) |
| RQAKIWFQNRRMKWKK (SEQ ID NO: 124) | MGLGLHLLVLAAALQGAWSQPKKKRKV (SEQ ID NO: 520) |
| RQIAIWFQNRRMKWKK (SEQ ID NO: 125) | MGLGLHLLVLAAALQGAKKKRKV (SEQ ID NO: 521) |
| RQIKAWFQNRRMKWKK (SEQ ID NO: 126) | WEAALAEALAEALAEHLAEALAEALEALAA (SEQ ID NO: |
| | 522) |
| RQIKIAFQNRRMKWKK (SEQ ID NO: 127) | GLFEALLELLESLWELLLEA (SEQ ID NO: 523) |
| RQIKIWAQNRRMKWKK (SEQ ID NO: 128) | GLFKALLKLLKSLWKLLLKA (SEQ ID NO: 524) |
| RQIKIWFANRRMKWKK (SEQ ID NO: 129) | GLFRALLRLLRSLWRLLLRA (SEQ ID NO: 525) |
| RQIKIWFQARRMKWKK (SEQ ID NO: 130) | CGAYDLRRRERQSRLRRRERQSR (SEQ ID NO: 526) |
| RQIKIWFQNARMKWKK (SEQ ID NO: 131) | RKKRRRESRKKRRRESC (SEQ ID NO: 527) |
| RQIKIWFQNRAMKWKK (SEQ ID NO: 132) | CVKRGLKLRHVRPRVTRDV (SEQ ID NO: 528) |
| RQIKIWFQNRRAKWKK (SEQ ID NO: 133) | CRQIKIWFQNRRMKWKK (SEQ ID NO: 529) |
| RQIKIWFQNRRMAWKK (SEQ ID NO: 134) | YARAAARQARA (SEQ ID NO: 530) |
| RQIKIWFQNRRMKAKK (SEQ ID NO: 135) | PPKKSAQCLRYKKPE (SEQ ID NO: 531) |
| RQIKIWFQNRRMKWAK (SEQ ID NO: 136) | DPVDTPNPTRRKPGK (SEQ ID NO: 532) |
| RQIKIWFQNRRMKWKA (SEQ ID NO: 137) | KRVSRNKSEKKRR (SEQ ID NO: 533) |
| CRQIKIWFPNRRMKWKKC (SEQ ID NO: | GRRHHCRSKAKRSRHH (SEQ ID NO: 534) |
| 138) | |
| RQIKIWFPNRRMKWKK (SEQ ID NO: 139) | SARHHCRSKAKRSRHH (SEQ ID NO: 535) |
| RQIKIWFQNRRMKWKK (SEQ ID NO: 140) | SRAHHCRSKAKRSRHH (SEQ ID NO: 536) |
| RQIKIFFQNRRMKFKK (SEQ ID NO: 141) | SRRAHCRSKAKRSRHH (SEQ ID NO: 537) |
| RQIRIWFQNRRMRWRR (SEQ ID NO: 142) | SRRHACRSKAKRSRHH (SEQ ID NO: 538) |
| RRRRRRRW (SEQ ID NO: 143) | SRRHHARSKAKRSRHH (SEQ ID NO: 539) |
| GRKKRRQRRRPWQ (SEQ ID NO: 144) | SRRHHCRAKAKRSRHH (SEQ ID NO: 540) |
| GRKKRRQRRRPWQ (SEQ ID NO: 145) | SRRHHCRSAAKRSRHH (SEQ ID NO: 541) |
| RQIRIWFQNRRMRWRR (SEQ ID NO: 146) | SRRHHCRSKAARSRHH (SEQ ID NO: 542) |
| RRWRRWWRRWWRRWRR (SEQ ID NO: 147) | SRRHHCRSKAKASRHH (SEQ ID NO: 543) |
| RQIKIWFQNMRRKWKK (SEQ ID NO: 148) | SRRHHCRSKAKRARHH (SEQ ID NO: 544) |
| KMDCRWRWKCCKK (SEQ ID NO: 149) | SRRHHCRSKAKRSAHH (SEQ ID NO: 545) |
| MDCRWRWKCCKK (SEQ ID NO: 150) | RRHHCRSKAKRSR (SEQ ID NO: 546) |
| DCRWRWKCCKK (SEQ ID NO: 151) | GRKGKHKRKKLP (SEQ ID NO: 547) |
| CRWRWKCCKK (SEQ ID NO: 152) | GKKKKKKKKK (SEQ ID NO: 548) |
| RWRWKCCKK (SEQ ID NO: 153) | GKRVAKRKLIEQNRERRR (SEQ ID NO: 549) |
| KMDCRWRWKCKK (SEQ ID NO: 154) | GRKLKKKKNEKEDKRPRT (SEQ ID NO: 550) |
| KMDCRWRWKKK (SEQ ID NO: 155) | GKKTNLFSALIKKKKTA (SEQ ID NO: 551) |
| KMDRWRWKKK (SEQ ID NO: 156) | GRRERNKMAAAKCRNRRR (SEQ ID NO: 552) |
| KDCRWRWKCCKK (SEQ ID NO: 157) | GKRARNTEAARRSRARKL (SEQ ID NO: 553) |
| KCRWRWKCCKK (SEQ ID NO: 158) | GRRRRATAKYRTAH (SEQ ID NO: 554) |
| KRWRWKCCKK (SEQ ID NO: 159) | GKRRRRATAKYRSAH (SEQ ID NO: 555) |
| MDCRWRWKXCKK (SEQ ID NO: 160) | GRRRRKRLSHRT (SEQ ID NO: 556) |
| DCRWRWKXCKK (SEQ ID NO: 161) | GRRRRRERNK (SEQ ID NO: 557) |
| DCRWRWKCXKK (SEQ ID NO: 162) | GKHRHERGHHRDRRER (SEQ ID NO: 558) |
| CRWRWKXCKK (SEQ ID NO: 163) | GKKKRKLSNRESAKRSR (SEQ ID NO: 559) |
| CRWRWKCXKK (SEQ ID NO: 164) | MITYRDLISH (SEQ ID NO: 560) |
| RWRWKXCKK (SEQ ID NO: 165) | MITYRDLIS (SEQ ID NO: 561) |
| MDCRWRWKXXKK (SEQ ID NO: 166) | MITYRDLI (SEQ ID NO: 562) |
| DCRWRWKXXKK (SEQ ID NO: 167) | IIYRDLISH (SEQ ID NO: 563) |
| CRWRWKXXKK (SEQ ID NO: 168) | MITYRDL (SEQ ID NO: 564) |
| RWRWKXXKK (SEQ ID NO: 169) | MITYRD (SEQ ID NO: 565) |
| CRWRWKCSKK (SEQ ID NO: 170) | IYRDLISH (SEQ ID NO: 566) |
| SRWRWKCCKK (SEQ ID NO: 171) | AITYRDLIS (SEQ ID NO: 567) |
| SRWRWKCSKK (SEQ ID NO: 172) | MAIYRDLIS (SEQ ID NO: 568) |
| SRWRWKSCKK (SEQ ID NO: 173) | MIAYRDLIS (SEQ ID NO: 569) |
| CRWRWKSSKK (SEQ ID NO: 174) | MIIARDLIS (SEQ ID NO: 570) |
| SRWRWKSSKK (SEQ ID NO: 175) | MITYADLIS (SEQ ID NO: 571) |
| CRFRWKCCKK (SEQ ID NO: 176) | MITYRALIS (SEQ ID NO: 572) |
| CRWRFKCCKK (SEQ ID NO: 177) | MITYRDAIS (SEQ ID NO: 573) |
| CRFRFKCCKK (SEQ ID NO: 178) | MITYRDLAS (SEQ ID NO: 574) |
| crwrwkcckk | MITYRDLIA (SEQ ID NO: 575) |
| KCCKWRWRCK (SEQ ID NO: 179) | MITYRDLISKK (SEQ ID NO: 576) |
| kcckwrwrck | MITYRDKKSH (SEQ ID NO: 577) |
| CrWRWKCCKK | MIIFRDLISH (SEQ ID NO: 578) |
| CRwRWKCCKK | MIISRDLISH (SEQ ID NO: 579) |
| CRWrWKCCKK | QIISRDLISH (SEQ ID NO: 580) |
| CRWRwKCCKK | CIISRDLISH (SEQ ID NO: 581) |
| CrwrwKCCKK | MITYRALISHKK (SEQ ID NO: 582) |
| CRWRWKCGCKK (SEQ ID NO: 180) | MITYRIAASHKK (SEQ ID NO: 583) |
| KCGCRWRWKCGCKK (SEQ ID NO: 181) | MIIRRDLISE (SEQ ID NO: 584) |
| CRWRWKCG (SEQ ID NO: 182) | MITYRAEISH (SEQ ID NO: 585) |
| KMDXRWRWKCCKK (SEQ ID NO: 183) | MIIYARRAEE (SEQ ID NO: 586) |
| KMDXRWRWKXCKK (SEQ ID NO: 184) | MIIFRIAASHKK (SEQ ID NO: 587) |
| KMDXRWRWKXXKK (SEQ ID NO: 185) | MIIFRALISHKK (SEQ ID NO: 588) |
| KMDXRWRWKCXKK (SEQ ID NO: 186) | MIIFRAAASHKK (SEQ ID NO: 589) |
| MDCRWRWKCXKK (SEQ ID NO: 187) | FIIFRIAASHKK (SEQ ID NO: 590) |
| KMDCRWRWKCSKK (SEQ ID NO: 188) | LIIFRIAASHKK (SEQ ID NO: 591) |
| KMDCRWRWKSCKK (SEQ ID NO: 189) | WIIFRIAASHKK (SEQ ID NO: 592) |
| KMDSRWRWKCCKK (SEQ ID NO: 190) | WIIFRAAASHKK (SEQ ID NO: 593) |
| KMDCRWRWKSSKK (SEQ ID NO: 191) | WIIFRALISHKK (SEQ ID NO: 594) |
| KMDSRWRWKSSKK (SEQ ID NO: 192) | MIIFRIAAYHKK (SEQ ID NO: 595) |
| KMDSRWRWKSCKK (SEQ ID NO: 193) | WIIFRIAAYHKK (SEQ ID NO: 596) |
| KMDSRWRWKCSKK (SEQ ID NO: 194) | MIIFRIAATHKK (SEQ ID NO: 597) |
| KMDCRWRPKCCKK (SEQ ID NO: 195) | WIIFRIAATHKK (SEQ ID NO: 598) |
| KMDCRPRPKCCKK (SEQ ID NO: 196) | MIIFKIAASHKK (SEQ ID NO: 599) |
| KMDXRPRPKCCKK (SEQ ID NO: 197) | WIIFKIAASHKK (SEQ ID NO: 600) |
| KMDXRPRPKXCKK (SEQ ID NO: 198) | MIIFAIAASHKK (SEQ ID NO: 601) |
| KMDXRPRPKCXKK (SEQ ID NO: 199) | LIIFRILISHKK (SEQ ID NO: 602) |
| KMDCRPRPKXCKK (SEQ ID NO: 200) | MIIFRILISHKK (SEQ ID NO: 603) |
| KMDCRPRPKCXKK (SEQ ID NO: 201) | LIIFRILISHRR (SEQ ID NO: 604) |
| RQIKIWFQNRRMKWKK (SEQ ID NO: 202) | LIIFRILISHHH (SEQ ID NO: 605) |
| rkkrrqrrr | LIIFRILISHK (SEQ ID NO: 606) |
| rrrqrrkkr | LIIFRILISHR (SEQ ID NO: 607) |
| rrrrrrrr | LIIFRILISH (SEQ ID NO: 608) |
| RKKRRRESRKKRRRES (SEQ ID NO: 203) | LIIFAIAASHKK (SEQ ID NO: 609) |
| GRPRESGKKRKRKRLKP (SEQ ID NO: 204) | LIIFAILISHKK (SEQ ID NO: 610) |
| GKRKKKGKLGKKRDP (SEQ ID NO: 205) | RILQQLLFIHFRIGCRHSRI (SEQ ID NO: 611) |
| GKRKKKGKLGKKRPRSR (SEQ ID NO: 206) | RILQQLLFIHFRIGCRH (SEQ ID NO: 612) |
| RKKRRRESRRARRSPRHL (SEQ ID NO: | RILQQLLFIHFRIGC (SEQ ID NO: 613) |
| 207) | |
| SRRARRSPRESGKKRKRKR (SEQ ID | RIFIHFRIGC (SEQ ID NO: 614) |
| NO: 208) | |
| VKRGLKLRHVRPRVTRMDV (SEQ ID | RIFIRIGC (SEQ ID NO: 615) |
| NO: 209) | |
| SRRARRSPRHLGSG (SEQ ID NO: 210) | RILQQLLFIHF (SEQ ID NO: 616) |
| LRRERQSRLRRERQSR (SEQ ID NO: | RIFIGC (SEQ ID NO: 617) |
| 211) | |
| GAYDLRRRERQSRLRRRERQSR (SEQ | FIRIGC (SEQ ID NO: 618) |
| ID NO: 212) | |
| GRKKRRQRRRPPQ (SEQ ID NO: 213) | DTWAGVEAIIRILQQLLFIHFR (SEQ ID NO: 619) |
| VPMLK (SEQ ID NO: 214) | IGCRH (SEQ ID NO: 620) |
| VPTLK (SEQ ID NO: 215) | RQIKIWFQNRRMKWKK (SEQ ID NO: 621) |
| VPALR (SEQ ID NO: 216) | GYGRKKRRGRRRTHRLPRRRRRR (SEQ ID NO: 622) |
| VSALK (SEQ ID NO: 217) | KRIIQRILSRNS (SEQ ID NO: 623) |
| PMLKE (SEQ ID NO: 218) | KRIHPRLTRSIR (SEQ ID NO: 624) |
| VPALK (SEQ ID NO: 219) | PPRLRKRRQLNM (SEQ ID NO: 625) |
| VSLKK (SEQ ID NO: 220) | PIRRRKKLRRLK (SEQ ID NO: 626) |
| VSGKK (SEQ ID NO: 221) | RRQRRTSKLMKR (SEQ ID NO: 627) |
| KLPVM (SEQ ID NO: 222) | MHKRPTTPSRKM (SEQ ID NO: 628) |
| IPMIK (SEQ ID NO: 223) | RQRSRRRPLNIR (SEQ ID NO: 629) |
| KLGVM (SEQ ID NO: 224) | RIRMIQNLIKKT (SEQ ID NO: 630) |
| KLPVT (SEQ ID NO: 225) | SRRKRQRSNMRI (SEQ ID NO: 631) |
| VPMIK (SEQ ID NO: 226) | QRIRKSKISRTL (SEQ ID NO: 632) |
| IPALK (SEQ ID NO: 627) | PSKRLLHNNLRR (SEQ ID NO: 633) |
| IPMLK (SEQ ID NO: 228) | HRHIRRQSLIML (SEQ ID NO: 634) |
| VPTLQ (SEQ ID NO: 229) | PQNRLQIRRHSK (SEQ ID NO: 635) |
| QLPVM (SEQ ID NO: 230) | PPHNRIQRRLNM (SEQ ID NO: 636) |
| ELPVM (SEQ ID NO: 231) | SMLKRNHSTSNR (SEQ ID NO: 637) |
| VPTLE (SEQ ID NO: 232) | GSRHPSLIIPRQ (SEQ ID NO: 638) |
| vptlk | SPMQKTMNLPPM (SEQ ID NO: 639) |
| RRRRRRRR (SEQ ID NO: 2) | NKRILIRIMTRP (SEQ ID NO: 640) |
| AYRIKPTFRRLKWKYKGKFW (SEQ ID | HGWZIHGLLHRA (SEQ ID NO: 641) |
| NO: 233) | |
| HARIKPTFRRLKWKYKGKFW (SEQ ID | AVPAKKRZKSV (SEQ ID NO: 642) |
| NO: 234) | |
| HYRIKPTARRLKWKYKGKFW (SEQ ID | PNTRVRPDVSF (SEQ ID NO: 643) |
| NO: 235) | |
| HYRIKPTFRRLAWKYKGKFW (SEQ ID | LTRNYEAWVPTP (SEQ ID NO: 644) |
| NO: 236) | |
| HYRIKPTFRRLKWKYKGKFA (SEQ ID | SAETVESCLAKSH (SEQ ID NO: 645) |
| NO: 237) | |
| VNADIKATTVFGGKYVSLTTP (SEQ ID | YSHIATLPFTPT (SEQ ID NO: 646) |
| NO: 238) | |
| GKYVSLTTPKNPTKRRITPKDV (SEQ ID | SYIQRTPSTTLP (SEQ ID NO: 647) |
| NO: 239) | |
| TKRRITPKDVIDVRSVTTEINT (SEQ ID | AVPAENALNNPF (SEQ ID NO: 648) |
| NO: 240) | |
| RSVTTEINTLFQTLTSIAEKVDP (SEQ ID | SFHQFARATLAS (SEQ ID NO: 649) |
| NO: 241) | |
| AEKVDPVKLNLTLSAAAEALTGLGDK | QSPTDFTFPNPL (SEQ ID NO: 650) |
| (SEQ ID NO: 242) | |
| GLGDKFGESIVNANTVLDDLNSRMPQS | HFAAWGGWSLVH (SEQ ID NO: 651) |
| RHDIQQL (SEQ ID NO: 243) | |
| GDVYADAAPDLFDFLDSSVTTARTINA | HIQLSPFSQSWR (SEQ ID NO: 652) |
| (SEQ ID NO: 244) | |
| ARTINAQQAELDSALLAAAGFGNTTAD | LTMPSDLQPVLW (SEQ ID NO: 653) |
| VFDRG (SEQ ID NO: 245) | |
| ADVFDRGGPYLQRGVADLVPTATLLDT | FQPYDHPAEVSY (SEQ ID NO: 654) |
| YSP (SEQ ID NO: 246) | |
| LDTYSPELFCTIRNFYDADRPDRGAAA | FDPFFWKYSPRD (SEQ ID NO: 655) |
| (SEQ ID NO: 247) | |
| TKRRITPKDVIDVRSVTTEINT (SEQ ID | FAPWDTASFMLG (SEQ ID NO: 656) |
| NO: 248) | |
| TKRRITPDDVIDVRSVTTEINT (SEQ ID | FTYKNFFWLPEL (SEQ ID NO: 657) |
| NO: 249) | |
| TKRRITPKKVIDVRSVTTEINT (SEQ ID | SATGAPWKMWVR (SEQ ID NO: 658) |
| NO: 250) | |
| TKRRITPKDVIDVRSVTTKINT (SEQ ID | SLGWMLPFSPPF (SEQ ID NO: 659) |
| NO: 251) | |
| TKRRITPKDVIDV (SEQ ID NO: 252) | SHAFTWPTYLQL (SEQ ID NO: 660) |
| TKRRITPKDVIDVESVTTEINT (SEQ ID | SHNWLPLWPLRP (SEQ ID NO: 661) |
| NO: 253) | |
| TARRITPKDVIDVRSVTTEINT (SEQ ID | SWLPYPWHVPSS (SEQ ID NO: 662) |
| NO: 254) | |
| TKAARITPKDVIDVRSVTTEINT (SEQ ID | SWWTPWHVHSES (SEQ ID NO: 663) |
| NO: 255) | |
| HHHHHHTKRRITPKDVIDVRSVTTEINT | SWAQHLSLPPVL (SEQ ID NO: 664) |
| (SEQ ID NO: 256) | |
| KLWMRWYSPTTRRYG (SEQ ID NO: 257) | SSSIFPPWLSFF (SEQ ID NO: 665) |
| DSLKSYWYLQKFSWR (SEQ ID NO: 258) | LNVPPSWFLSQR (SEQ ID NO: 666) |
| RTLVNEYKNTLKFSK (SEQ ID NO: 259) | LDITPFLSLTLP (SEQ ID NO: 667) |
| IPSRWKDQFWKRWHY (SEQ ID NO: 260) | LPHPVLHMGPLR (SEQ ID NO: 668) |
| GYGNCRHFKQKPRRD (SEQ ID NO: 261) | VSKQPYYMWNGN (SEQ ID NO: 669) |
| KNAWKHSSCHHRHQI (SEQ ID NO: 262) | NYTTYKSHFQDR (SEQ ID NO: 670) |
| RVREWWYTITLKQES (SEQ ID NO: 263) | AIPNNQLGFPFK (SEQ ID NO: 671) |
| QQHLLIAINGYPRYN (SEQ ID NO: 264) | NIENSTLATPLS (SEQ ID NO: 672) |
| WKCRRQCFRVLHHWN (SEQ ID NO: 265) | YPYDANHTRSPT (SEQ ID NO: 673) |
| RLWMRWYSPTTRRYG (SEQ ID NO: 266) | DPATNPGPHFPR (SEQ ID NO: 674) |
| KLWMRWYSATTRRYG (SEQ ID NO: 267) | TLPSPLALLTVH (SEQ ID NO: 675) |
| KLWMRWYSPWTRRYG (SEQ ID NO: 268) | HPGSPFPPEHRP (SEQ ID NO: 676) |
| RLWMRWYSPWTRRYG (SEQ ID NO: 269) | TSHTDAPPARSP (SEQ ID NO: 677) |
| RLWMRWYSPWTRRWG (SEQ ID NO: 270) | MTPSSLSTLPWP (SEQ ID NO: 678) |
| ALWMRWYSPTTRRYG (SEQ ID NO: 271) | VLGQSGYLMPMR (SEQ ID NO: 679) |
| RAWMRWYSPTTRRYG (SEQ ID NO: 272) | QPIIITSPYLPS (SEQ ID NO: 680) |
| RLAMRWYSPTTRRYG (SEQ ID NO: 273) | TPKTMTQTYDFS (SEQ ID NO: 681) |
| RLWARWYSPTTRRYG (SEQ ID NO: 274) | NSGTMQSASRAT (SEQ ID NO: 682) |
| RLWMAWYSPTTRRYG (SEQ ID NO: 275) | QAASRVENYMHR (SEQ ID NO: 683) |
| RLWMRAYSPTTRRYG (SEQ ID NO: 276) | HQHKPPPLTNNW (SEQ ID NO: 684) |
| RLWMRWASPTTRRYG (SEQ ID NO: 277) | SNPWDSLLSVST (SEQ ID NO: 685) |
| RLWMRWYAPTTRRYG (SEQ ID NO: 278) | KTIEAHPPYYAS (SEQ ID NO: 686) |
| RLWMRWYSPATRRYG (SEQ ID NO: 279) | EPDNWSLDFPRR (SEQ ID NO: 687) |
| RLWMRWYSPTARRYG (SEQ ID NO: 280) | HQHKPPPLTNNW (SEQ ID NO: 688) |
| RLWMRWYSPTTARYG (SEQ ID NO: 281) | GLWRALWRLLRSLWRLLWKA (SEQ ID NO: 689) |
| RLWMRWYSPTTRAYG (SEQ ID NO: 282) | GLWRALWRALWRSLWKLKRKV (SEQ ID NO: 690) |
| RLWMRWYSPTTRRAG (SEQ ID NO: 283) | GLWRALWRALRSLWKLKRKV (SEQ ID NO: 691) |
| RLWMRWYSPTTRRYA (SEQ ID NO: 284) | GLWRALWRGLRSLWKLKRKV (SEQ ID NO: 692) |
| RLLMRLYSPTTRRYG (SEQ ID NO: 285) | GLWRALWRGLRSLWKKKRKV (SEQ ID NO: 693) |
| RLFMRFYSPTTRRYG (SEQ ID NO: 286) | GLWRALWRALWRSLWKLKWKV (SEQ ID NO: 694) |
| RLIMRIYSPTTRRYG (SEQ ID NO: 287) | GLWRALWRALWRSLWKSKRKV (SEQ ID NO: 695) |
| RLVMRVYSPTTRRYG (SEQ ID NO: 288) | GLWRALWRALWRSLWKKKRKV (SEQ ID NO: 696) |
| RLYMRYYSPTTRRYG (SEQ ID NO: 289) | GLWRALWRALWRSLWKLKRKV (SEQ ID NO: 697) |
| YGRKKKRRQRRR (SEQ ID NO: 290) | GLWRALWRLLRSLWRLLWSQPKKKRKV (SEQ ID NO: 698) |
| LLIILRRRIRKQAHAHSK (SEQ ID NO: | YARAARRAARR (SEQ ID NO: 699) |
| 291) | |
| ALIILRRRIRKQAHAHSK (SEQ ID NO: | PARAARRAARR (SEQ ID NO: 700) |
| 292) | |
| LAIILRRRIRKQAHAHSK (SEQ ID NO: | YPRAARRAARR (SEQ ID NO: 701) |
| 293) | |
| LLAILRRRIRKQAHAHSK (SEQ ID NO: | YRRAARRAARA (SEQ ID NO: 702) |
| 294) | |
| LLIALRRRIRKQAHAHSK (SEQ ID NO: | YGRAARRAARR (SEQ ID NO: 703) |
| 295) | |
| LLIIARRRIRKQAHAHSK (SEQ ID NO: | YAREARRAARR (SEQ ID NO: 704) |
| 296) | |
| LLIILARRIRKQAHAHSK (SEQ ID NO: | YEREARRAARR (SEQ ID NO: 705) |
| 297) | |
| LLIILRARIRKQAHAHSK (SEQ ID NO: | YKRAARRAARR (SEQ ID NO: 706) |
| 298) | |
| LLIILRRAIRKQAHAHSK (SEQ ID NO: | YARKARRAARR (SEQ ID NO: 707) |
| 299) | |
| LLIILRRRARKQAHAHSK (SEQ ID NO: | YKRKARRAARR (SEQ ID NO: 708) |
| 300) | |
| LLIILRRRIARKQAHAHSK (SEQ ID NO: | YGRRARRAARR (SEQ ID NO: 709) |
| 301) | |
| LLIILRRRIRAQAHAHSK (SEQ ID NO: | YGRRARRRARR (SEQ ID NO: 710) |
| 302) | |
| LLIILRRRIRKAAHAHSK (SEQ ID NO: | YGRRARRRRRR (SEQ ID NO: 711) |
| 303) | |
| LLIILRRRIRKQaHAHSK | YGRRRRRRRRR (SEQ ID NO: 712) |
| LLIILRRRIRKQAAAHSK (SEQ ID NO: | YRRRRRRRRRR (SEQ ID NO: 713) |
| 304) | |
| LLIILRRRIRKQAHaHSK | GKINLKALAALAKKIL (SEQ ID NO: 714) |
| LLIILRRRIRKQAHAASK (SEQ ID NO: | RVIRVWFQNKRCKDKK (SEQ ID NO: 715) |
| 305) | |
| LLIILRRRIRKQAHAHAK (SEQ ID NO: | GRKKRRQRRRPPQGRKKRRQRRRPPQGRKKRRQRRRPPQ |
| 306) | (SEQ ID NO: 716) |
| LLIILRRRIRKQAHAHSA (SEQ ID NO: | GEQIAQLIAGYIDIILKKKKSK (SEQ ID NO: 717) |
| 307) | |
| KSHAHAQKRIRRRLIILL (SEQ ID NO: | GRKKRRQRRRPPQC (SEQ ID NO: 718) |
| 308) | |
| lliilrrrirkqahahsk | AAVALLPAVLLALLAPRKKRRQRRRPPQ (SEQ ID NO: 719) |
| RRIRPRP (SEQ ID NO: 309) | AAVALLPAVLLALLAPRKKRRQRRRPPQC (SEQ ID NO: |
| | 720) |
| RRIRPRPPRLPRPRP (SEQ ID NO: 310) | AAVALLPAVLLALLAPRKKRRQRRRPPQ (SEQ ID NO: 721) |
| RRIRPRPPRLPRPRPRPLPFPRPG (SEQ | RKKRRQRRRPPQCAAVALLPAVLLALLAP (SEQ ID NO: |
| ID NO: 311) | 722) |
| RRIRPRPPRLPRPRPRP (SEQ ID NO: 312) | RRRQRRKRGGDIMGEWGNEIFGAIAGFLG (SEQ ID NO: |
| | 723) |
| PRPPRLPRPRPRPLPFPRPG (SEQ ID NO: | RRRQRRKRGGDIMGEWGNEIFGAIAGFLG (SEQ ID NO: |
| 313) | 723) |
| PPRLPRPRPRPLPFPRPG (SEQ ID NO: | YGRKKRRQRRRGCYGRKKRRQRRRG (SEQ ID NO: 724) |
| 314) | |
| RLPRPRPRPLPFPRPG (SEQ ID NO: 315) | GRKKRRQRRRPPQ (SEQ ID NO: 725) |
| PRPRPRPLPFPRPG (SEQ ID NO: 316) | AAVALLPAVLLALLAPRRRRRR (SEQ ID NO: 726) |
| PRPRPLPFPRPG (SEQ ID NO: 317) | RLWRALPRVLRRLLRP (SEQ ID NO: 727) |
| PRPLPFPRPG (SEQ ID NO: 318) | AAVALLPAVLLALLAPSGASGLDKRDYV (SEQ ID NO: 728) |
| RKKRRQRRR (SEQ ID NO: 319) | LLETLLKPFQCRICMRNFSTRQARRNHRRRHRR (SEQ ID |
| | NO: 729) |
| RQGAARVTSWLGRQLRIAGKRLEGRSK | AAVACRICMRNFSTRQARRNHRRRHRR (SEQ ID NO: 730) |
| (SEQ ID NO: 320) | |
| RVTSWLGRQLRIAGKRLEGRSK (SEQ | RQIKIWFQNRRMKWKK (SEQ ID NO: 731) |
| ID NO: 321) | |
| GROLRIAGKRLEGRSK (SEQ ID NO: | RQIKIWFQNRRMKWKK (SEQ ID NO: 731) |
| 322) | |
| RRVTSWLGRQLRIAGKRLEGRSK (SEQ | RQIKIWFQNRRMKWKKDIMGEWGNEIFGAIAGFLG (SEQ |
| ID NO: 323) | ID NO: 732) |
| RVRSWLGRQLRIAGKRLEGRSK (SEQ | SGRGKQGGKARAKAKTRSSRAGLQFPVGRVHRLLRKG |
| ID NO: 324) | (SEQ ID NO: 733) |
| GRQLRIAGKRLRGRSK (SEQ ID NO: 325) | SGRGKQGGKARAKAKTRSSRAGLQFPVGRVHRLLRKGC |
| | (SEQ ID NO: 734) |
| GRQLRIAGRRLRGRSR (SEQ ID NO: 326) | KKDGKKRKRSRKESYSVYVYKVLKQ (SEQ ID NO: 735) |
| GRQLRRAGRRLRGRSR (SEQ ID NO: 327) | KGSKKAVTKAQKKDGKKRKRSRKESYSVYVYKVLKQ |
| | (SEQ ID NO: 736) |
| GRQLRIAGRRLRRRSR (SEQ ID NO: 328) | GWTLNSAGYLLGKINLKALAALAKKIL (SEQ ID NO: 737) |
| GRQLRRA GRRLRRRSR (SEQ ID NO: | KLALKLALKALKAALKLA (SEQ ID NO: 738) |
| 329) | |
| RQLRIAGRRLRGRSR (SEQ ID NO: 330) | KETWWETWWTEWSQPKKKRKV (SEQ ID NO: 739) |
| rsrgrlrrgairlqrg | KETWWETWWTEWSQPGRKKRRQRRRPPQ (SEQ ID NO: |
| | 740) |
| KLIKGRTPIKFGKADCDRPPKHSQNGM | RVIRWFQNKRCKDKK (SEQ ID NO: 741) |
| GK (SEQ ID NO: 331) | |
| KLIKGRTPIKFGKADCDRPPKHSQNGM | LGLLLRHLRHHSNLLANI (SEQ ID NO: 742) |
| (SEQ ID NO: 332) | |
| KLIKGRTPIKFGKADCDRPPKHSQNGK | KLWSAWPSLWSSLWKP (SEQ ID NO: 743) |
| (SEQ ID NO: 333) | |
| KGRTPIKFGKADCDRPPKHSQNGMGK | GLGSLLKKAGKKLKQPKSKRKV (SEQ ID NO: 744) |
| (SEQ ID NO: 334) | |
| KLIKGRTPIKFGKADCDRPPKHSGK | FKQqQqQqQqQq |
| (SEQ ID NO: 335) | |
| KLIKGRTPIKFGKARCRRPPKHSGK | YRFK (SEQ ID NO: 745) |
| (SEQ ID NO: 336) | |
| KLIKGRTPIKFGK (SEQ ID NO: 337) | YRFKYRFKYRLFK (SEQ ID NO: 746) |
| KRIPNKKPGKKTTTKPTKKPTIKTTKKD | WRFKKSKRKV (SEQ ID NO: 747) |
| LKPQTTKPK (SEQ ID NO: 338) | |
| KRIPNKKPGKKTTTKPTKKPTIKTTKKD | WRFKAAVALLPAVLLALLAP (SEQ ID NO: 748) |
| LK (SEQ ID NO: 339) | |
| KRIPNKKPGKKTTTKPTKKPTIKTTKK | WRFKWRFK (SEQ ID NO: 749) |
| (SEQ ID NO: 340) | |
| KRIPNKKPGKKTTTKPTKKPTIK (SEQ | WRFKWRFKWRFK (SEQ ID NO: 750) |
| ID NO: 341) | |
| KRIPNKKPGKKTTTKPTKK (SEQ ID NO: | KGSKKAVTKAQKKDGKKRKRSRKESYSVYVYKVLKQ |
| 342) | (SEQ ID NO: 751) |
| KRIPNKKPGKKT (SEQ ID NO: 343) | RGSRRAVTRAQRRDGRRRRRSRRESYSVYVYRVLRQ (SEQ |
| | ID NO: 752) |
| KRIPNKKPGKK (SEQ ID NO: 344) | RVIRWFQNKRSKDKK (SEQ ID NO: 753) |
| KRIPNKKPKK (SEQ ID NO: 345) | GWTLNSAGYLLGKINLKALAALAKKIL (SEQ ID NO: 754) |
| RRIPNRRPRR (SEQ ID NO: 346) | AAVALLPAVLLALLAPRKKRRQRRRPPQ (SEQ ID NO: 755) |
| KKPGKKTTTKPTKKPTIKTTKK (SEQ ID | CWKKK (SEQ ID NO: 756) |
| NO: 347) | |
| KKPGKKTTTKPTKK (SEQ ID NO: 348) | CWKKKKKKKK (SEQ ID NO: 757) |
| KKPTIKTTKK (SEQ ID NO: 349) | CWKKKKKKKKKKKKK (SEQ ID NO: 758) |
| KKTTTKPTKK (SEQ ID NO: 350) | CWKKKKKKKKKKKKKKKKKK (SEQ ID NO: 759) |
| KSICKTIPSNKPKKK (SEQ ID NO: 351) | KKKKKKKKKKKKKKKKKKK (SEQ ID NO: 760) |
| KTIPSNKPKKK (SEQ ID NO: 352) | kkwkmrrGaGrrrrrrrrr |
| KPRSKNPPKKPK (SEQ ID NO: 353) | APWHLSSQYSRT (SEQ ID NO: 761) |
| DRDDRDDRDDRDDRDDR (SEQ ID NO: | AAVALLPAVLLALLAKKNNLKDCGLF (SEQ ID NO: 762) |
| 354) | |
| ERERERERERERER (SEQ ID NO: 355) | AAVALLPAVLLALLAKKNNLKECGLY (SEQ ID NO: 763) |
| WRWRWRWRWRWRWR (SEQ ID NO: 356) | AAVALLPAVLLALLAVTDQLGEDFFAVDLEAFLQEFGLLP |
| | EKE (SEQ ID NO: 764) |
| DRDRDRDRDR (SEQ ID NO: 357) | AAVALLPAVLLALLAK (SEQ ID NO: 765) |
| GALFLGFLGAAGSTMGAWSQPKKKRKV | AHALCLTERQIKIWFQNRRMKWKKEN (SEQ ID NO: 766) |
| (SEQ ID NO: 358) | |
| DRRRRGSRPSGAERRRRRAAAA (SEQ | AHALCPPERQIKIWFQNRRMKWKKEN (SEQ ID NO: 767) |
| ID NO: 359) | |
| DRRRRGSRPSGAERRRR (SEQ ID NO: 360) | AYALCLTERQIKIWFANRRMKWKKEN (SEQ ID NO: 768) |
| QTRRRERRAEKQAQW (SEQ ID NO: 361) | GGVCPKILKKCRRDSDCPGACICRGNGYCGSGSD (SEQ ID |
| | NO: 769) |
| RRRERRAEK (SEQ ID NO: 362) | GGVCPKILAACRRDSDCPGACICRGNGYCGSGSD (SEQ ID |
| | NO: 770) |
| NRARRNRRRVR (SEQ ID NO: 363) | GGVCPAILKKCRRDSDCPGACICRGNGYCGSGSD (SEQ ID |
| | NO: 771) |
| RTRRNRRRVR (SEQ ID NO: 364) | GGVCPKILAKCRRDSDCPGACICRGNGYCGSGSD (SEQ ID |
| | NO: 772) |
| RNRSRHRR (SEQ ID NO: 365) | GGVCPKILKACRRDSDCPGACICRGNGYCGSGSD (SEQ ID |
| | NO: 773) |
| KCPSRRPKR (SEQ ID NO: 366) | GLPVCGETCVGGTCNTPGCKCSWPVCTRN (SEQ ID NO: |
| | 774) |
| KRPAAIKKAGQAKKKK (SEQ ID NO: 367) | GLPVCGETCVGGTCNTPGCTCSWPKCTRN (SEQ ID NO: |
| | 775) |
| TRRSKRRSHRKF (SEQ ID NO: 368) | GRCTKSIPPICFPD (SEQ ID NO: 776) |
| RAGLQFPVGR VHRLLRK (SEQ ID NO: | RQIKIWFQNRRMKWKK (SEQ ID NO: 777) |
| 369) | |
| MVRRFLVTLRIRRACGPPRVRV (SEQ | RQIKIWFQNRRMKWKKTYADFIASGRTGRRNAI (SEQ ID |
| ID NO: 370) | NO: 778) |
| FVTRGCPRRLVARLIRVMVPRR (SEQ | GRKKRRQRRRPPQ (SEQ ID NO: 779) |
| ID NO: 371) | |
| VRRFLVTLRIRRA (SEQ ID NO: 372) | GRKKRRQRRRPPQTYADFIASGRTGRRNAI (SEQ ID NO: |
| | 780) |
| RVRILARFLRTRV (SEQ ID NO: 373) | AGYLLGKINLKALAALAKKIL (SEQ ID NO: 781) |
| RVRVFVVHIPRLT (SEQ ID NO: 374) | AGYLLGKINLKALAALAKKILTYADFIASGRTGRRNAI |
| | (SEQ ID NO: 782) |
| VIRVHFRLPVRTV (SEQ ID NO: 375) | RRRRRRRRRRR (SEQ ID NO: 51) |
| MVRRFLVTLRIRRACGPPRVRVFVVHIP | RRRRRRRRRRRTYADFIASGRTGRRNAI (SEQ ID NO: 783) |
| RLTGEWAAP (SEQ ID NO: 376) | |
| FRVPLRIRPCVVAPRLVMVRHTFGRIAR | RRRRRRRRR (SEQ ID NO: 50) |
| WVAGPLETR (SEQ ID NO: 377) | |
| AGYLLGKINLKALAALAKKIL (SEQ ID | RRRRRRRRR (SEQ ID NO: 50) |
| NO: 378) | |
| GTKMIFVGIKKKEERADLIAYLKKA | RRRRRRRRR (SEQ ID NO: 50) |
| (SEQ ID NO: 379) | |
| KKKEERADLIAYLKKA (SEQ ID NO: 380) | rrrrrrrrr |
| KMIFVGIKKKEERA (SEQ ID NO: 381) | rrrrrrrrr |
| KMIFVGIKKK (SEQ ID NO: 382) | rrrrrrrrr |
| EKGKKIFIMK (SEQ ID NO: 383) | rrrrrrrrrk |
| KGKKIFIMK (SEQ ID NO: 384) | rRRRRRRRr |
| RRRRNRTRRNRRRVRGC (SEQ ID NO: 385) | rRrRrRrRr |
| TRRQRTRRARRNRGC (SEQ ID NO: 386) | RQIKIWFQNRRMKWKK (SEQ ID NO: 784) |
| KMTRAQRRAAARRNRWTARGC (SEQ | RQIKIWFQNRRMKWKK (SEQ ID NO: 784) |
| ID NO: 387) | |
| KLTRAQRRAAARKNKRNTRGC (SEQ | rqikiwfqnrrmkwkk |
| ID NO: 388) | |
| NAKTRRHERRRKLAIERGC (SEQ ID | rqikiwfqnrrmkwkk |
| NO: 389) | |
| MDAQTRRRERRAEKQAQWKAANGC | KCFQWQRNMRKVRGPPVSCIKR (SEQ ID NO: 785) |
| (SEQ ID NO: 390) | |
| TAKTRYKARRAELIAERRGC (SEQ ID | KCFQWQRNMRKVRGPPVSCIKR (SEQ ID NO: 785) |
| NO: 391) | |
| SQMTRQARRLYBGC (SEQ ID NO: 392) | kcfqwqrnmrkvrgppvscikr |
| KRRIRRERNKMAAAKSRNRRRELTDTG | kcfqwqrnmrkvrgppvscikr |
| C (SEQ ID NO: 393) | |
| RIKAERKRMRNRIAASKSRKRKLERIAR | KLALKLALKALKAALKLAGC (SEQ ID NO: 786) |
| GC (SEQ ID NO: 394) | |
| KRARNTEAARRSRARKLQRMKQGC | KLULKLULKULKAULKLUGC |
| (SEQ ID NO: 395) | |
| KCFQWQRNMRKVRGPPVSCIKR (SEQ | GGGARKKAAKAARKKAAKAARKKAAKAARKKAAKA |
| ID NO: 396) | (SEQ ID NO: 787) |
| KCFQWQRNMRKVRGPPVSC (SEQ ID | GRKKRRQRRRPPQC (SEQ ID NO: 788) |
| NO: 397) | |
| KCFQWQRNMRKVRGPPVSSIKR (SEQ | TRQARRNRRRRWRERQRGC (SEQ ID NO: 789) |
| ID NO: 398) | |
| KCFQWQRNMRKVR (SEQ ID NO: 399) | RRRRNRTRRNRRRVRGC (SEQ ID NO: 790) |
| FQWQRNMRKVRGPPVS (SEQ ID NO: 400) | KMTRAQRRAAARRNRWTARGC (SEQ ID NO: 791) |
| QWORNMRKVRGPPVSCIKR (SEQ ID | TRRQRTRRARRNRGC (SEQ ID NO: 792) |
| NO: 401) | |
| QWORNMRKVR (SEQ ID NO: 402) | RIKAERKRMRNRIAASKSRKRKLERIARGC (SEQ ID NO: |
| | 793) |
| RRRRRRRRR (SEQ ID NO: 50) | KRRIRRERNKMAAAKSRNRRRELTDTGC (SEQ ID NO: 794) |
| RQIKIWFQNRRMKWKK (SEQ ID NO: 403) | WLRRIKAWLRRIKALNRQLGVAA (SEQ ID NO: 795) |
| KCFMWQEMLNKAGVPKLRCARK (SEQ | crkkrrqrrr |
| ID NO: 404) | |
| KETWWETWWTEWSQPKKKRKV (SEQ | crrrrrrrrr |
| ID NO: 405) | |
| KETWFETWFTEWSQPKKKRKV (SEQ | ckkkkkkkkk |
| ID NO: 406) | |
| KWFETWFTEWPKKRK (SEQ ID NO: 407) | GRKKRRQRRRPP (SEQ ID NO: 796) |
| GLWRALWRLLRSLWRLLWRA (SEQ ID | RRRRRRRRR (SEQ ID NO: 50) |
| NO: 408) | |
| GLWWRLWWRLRSWFRLWFRA (SEQ | RRRRRRRR (SEQ ID NO: 2) |
| ID NO: 409) | |
| DAATATRGRSAASRPTQRPRAPARSAS | rrrrrrrr |
| RPRRPVE (SEQ ID NO: 410) | |
| GALFLGFLGAAGSTMGAWSQPKKKRKV | AKVKDEPQRRSARLSAKPAPPKPEPKPKKAPAKK (SEQ ID |
| (SEQ ID NO: 411) | NO: 797) |
| GALFLGFLGAAGSTMGAWSQPKSKRKV | |
| (SEQ ID NO: 412) | |

Table 5 shows the selection examples of the targeting peptide, including but not limited to the target proteins indicated by “target protein name” and the peptide sequences corresponding to the target proteins. There are about 19,813 target proteins in total, including all known target proteins and all targeting peptides targeting these target proteins. Due to their large size, the inventors only screened dozens of representative target proteins as examples. However, the claimed target proteins in the present invention are all target proteins and targeting peptides known in the art, and is not limited to these dozens of target proteins, as shown in Table 5 below.

| Target protein name | Peptide sequence |
|---|---|
| A COVALENT ENZYME-SUBSTRATE | AXXXX (SEQ ID NO: 798) |
| INTERMEDIATE WITH SACCHARIDE | |
| DISTORTION IN A MUTANT T4 | |
| LYSOZYME | |
| C-SRC (SH2 DOMAIN) COMPLEXED | XEX |
| WITH ACE-MALONYL TYR-GLU-(N,N- | |
| DIPENTYL AMINE) | |
| MHC CLASS I MOLECULE B*5301 | TPYDINQML (SEQ ID NO: 799) |
| COMPLEXED WITH PEPTIDE | |
| TPYDINQML FROM GAG PROTEIN OF | |
| HIV2 | |
| MHC CLASS I MOLECULE B*3501 | VPLRPMTY (SEQ ID NO: 800) |
| COMPLEXED WITH PEPTIDE VPLRPMTY | |
| FROM THE NEF PROTEIN (75-82) OF | |
| HIV1 | |
| MHC CLASS I MOLECULE B*5301 | KPIVQYDNF (SEQ ID NO: 801) |
| COMPLEXED WITH PEPTIDE LS6 | |
| (KPIVQYDNF) FROM THE MALARIA | |
| PARASITE P. FALCIPARUM | |
| HCV NS3 PROTEASE DOMAIN:NS4A | GSVVIVGRIVLSGKPA (SEQ ID NO: 802) |
| PEPTIDE COMPLEX | |
| HCV NS3 PROTEASE DOMAIN:NS4A | GSVVIVGRIVLSGKPA (SEQ ID NO: 802) |
| PEPTIDE COMPLEX | |
| HCV NS3 PROTEASE DOMAIN:NS4A | KGSVVIVGRIVLSGKPAIIPK (SEQ ID NO: 803) |
| PEPTIDE COMPLEX | |
| HCV NS3 PROTEASE DOMAIN:NS4A | KGSVVIVGRIVLSGKPAIIPK (SEQ ID NO: 803) |
| PEPTIDE COMPLEX | |
| STRUCTURE OF THROMBIN INHIBITED | TFGSGEADCGLRPLFEKKSLEDKTERELLESYIDGR |
| BY AERUGINOSAN298-A FROM A BLUE- | (SEQ ID NO: 804) |
| GREEN ALGA | |
| STRUCTURE OF THROMBIN INHIBITED | DFEEIPEEXL (SEQ ID NO: 805) |
| BY AERUGINOSAN298-A FROM A BLUE- | |
| GREEN ALGA | |
| STRUCTURE OF THROMBIN INHIBITED | XLXX (SEQ ID NO: 806) |
| BY AERUGINOSAN298-A FROM A BLUE- | |
| GREEN ALGA | |
| COMPLEX OF TROPONIN C WITH A 47 | EEKRNRAITARRQHLKSVMLQIAATELEKEE (SEQ |
| RESIDUE (1-47) FRAGMENT OF | ID NO: 807) |
| TROPONIN I | |
| HIV-1 PROTEASE COMPLEXED WITH A | EDL |
| TRIPEPTIDE INHIBITOR | |
| HIV-1 PROTEASE COMPLEXED WITH A | EDL |
| TRIPEPTIDE INHIBITOR | |
| COMPLEX OF HUMAN ALPHA- | ADCGLRPLFEKKSLEDKTERELLESYI (SEQ ID NO: |
| THROMBIN WITH THE BIFUNCTIONAL | 808) |
| BORONATE INHIBITOR BOROLOG1 | |
| CRYSTAL STRUCTURE OF BOVINE | TPGVY (SEQ ID NO: 809) |
| GAMMA-CHYMOTRYPSIN | |
| CRYSTAL STRUCTURE OF BOVINE | TPGVY (SEQ ID NO: 809) |
| GAMMA-CHYMOTRYPSIN | |
| STRUCTURE OF THE HIRULOG 3- | SGEADCGLRPLFEKKSLEDKTERELLESYIDGR (SEQ |
| THROMBIN COMPLEX AND NATURE OF | ID NO: 810) |
| THE S' SUBSITES OF SUBSTRATES AND | |
| INHIBITORS | |
| STRUCTURE OF THE HIRULOG 3- | XPXGGGGGNGDXEEIPEEYL (SEQ ID NO: 811) |
| THROMBIN COMPLEX AND NATURE OF | |
| THE S' SUBSITES OF SUBSTRATES AND | |
| INHIBITORS | |
| STRUCTURE OF THE HIRULOG 3- | ADCGLRPLFEKKSLEDKTERELLESYI (SEQ ID NO: |
| THROMBIN COMPLEX AND NATURE OF | 812) |
| SUBSITES OF SUBSTRATES INHIBITORS | |
| NMR SOLUTION STRUCTURE OF AN | KHWVYY (SEQ ID NO: 813) |
| ALPHA-BUNGAROTOXIN(SLASH) | |
| NICOTINIC RECEPTOR PEPTIDE | |
| COMPLEX | |
| COMPLEX OF THROMBIN WITH AND | EADCGLRPLFEKKSLEDKTERELLESYI (SEQ ID NO: |
| INHIBITOR CONTAINING A NOVEL P1 | 814) |
| MOIETY | |
| COMPLEX OF THROMBIN WITH AND | DFEEIPEEXL (SEQ ID NO: 815) |
| INHIBITOR CONTAINING A NOVEL P1 | |
| MOIETY | |
| HUMAN ALPHA-THROMBIN INHIBITION | TFGSGEADCGLRPLFEKKSLEDKTERELLESYIDGR |
| BY EOC-D-PHE-PRO-AZALYS-ONP | (SEQ ID NO: 816) |
| HUMAN ALPHA-THROMBIN INHIBITION | DFEEIPEEXL (SEQ ID NO: 815) |
| BY EOC-D-PHE-PRO-AZALYS-ONP | |
| HUMAN ALPHA-THROMBIN INHIBITION | TFGSGEADCGLRPLFEKKSLEDKTERELLESYIDGR |
| BY CBZ-PRO-AZALYS-ONP | (SEQ ID NO: 817) |
| HUMAN ALPHA-THROMBIN INHIBITION | DFEEIPEEXL (SEQ ID NO: 815) |
| BY CBZ-PRO-AZALYS-ONP | |
| CRYSTAL STRUCTURE OF BOVINE | CGVPAIQPVL (SEQ ID NO: 818) |
| GAMMA-CHYMOTRYPSIN COMPLEXED | |
| WITH A SYNTHETIC INHIBITOR | |
| CRYSTAL STRUCTURE OF BOVINE | CGVPAIQPVL (SEQ ID NO: 818) |
| GAMMA-CHYMOTRYPSIN COMPLEXED | |
| WITH A SYNTHETIC INHIBITOR | |
| ANTAGONIST HIV-1 GAG PEPTIDES | GGRKKYKL (SEQ ID NO: 819) |
| INDUCE STRUCTURAL CHANGES IN | |
| HLA B8-HIV-1 GAG PEPTIDE | |
| (GGRKKYKL-3R MUTATION) | |
| ANTAGONIST HIV-1 GAG PEPTIDES | GGKKKYQL (SEQ ID NO: 820) |
| INDUCE STRUCTURAL CHANGES IN | |
| HLA B8-HIV-1 GAG PEPTIDE | |
| (GGKKKYQL-7Q MUTATION) | |
| ANTAGONIST HIV-1 GAG PEPTIDES | GGKKKYKL (SEQ ID NO: 821) |
| INDUCE STRUCTURAL CHANGES IN | |
| HLA B8-HIV-1 GAG PEPTIDE | |
| (GGKKKYKL-INDEX PEPTIDE) | |
| ANTAGONIST HIV-1 GAG PEPTIDES | GGKKKYRL (SEQ ID NO: 822) |
| INDUCE STRUCTURAL CHANGES IN | |
| HLA B8-HIV-1 GAG PEPTIDE | |
| (GGKKKYRL-7R MUTATION) | |
| ANTAGONIST HIV-1 GAG PEPTIDES | GGKKRYKL (SEQ ID NO: 823) |
| INDUCE STRUCTURAL CHANGES IN | |
| HLA B8-HIV-1 GAG PEPTIDE | |
| (GGKKRYKL-5R MUTATION) | |
| CRYSTAL STRUCTURE OF HUMAN | EADCGLRPLFEKKSLEDKTERELLESYI (SEQ ID NO: |
| ALPHA-THROMBIN COMPLEXED WITH | 824) |
| HIRUGEN AND P- | |
| AMIDINOPHENYLPYRUVATE AT 1.6 | |
| ANGSTROMS RESOLUTION | |
| CRYSTAL STRUCTURE OF HUMAN | DFEEIPEEXL (SEQ ID NO: 825) |
| ALPHA-THROMBIN COMPLEXED WITH | |
| HIRUGEN AND P- | |
| AMIDINOPHENYLPYRUVATE AT 1.6 | |
| ANGSTROMS RESOLUTION | |
| HLA-DR1 (DRA, DRB1 0101) HUMAN | SDWRFLRGYHQYA (SEQ ID NO: 826) |
| CLASS II HISTOCOMPATIBILITY | |
| PROTEIN (EXTRACELLULAR DOMAIN) | |
| COMPLEXED WITH ENDOGENOUS | |
| PEPTIDE | |
| HLA-DR1 (DRA, DRB1 0101) HUMAN | GSDWRFLRGYHQYA (SEQ ID NO: 827) |
| CLASS II HISTOCOMPATIBILITY | |
| PROTEIN (EXTRACELLULAR DOMAIN) | |
| COMPLEXED WITH ENDOGENOUS | |
| PEPTIDE | |
| CLEAVED ANTICHYMOTRYPSIN A349R | GTIVRFNRPFLMIIVPTDTQNIFFMSKVTNPKQ (SEQ |
| | ID NO: 828) |

In Table 1-Table 5, “e3 ligand” in Table 3 represents all the currently applicable small molecule ligands of E3 ligase. There are two types of linkers, one is “PEG linkers” shown in Table 2, and the other linker is collected in “linkers” in Table 1. “CPP list” in Table 4 is all the currently applicable cell-penetrating peptides. “Target interacting peptide” in Table 5 is the currently applicable targeting peptide examples of all targets.

At present, due to technical limitations, only about 10-20% of the targets can be developed. However, for the CePPiTAC technology provided by the present invention, peptides are used instead of small molecules to target the target protein, and connected to the cell-penetrating peptide sequence to ensure the complex is able to enter the cell membrane. Since any target protein can be screened for binding to the resistant peptides that can be linked to it, in theory, any target protein can be targeted to be degraded by proteases. Therefore, the application market is extremely broad. The previous “non-targetable” target proteins may be degraded by the drugs developed by the technology. Moreover, since the interactions between many proteins and proteins have long been established, it is very convenient to screen ligand peptides. There are now a number of high-efficiency small molecule E3 ligase ligands, which are very simple to connect with peptides and will be very convenient to design drugs with time and effort saved and develop various new drugs quickly and economically according to the present invention.

### Example 3

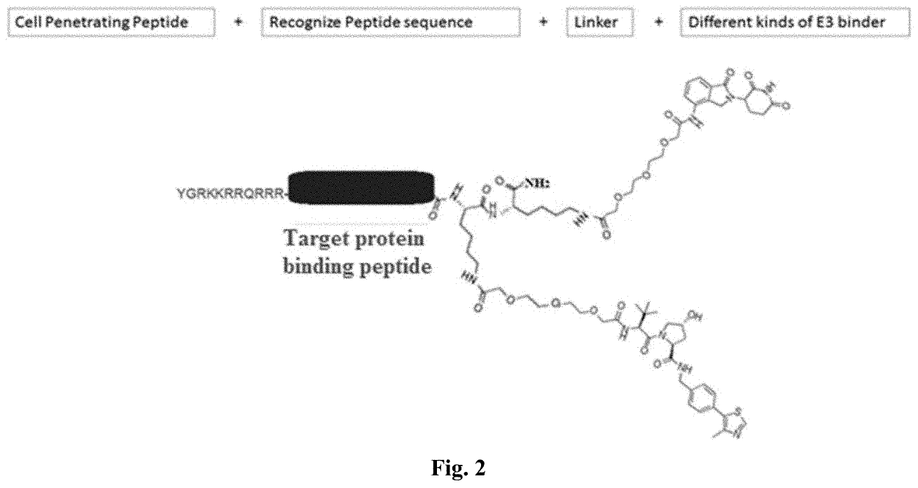

The inventors also combine the targeting peptide with two or multiple different E3 ligase conjugates as shown below to efficiently degrade target proteins, which is specifically as shown in FIG. 2.

### Example 4

The inventors also contemplate degrading multiple targets related to the formation of pathogenic protein-protein complexes to inhibit entire pathogenic pathways as well as degrading multiple targets to completely inhibit a specific disease. To achieve this, the inventors designed the peptide degrader as shown in FIG. 3.

### Example 5

A representative PROTAC peptide conjugate PEN-FFW-LINK-LEN is synthesized, wherein PEN, FFW and LEN represent cell-penetrating peptide, targeting peptide and small molecule ligand, respectively.

a. Solid-Phase Synthesis of Peptide 1:

Scheme 1:

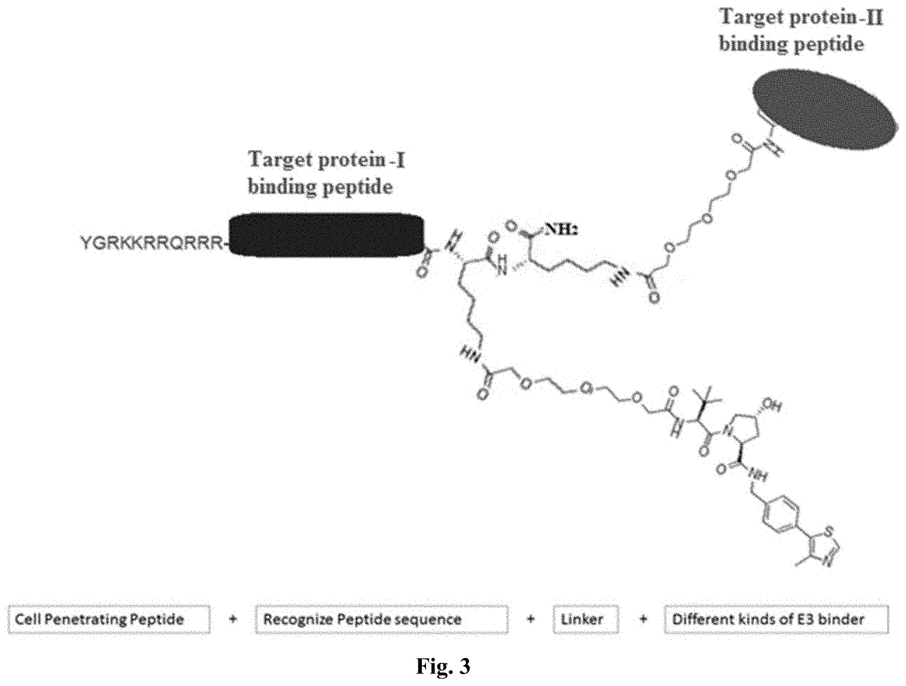

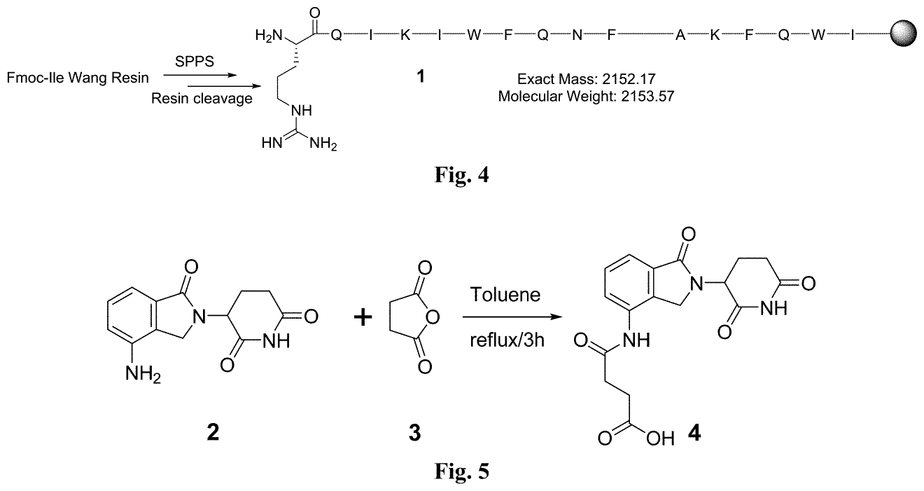

As shown in FIG. 4, peptide 1 was synthesized at 0.15 mmol.

The SYRO automated peptide synthesizer was used to assist in elongation of the full sequence. 0.5 g Fmoc-Ile Wang resin (0.3 mmol/g) was swollen in DMF, and the Fmoc-moiety was deprotected using 20% piperidine/DMF (2 times for 5 min and 20 min, respectively). After each deprotection, the resin was washed with DMF (3×10 mL). On the synthesizer, each fmoc-amino acid residue (4 eq, 0.6 mmol) was treated with two different activator, DIC/Oxyma (4 eq, 0.6 mmol, 30 min) and HATU/DIPEA (4 eq, 0.6 mmol, 45 min) in DMF, to double-couple resins. The final Fmoc-moiety was deprotected using 20% piperidine/DMF (2 times for 5 min and 20 min, respectively) and washed with DMF (3×10 mL) to provide resin-bound linear peptide 1. The desired mass was determined by microlysis.

b. Synthesis of Lenalidomide-Conjugated Succinic Anhydride:

Scheme 1:

As shown in FIG. 5, lenalidomide 2 (200 mg, 0.77 mmol) was added to a round bottom flask containing a solution of succinic anhydride 3 (90 mg, 0.93 mmol) in toluene (8 mL) and equipped with a reflux condenser. The mixture was refluxed for 3 h, and the precipitate was separated by vacuum filtration. The filter cake was washed with ethyl acetate (20 mL×2) and dried under vacuum to obtain product 4: 4-(2-(2,6-dioxopiperidin-3-yl)-1-oxyisoindol-4-yl)amino)-4-oxobutyric acid. Yield: 120 mg, 43.4%.

c. Solid-Phase Synthesis and Resin Cleavage of LEN-Conjugated Peptides:

Scheme 3:

1. Solid-Phase Synthesis:

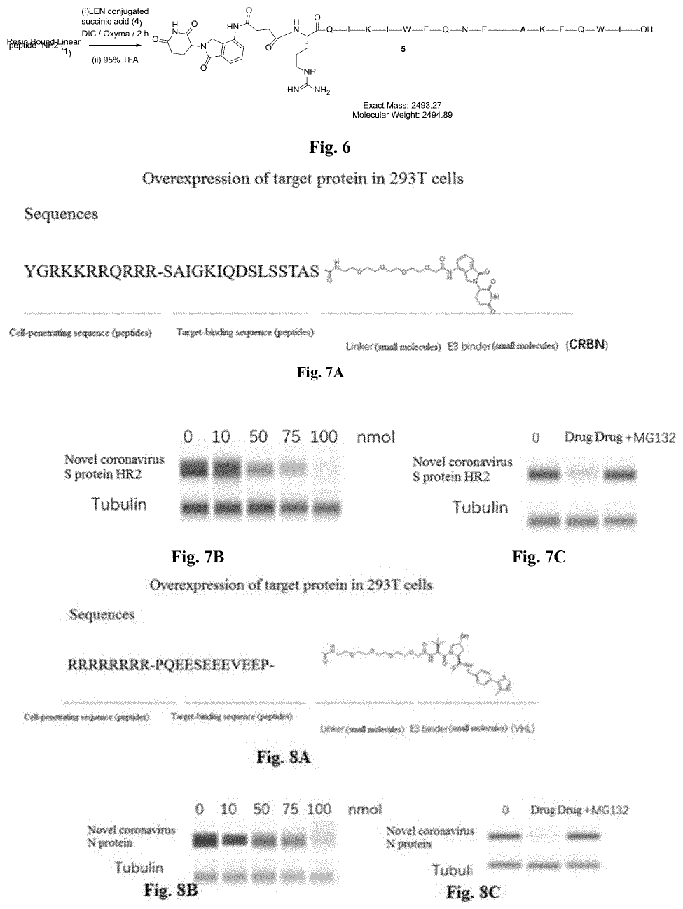

As shown in FIG. 6, lenalidomide-conjugated succinic anhydride (4) was activated with DIC/Oxyma (4 eq) in DMF, added to the pre-treated resin-bound amine (200 mg, 0.06 mmol), and shaken well or for 2 h. with The resin was filtered and washed with DMF (3×10 mL) and DCM (3×10 mL), finally washed with diethyl ether (2×10 mL) and dried under vacuum for resin cleavage.

2. Resin Cleavage:

The product was isolated from 10 ml of resin containing trifluoroacetic acid, triisopropylsilane, and water (95:2.5:2.5) to provide 120 mg of crude peptide, which was purified by reversed-phase high performance liquid chromatography (HPLC) to obtain 10 mg of diastereomeric mixture 5 with a maximum purity of 97.15% and a purity of 93.19% at 214 nm. Yield: 10 mg, 6.68%.

Table 6 shows the preparation conditions for HPLC.

| Instrument | Agilent Technologies 1260 infinity | |
|---|---|---|
| Column | X-Select CSH C18 (250*19) mm 5 μm | |
| Mobile phase A | 0.1% TFA aqueous solution | |
| Mobile phase B | Acetonitrile | |
| Flow rate | 15 | mL/min |
| Reaction time | 22 | mins |
| Load capacity | 10 | mg/injection |

Table 7 shows a gradient table.

| Time | % of mobile | % of mobile |
|---|---|---|
| (min) | phase A | phase B |
| 0.0 | 90 | 10 |
| 15 | 50 | 50 |
| 15.1 | 0 | 100 |
| 19 | 0 | 100 |
| 19.1 | 90 | 10 |
| 22 | 90 | 10 |

### Example 6

1. The target proteins of many diseases are membrane proteins, such as PD-1 and PD-L1. Although the inhibitor drugs against them are very commonly used, they have low efficacy and are prone to drug resistance. It is because target proteins PD-1 and PD-L1 are on the cell membrane, and it is difficult to develop small-molecule PD-1/PD-L1 degraders. However, the technology according to the present invention can target the intracellular parts of these two proteins and then degrade these two proteins.

2. Some disease targets are difficult to bind to small molecules due to their structure. Therefore, it is difficult to design for ordinary small molecules by PROTAC, such as the G12V variant of Kras protein. However, the technology according to the present invention can use peptides to bind to them and then degrade them.

3. Some virus-related proteins such as the novel coronavirus proteins or HIV virus-related proteins have few targets since conventional viral drugs focus on neutralizing antibodies or virus-inhibiting proteases to inhibit the virus, and once the virus mutates, the drugs developed will be useless. The CePPiTAC technology provided by the present invention can select viral structural proteins or proteases to be bound and targeted by peptides and then degrade them, so that the protein synthesis is damaged or the virus packaging fails. This will firstly expand the number of targets for viral drug research and development since many viral proteins that could not be targeted in the past can be targeted. Secondly, the structural protein that is not easily mutated can be bound to degrade the entire target protein, so the drug can be prepared without worries about any virus mutations.

### Example 7

In order to illustrate the modulating targeting chimera molecule induced by a cell-penetrating peptide given in this application, the random combination of four “modules” in its basic structure and various selections, the inventors designed a modulating targeting chimera molecule for degrading novel coronavirus S protein HR2 (FIG. 7A), in which the cell-penetrating peptide module has a sequence of YGRKKRRQRRR (SEQ ID NO: 1), the targeting peptide module has a sequence of SAIGKIQDSLSSTAS (SEQ ID NO: 4), the Linker module is a small molecule composed of (PEG)4 with a structural formula of

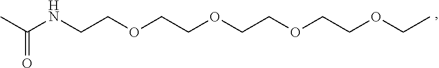 [SMILES: CCOCCOCCOCCOCCNC(C)=O]

and E3 small molecule ligand module is an E3 ligand targeting CRBN with a structural formula of

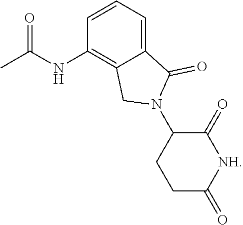 [SMILES: CC(=O)Nc1cccc2c1CN(C1CCC(=O)NC1=O)C2=O]

The degradation effect of the targeting chimera molecule with different dosages (nmol) on protein was verified by Western Blot (FIG. 7B). It can be seen that with the increase of dosage, the protein has been degraded (no expression).

In order to verify that the targeting chimera molecule provided in this application serves to degrade rather than inhibit, the inventor also designed and added the protease inhibitor MG132, which can inhibit the effect of the targeting chimera provided in this application. From the experimental results (FIG. 7C), it can be seen that the protein only added with the targeting chimera molecule still cannot be expressed, but the protein added with the targeting chimera molecule+MG132 can be expressed normally with an expression level comparable to that of the protein without adding the targeting chimera molecule. These results demonstrate that the role of the targeting chimera molecule is to degrade rather than inhibit the protein.

Studies have shown that the S protein on the surface of the coronavirus mediates the infection of target cells by the virus. It consists of two subunits, S1 and S2. The S1 subunit is responsible for binding to the receptor on the cell surface, and the S2 subunit functions to fuse the virus with the cell membrane. The S2 subunit contains important functional regions such as heptapeptide repeat domain 1 (HR1) and heptapeptide repeat domain 2 (HR2). During the fusion of the viral membrane, HR1 and HR2 fold to form a six-helix bundle structure (6HB) to bring the viral membrane and the cell membrane closer together for fusion reaction, so that the genetic material of the virus enters the target cell through the fusion hole. The targeting chimera molecule can bind to the HR2 subunit of the novel coronavirus S protein and degrade it, which inhibits the formation of the six-helix bundle structure, thereby interferes with the fusion of the virus and the cell membrane, prevents the virus from invading cells, and fundamentally achieves the purpose of preventing and treating the novel coronavirus disease.

### Example 8

Similar to Example 7, in order to illustrate the “modulating” design of the present application, the inventors also designed modulating targeting chimera molecules for degrading novel coronavirus N protein, novel coronavirus M protein, novel coronavirus E protein, novel coronavirus Orf6 protein, Lag-3 protein, Her2 protein, SHP-2 protein, STAT5B protein, MUC16 protein, CTLA-4 protein, PCSK9 protein, PD-1 protein and PD-L1 protein, respectively (FIG. 8A-FIG. 20A).

In the modulating targeting chimera molecule for degrading novel coronavirus N protein, the cell-penetrating peptide module has a sequence of RRRRRRRR (SEQ ID NO: 2), the targeting peptide module has a sequence of PQEESEEEVEEP (SEQ ID NO: 5), the Linker module is a small molecule composed of (PEG)4 with a structural formula of

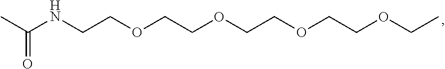 [SMILES: CCOCCOCCOCCOCCNC(C)=O]

and E3 small molecule ligand module is an E3 ligand targeting VHL with a structural formula of

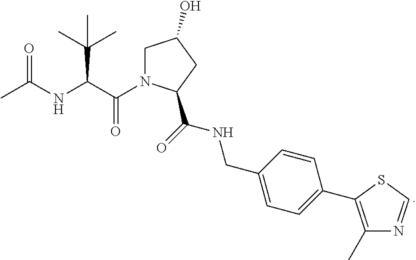 [SMILES: CC(=O)N[C@H](C(=O)N1C[C@H](O)C[C@H]1C(=O)NCc1ccc(-c2scnc2C)cc1)C(C)(C)C]

In the modulating targeting chimera molecule for degrading novel coronavirus M protein, the cell-penetrating peptide module has a sequence of YGRKKRRQRRR (SEQ ID NO: 1), the targeting peptide module has a sequence of PQEESEEEVEEP (SEQ ID NO: 5), the Linker module is a small molecule composed of (PEG)4 with a structural formula of

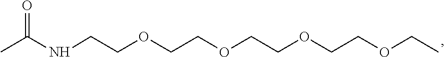 [SMILES: CCOCCOCCOCCOCCNC(C)=O]

and E3 small molecule ligand module is an E3 ligand targeting IAP with a structural formula of

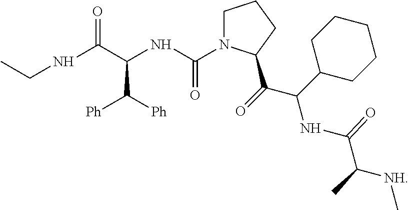 [SMILES: CCNC(=O)[C@@H](NC(=O)N1CCC[C@H]1C(=O)C(NC(=O)[C@H](C)NC)C1CCCCC1)C(c1ccccc1)c1ccccc1]

In the modulating targeting chimera molecule for degrading novel coronavirus E protein, the cell-penetrating peptide module has a sequence of YGRKKRRQRRR (SEQ ID NO: 1), the targeting peptide module has a sequence of GGKGLGKacGGA, the Linker module is a small molecule composed of (PEG)4 with a structural formula of

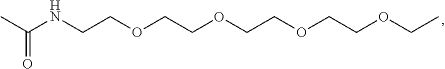 [SMILES: CCOCCOCCOCCOCCNC(C)=O]

and E3 small molecule ligand module is an E3 ligand targeting CRBN with a structural formula of

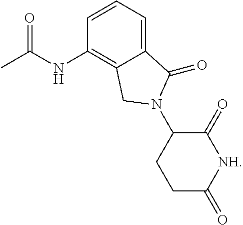 [SMILES: CC(=O)Nc1cccc2c1CN(C1CCC(=O)NC1=O)C2=O]

In the modulating targeting chimera molecule for degrading novel coronavirus Orf6 protein, the cell-penetrating peptide module has a sequence of YGRKKRRQRRR (SEQ ID NO: 1), the targeting peptide module has a sequence of DTMVGWDKDARTK (SEQ ID NO: 7), the Linker module is a small molecule composed of (PEG)4 with a structural formula of

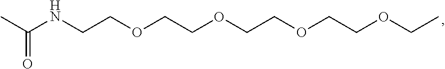 [SMILES: CCOCCOCCOCCOCCNC(C)=O]

and E3 small molecule ligand module is an E3 ligand targeting VHL with a structural formula of

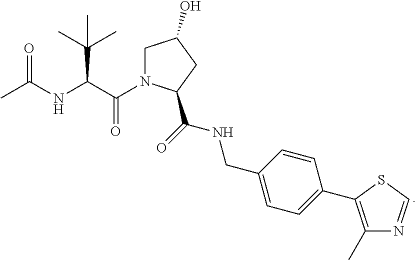 [SMILES: CC(=O)N[C@H](C(=O)N1C[C@H](O)C[C@H]1C(=O)NCc1ccc(-c2scnc2C)cc1)C(C)(C)C]

In the modulating targeting chimera molecule for degrading Lag-3 protein, the cell-penetrating peptide module has a sequence of YGRKKRRQRRR (SEQ ID NO: 1), the targeting peptide module has a sequence of FNGARSFIDI (SEQ ID NO: 8), the Linker module is a small molecule composed of (PEG)4 with a structural formula of

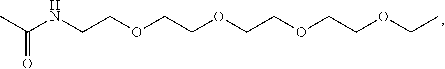 [SMILES: CCOCCOCCOCCOCCNC(C)=O]

and E3 small molecule ligand module is an E3 ligand targeting CRBN with a structural formula of

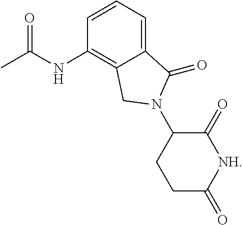 [SMILES: CC(=O)Nc1cccc2c1CN(C1CCC(=O)NC1=O)C2=O]

In the modulating targeting chimera molecule for degrading Her2 protein, the cell-penetrating peptide module has a sequence of YGRKKRRQRRR (SEQ ID NO: 1), the targeting peptide module has a sequence of WARLWNYLYR (SEQ ID NO: 9), the Linker module is a small molecule composed of (PEG)4 with a structural formula of

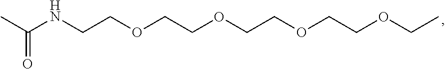 [SMILES: CCOCCOCCOCCOCCNC(C)=O]

and E3 small molecule ligand module is an E3 ligand targeting VHL with a structural formula of

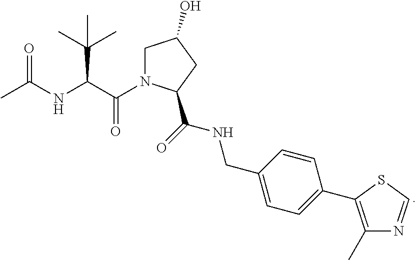 [SMILES: CC(=O)N[C@H](C(=O)N1C[C@H](O)C[C@H]1C(=O)NCc1ccc(-c2scnc2C)cc1)C(C)(C)C]

In the modulating targeting chimera molecule for degrading SHP-2 protein, the cell-penetrating peptide module has a sequence of YGRKKRRQRRR (SEQ ID NO: 1), the targeting peptide module has a sequence of RSFIDIGSGT (SEQ ID NO: 10), the Linker module is a small molecule composed of (PEG)4 with a structural formula of

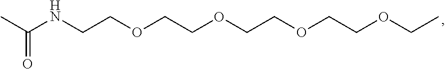 [SMILES: CCOCCOCCOCCOCCNC(C)=O]

and E3 small molecule ligand module is an E3 ligand targeting CRBN with a structural formula of

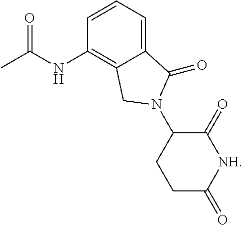 [SMILES: CC(=O)Nc1cccc2c1CN(C1CCC(=O)NC1=O)C2=O]

In the modulating targeting chimera molecule for degrading STAT5B protein, the cell-penetrating peptide module has a sequence of YGRKKRRQRRR (SEQ ID NO: 1), the targeting peptide module has a sequence of KAVDG(p)YVKPQI (SEQ ID NO: 11), the Linker module is a small molecule composed of (PEG)4 with a structural formula of

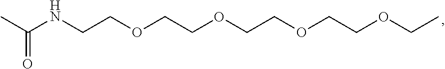 [SMILES: CCOCCOCCOCCOCCNC(C)=O]

and E3 small molecule ligand module is an E3 ligand targeting IAP with a structural formula of

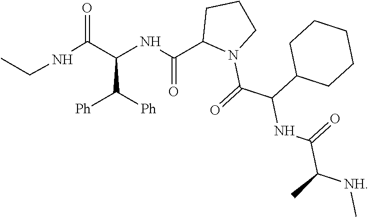 [SMILES: CCNC(=O)[C@@H](NC(=O)C1CCCN1C(=O)C(NC(=O)[C@H](C)NC)C1CCCCC1)C(c1ccccc1)c1ccccc1]

In the modulating targeting chimera molecule for degrading MUC16 protein, the cell-penetrating peptide module has a sequence of YGRKKRRQRRR (SEQ ID NO: 1), the targeting peptide module has a sequence of WIDPVNGDTE (SEQ ID NO: 12), the Linker module is a small molecule composed of (PEG)4 with a structural formula of

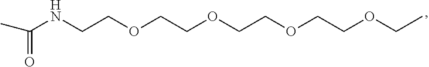 [SMILES: CCOCCOCCOCCOCCNC(C)=O]

and E3 small molecule ligand module is an E3 ligand targeting VHL with a structural formula of

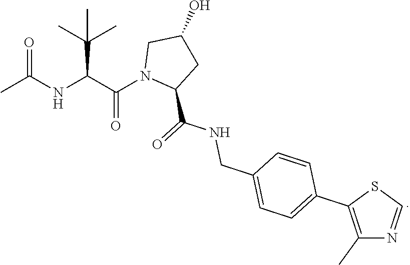 [SMILES: CC(=O)N[C@H](C(=O)N1C[C@H](O)C[C@H]1C(=O)NCc1ccc(-c2scnc2C)cc1)C(C)(C)C]

In the modulating targeting chimera molecule for degrading CTLA-4 protein, the cell-penetrating peptide module has a sequence of YGRKKRRQRRR (SEQ ID NO: 1), the targeting peptide module has a sequence of ARHPSWYRPFEGCG (SEQ ID NO: 13), the Linker module is a small molecule composed of (PEG)4 with a structural formula of

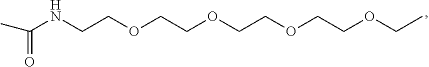 [SMILES: CCOCCOCCOCCOCCNC(C)=O]

and E3 small molecule ligand module is an E3 ligand targeting VHL with a structural formula of

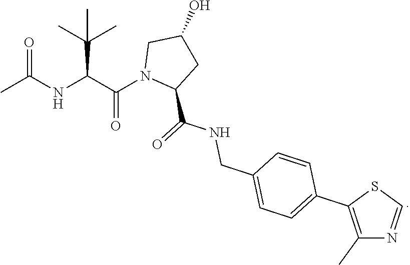 [SMILES: CC(=O)N[C@H](C(=O)N1C[C@H](O)C[C@H]1C(=O)NCc1ccc(-c2scnc2C)cc1)C(C)(C)C]

In the modulating targeting chimera molecule for degrading PCSK9 protein, the cell-penetrating peptide module has a sequence of RQIKIWFQNRRMKWK (SEQ ID NO: 3), the targeting peptide module has a sequence of MESFPGWNLV(homoR)IGLLR (SEQ ID NO: 14), the Linker module is a small molecule composed of (PEG)4 with a structural formula of

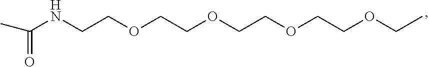 [SMILES: CCOCCOCCOCCOCCNC(C)=O]

and E3 small molecule ligand module is an E3 ligand targeting IAP with a structural formula of

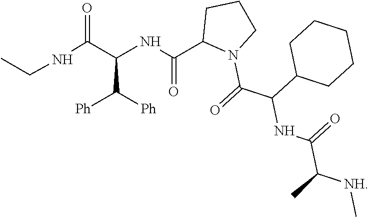 [SMILES: CCNC(=O)[C@@H](NC(=O)C1CCCN1C(=O)C(NC(=O)[C@H](C)NC)C1CCCCC1)C(c1ccccc1)c1ccccc1]

In the modulating targeting chimera molecule for degrading PD-1 protein, the cell-penetrating peptide module has a sequence of YGRKKRRQRRR (SEQ ID NO: 1), the targeting peptide module has a sequence of FNWDYSLEELREKAKYK (SEQ ID NO: 15), the Linker module is a small molecule composed of (PEG)4 with a structural formula of

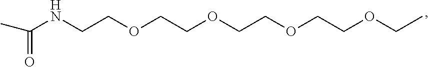 [SMILES: CCOCCOCCOCCOCCNC(C)=O]

and E3 small molecule ligand module is an E3 ligand targeting CRBN with a structural formula of

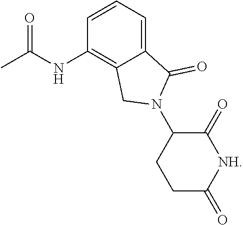 [SMILES: CC(=O)Nc1cccc2c1CN(C1CCC(=O)NC1=O)C2=O]

In the modulating targeting chimera molecule for degrading PD-L1 protein, the cell-penetrating peptide module has a sequence of YGRKKRRQRRR (SEQ ID NO: 1), the targeting peptide module has a sequence of MPIFLDHILNKFWILHYA (SEQ ID NO: 16), the Linker module is a small molecule composed of (PEG)4 with a structural formula of

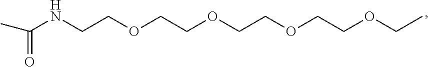 [SMILES: CCOCCOCCOCCOCCNC(C)=O]

and E3 small molecule ligand module is an E3 ligand targeting CRBN with a structural formula of

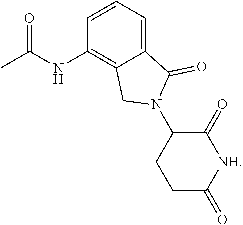 [SMILES: CC(=O)Nc1cccc2c1CN(C1CCC(=O)NC1=O)C2=O]

Likewise, the degradation effect of the targeting chimera molecules with different dosages (nmol) on novel coronavirus N protein, novel coronavirus M protein, novel coronavirus E protein, novel coronavirus Orf6 protein, Lag-3 protein, Her2 protein, SHP-2 protein, STAT5B protein, MUC16 protein and CTLA-4 protein was verified by Western Blot (FIG. 8B-FIG. 17B). It can be seen that with the increase of dosage, the protein has been degraded (no expression).

Likewise, in order to verify that the targeting chimera molecule provided in this application serves to degrade rather than inhibit, the inventor also designed and added the protease inhibitor MG132, which can inhibit the effect of the targeting chimera provided in this application. From the experimental results (FIG. 8C-FIG. 16C and FIGS. 18B-20B), it can be seen that the protein only added with the targeting chimera molecule still cannot be expressed, but the protein added with the targeting chimera molecule+MG132 can be expressed normally with an expression level comparable to that of the protein without adding the targeting chimera molecule. These results demonstrate that the role of the targeting chimera molecule is to degrade rather than inhibit the protein.

The novel coronavirus N protein, which is abundant in coronaviruses, is a highly immunogenic protein involved in genome replication and regulation of cell signaling pathways. Degrading this protein with the targeting chimera molecule of this application can effectively inhibit the novel coronavirus and treat the disease caused by it.

The novel coronavirus M protein, as a membrane glycoprotein (M), is an integral part of the viral particle envelope. M protein is involved in the assembly and release of the next generation of viral particles, and plays an important role in the structural stability and functional expression of other structural proteins (S, E, N proteins). Degrading this protein with the targeting chimera molecule can effectively destroy the stability of the viral structure and inhibit viral function.

The novel coronavirus E protein (E, Envelope Protein) is an integral part of the viral particle envelope and is a small envelope glycoprotein. The main function of the E protein is to protect the RNA gene strand inside the virus. Degrading this protein with the targeting chimera molecule can reduce or even remove the protective mechanism of viral RNA, making the RNA strand more prone to breakage, thereby effectively inhibiting viral function.

The novel coronavirus Orf6 protein is the most toxic to human cells among the novel coronavirus proteins. Existing research has found that it can kill about half of human cells after being introduced into human cells and can effectively inhibit the innate immunity of host cells. Degrading this protein with the targeting chimera molecule can greatly reduce the toxicity of the novel coronavirus to the human immune system.

Lag-3 protein, lymphocyte activation gene 3, also known as CD233, is a type I transmembrane protein, which belongs to the immunoglobulin (Ig) superfamily and mainly expressed on the surface of activated T cells and NK cells. LAG-3 is a very promising immunotherapy target. Degrading this protein with the targeting chimera molecule can effectively block the inhibitory signal in the interaction between tumor cells and TIL in the tumor microenvironment, restore the immune surveillance function of TIL on tumor cells, and achieve antitumor effects.

Her2 protein, a transmembrane protein with tyrosine protein kinase activity, is a member of the EGFR family. HER2 gene amplification is one of the most important factors affecting the growth and metastasis of breast cancer. Degrading this protein with the targeting chimera molecule can promote the apoptosis of and inhibit the proliferation of breast tumors.

SHP-2 protein, encoded by protein tyrosine phosphatase nonreceptor 11 (PTPN11), is a well-validated PTP oncoprotein in humans and is emerging as an important target for the treatment of cancer. Hyperactivation of SHP2 plays a crucial pathogenic role. Degrading this protein with the targeting chimera molecule can effectively block or inhibit the activation of the SHP2 pathway, thereby significantly improving tumor treatment.

STAT5B protein is signal transducer and activator of transcription-5b. STAT signal is a regulatory signal of various tumors. Degrading this protein with the targeting chimera molecule can effectively cause the dysregulation of STAT signal, thereby inhibiting the proliferation and clone formation of tumor cells (such as osteosarcoma cells) and inducing cell arrest and apoptosis at G0/G1 phase.

MUC16 protein, the largest transmembrane mucin, is a well-established serum biomarker for ovarian cancer since MUC16 is known to be overexpressed on the surface of ovarian cancer cells and split/shed into the blood. It is believed to play an anti-apoptotic role in cancer cells. The ectopic expression of its C-terminal domain induces resistance to cisplatin in ovarian cancer cells, and this effect is mediated by the inhibition of p53. Degrading this protein with the targeting chimera molecule can effectively regulate the apoptosis of and inhibit the proliferation of cancer cells.

CTLA-4 protein, cytotoxic T lymphocyte-associated protein 4, also known as CD152 (cluster of differentiation 152), is a protein receptor that functions as an immune checkpoint and downregulates immune responses. Mutations in the CTLA-4 gene are not only associated with cancer, but also with type 1 diabetes, Graves' disease, Hashimoto's thyroiditis, celiac disease, systemic lupus erythematosus, thyroid-related orbitopathy, primary biliary cirrhosis and other autoimmune diseases. Degrading this protein with the targeting chimera molecule can effectively increase the immune activity of the body.

### Example 9

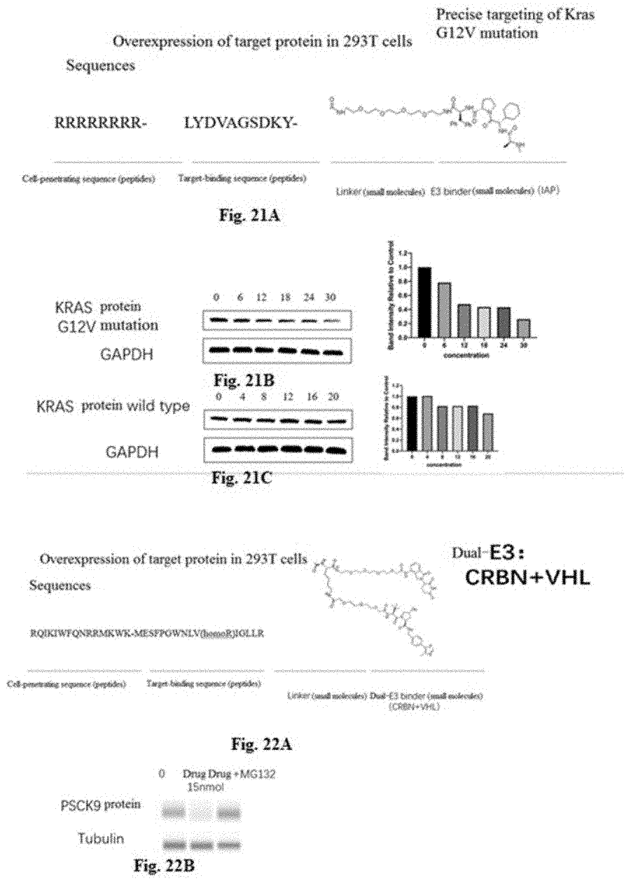

In particular, in order to illustrate that the “modulating” design given in this application can also precisely target proteins with mutated amino acids, the inventors designed a modulating targeting chimera molecule for degrading KRAS protein with G12V mutation (FIG. 21A), in which the cell-penetrating peptide module has a sequence of RRRRRRRR (SEQ ID NO: 2), the targeting peptide module has a sequence of LYDVAGSDKY (SEQ ID NO: 17), the Linker module is a small molecule composed of (PEG)4 with a structural formula of

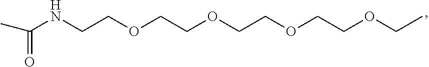 [SMILES: CCOCCOCCOCCOCCNC(C)=O]

and E3 small molecule ligand module is an E3 ligand targeting IAP with a structural formula of

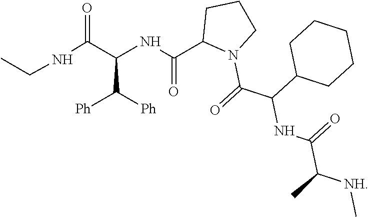 [SMILES: CCNC(=O)[C@@H](NC(=O)C1CCCN1C(=O)C(NC(=O)[C@H](C)NC)C1CCCCC1)C(c1ccccc1)c1ccccc1]

Likewise, the degradation effect of the targeting chimera molecule with different dosages (nmol) on KRAS protein with G12V mutation was verified by Western Blot and the expression level was recorded (FIG. 21B). It can be seen that with the increase of dosage, the protein has been degraded (the expression level gradually decreased).

In order to verify the precise targeting of the targeting chimera molecule, the inventors also verified the degradation effect of the targeting chimera molecule with different dosages (nmol) on non-mutated KRAS protein (wild type) by Western Blot and recorded the expression level (FIG. 21C). It can be seen that with the increase of dosage, the protein changes are not obvious (the expression level was slightly reduced, but not obvious), which fully shows that the targeting chimera molecule of this design can accurately target the KRAS protein with G12V mutation without basically degrading wild-type KRAS protein, indicating an extremely high accuracy of targeting and degradation.

KRAS (Kirsten Rat Sarcoma Viral Oncogene Homolog) is a GDP/GTP binding protein. KRAS is in an activated state when binding to GTP, and in an off state when binding to GDP. KRAS can be temporarily activated by growth factors or tyrosine kinases (such as EGFR). The activated KRAS can activate downstream such as the PI3K-AKT-mTOR signaling pathway that controls cell production, and the RAS-RAF-MEK-ERK signaling pathway that controls cell proliferation. The mutant KRAS will continue to activate even without the activation of kinases such as EGFR, leading to continuous proliferation of cells and eventually cancer. KRAS mutants are found in a variety of tumors, the most common including lung cancer, pancreatic cancer, etc. The targeting chimera molecule can accurately degrade the KRAS protein with G12V mutation, but has no effect on the wild-type KRAS protein, which greatly improves the targeted treatment efficiency of the mutant protein.

### Example 10

In particular, in order to illustrate that the “modulating” design given in this application can also connect the dual-E3 ligand structure and increase the degradation efficiency of the protein, the inventors designed a modulating dual-E3 ligand targeting chimera molecule for degrading PCSK9 (FIG. 22A), in which the cell-penetrating peptide module has a sequence of RQIKIWFQNRRMKWK (SEQ ID NO: 3), the targeting peptide module has a sequence of MESFPGWNLV(homoR)IGLLR (SEQ ID NO: 14) and is connected to two different E3 small molecule ligand modules via two Linker modules, respectively, two different E3 small molecule ligand modules being the E3 ligands targeting CRBN and TAP, respectively, and the Linker modules and the E3 small molecule ligand modules have an overall structural formula of

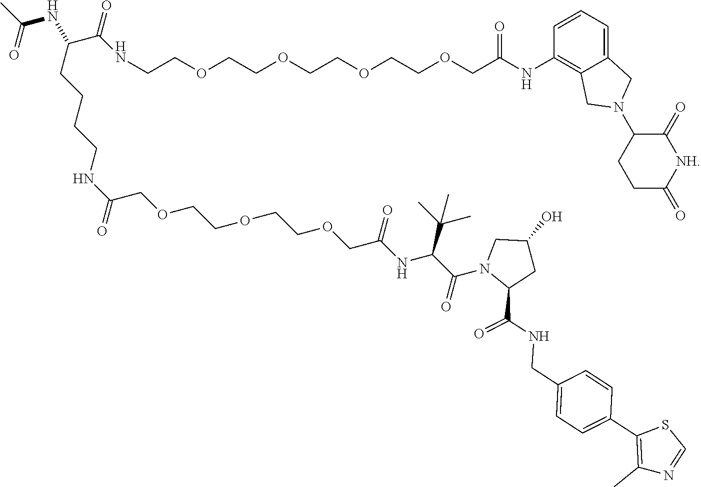

Likewise, in order to verify that the targeting chimera molecule provided in this application serves to degrade rather than inhibit, the inventor also designed and added the protease inhibitor MG132, which can inhibit the effect of the targeting chimera provided in this application. From the experimental results (FIG. 22B), it can be seen that the protein only added with the targeting chimera molecule still cannot be expressed, but the protein added with the targeting chimera molecule+MG132 can be expressed normally with an expression level comparable to that of the protein without adding the targeting chimera molecule. These results demonstrate that the role of the targeting chimera molecule is to degrade rather than inhibit the protein.

In addition, compared with FIG. 18A and FIG. 18B (the targeting chimera molecule for degrading PCSK9 protein also), the targeting chimera molecule with dual-E3 ligands has a lower dosage (from 25 nmol to 15 nmol) under the same degradation effect. Besides, since this targeting chimera molecule has dual-E3 ligands, after one of the linked E3 ubiquitinases mutates and develops drug resistance, the other E3 ubiquitinase can continue to function, increasing the reliability of the targeting chimera molecule.

### Example 11

In particular, in order to illustrate that the “modulating” design given in this application can also target different protein targets and degrade two or even multiple proteins simultaneously, the inventors designed a dual-target modulating targeting chimera molecule for degrading the novel coronavirus HR2 protein and the novel coronavirus N protein simultaneously (FIG. 23A), in which the cell-penetrating peptide module has a sequence of YGRKKRRQRRR (SEQ ID NO: 1), the targeting peptide modules have sequences of SAIGKIQDSLSSTAS (SEQ ID NO: 4) and PQEESEEEVEEP (SEQ ID NO: 5) respectively, the Linker module is a small molecule composed of (PEG)4 with a structural formula of

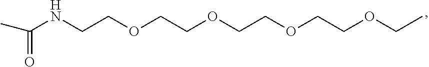 [SMILES: CCOCCOCCOCCOCCNC(C)=O]

and E3 small molecule ligand module is an E3 ligand targeting CRBN with a structural formula of

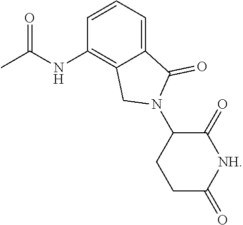 [SMILES: CC(=O)Nc1cccc2c1CN(C1CCC(=O)NC1=O)C2=O]

Likewise, in order to verify that the dual-target targeting chimera molecule provided in this application serves to degrade rather than inhibit, the inventor also designed and added the protease inhibitor MG132, which can inhibit the effect of the targeting chimera provided in this application. From the experimental results (FIG. 23B), it can be seen that the protein only added with the targeting chimera molecule still cannot be expressed, but the protein added with the targeting chimera molecule+MG132 can be expressed normally with an expression level comparable to that of the protein without adding the targeting chimera molecule. These results demonstrate that the role of the targeting chimera molecule is to degrade rather than inhibit the protein.

In addition, the use of this dual-target targeting chimera molecule can degrade HR2 protein and N protein simultaneously. Based on this consideration, the inventors can also design targeting chimera molecules targeting three targets, four targets or even more targets, which can degrade a variety of proteins simultaneously to achieve better applicability and wider use conditions. Considering the space limitation of the application, it will not be repeated here, but the technical protection of multi-target targeting chimera molecule should not be limited by the dual-target targeting chimera molecule described in this example.

### Example 12

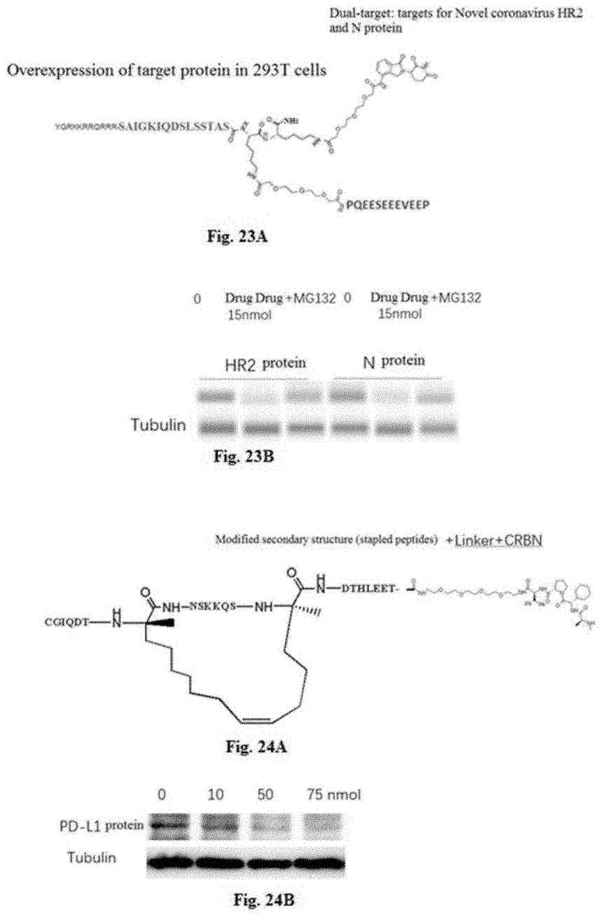

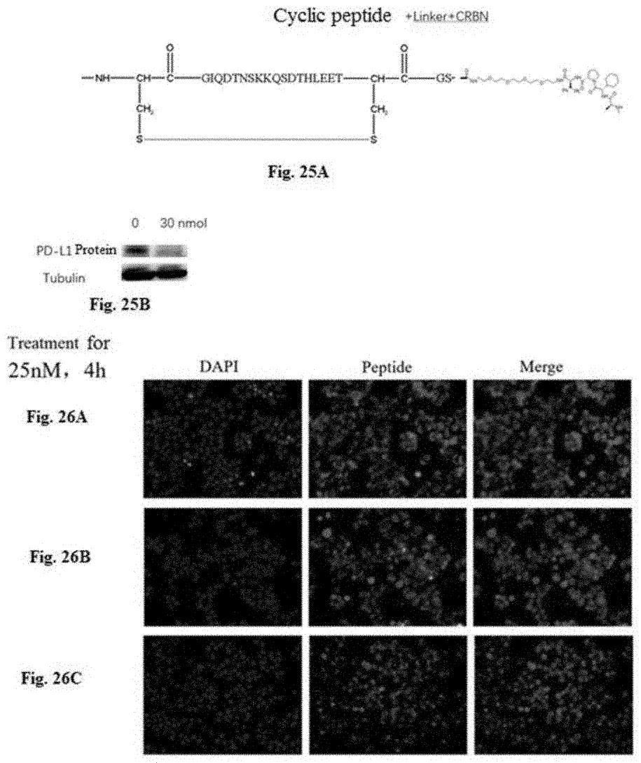

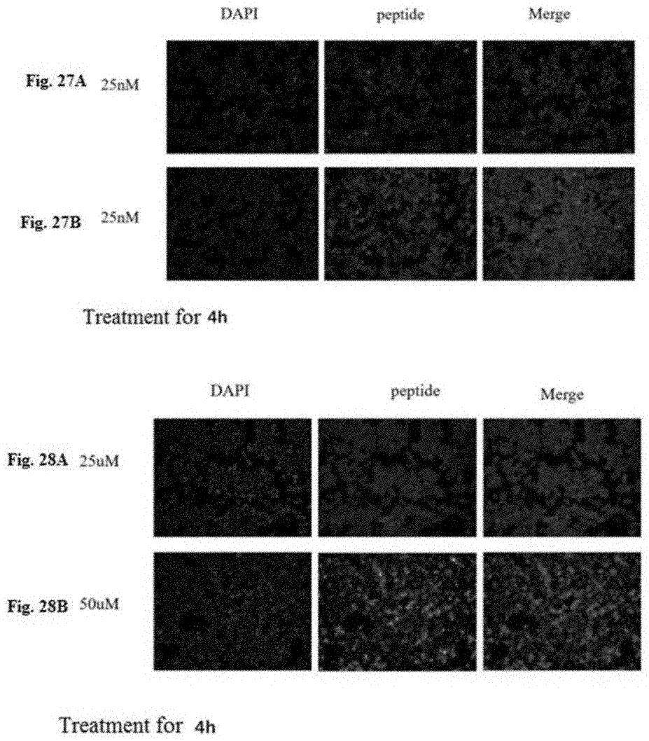

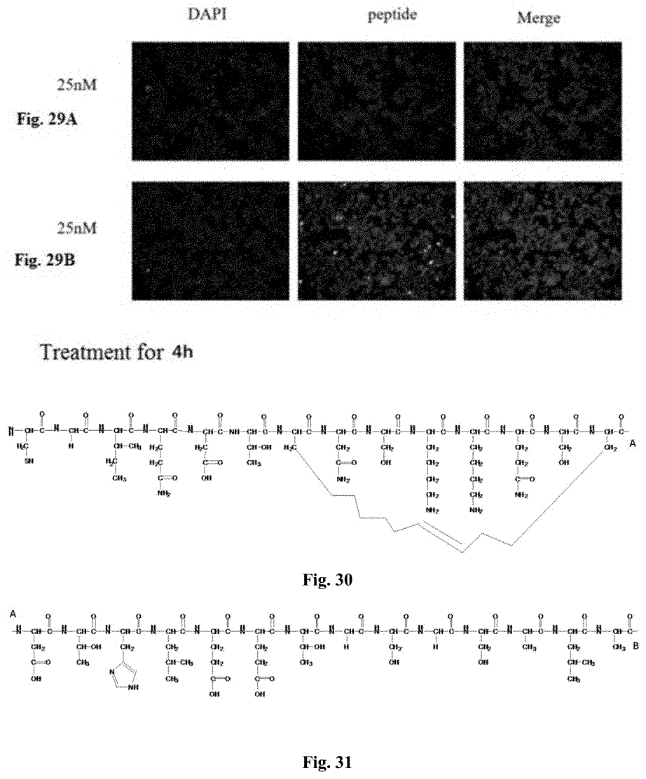

In particular, in order to illustrate that the “modulating” design given in this application can also modify the targeting peptide module to achieve the purpose of eliminating cell-penetrating peptide connection or increasing structural stability, the inventors designed a modulating targeting chimera molecule with stapled peptide modification and cyclic peptide modification (FIG. 24A and FIG. 25A).

Likewise, the degradation effect of the targeting chimera molecule with different dosages (nmol) on PD-L1 protein was verified by Western Blot (FIG. 24B and FIG. 25B). It can be seen that with the increase of dosage, the protein has been degraded (the expression level gradually decreased).

The principle of the stapled peptide modification and the cyclic peptide modification is to make the targeting peptide module assume a state similar to the secondary structure of the protein, forming a “mini-protein”, which can still avoid the decomposition of the peptide during cell-penetrating in the case of not connecting the cell-penetrating peptide module, effectively increasing the stability of the targeting peptide module.

The modification process of the stapled peptide (targeting peptide module) includes: modifying and linking CGIQDTNSKKQSDTHLEETC (SEQ ID NO: 831) to two compounds R8 and S5 (the structures are as follows), so that the peptide becomes:

CGIQDT(R8)NSKKQS(S5)DTHLEET-.

R8 is Fmoc-R8-OH, and the structural formula is as follows:

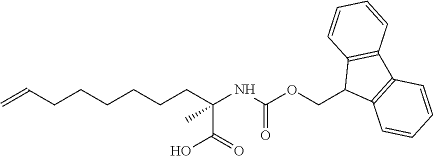 [SMILES: C=CCCCCCC[C@@](C)(NC(=O)OCC1c2ccccc2-c2ccccc21)C(=O)O]

S5 is Fmoc-S5-0H, and the structural formula is as follows:

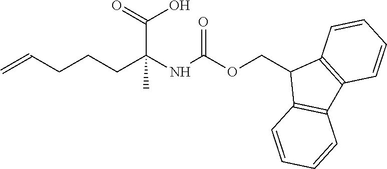 [SMILES: C=CCCC[C@](C)(NC(=O)OCC1c2ccccc2-c2ccccc21)C(=O)O]

Stapled peptide (or mini-protein)+E3 small molecule ligand chimeric structure:

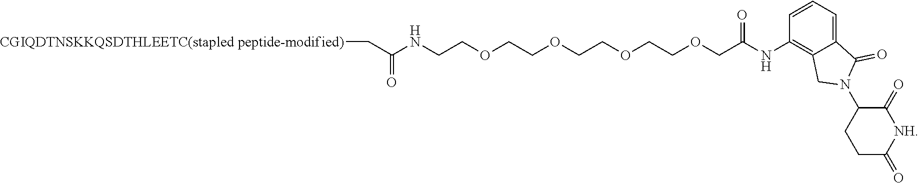

The targeting chimera molecule containing stapled peptide has a structural formula of:

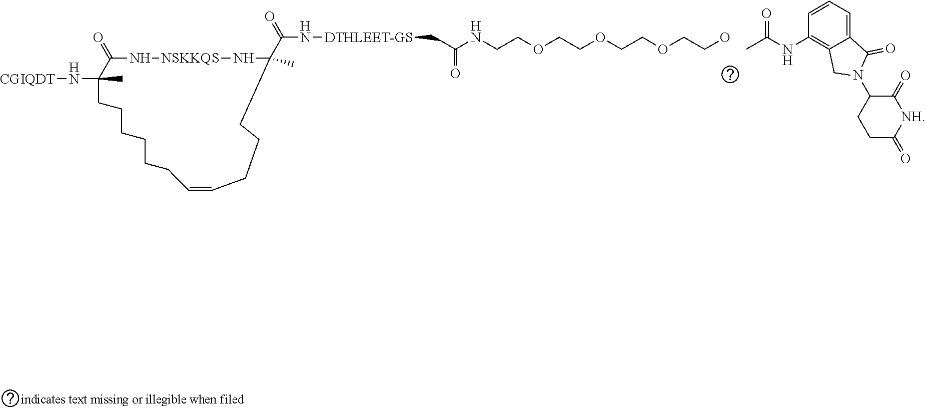

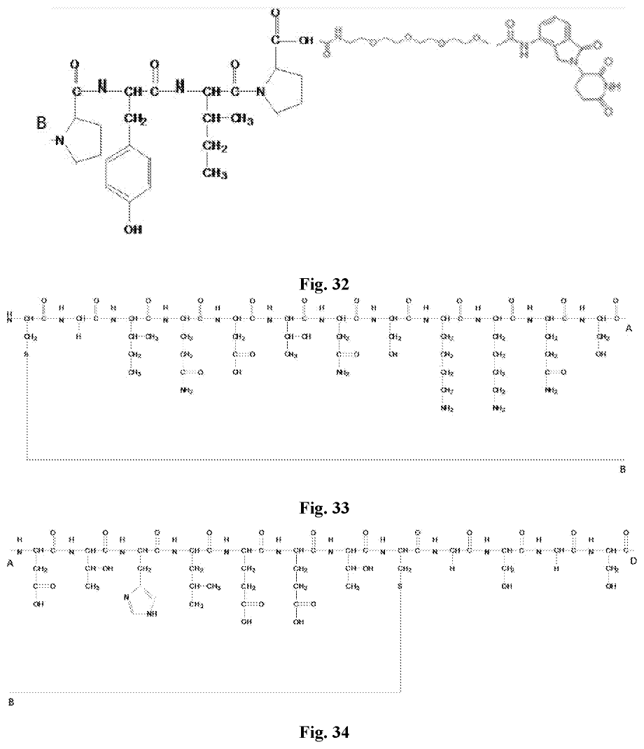

The overall structure is shown in FIGS. 30-32. FIGS. 30-32 show a structural representation of a stapled peptide+small molecule ligand chimera. Among them, terminal A in FIG. 30 is connected to terminal A in FIG. 31, and terminal B in FIG. 31 is connected to terminal B in FIG. 32. The combination of FIGS. 30-32 shows the structure of a chimera molecule compound containing a stapled peptide.

Cyclic Peptide+Small Molecule E3 Ligand Chimera Structure:

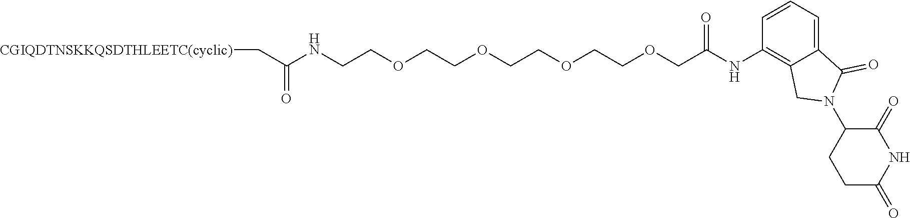

The structural formula of cyclic peptide (targeting peptide module): Linker-3PEG, Binder (ligand): CRBN (full name Cereblon):

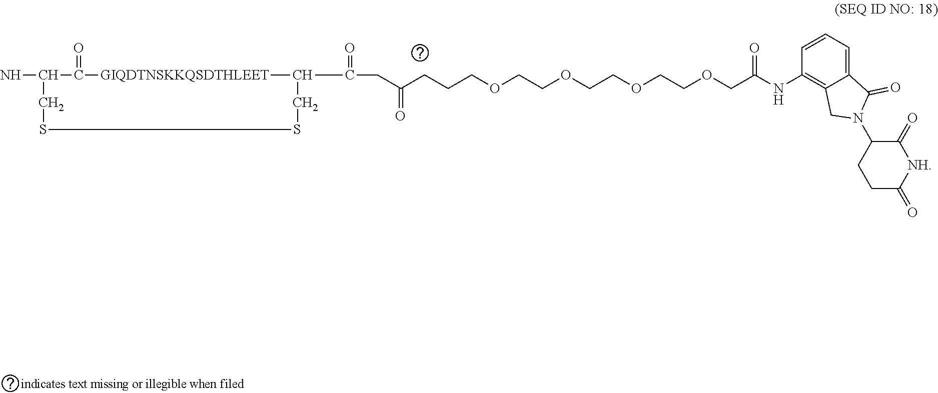

Linker module is a small molecule composed of (PEG)4 with a structural formula of

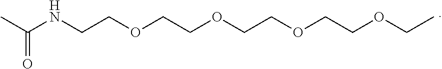 [SMILES: CCOCCOCCOCCOCCNC(C)=O]

E3 small molecule ligand module is an E3 ligand targeting CRBN with a structural formula of

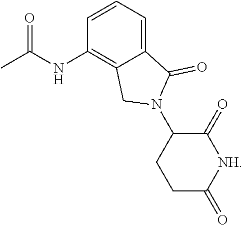 [SMILES: CC(=O)Nc1cccc2c1CN(C1CCC(=O)NC1=O)C2=O]

Cyclization method: The two cysteine disulfide bonds in the above figure form a ring.

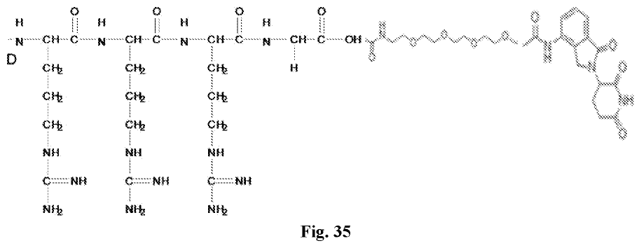

The overall structure is shown in FIGS. 33-35. FIGS. 33-35 show another structural representation of a cyclic peptide+small molecule ligand chimera. Among them, terminal Ain FIG. 33 is connected to terminal A in FIG. 34, terminal B in FIG. 33 is connected to terminal B in FIG. 34, and terminal D in FIG. 34 is connected to terminal D in FIG. 35. The combination of FIGS. 33-35 shows the structure of a chimera molecule compound containing a cyclic peptide.

### Example 13

The applicant has retrieved three prior art documents related to the technology in the present invention, and compared the technology with the technical solution of the present invention for the technical effect.

Document 1:

Specific Knockdown of a-Synuclein by Peptide-Directed Proteasome Degradation Rescued ItsAssociated Neurotoxicity (Jing Qu, Xiaoxi Ren, Fenqin Xue, Haixia Huang, Wei Wang, Jianliang Zhang, Cell Chemical Biology, 2020).

Document 2:

Specific Knockdown of Endogenous Tau Protein by Peptide-Directed Ubiquitin-ProteasomeDegradation (Ting-Ting Chu, Na Gao, Qian-Qian Li, . . . , Yong-Xiang Chen, Yu-Fen Zhao, Yan-Mei Li, Cell Chemical Biology, 2016).

Document 3:

A PROTAC peptide induces durable β-catenin degradation and suppresses Wnt-dependentintestinal cancer (Hongwei Liao 1, Xiang Li 2, Lianzheng Zhao 1, Yalong Wang 1, Xiaodan Wang 1, Ye Wu 2, Xin Zhou 3, Wei Fu 3, Lei Liu 4, Hong-Gang Hu 2,5 and Ye-Guang Chen 1, Cell Discovery, 2020).

Document 1 and Document 2 disclose the composition of cell-penetrating peptide+targeting peptide+peptide Linker+peptide Binder, in which Document 1 does not a significant degradation effect on target protein until 50 μm (FIG. 3), and Document 2 does not a significant effect on the target protein until 100 μm (FIG. 2).

Document 3 discloses the composition of stapled peptide+peptide Linker+polypeptide Binder, and Document 3 does not have a significant degradation effect on the target until 70 μm (FIG. 1).

The targeting chimera molecule provided by the technical solution of the present invention can degrade the target protein at the nm level, and the specific comparison is shown in Table 8.

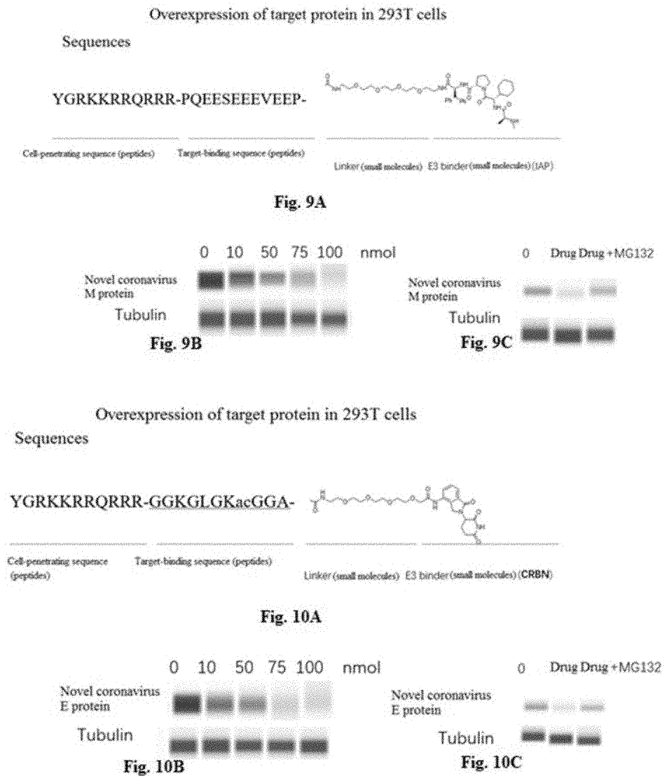

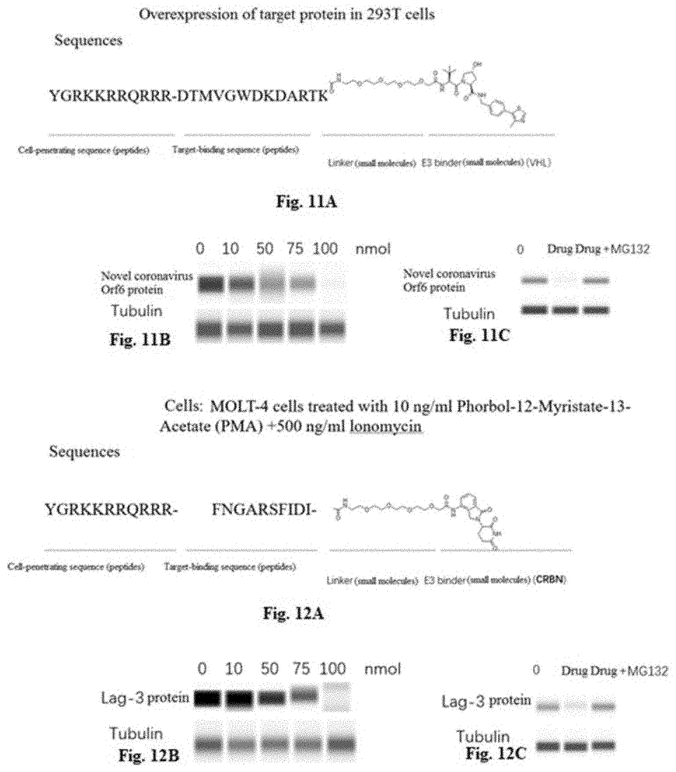

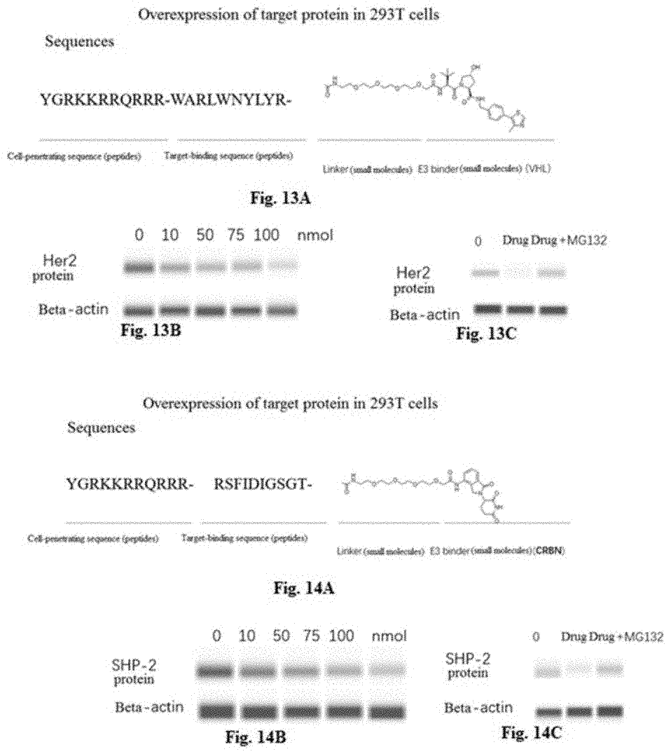

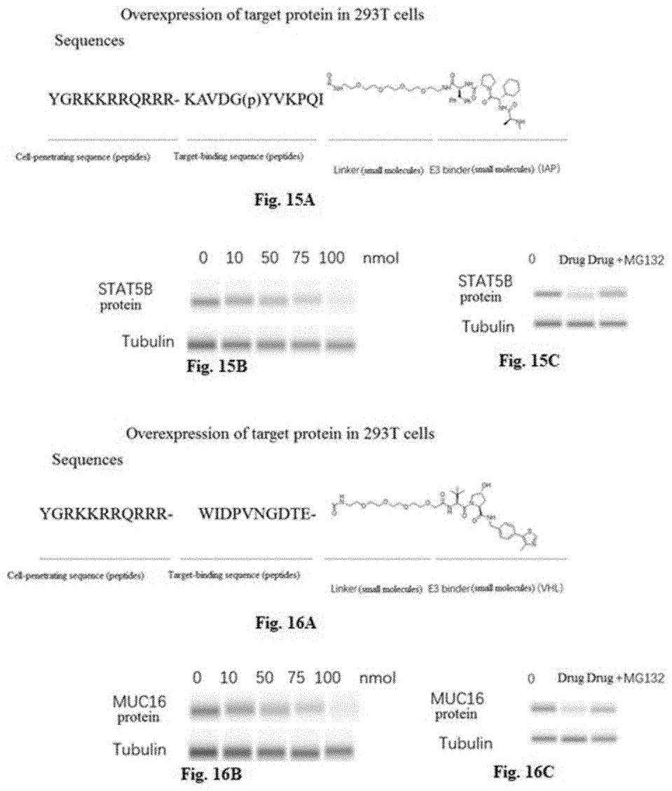

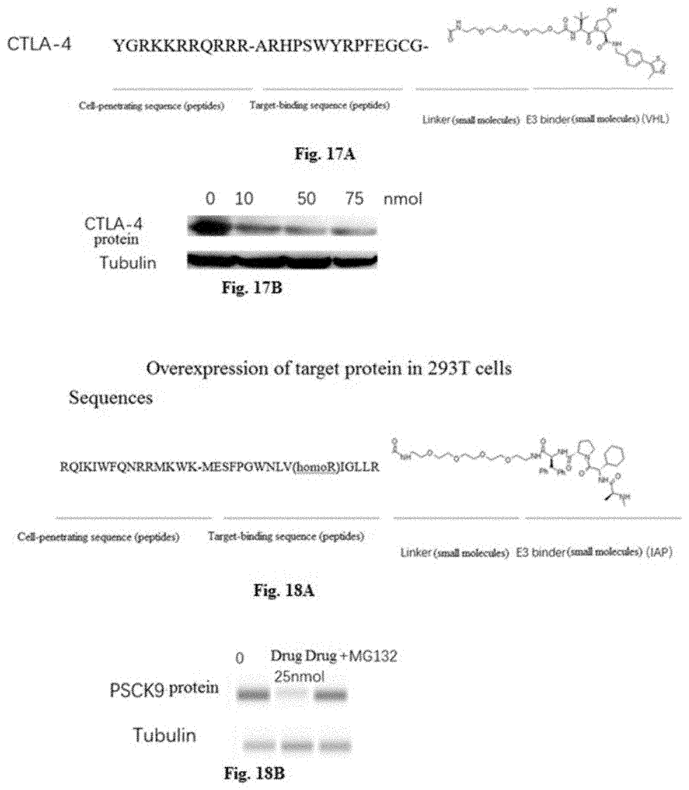

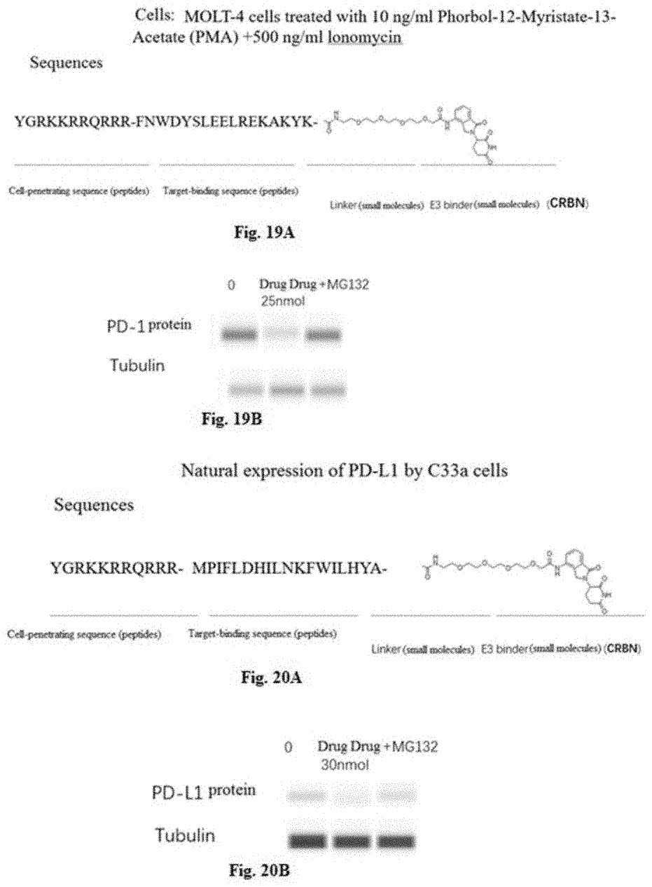

TABLE 8

| | Dosage of the drug when the | |
|---|---|---|
| | target protein is degraded | |
| Example 7 (novel coronavirus S protein HR2, FIGS. 7A-C) | 50-100 | nmol |
| Example 8 (novel coronavirus N protein, FIGS. 8A-C) | 50-100 | nmol |
| Example 8 (novel coronavirus M protein, FIGS. 9A-C) | 50-100 | nmol |
| Example 8 (novel coronavirus E protein, FIGS. 10A-C) | 10-100 | nmol |
| Example 8 (novel coronavirus Orf6 protein, FIGS. 11A-C) | 50-100 | nmol |
| Example 8 (Lag-3 protein, FIGS. 12A-C) | 75-100 | nmol |
| Example 8 (Her2 protein, FIGS. 13A-C) | 10-100 | nmol |
| Example 8 (SHP-2 protein, FIGS. 14A-C) | 10-100 | nmol |
| Example 8 (STAT5B protein, FIGS. 15A-C) | 75-100 | nmol |
| Example 8 (MUC16 protein, FIGS. 16A-C) | 50-100 | nmol |
| Example 8 (CTLA-4 protein, FIGS. 17A-B) | 10-75 | nmol |
| Example 8 (PCSK9 protein, FIGS. 18A-B) | 25 | nmol |
| Example 8 (PD-1 protein, FIGS. 19A-B) | 25 | nmol |
| Example 8 (PD-L1 protein, FIGS. 20A-B) | 30 | nmol |
| Example 9 (KRAS protein G12V mutation, FIGS. 21A-C) | 30 | nmol |
| Example 10 (dual-E3, PCSK9 protein, FIGS. 22A-C) | 15 | nmol |
| Example 11 (dual-target, novel coronavirus HR2 protein + | 15 | nmol |
| novel coronavirus N protein, FIGS. 23A-C) | | |
| Example 12 (stapled peptide-modified, PD-L1 protein, | 50-75 | nmol |
| FIGS. 24A-C) | | |
| Example 12 (cyclic peptide-modified, PD-L1 protein, | 30 | nmol |
| FIGS. 25A-C) | | |
| Document 1 | 50 | μmol |
| Document 2 | 100 | μmol |
| Document 3 | 70 | μmol |

It can be seen from Table 8 that the targeting chimera molecule provided in this application can degrade the target protein in the order of nmol (the highest is 100 nmol, i.e., 0.1 μmol), while in Documents 1-3, the minimum level of 50 μmol is required to produce significant degradation effect on the target protein. The difference between the two is at least 500 times in the dosage of the degrader, with a very obvious difference in effect.

Finally, it should be noted that the above embodiments are only used to illustrate the technical solutions of the present invention, but not to limit them. Although the present invention has been described in detail in conjunction with the foregoing embodiments, those of ordinary skill in the art should understand that the technical solutions described in the foregoing embodiments can still be modified, or some or all of the technical features thereof can be equivalently replaced; and these modifications or replacements will not make the spirit of the corresponding technical solutions depart from the scope of the technical solutions of the embodiments of the present invention.

1. A modulating targeting chimera molecule induced by a cell-penetrating peptide, comprising at least one cell-penetrating peptide module, at least one targeting peptide module and at least one small molecule ligand module connected with each other, wherein the targeting peptide module is a peptide sequence that can bind to a targeted protein.

2. The modulating targeting chimera molecule induced by the cell-penetrating peptide according to claim 1, further comprising at least one Linker module, wherein the targeting peptide module is chimeric with the small molecule ligand module through the Linker module.

3. The modulating targeting chimera molecule induced by the cell-penetrating peptide according to claim 2, wherein the cell-penetrating peptide module is connected to the free end of the targeting peptide module and used to guide the targeting chimera molecule for penetrating the cell membrane.

4. The modulating targeting chimera molecule induced by the cell-penetrating peptide according to claim 3, wherein the small molecule ligand module is a small molecule E3 ligand that can bind to E3 ligase, preferably, the protease degrader adapted to the small molecule E3 ligand is one or more of CRBN (Cereblon protein), VHL (von Hippel-Lindau) and IAP (Inhibitor of apoptosis proteins).

5. The modulating targeting chimera molecule induced by the cell-penetrating peptide according to claim 1, wherein the cell-penetrating peptide module has an amino acid sequence of any one of SEQ ID No.1-SEQ ID No.3.

6. The modulating targeting chimera molecule induced by the cell-penetrating peptide according to claim 1, wherein the targeting peptide module has an amino acid sequence of any one or more of SEQ ID No.4-SEQ ID No.17.

7. The modulating targeting chimera molecule induced by the cell-penetrating peptide according to claim 1, wherein the Linker module is a small molecule compound with a structural formula shown in formula I:

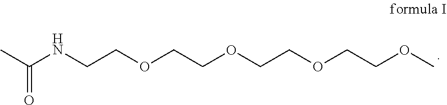 [SMILES: COCCOCCOCCOCCNC(C)=O]

8. The modulating targeting chimera molecule induced by the cell-penetrating peptide according to claim 1, wherein:
when the adapted protease degrader is CRBN, the structural formula of the small molecule ligand module is shown in formula II: 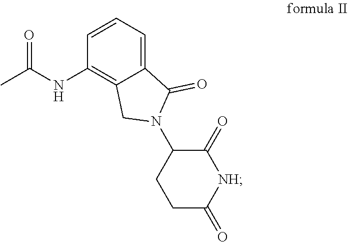 [SMILES: CC(=O)Nc1cccc2c1CN(C1CCC(=O)NC1=O)C2=O]
when the adapted protease degrader is VHL, the structural formula of the small molecule ligand module is shown in formula III: 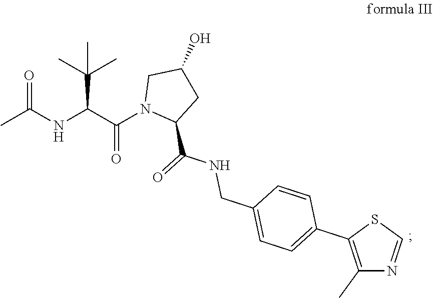 [SMILES: CC(=O)N[C@H](C(=O)N1C[C@H](O)C[C@H]1C(=O)NCc1ccc(-c2scnc2C)cc1)C(C)(C)C]
and
when the adapted protease degrader is TAP, the structural formula of the small molecule ligand module is as shown in formula IV:

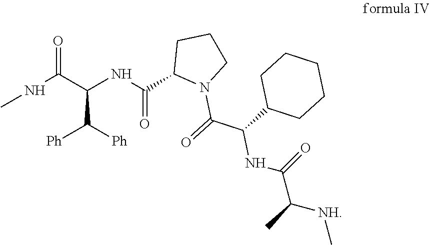

9. The modulating targeting chimera molecule induced by the cell-penetrating peptide according to claim 5, having a structure of any one or more of the following structures:
1) the cell-penetrating peptide of SEQ ID No.1+the targeting peptide of SEQ ID No.4+the Linker of formula I+the small molecule ligand of formula II;
2) the cell-penetrating peptide of SEQ ID No.2+the targeting peptide of SEQ ID No.5+the Linker of formula I+the small molecule ligand of formula III;
3) the cell-penetrating peptide of SEQ ID No.1+the targeting peptide of SEQ ID No.5+the Linker of formula I+the small molecule ligand of formula IV;
4) the cell-penetrating peptide of SEQ ID No.1+the targeting peptide of SEQ ID No.6+the Linker of formula I+the small molecule ligand of formula II;
5) the cell-penetrating peptide of SEQ ID No.1+the targeting peptide of SEQ ID No.7+the Linker of formula I+the small molecule ligand of formula III;
6) the cell-penetrating peptide of SEQ ID No.1+the targeting peptide of SEQ ID No.8+the Linker of formula I+the small molecule ligand of formula II;
7) the cell-penetrating peptide of SEQ ID No.1+the targeting peptide of SEQ ID No.9+the Linker of formula I+the small molecule ligand of formula III;
8) the cell-penetrating peptide of SEQ ID No.1+the targeting peptide of SEQ ID No.10+the Linker of formula I+the small molecule ligand of formula II;
9) the cell-penetrating peptide of SEQ ID No.1+the targeting peptide of SEQ ID No.11+the Linker of formula I+the small molecule ligand of formula IV;
10) the cell-penetrating peptide of SEQ ID No.1+the targeting peptide of SEQ ID No.12+the Linker of formula I+the small molecule ligand of formula III;
11) the cell-penetrating peptide of SEQ ID No.1+the targeting peptide of SEQ ID No.13+the Linker of formula I+the small molecule ligand of formula III;
12) the cell-penetrating peptide of SEQ ID No.3+the targeting peptide of SEQ ID No.14+the Linker of formula I+the small molecule ligand of formula IV;
13) the cell-penetrating peptide of SEQ ID No.1+the targeting peptide of SEQ ID No.15+the Linker of formula I+the small molecule ligand of formula II;
14) the cell-penetrating peptide of SEQ ID No.1+the targeting peptide of SEQ ID No.16+the Linker of formula I+the small molecule ligand of formula II;
15) the cell-penetrating peptide of SEQ ID No.2+the targeting peptide of SEQ ID No.17+the Linker of formula I+the small molecule ligand of formula IV;
16) the cell-penetrating peptide of SEQ ID No.3+the targeting peptide of SEQ ID No.14+the Linker of formula I+(dual E3 ligands: the small molecule ligand of formula II+the small molecule ligand of formula III); and
17) the cell-penetrating peptide of SEQ ID No.1+(dual targets: the targeting peptide of SEQ ID No.4+the targeting peptide of SEQ ID No.5)+the Linker of formula I+the small molecule ligand of formula II.

10. The modulating targeting chimera molecule induced by the cell-penetrating peptide according to claim 5, wherein the targeting peptide module further comprises a modified stapled peptide sequence or circular peptide sequence, and the stapled peptide sequence or circular peptide sequence has a function of cell penetration.

11. The modulating targeting chimera molecule induced by the cell-penetrating peptide according to claim 10, wherein the stapled peptide has a structural formula shown in formula V: 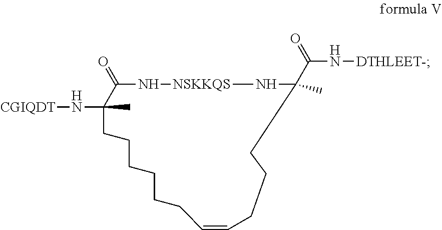
and
the cyclic peptide has a structural formula shown in formula VI:

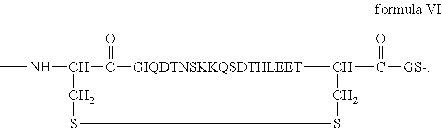

12. The modulating targeting chimera molecule induced by the cell-penetrating peptide according to claim 11, wherein the modulating targeting chimera molecule induced by the cell-penetrating peptide containing the stapled peptide has the structure as follows: the stapled peptide of formula V+the Linker of formula I+the small molecule ligand of formula II.

13. The modulating targeting chimera molecule induced by the cell-penetrating peptide according to claim 11, wherein the modulating targeting chimera molecule induced by the cell-penetrating peptide containing the circular peptide has the structure as follows: the circular peptide of formula VI+the Linker of formula I+the small molecule ligand of formula II.

14. A method of preparing a product for degrading a targeted protein or a product for degrading a targeted protein with a mutant amino acid position with the modulating targeting chimera molecule induced by the cell-penetrating peptide of claim 1.

15. The method according to claim 14, wherein the degraded targeted protein comprises one or more of the novel coronavirus S protein HR2, novel coronavirus N protein, novel coronavirus M protein, novel coronavirus E protein, novel coronavirus Orf6 protein, LAG-3 protein, Her2 protein, SHP-2 protein, STAT5B protein, MUC16 protein, CTLA-4 protein, PCSK9 protein, PD-1 protein, PD-L1 protein and KRAS protein with G12V mutation.
